(19) United States
(12) Patent Application Publication
(10) Pub. No.: US 2026/0339203 A1
(43) Pub. Date: Oct. 29, 2026

(54) ONIUM SALT, PHOTO-ACID GENERATOR, CHEMICALLY-AMPLIFIED RESIST COMPOSITION, AND PATTERNING PROCESS

(71) Applicant: SHIN-ETSU CHEMICAL CO., LTD., Tokyo (JP)

(72) Inventors: Ryo SUDO, Joetsu-shi (JP); Sotaro TAKAHASHI, Joetsu-shi (JP); Gentaro HIDA, Joetsu-shi (JP)

(73) Assignee: SHIN-ETSU CHEMICAL CO., LTD., Tokyo (JP)

(21) Appl. No.: 19/643,365

(22) Filed: Apr. 9, 2026

(30) Foreign Application Priority Data
Apr. 15, 2025 (JP) 2025-067146

(51) Int. Cl. G03F 7/004; C07C 25/18; C07C 309/32; C07C 381/12; C08F 220/68; C08F 222/10; G03F 7/00; G03F 7/039; G03F 7/20

(52) U.S. Cl. CPC G03F 7/0045; C07C 25/18; C07C 309/32; C07C 381/12; C08F 220/68; C08F 222/10; G03F 7/0397; G03F 7/2004; G03F 7/70033

(57) ABSTRACT

An onium salt represented by following formula (A), where “m1” represents 0 or 1; “m2” represents 2, 3, 4, or 5; “m3” represents 0, 1, 2, 3, or 4; R represents a halogen atom other than an iodine atom, a nitro group, a hydroxy group, a cyano group, a hydrocarbyl group, a hydrocarbyloxy group, or a hydrocarbylthio group, provided that not both of two carbon atoms adjacent to carbon atom having a substituent which is sulfonic acid “SO₃⁻” are bonded to iodine atoms; L represents a single bond, an ester group, an ether group, an amide group, a sulfonic acid ester group, or an alkylene group; W represents a hydrocarbyl group containing at least one aromatic ring; and Z⁺ represents a sulfonium cation or an iodonium cation. This can provide: a resist material having high sensitivity and improved LWR and improved CDU; and a patterning process using the material.

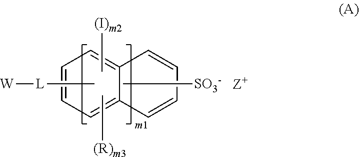

### TECHNICAL FIELD

The present invention relates to: an onium salt; a photo-acid generator; a chemically-amplified resist composition; and a patterning process.

### BACKGROUND ART

In recent years, as LSIs advance toward higher integration and higher processing speed, fine patterning is required, and in pattern processing of 0.2 μm or less, an acid-catalyzed chemically-amplified resist composition is used. In addition, as an exposure source in this event, a high-energy beam, such as ultraviolet ray, deep ultraviolet ray, and electron beam, is used. As an advanced miniaturization technique, mass production by ArF immersion lithography, where exposure is performed with a liquid such as water interposed between a projection lens and a substrate, is carried out, and active research efforts have been made on multiple exposure by ArF lithography, extreme ultraviolet ray (EUV) lithography of wavelength 13.5 nm, etc.

As the patterns are miniaturized, the line width roughness (LWR) of line patterns and the critical dimension uniformity (CDU) of hole patterns and dot patterns are regarded significant. In addition, it is pointed out that these factors are affected by the segregation or agglomeration of a base polymer and acid generator and the diffusion of acid. Furthermore, there is a tendency that as the resist film becomes thinner, LWR and CDU become greater, and film thickness reduction to comply with the progress of miniaturization causes degradation of LWR and CDU, which is a serious problem.

Resist compositions for EUV lithography need to meet high sensitivity, high resolution, and low LWR at the same time. As the acid diffusion distance is reduced, LWR is reduced, but sensitivity becomes lower. For example, when the post-exposure baking (PEB) temperature is lowered, the outcome is a reduced LWR, but a lower sensitivity. When the amount of quencher added is increased, the outcome is a reduced LWR, but a lower sensitivity. It is necessary to overcome the tradeoff relation between sensitivity and LWR.

Iodine atoms very greatly absorb EUV having a wavelength of 13.5 nm, and therefore, an effect that secondary electrons are generated from iodine atoms during exposure is observed, and iodine atoms are attracting attention in EUV lithography. Patent Document 1 discloses a photo-acid generator having an iodine atom introduced into the anion, and Patent Document 2 discloses a polymerizable-group-containing photo-acid generator having an iodine atom introduced into the anion.

Meanwhile, the effect of perfluoroalkyl compounds (PFAS) on health has been pointed out, and there is a movement in the European REACH to restrict the production and sale of PFAS compounds. Many compounds containing PFAS are currently used in relation with semiconductor lithography. For example, materials containing such compounds are used in surfactants, acid generators, etc. The above-described photo-acid generators disclosed in Patent Documents 1 and 2 are all based on alkanesulfonic acid units, and it is pointed out that these photo-acid generators greatly affect health and the environment.

Patent Document 3 discloses an onium salt containing fluorobenzenesulfonic acid as an alternative to PFAS. In addition, Patent Document 4 discloses an onium salt containing a benzenesulfonic acid. On the other hand, in such benzenesulfonic acid units, the acidity of the sulfonic acid is low, and improvement is required.

### CITATION LIST

### Patent Literature

Patent Document 1: JP4425776B2
Patent Document 2: JP6720926B2
Patent Document 3: JP2018-159744A
Patent Document 4: JP7531671B2
Patent Document 5: JP7032549B2

### SUMMARY OF INVENTION

### Technical Problem

In the field of acid-catalyzed chemically-amplified resist compositions, there are demands for the development of a resist composition that has higher sensitivity, also allows the improvement of the LWR of line patterns and the CDU of hole patterns, and is also excellent in resolution.

The present invention has been made in view of the above-described circumstances. An object of the present invention is to provide: a resist material having high sensitivity and improved LWR and improved CDU; and a patterning process using the material.

### Solution to Problem

To achieve the object, the present invention provides
an onium salt represented by the following formula (A),

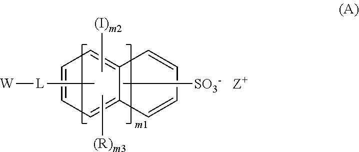

wherein “m1” represents an integer of 0 or 1, “m2” represents an integer of 2 or 3 when “m1” is 0 and represents an integer of 2, 3, 4, or 5 when “m1” is 1, and “m3” represents an integer of 0, 1, or 2 when “m1” is 0 and represents an integer of 0, 1, 2, 3, or 4 when “m1” is 1, provided that when “m1” is 0, 2≤m2+m3≤4 and when “m1” is 1, 2≤m2+m3≤6;
R represents a halogen atom other than an iodine atom, a nitro group, a hydroxy group, a cyano group, a hydrocarbyl group having 1 to 20 carbon atoms and optionally containing a heteroatom, a hydrocarbylcarbonyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, when “m3” is 2 or more, the Rs being identical to or different from each other and the Rs optionally being bonded to each other to form a ring together with the carbon atoms bonded thereto, provided that not both of the two carbon atoms adjacent to the carbon atom having a substituent which is the sulfonic acid “SO₃⁻” are bonded to iodine atoms;
L represents a single bond, an ester group, an ether group, an amide group, a sulfonic acid ester group, or a linear, branched, or cyclic alkylene group having 1 to 12 carbon atoms, the alkylene group optionally containing one or more kinds selected from an ester group, an ether group, an amide group, and a sulfonic acid ester group;
W represents a hydrocarbyl group having 6 to 60 carbon atoms and containing at least one aromatic ring and optionally contains a heteroatom, the aromatic ring optionally having a substituent which is a halogen atom, a nitro group, a hydroxy group, a cyano group, a pentafluorosulfanyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom; and
Z⁺ represents a sulfonium cation represented by the following formula (Z-1) or an iodonium cation represented by the following formula (Z-2),

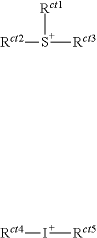

wherein R^{ct1} to R^{ct5} each independently represent a halogen atom or a hydrocarbyl group having 1 to 30 carbon atoms and optionally containing a heteroatom, the R^{ct1} and the R^{ct2} optionally being bonded to each other to form a ring together with the sulfur atom bonded thereto.

By using the inventive onium salt, it is possible to provide a resist material having high sensitivity and improved LWR and improved CDU and provide a patterning process using the material in the case of either a positive type or a negative type.

The inventive onium salt is preferably one represented by the following formula (A1),

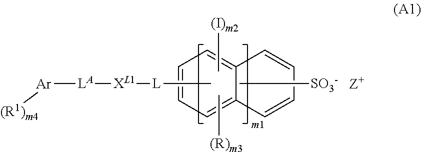

wherein “m4” represents 0 to 9; X^{L1} represents a single bond or a hydrocarbylene group having 1 to 40 carbon atoms and optionally containing a heteroatom; L^{A} represents a single bond or a linear, branched, or cyclic alkyl group having 1 to 12 carbon atoms and optionally containing one or more kinds selected from an ester group, an ether group, an amide group, and a sulfonic acid ester group; Ar represents an aromatic hydrocarbon group; R¹ represents a halogen atom, a nitro group, a hydroxy group, a cyano group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, when “m4” is 2 or more, the R¹s being identical to or different from each other and the R¹s optionally being bonded to each other to form a ring together with the carbon atoms bonded thereto; and “m1” to “m3”, L, R, and Z⁺ are as defined above.

In this case, the inventive onium salt is further preferably one represented by the following formula (A2),

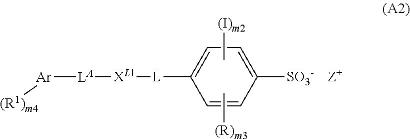

wherein “m2” to “m4”, L, L^{A}, X^{L1}, R, R¹, Ar, and Z⁺ are as defined above.

In this case, the inventive onium salt is extremely preferably one represented by the following formula (A3),

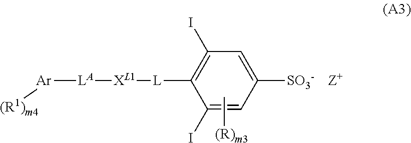

wherein “m3”, “m4”, L, L^{A}, X^{L1}, R, R¹, Ar, and Z⁺ are as defined above.

When the structure is as described above, the advantageous effects of the present invention can be exhibited more fully.

The present invention also provides a photo-acid generator comprising the above-described onium salt.

The inventive onium salt can be used suitably as a photo-acid generator.

The present invention also provides a chemically-amplified resist composition comprising the above-described photo-acid generator.

The inventive chemically-amplified resist composition is a resist material having high sensitivity and improved LWR and improved CDU in the case of either a positive type or a negative type.

The inventive chemically-amplified resist composition preferably further comprises a base polymer.

Such a material can be used more suitably as a chemically-amplified resist composition.

In this case, the base polymer preferably includes a repeating unit represented by the following formula (a1) or (a2),

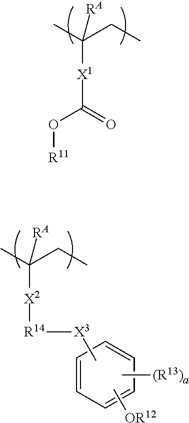

wherein each R^{A} independently represents a hydrogen atom or a methyl group;
X¹ represents a single bond, a phenylene group, a naphthylene group, or a linking group having 1 to 12 carbon atoms and containing at least one kind selected from an ester bond, an ether bond, and a lactone ring, the phenylene group, the naphthylene group, and the linking group optionally containing at least one selected from a hydroxy group, a halogen atom, a saturated hydrocarbyloxy group having 1 to 8 carbon atoms, and a saturated hydrocarbylcarbonyloxy group having 2 to 8 carbon atoms;
X² represents a single bond or an ester bond;
X³ represents a single bond, an ether bond, or an ester bond;
R¹¹ and R¹² each independently represent an acid-labile group;
R¹³ represents a saturated hydrocarbyl group having 1 to 4 carbon atoms, a halogen atom, a saturated hydrocarbylcarbonyl group having 2 to 5 carbon atoms, a cyano group, or a saturated hydrocarbyloxycarbonyl group having 2 to 5 carbon atoms;
R¹⁴ represents a single bond or an alkanediyl group having 1 to 6 carbon atoms, the alkanediyl group optionally containing at least one selected from a hydroxy group, a saturated hydrocarbyloxy group having 1 to 8 carbon atoms, a saturated hydrocarbylcarbonyloxy group having 2 to 8 carbon atoms, an ether bond, and an ester bond; and
“a” represents an integer of 0 to 4.

When the base polymer is as described, the advantageous effects of the present invention can be exhibited more fully.

The inventive chemically-amplified resist composition preferably further comprises at least one kind selected from an organic solvent, a quencher, a surfactant, and a dissolution inhibitor.

The inventive chemically-amplified resist composition may contain these components as necessary.

The present invention also provides a patterning process comprising the steps of:
forming a resist film on a substrate by using the above-described chemically-amplified resist composition;
exposing the resist film by using a high-energy beam; and
developing the exposed resist film by using a developer.

According to the inventive patterning process, it is possible to form a pattern having high sensitivity and improved LWR and improved CDU in the case of either a positive type or a negative type.

It is preferable that the high-energy beam is a KrF excimer laser beam, an ArF excimer laser beam, an electron beam, or an extreme ultraviolet ray having a wavelength of 3 to 15 nm.

When such high-energy beams are used, the inventive patterning process can exhibit the advantageous effects of the present invention more fully.

### Advantageous Effects of Invention

In a photo-acid generator that contains a benzenesulfonic acid having multiple substituents which are iodine atoms, secondary electrons are generated from the iodine atoms during exposure in EUV lithography of wavelength 13.5 nm, since iodine atoms very greatly absorb EUV. By virtue of the resonance effect of the aromatic group, a stronger acid can be achieved when substituents which are iodine atoms are contained compared to an arylsulfonic acid only containing substituents which are fluorine atoms, and also by virtue of the high EUV absorption property of iodine atoms, acidity is enhanced. Furthermore, the mobility of the sulfonic acid is reduced by iodine atoms, having a great atomic weight, being present nearby, and acid diffusion can be suppressed and it is possible to improve LWR and CDU. There is a characteristic that out of the two carbon atoms of the ortho positions of the sulfonate anion, one is not bonded to an iodine atom, and therefore, the sulfonic acid is not affected by the sterically bulky electron clouds of iodine atoms, and favorable sensitivity and favorable resolution can be exhibited.

### DESCRIPTION OF EMBODIMENTS

As described above, there have been demands for the development of: a resist material having high sensitivity and improved LWR and improved CDU; and a patterning process using the material.

Meanwhile, Patent Document 5 shows a structure in which the ortho positions of a benzenesulfonic acid are each substituted with an aryl group. It is possible to use such a structure as an alternative to PFAS, but it can be assumed that sensitivity is not sufficient due to the presence of sterically bulky groups near the sulfonate anion.

To achieve the object, the present inventors have studied earnestly and found out that, by using, as a photo-acid generator, an onium salt which contains a benzenesulfonic acid having multiple substituents which are iodine atoms, at least one of the two carbon atoms in the ortho positions of the sulfonate anion not being bonded to an iodine atom, it is possible to obtain a chemically-amplified resist composition having high sensitivity, high contrast, high resolution, and improved lithography performance such as LWR and CDU. Thus, the present invention has been completed.

That is, the present invention is an onium salt represented by the following formula (A),

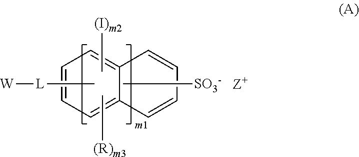

wherein “m1” represents an integer of 0 or 1, “m2” represents an integer of 2 or 3 when “m1” is 0 and represents an integer of 2, 3, 4, or 5 when “m1” is 1, and “m3” represents an integer of 0, 1, or 2 when “m1” is 0 and represents an integer of 0, 1, 2, 3, or 4 when “m1” is 1, provided that when “m1” is 0, 2≤m2+m3≤4 and when “m1” is 1, 2≤m2+m3≤6;
R represents a halogen atom other than an iodine atom, a nitro group, a hydroxy group, a cyano group, a hydrocarbyl group having 1 to 20 carbon atoms and optionally containing a heteroatom, a hydrocarbylcarbonyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, when “m3” is 2 or more, the Rs being identical to or different from each other and the Rs optionally being bonded to each other to form a ring together with the carbon atoms bonded thereto, provided that not both of the two carbon atoms adjacent to the carbon atom having a substituent which is the sulfonic acid “SO₃⁻” are bonded to iodine atoms;
L represents a single bond, an ester group, an ether group, an amide group, a sulfonic acid ester group, or a linear, branched, or cyclic alkylene group having 1 to 12 carbon atoms, the alkylene group optionally containing one or more kinds selected from an ester group, an ether group, an amide group, and a sulfonic acid ester group;
W represents a hydrocarbyl group having 6 to 60 carbon atoms and containing at least one aromatic ring and optionally contains a heteroatom, the aromatic ring optionally having a substituent which is a halogen atom, a nitro group, a hydroxy group, a cyano group, a pentafluorosulfanyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom; and
Z⁺ represents a sulfonium cation represented by the following formula (Z-1) or an iodonium cation represented by the following formula (Z-2),

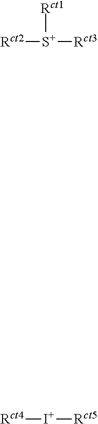

wherein R^{ct1} to R^{ct5} each independently represent a halogen atom or a hydrocarbyl group having 1 to 30 carbon atoms and optionally containing a heteroatom, the R^{ct1} and the R^{ct2} optionally being bonded to each other to form a ring together with the sulfur atom bonded thereto.

Hereinafter, the present invention will be described in detail, but the present invention is not limited thereto.

### [Onium Salt]

The inventive onium salt is represented by the following formula (A).

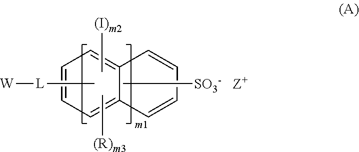

In the formula (A), “m1” represents an integer of 0 or 1. When “m1” is 0, a benzene ring is indicated, and when “m1” is 1, a naphthalene ring is indicated, and from the viewpoint of solvent solubility, a benzene ring, where “m1” is 0, is preferable. “m2” represents an integer of 2 or 3 when “m1” is 0 and represents an integer of 2, 3, 4, or 5 when “m1” is 1. From the viewpoint of the procurement of raw materials, “m2” is preferably 2. “m3” represents an integer of 0, 1, or 2 when “m1” is 0 and represents an integer of 0, 1, 2, 3, or 4 when “m1” is 1, provided that when “m1” is 0, 2≤m2+m3≤4 and when “m1” is 1, 2≤m2+m3≤6. By multiple hydrogen atoms on the benzenesulfonic acid being substituted with iodine atoms, absorption of EUV light is enhanced, furthermore, the degree of freedom of the sulfonic acid is reduced by the influence of the volume of the iodine atoms, and thus, a material having high sensitivity and low acid diffusion can be achieved. Meanwhile, at least one ortho position of SO₃⁻ is not substituted with an iodine atom, and it is further preferable that neither of the ortho positions is substituted with an iodine atom. Such an onium salt can reduce the steric hindrance around the sulfonate salt and can generate an acid effectively, and therefore, it is possible to provide a resist composition excellent in sensitivity and resolution.

In the formula (A), R represents a halogen atom other than an iodine atom, a nitro group, a hydroxy group, a cyano group, a hydrocarbyl group having 1 to 20 carbon atoms and optionally containing a heteroatom, a hydrocarbylcarbonyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, when “m3” is 2 or more, the Rs being identical to or different from each other and the Rs optionally being bonded to each other to form a ring together with the carbon atoms bonded thereto. Here, as the R, preferable are a halogen atom other than an iodine atom, a nitro group, a hydroxy group, a cyano group, and a hydrocarbyl group having 1 to 3 carbon atoms and optionally containing a heteroatom; and further preferable are a fluorine atom, a nitro group, a cyano group, and a hydrocarbyl group having 1 to 3 carbon atoms and optionally containing a heteroatom. In such an onium salt, the surroundings of the sulfonate anion are not sterically crowded, and therefore, such an onium salt is excellent in sensitivity and resolution. Particularly when R has a hydrogen bond donor, such as carboxylic acid, alcohol, and aldehyde, the R is preferably present in an m-position or p-position of the sulfonate anion. When a hydrogen bond donor is present in an m-position or p-position of the sulfonate anion, there is no risk of sensitivity being degraded due to uneven distribution of generated acid caused by intramolecular hydrogen bonds.

In the formula (A), W represents a hydrocarbyl group having 6 to 60 carbon atoms, preferably 6 to 40 carbon atoms and containing at least one aromatic ring and optionally contains a heteroatom, the aromatic ring optionally having a substituent which is a halogen atom, a nitro group, a hydroxy group, a cyano group, a pentafluorosulfanyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom.

Specific examples of the aromatic ring include benzene, naphthalene, anthracene, and pyrene. To enhance EUV absorption and enhance solvent solubility, it is preferable that an iodinated aromatic ring or a fluorinated aromatic ring is contained.

In the formula (A), L represents a single bond, an ester group, an ether group, an amide group, a sulfonic acid ester group, or a linear, branched, or cyclic alkylene group having 1 to 12 carbon atoms, the alkylene group optionally containing one or more kinds selected from an ester group, an ether group, an amide group, and a sulfonic acid ester group. L is preferably an ester group or a sulfonic acid ester group, more preferably an ester group.

In a photo-acid generator that contains a benzenesulfonic acid having multiple substituents which are iodine atoms, secondary electrons are generated from the iodine atoms during exposure in EUV lithography of wavelength 13.5 nm, since iodine atoms very greatly absorb EUV. By virtue of the resonance effect of the aromatic group, a stronger acid can be achieved when substituents which are iodine atoms are contained compared to an arylsulfonic acid only containing substituents which are fluorine atoms, and also by virtue of the high EUV absorption property of iodine atoms, acidity is enhanced. Furthermore, the mobility of the sulfonic acid is reduced by iodine atoms, having a great atomic weight, being present nearby, and acid diffusion can be suppressed and it is possible to improve LWR and CDU. Furthermore, in the inventive onium salt, not both of the two carbon atoms adjacent to the carbon atom having a substituent which is the sulfonic acid “SO₃⁻” are bonded to iodine atoms. That is, there is a characteristic that at least one of the two carbon atoms in the ortho positions of the sulfonate anion is not bonded to an iodine atom, and therefore, the sulfonic acid is not affected by the sterically bulky electron clouds of iodine atoms, and favorable sensitivity and favorable resolution can be exhibited.

As the onium salt represented by the formula (A), one represented by the following formula (A1) is preferable.

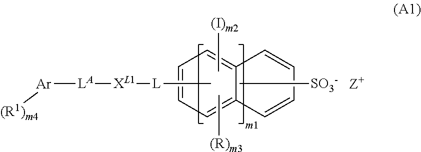

In the formula, “m4” represents 0 to 9; X^{L1} represents a single bond or a hydrocarbylene group having 1 to 40 carbon atoms and optionally containing a heteroatom; L^{A} represents a single bond or a linear, branched, or cyclic alkyl group having 1 to 12 carbon atoms and optionally containing one or more kinds selected from an ester group, an ether group, an amide group, and a sulfonic acid ester group; Ar represents an aromatic hydrocarbon group; R¹ represents a halogen atom, a nitro group, a hydroxy group, a cyano group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, when “m4” is 2 or more, the R¹s being identical to or different from each other and the R¹s optionally being bonded to each other to form a ring together with the carbon atoms bonded thereto; and “m1” to “m3”, L, R, and Z⁺ are as defined above.

As the onium salts represented by the formulae (A) and (A1), one represented by the following formula (A2) is preferable.

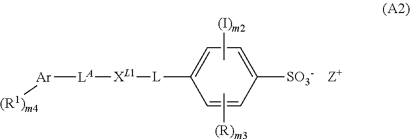

In the formula, “m2” to “m4”, L, L^{A}, X^{L1}, R, R¹, Ar, and Z⁺ are as defined above.

As the onium salts represented by the formulae (A), (A1), and (A2), one represented by the following formula (A3) is preferable.

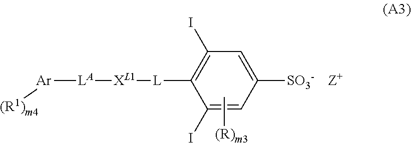

In the formula, “m3”, “m4”, L, L^{A}, X^{L1}, R, R¹, Ar, and Z⁺ are as defined above.

Specific examples of the anion of the onium salt represented by the formula (A) include ones shown below, but are not limited thereto. Note that Me represents a methyl group and Ac represents an acetyl group. Furthermore, the attachment positions of the substituents on the aromatic rings may change places with each other.

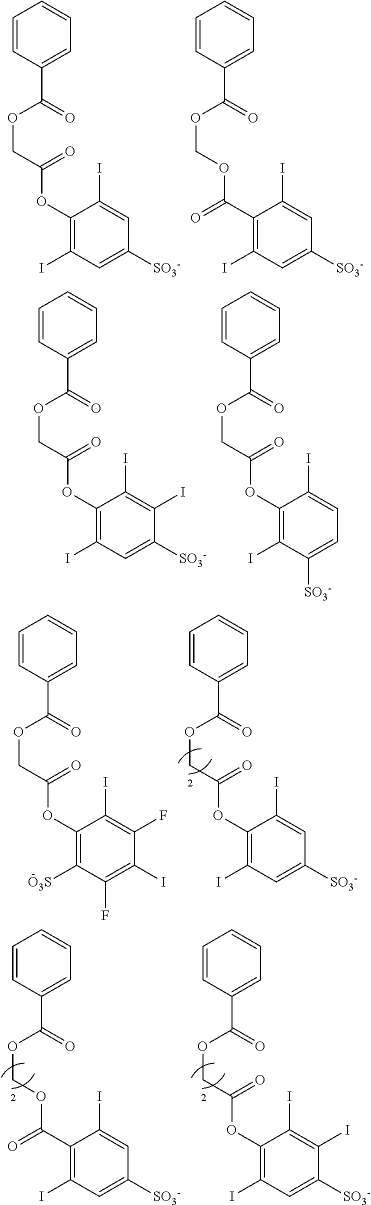

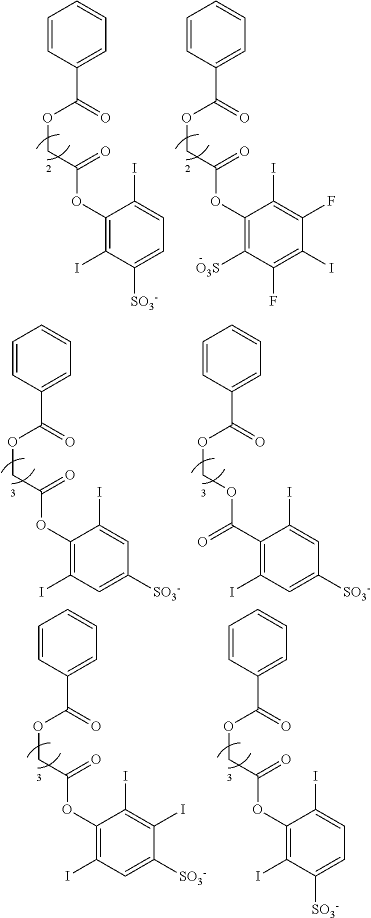

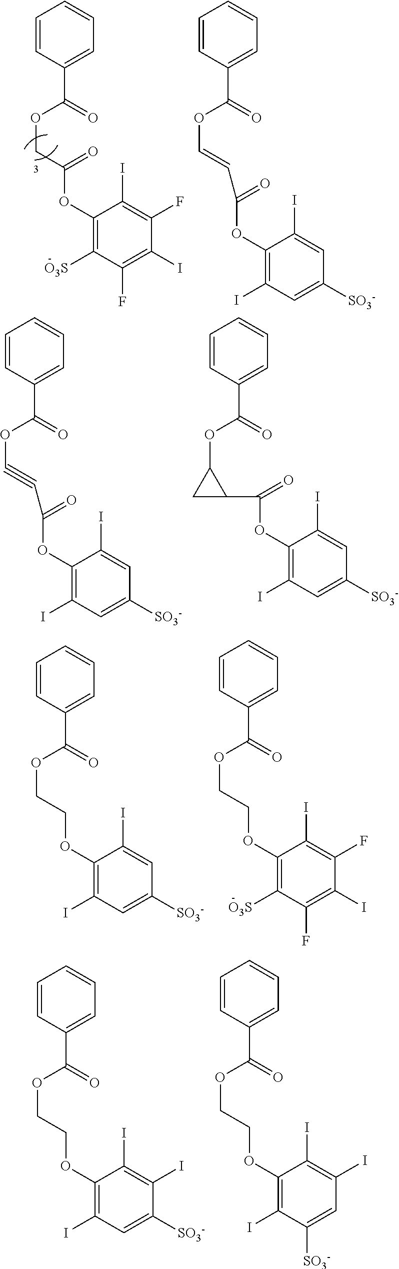

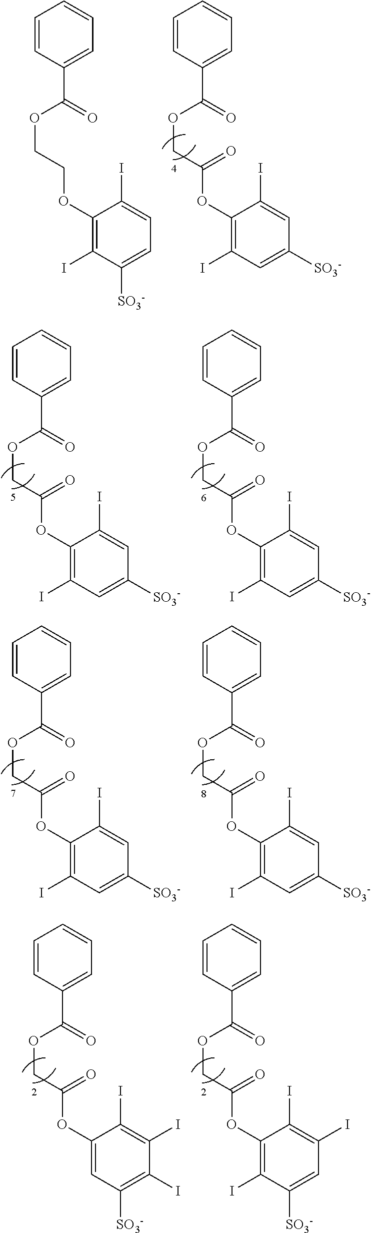

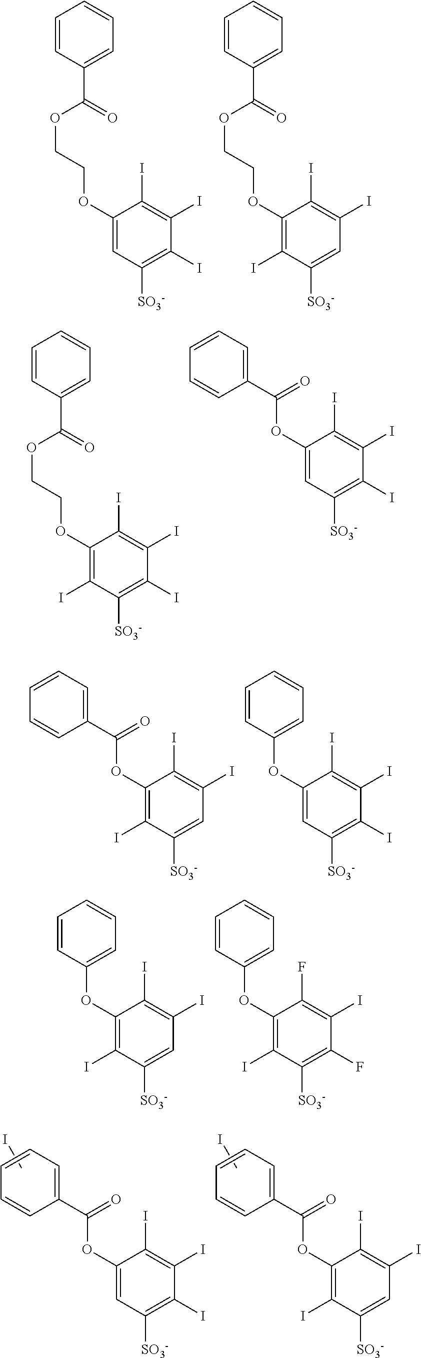

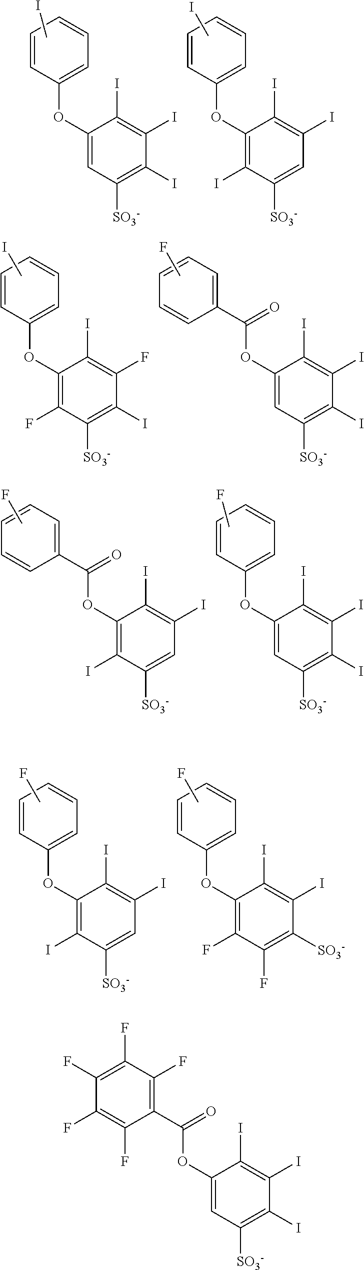

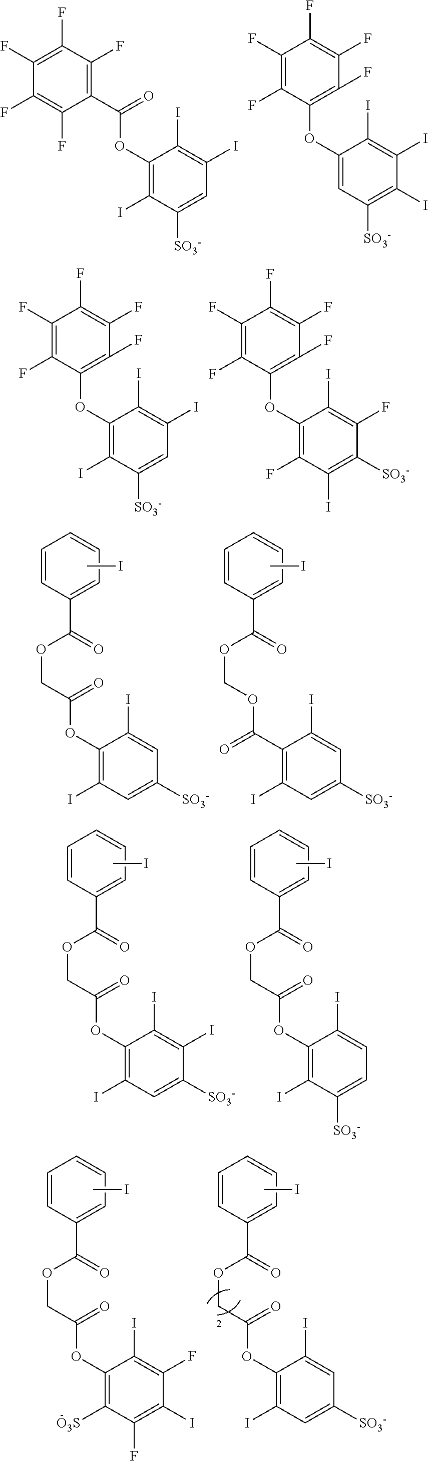

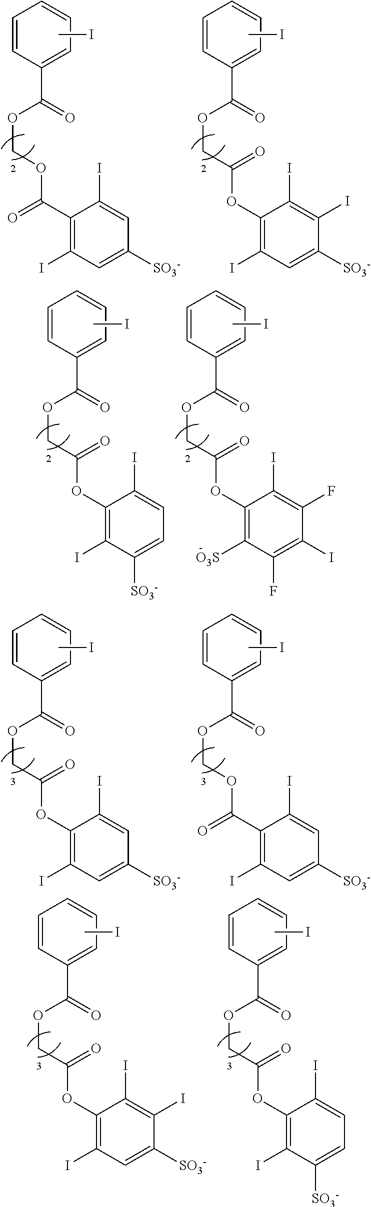

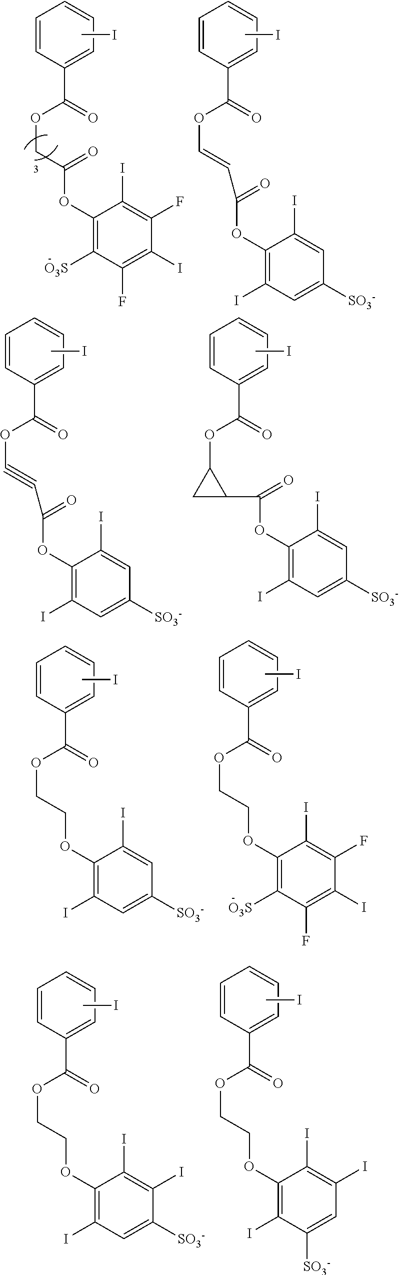

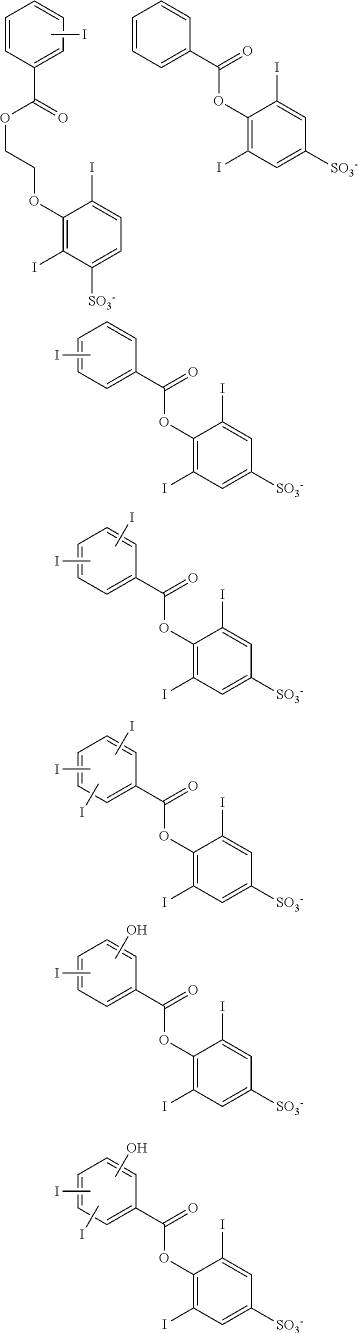

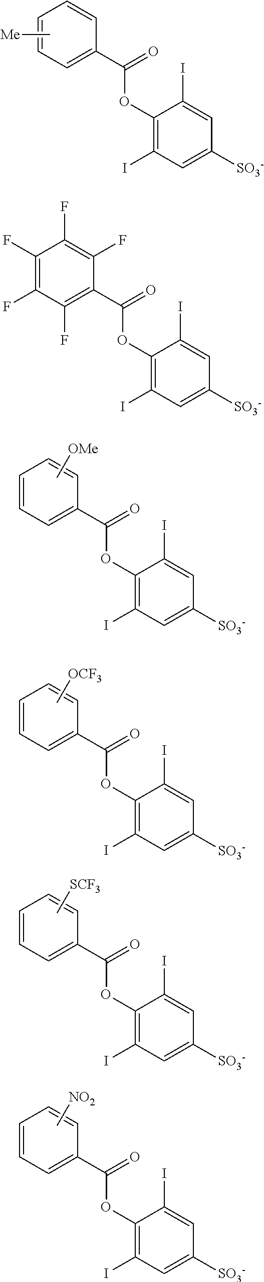

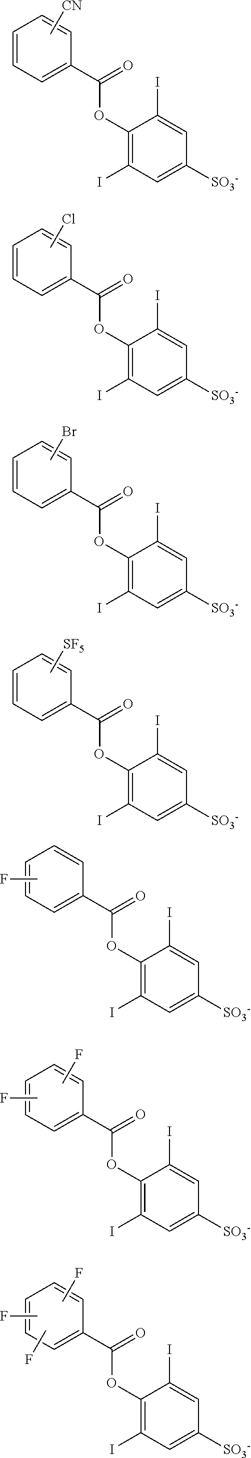

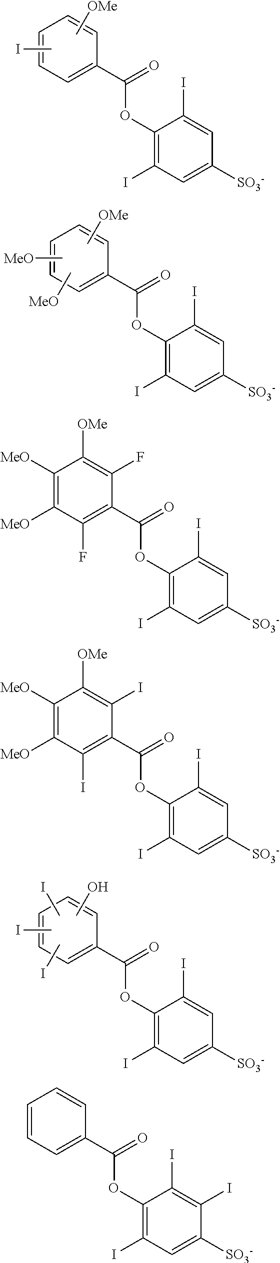

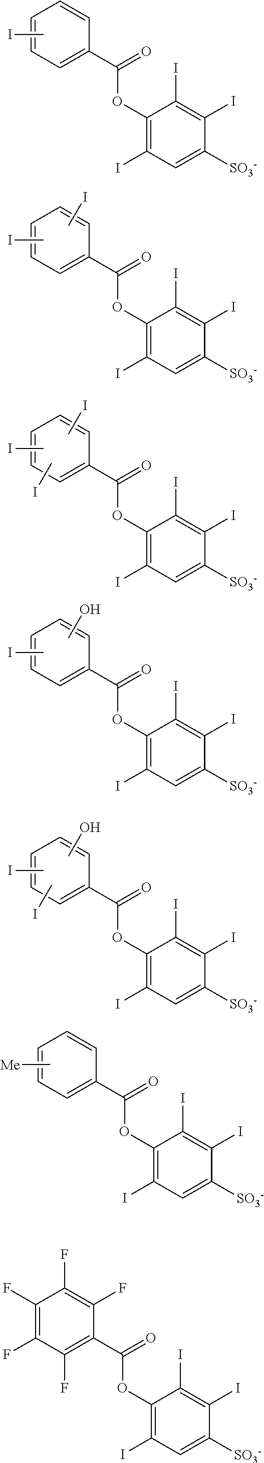

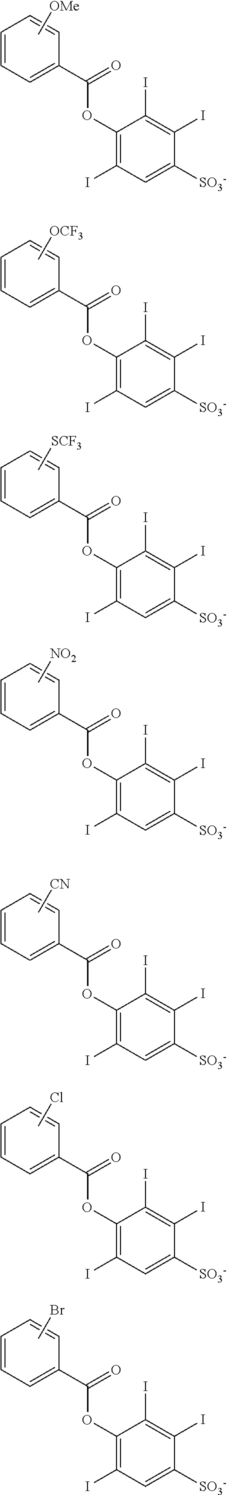

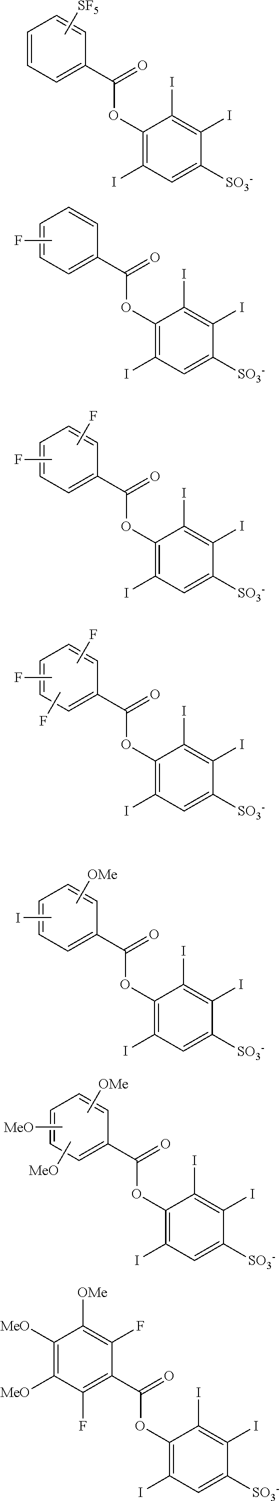

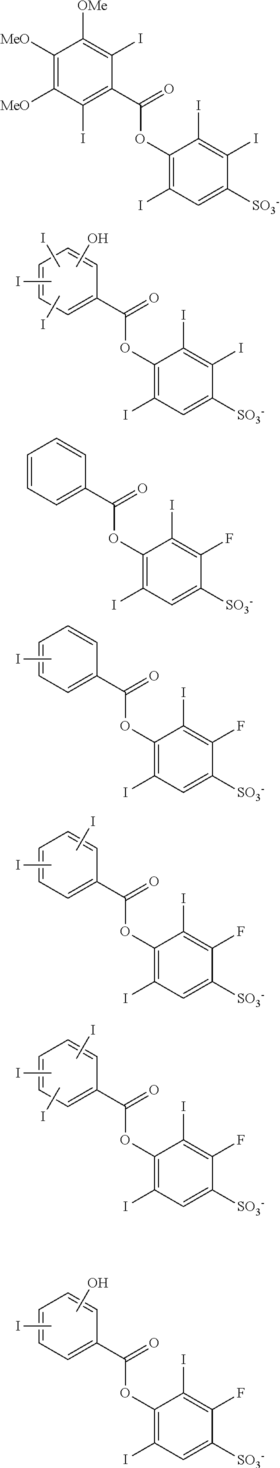

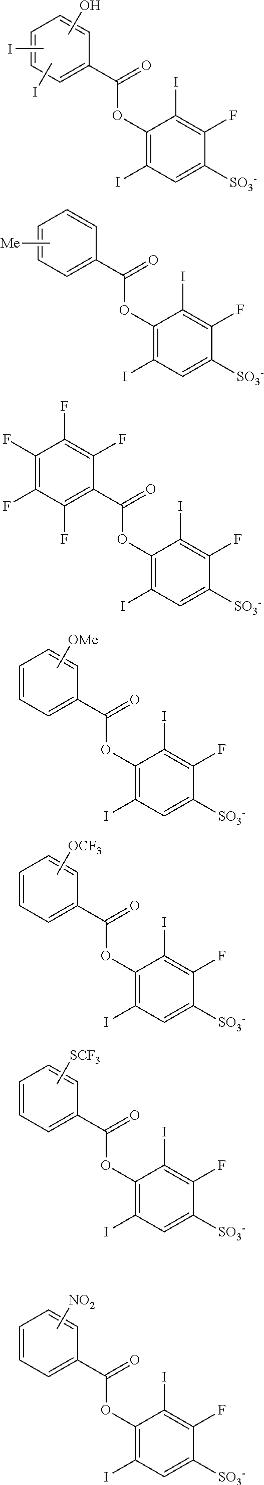

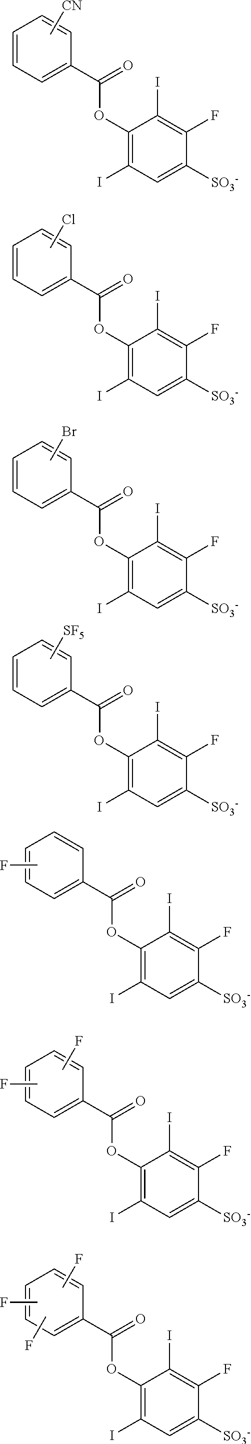

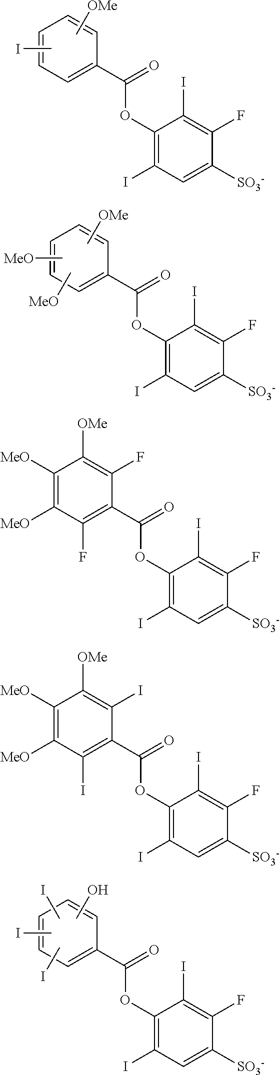

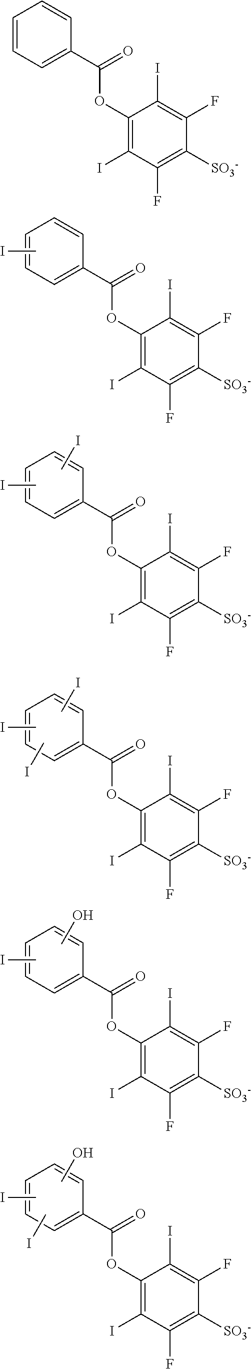

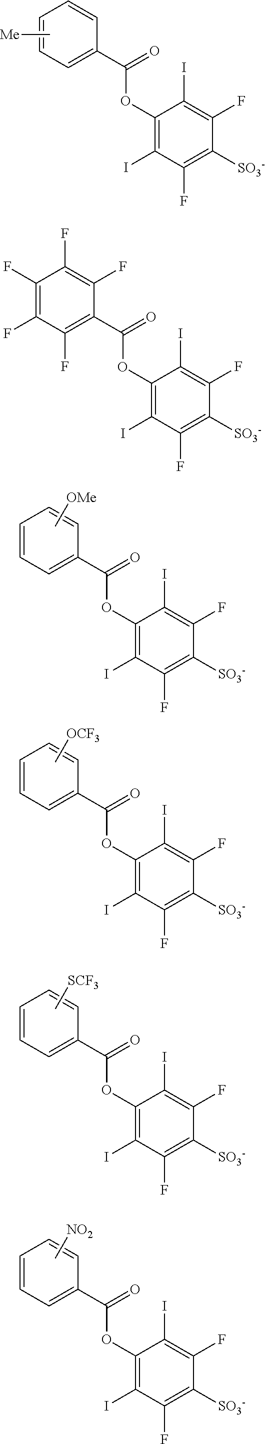

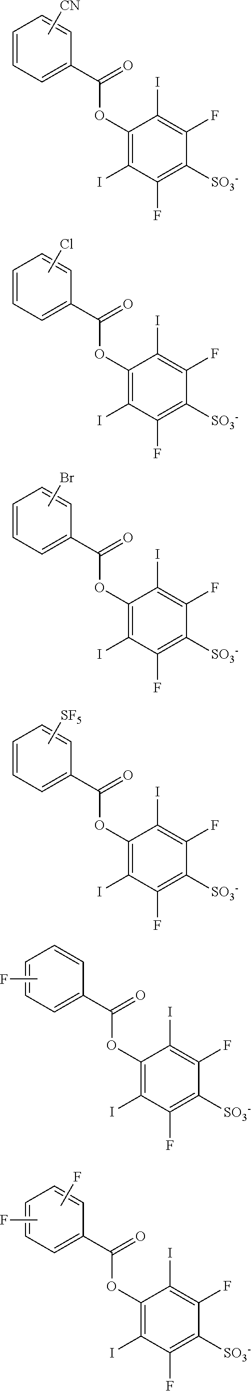

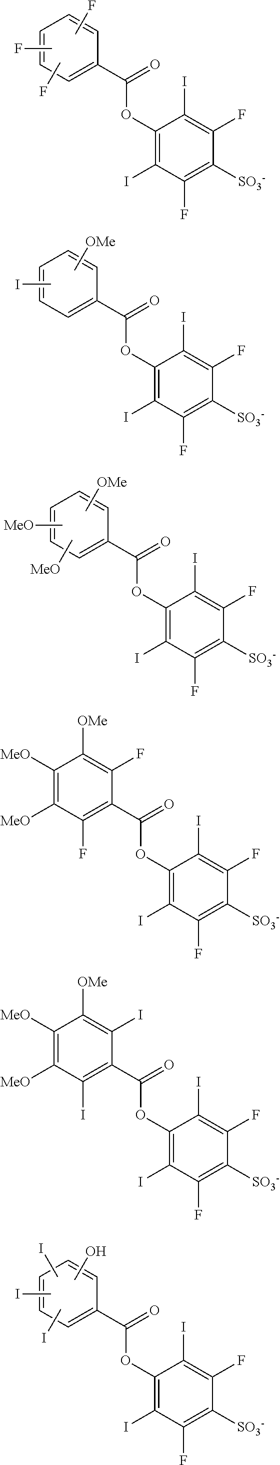

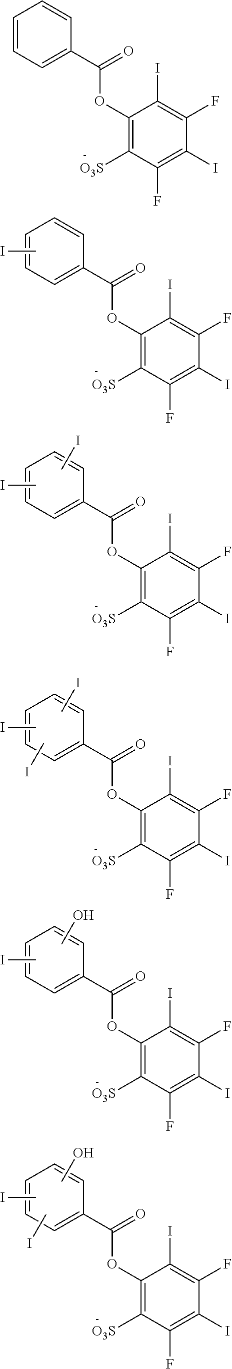

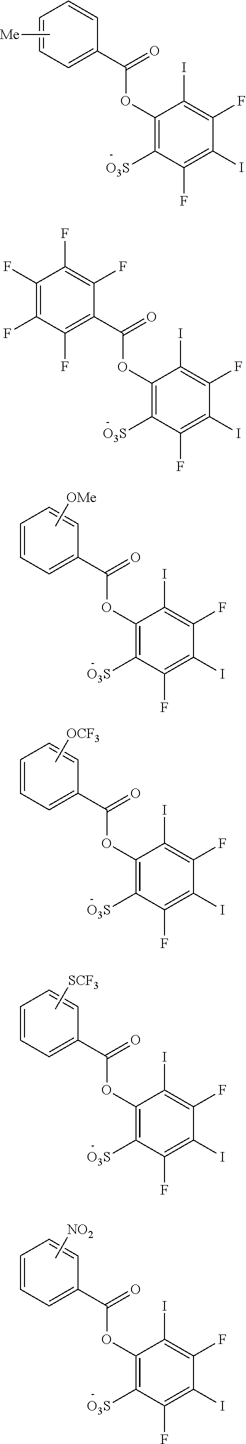

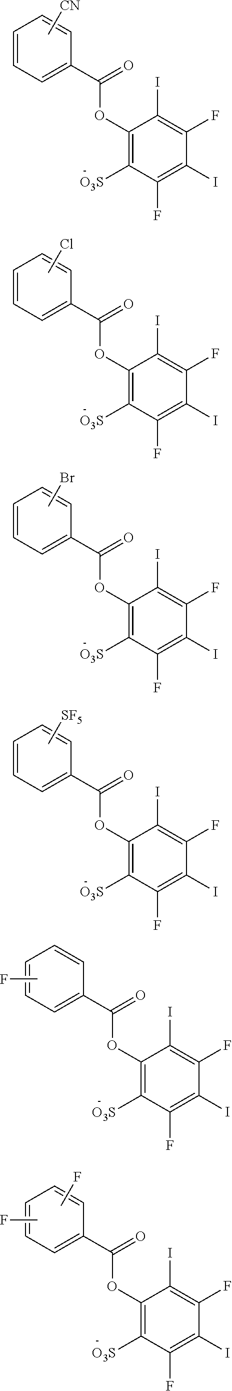

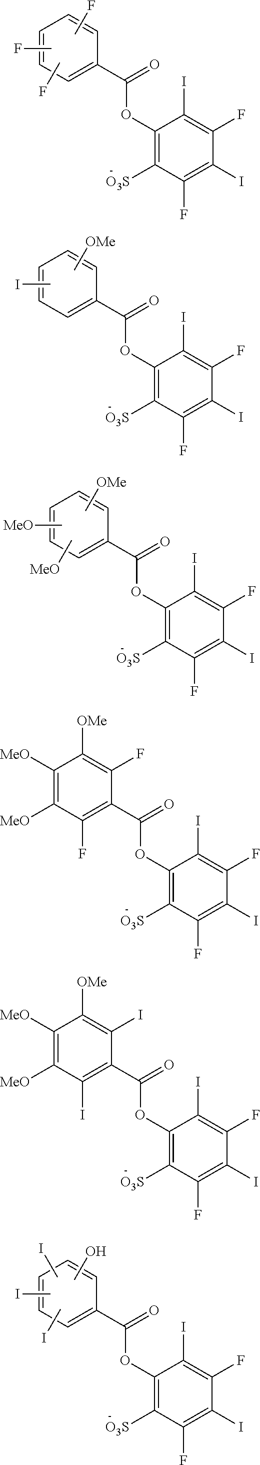

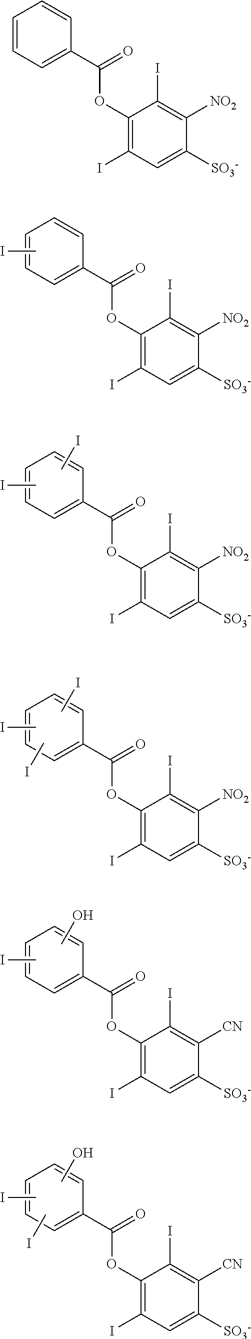

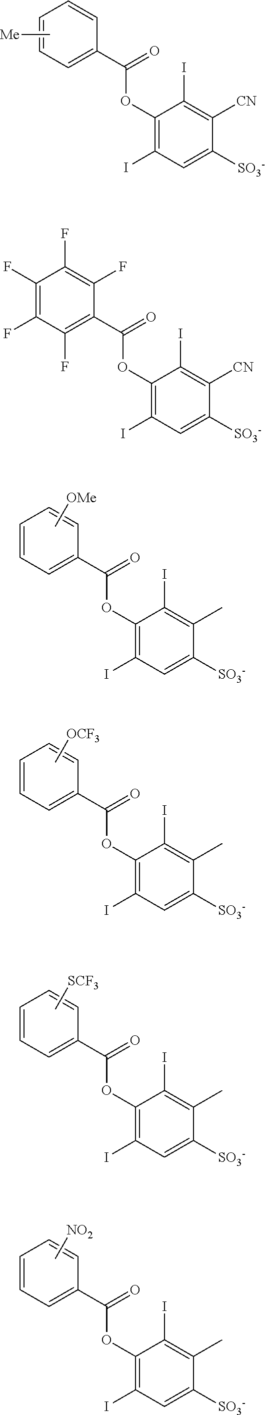

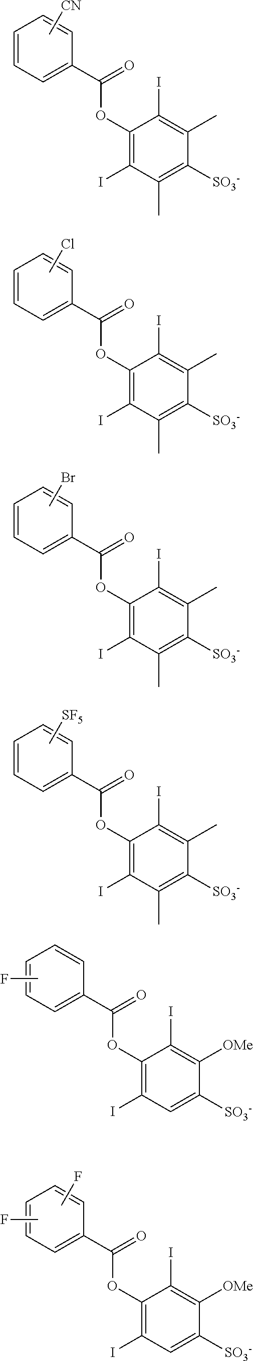

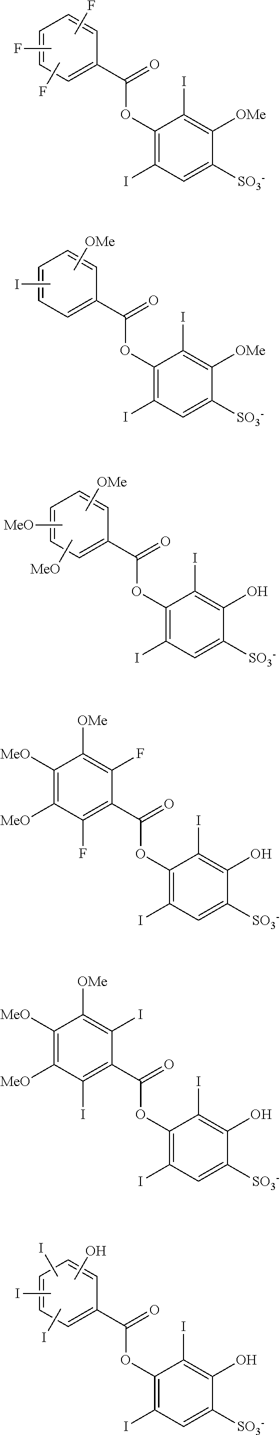

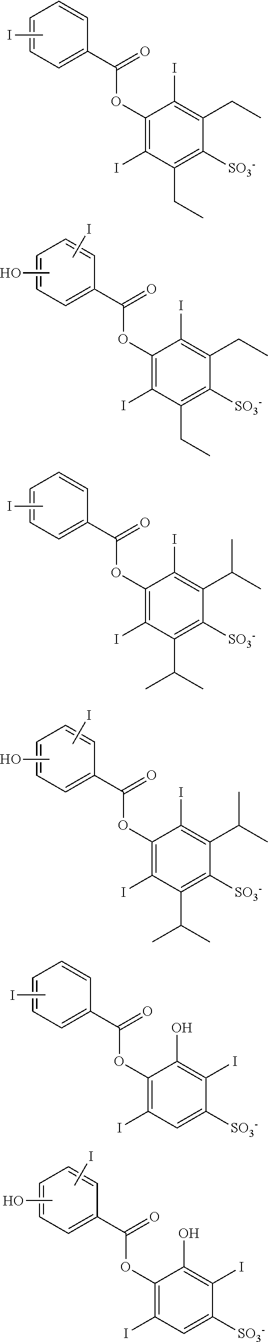

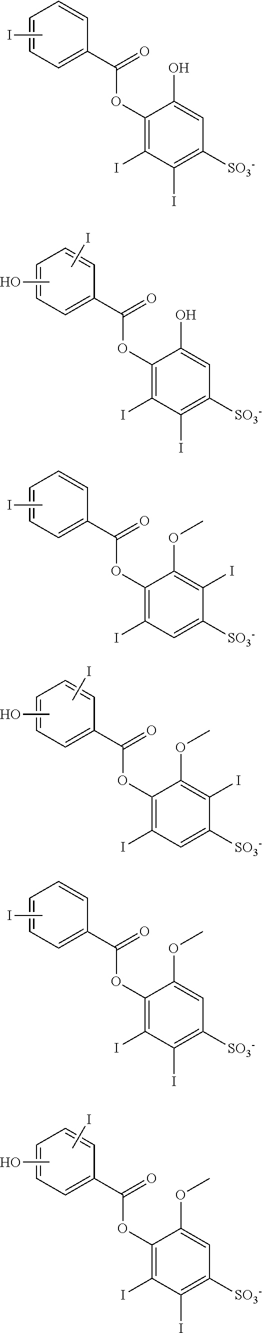

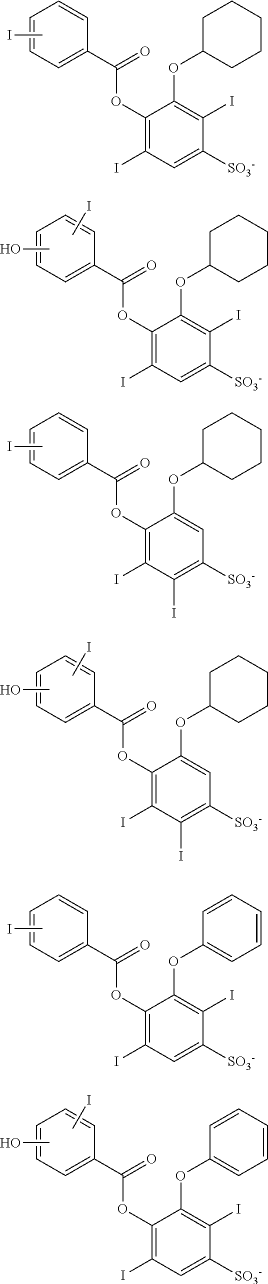

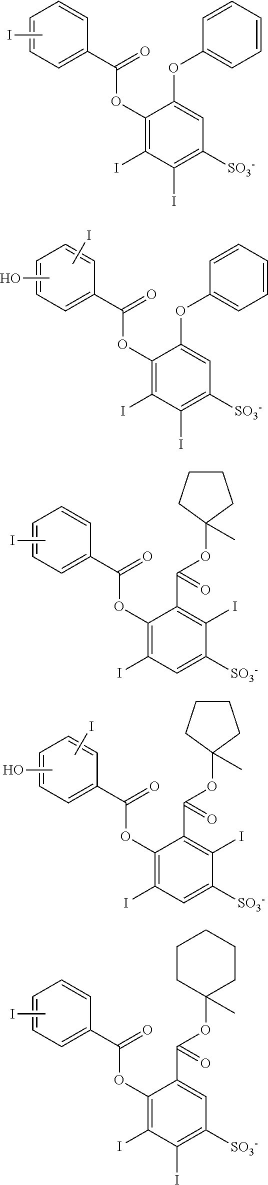

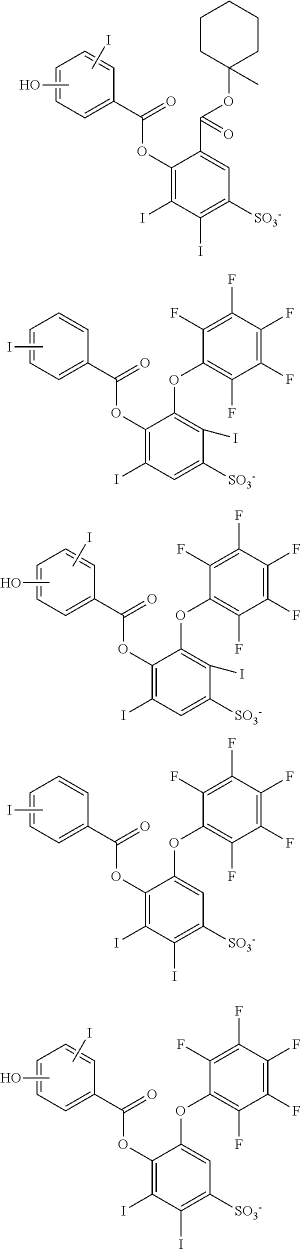

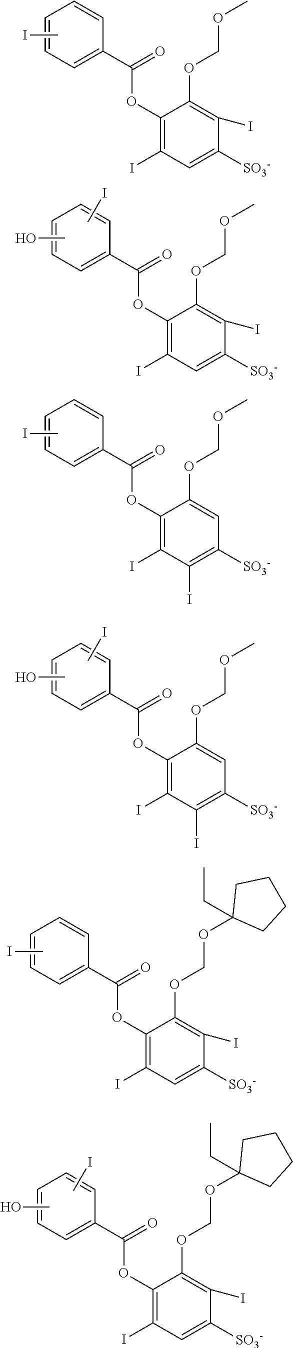

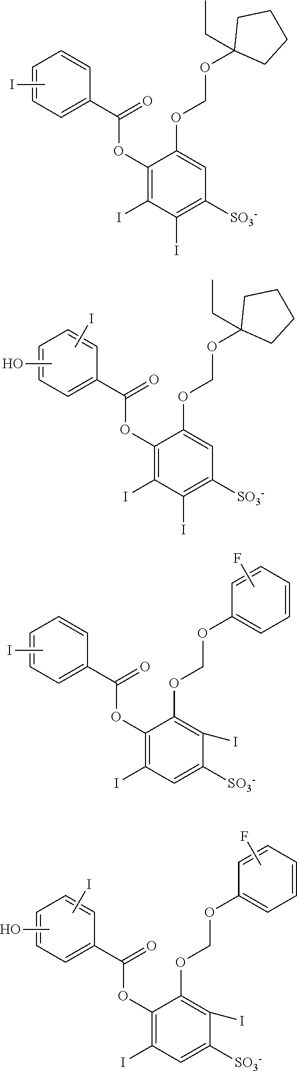

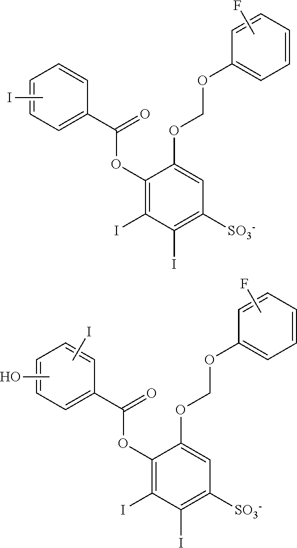

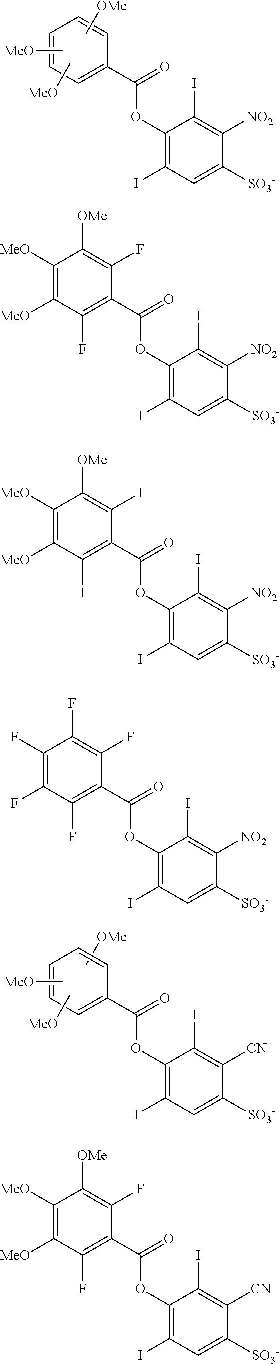

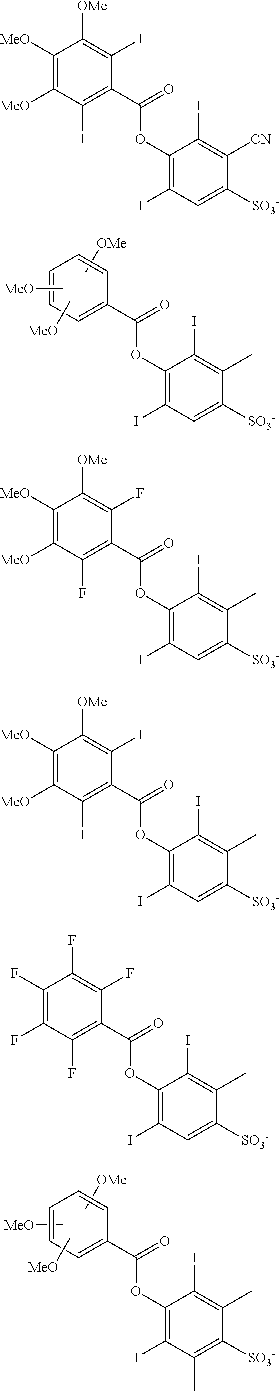

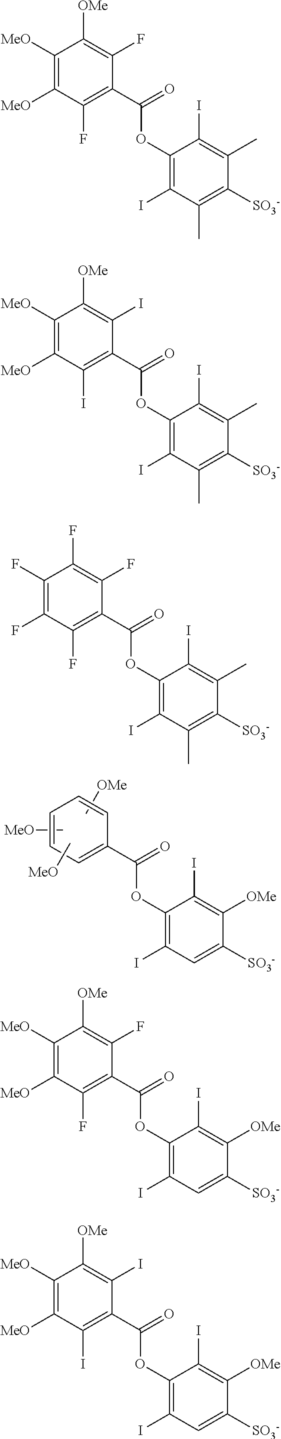

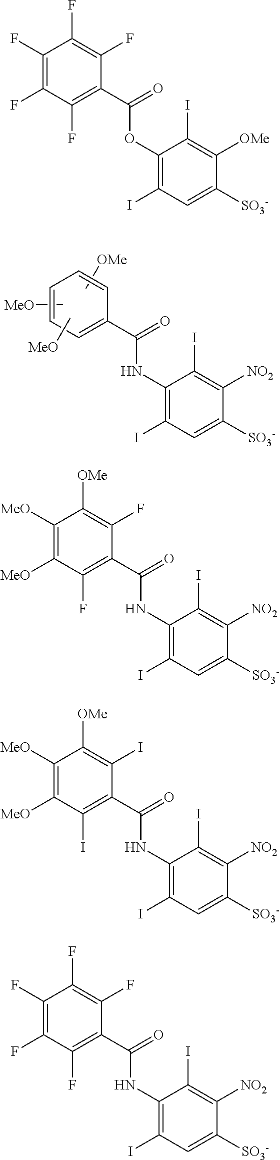

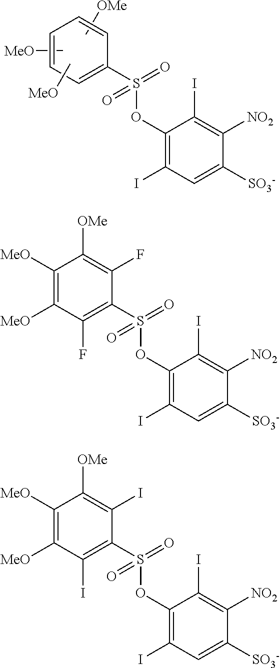

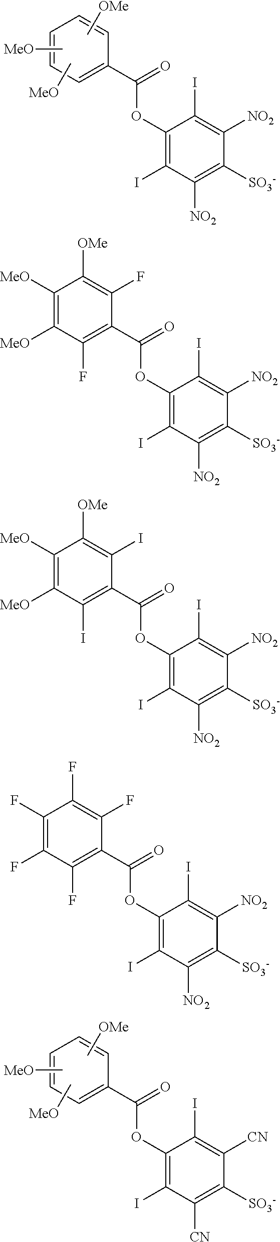

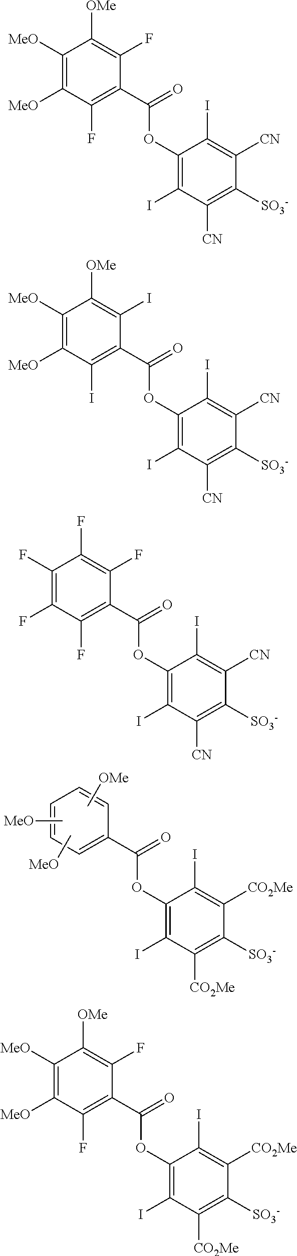

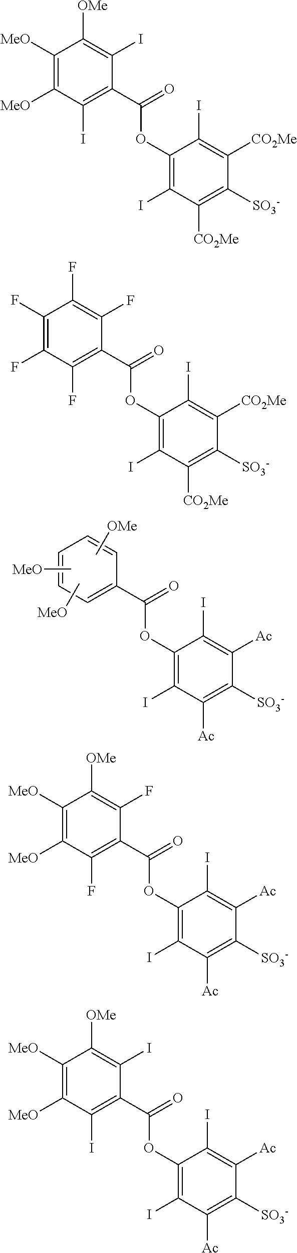

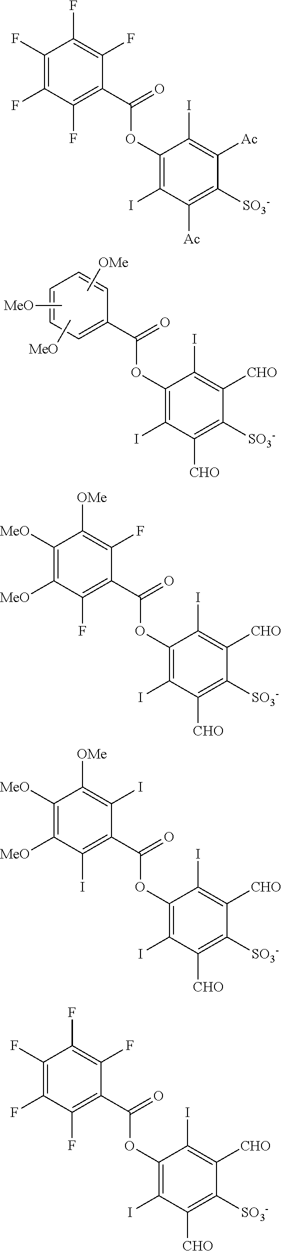

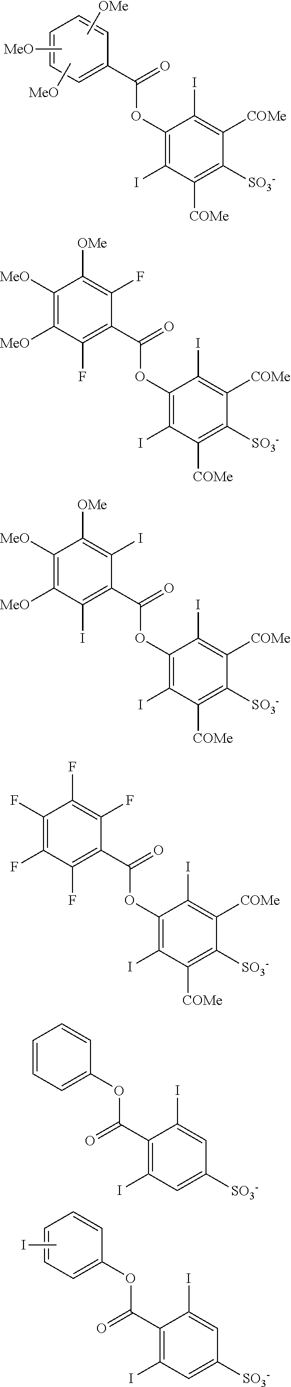

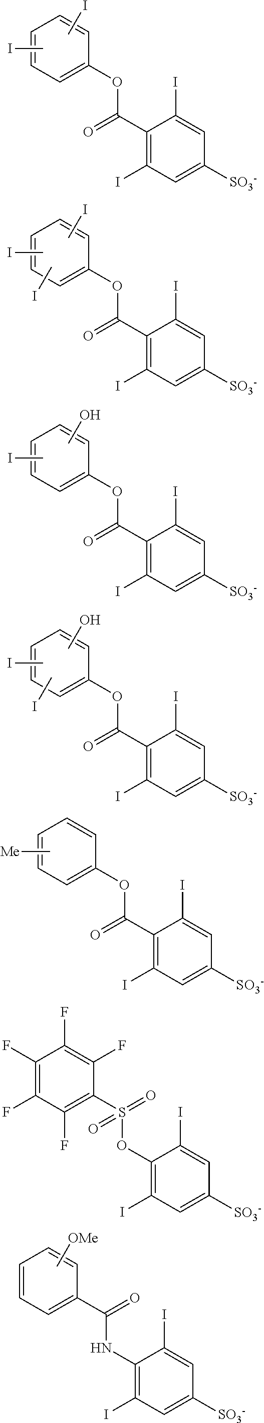

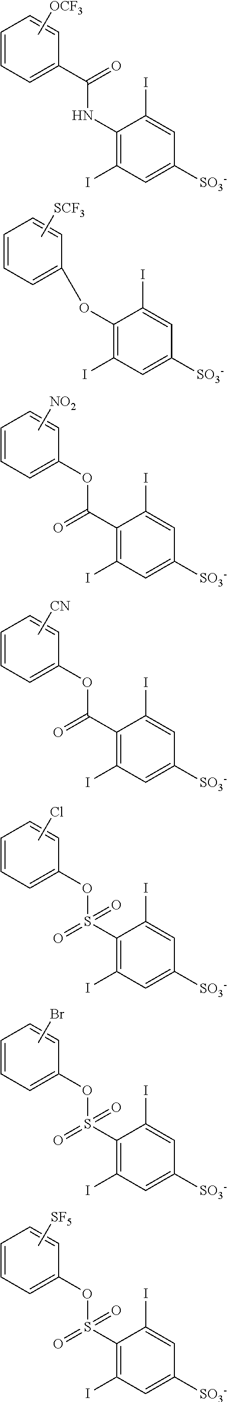

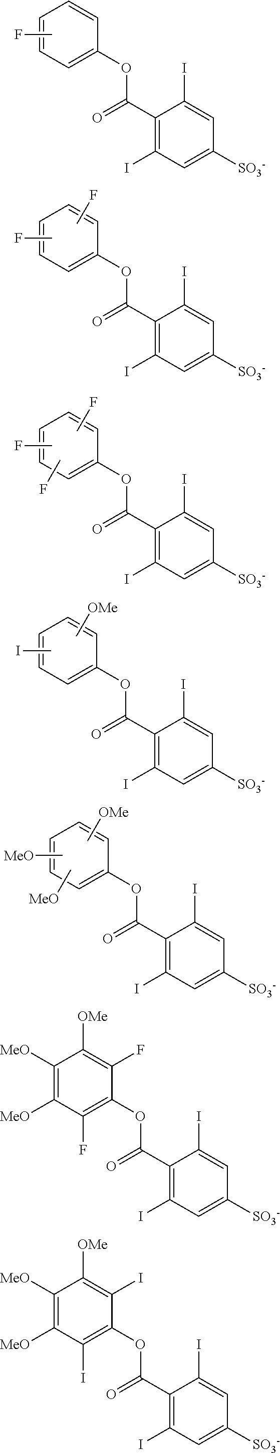

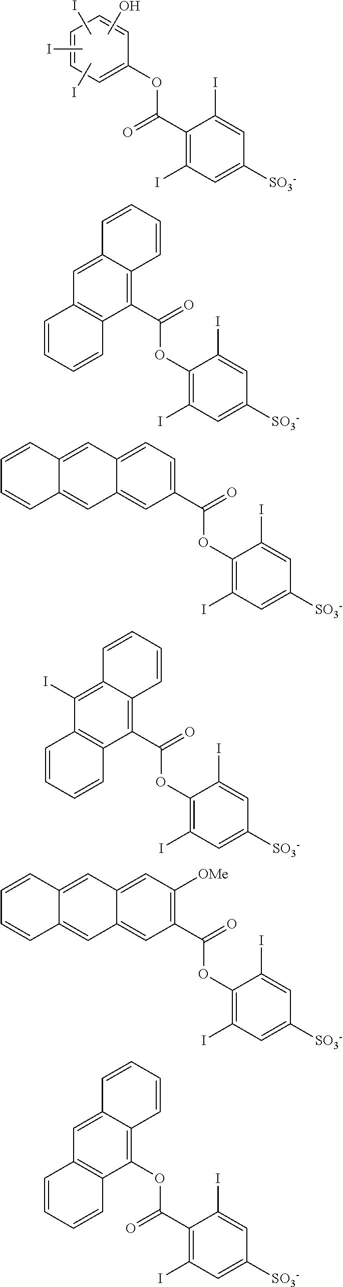

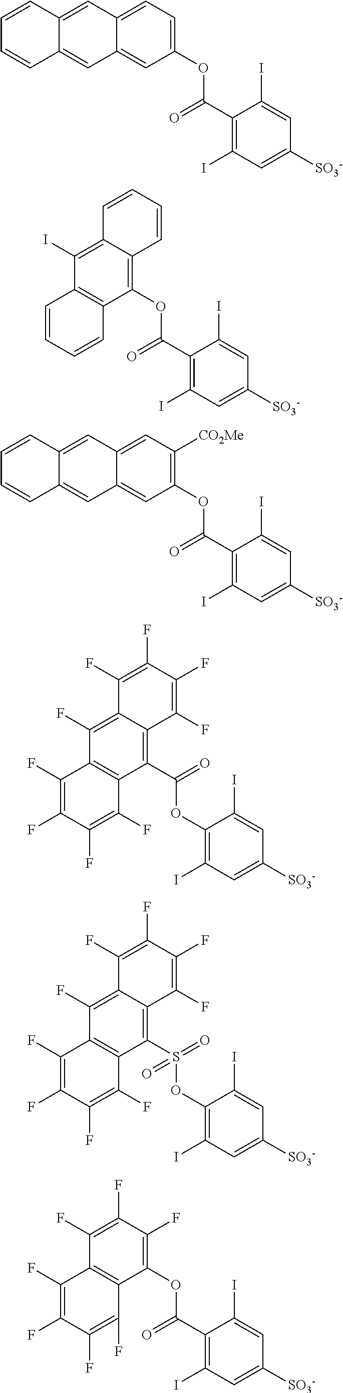

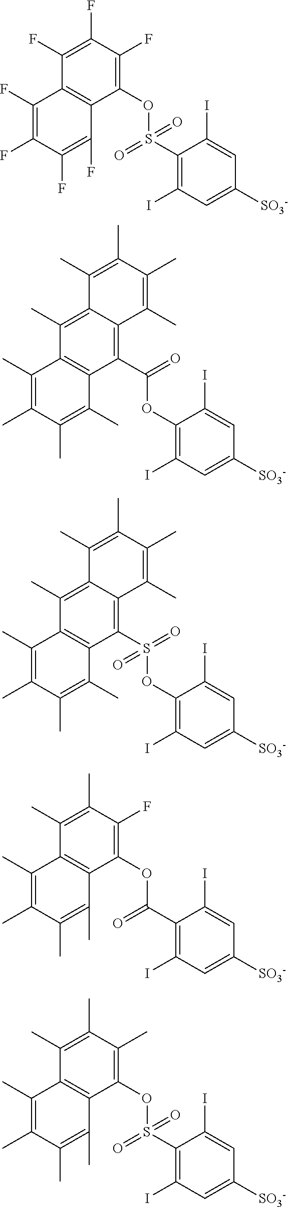

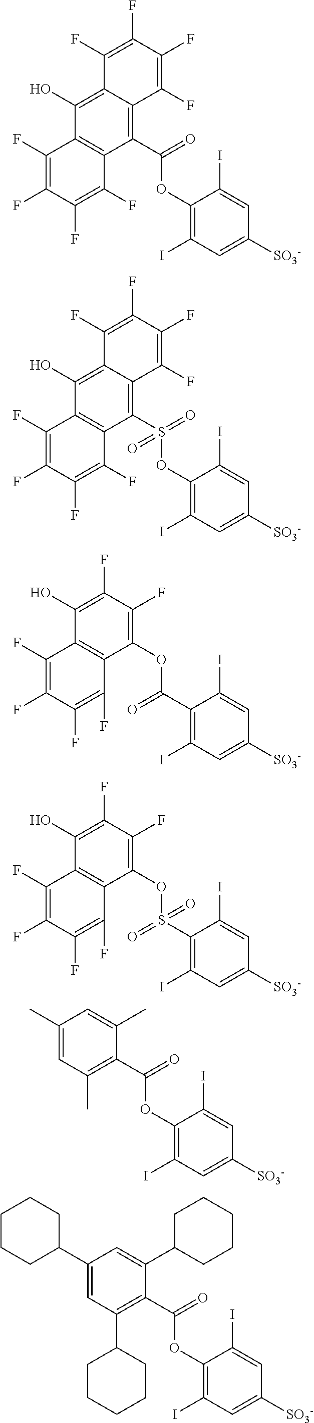

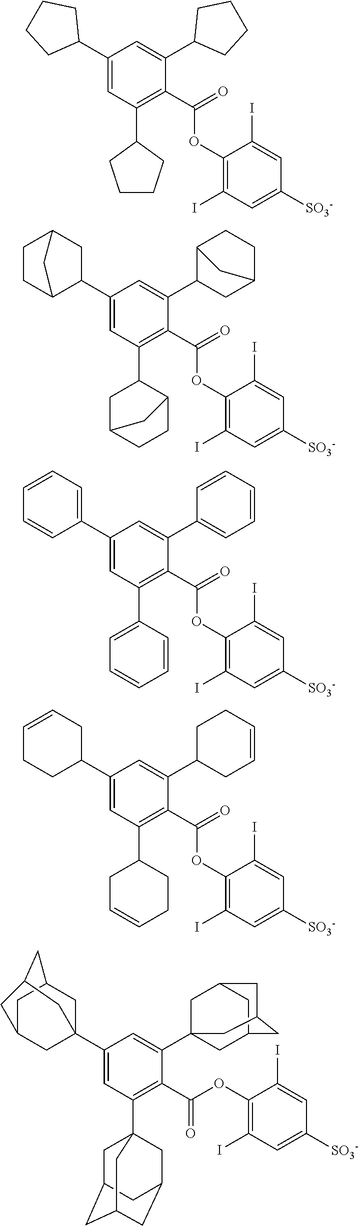

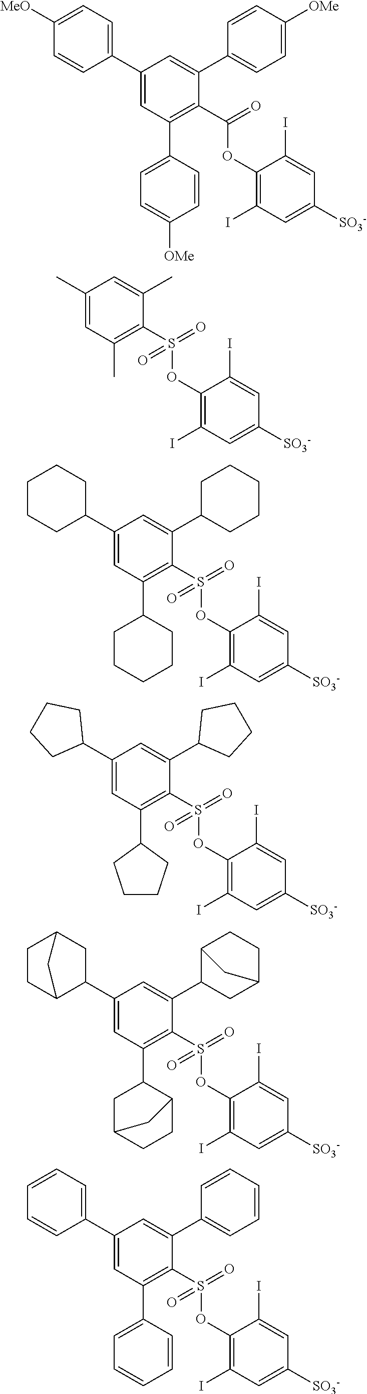

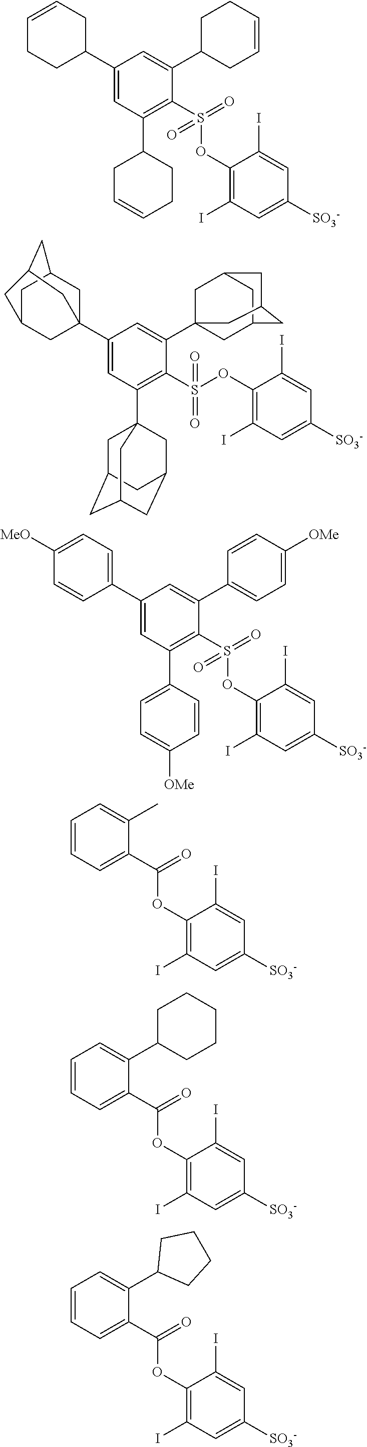

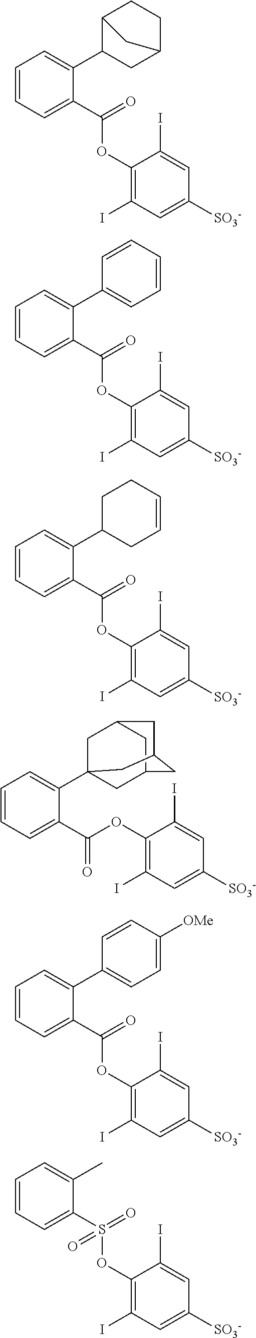

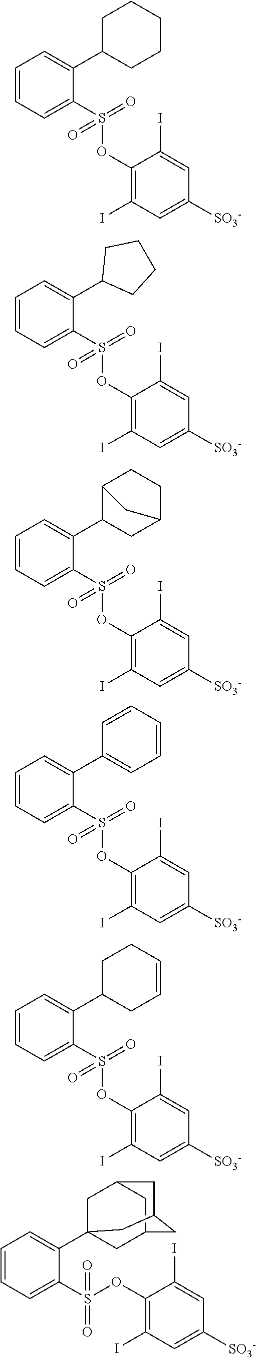

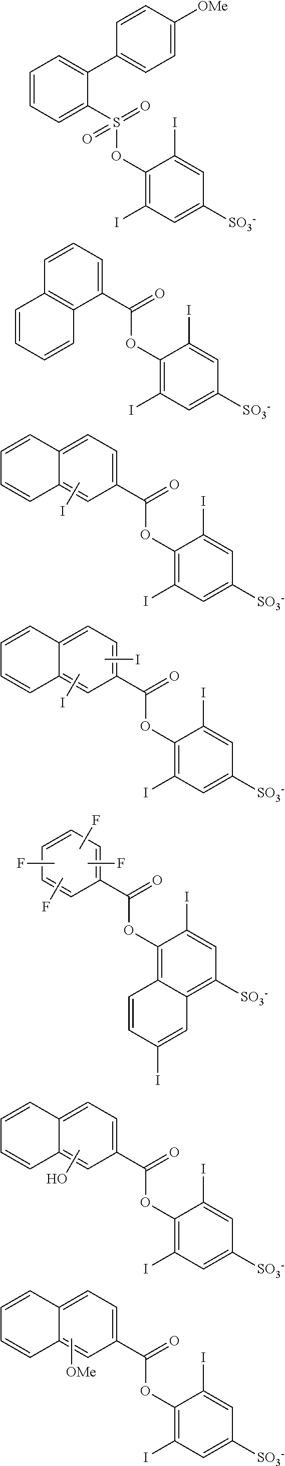

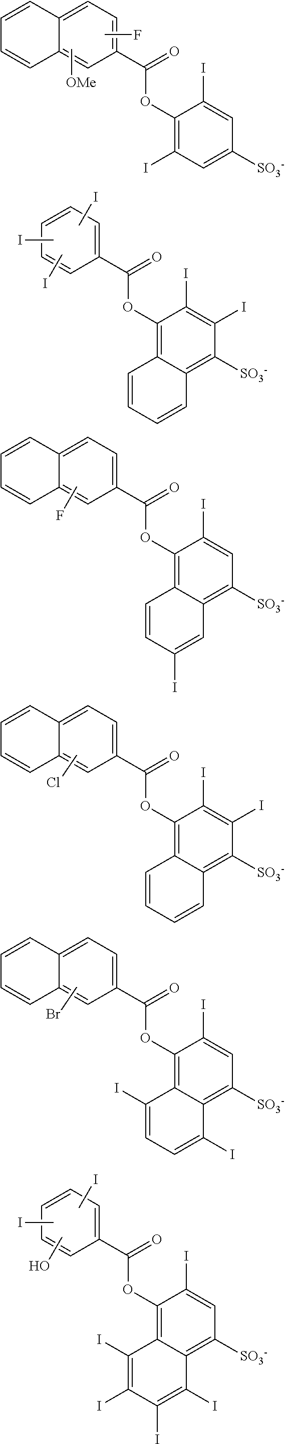

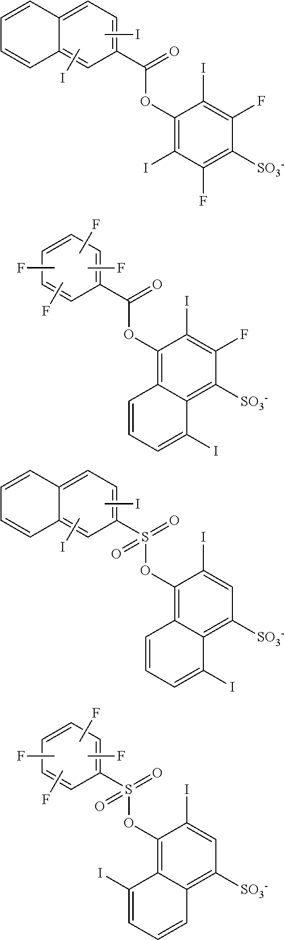

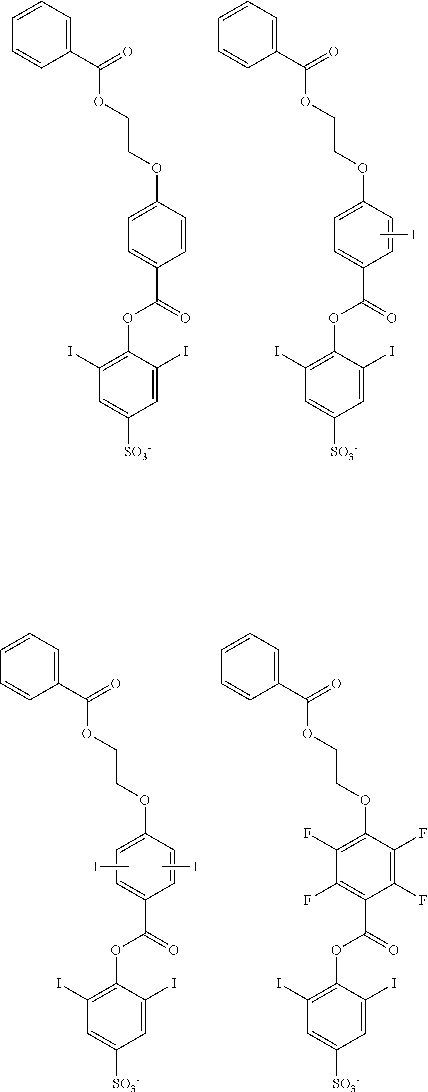

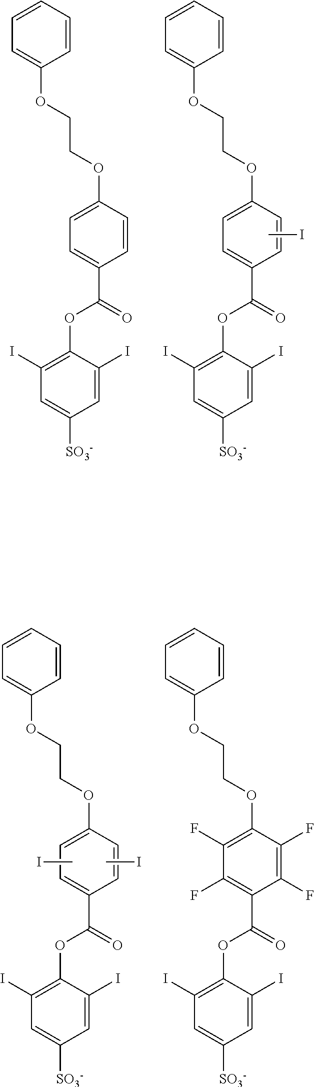

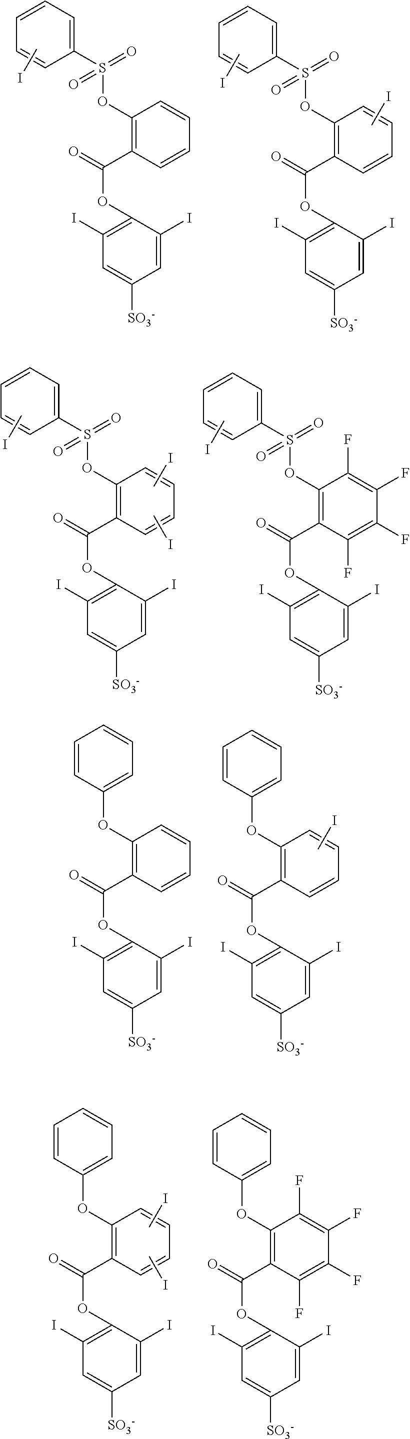

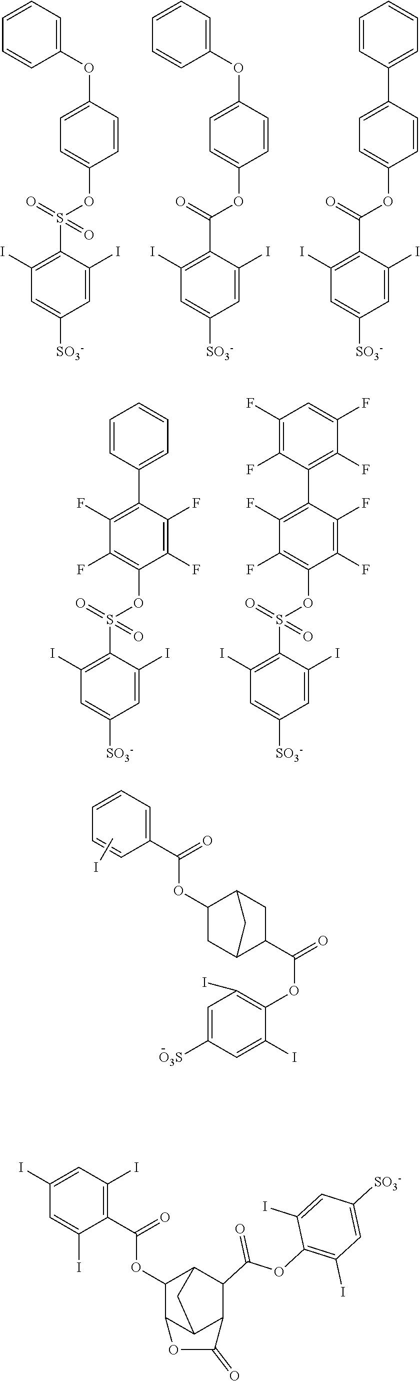

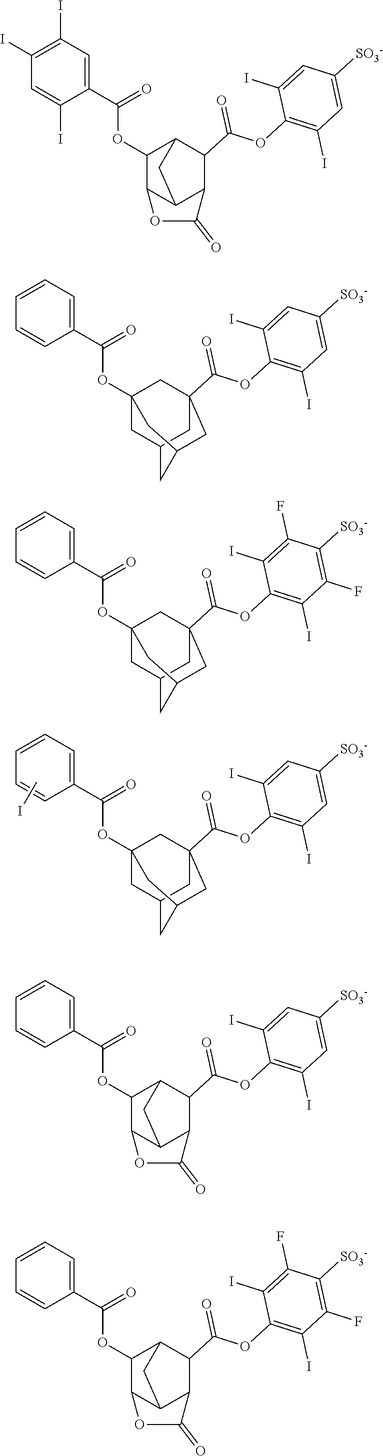

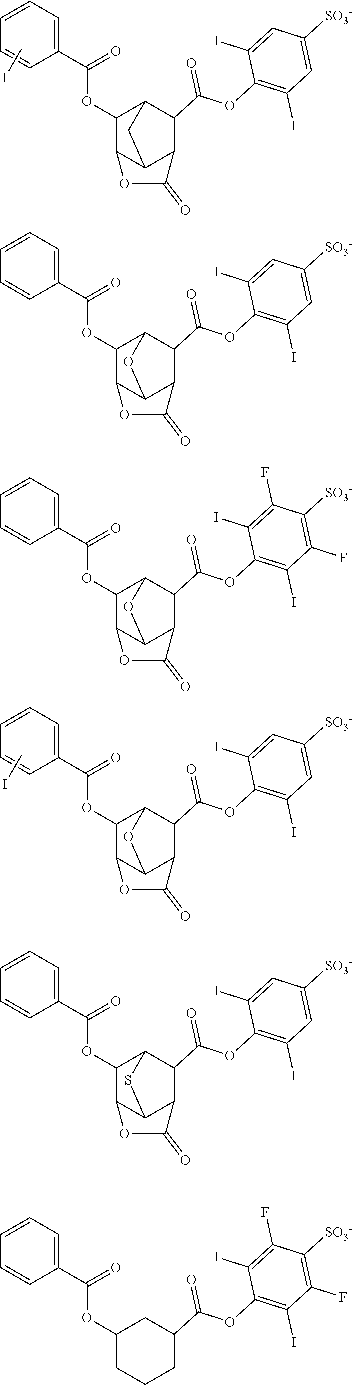

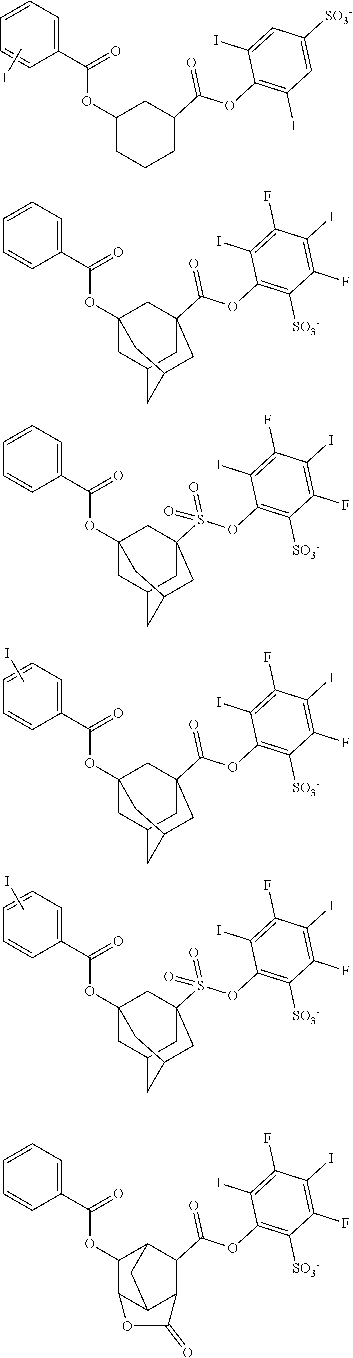

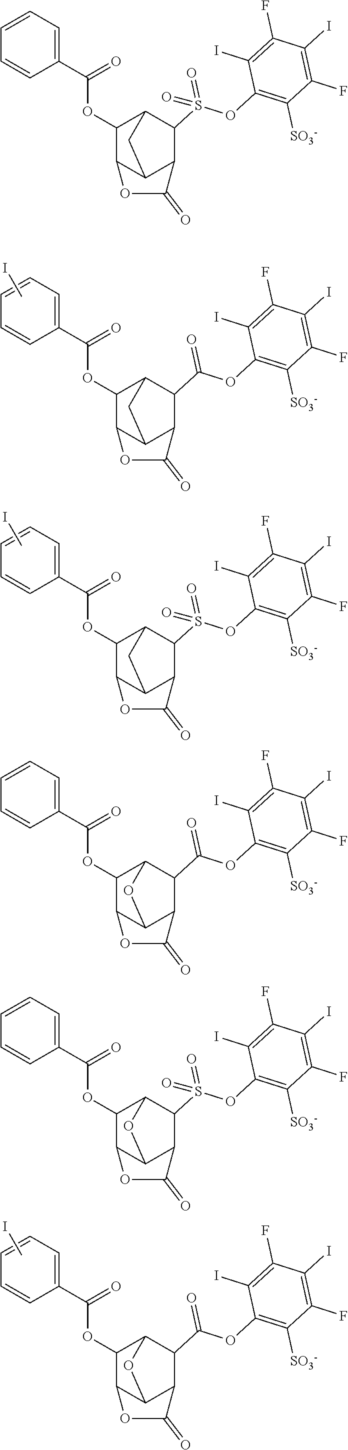

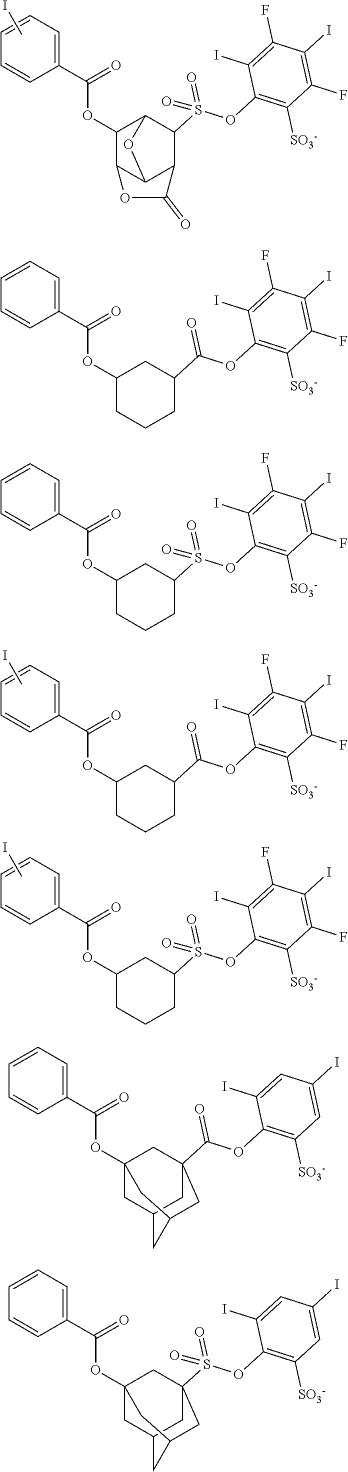

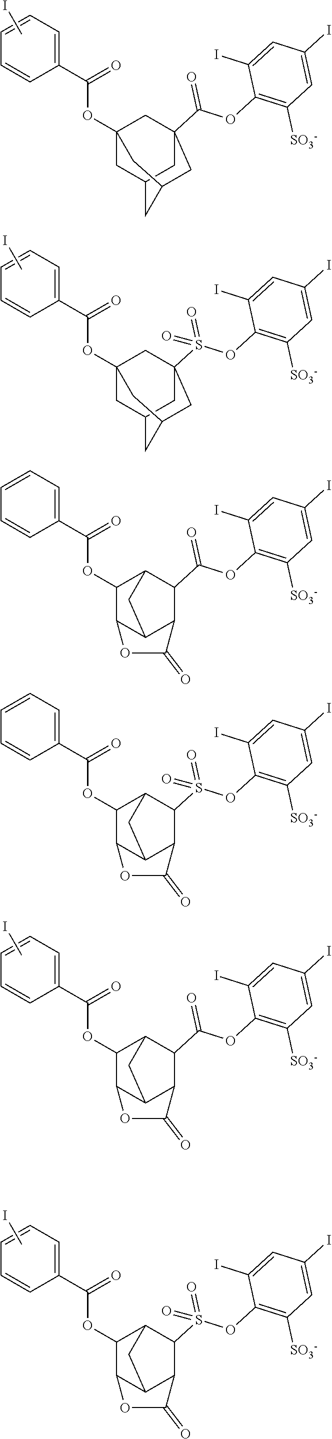

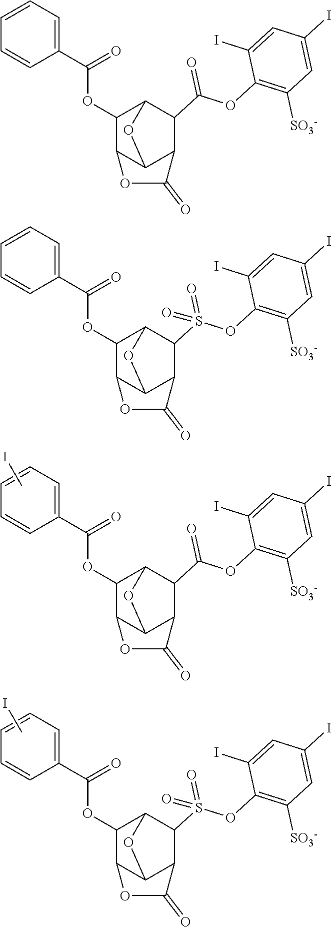

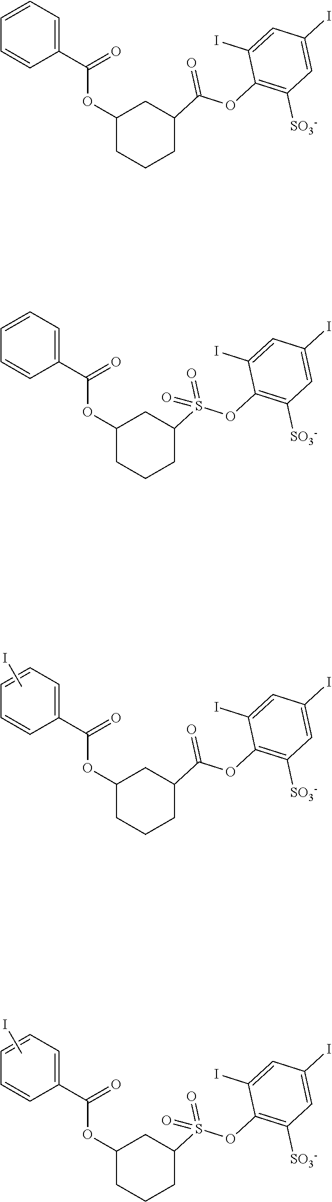

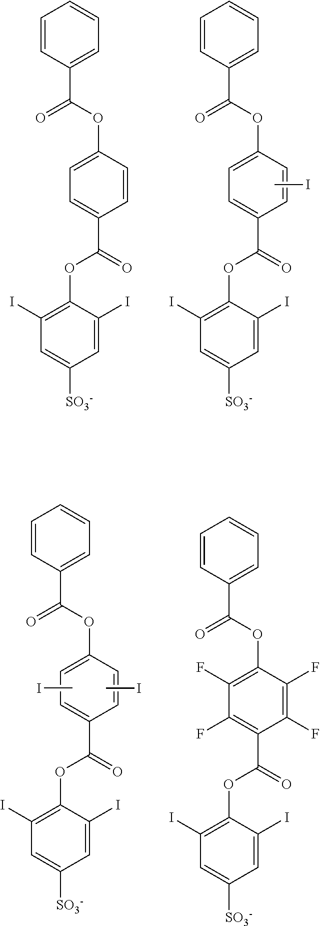

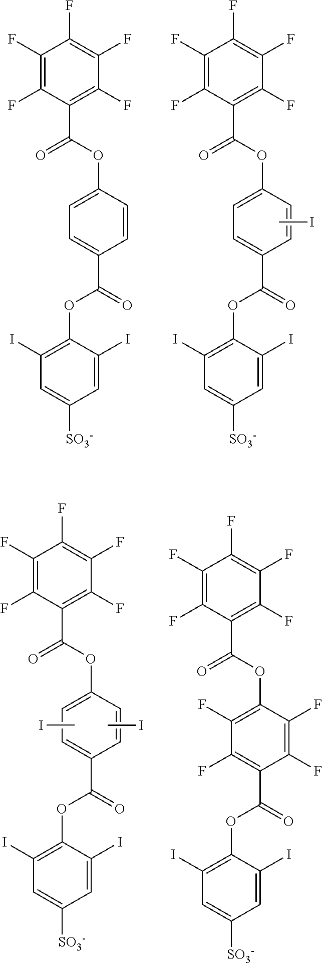

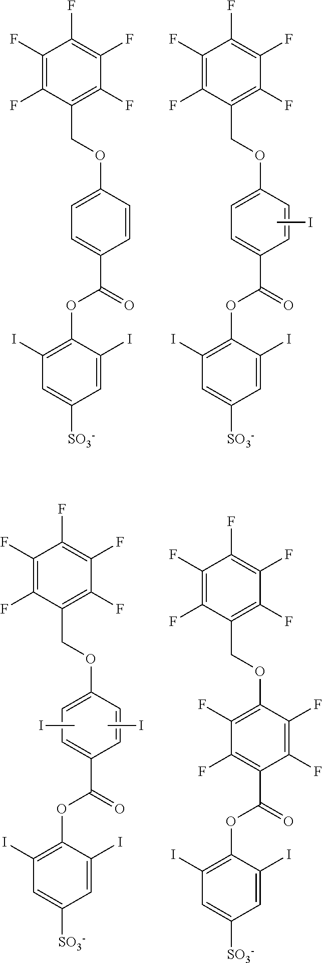

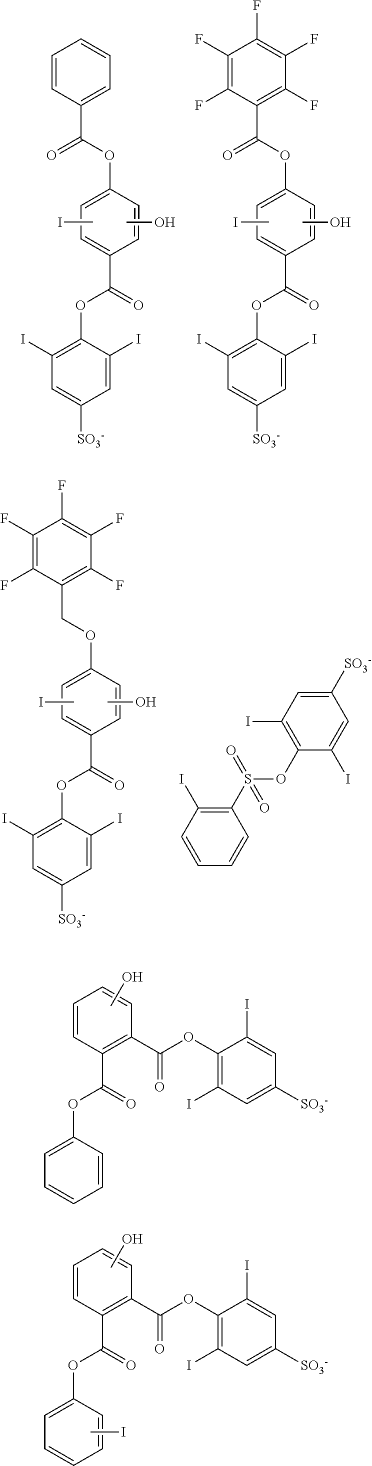

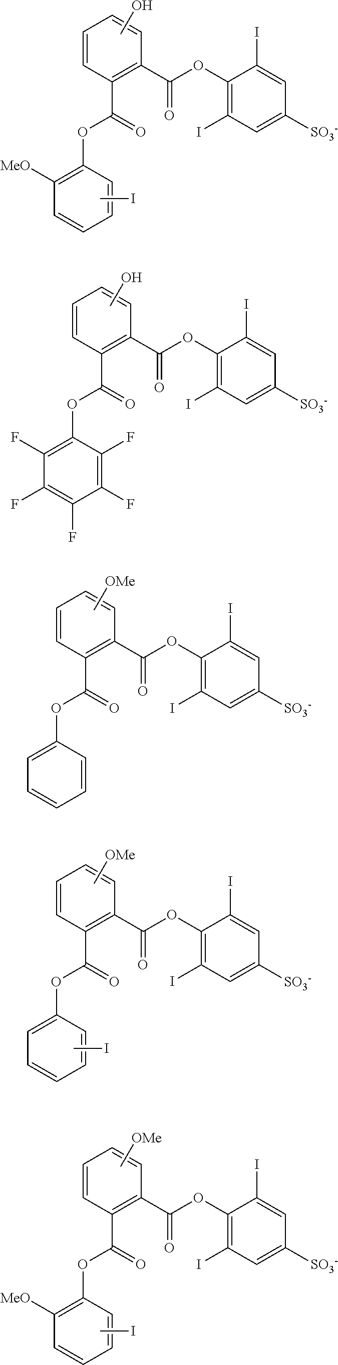

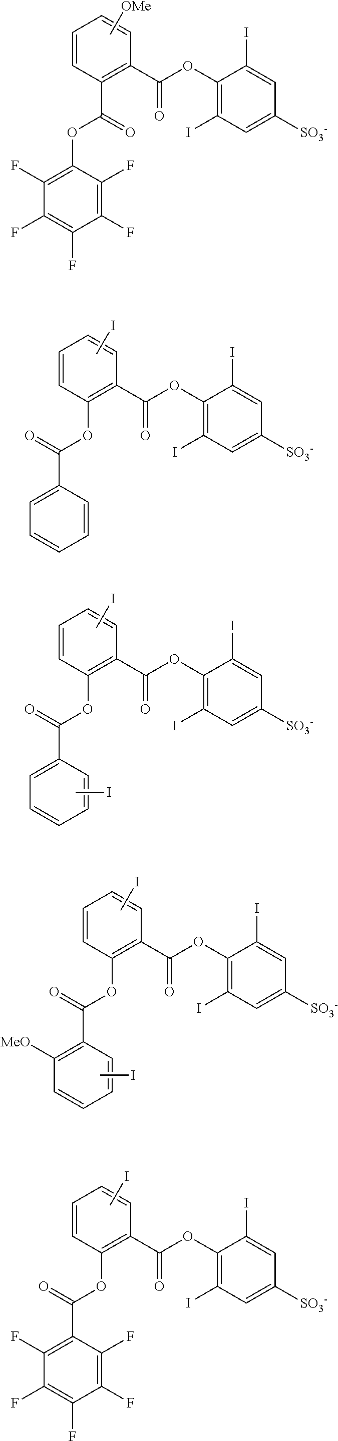

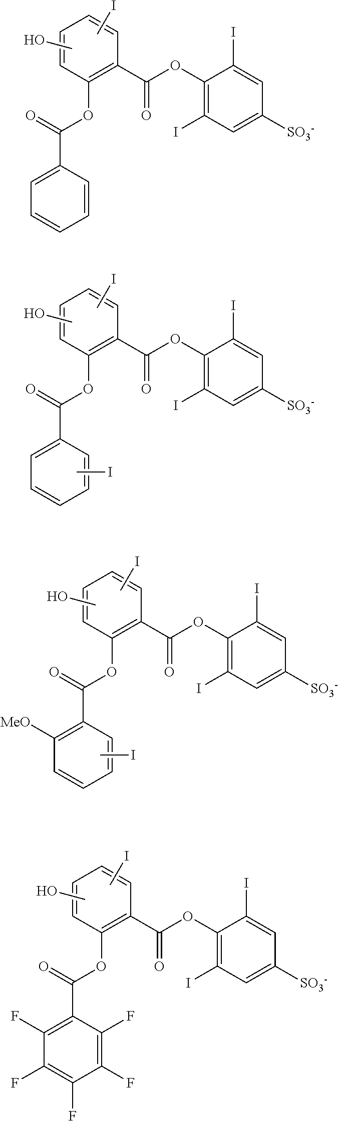

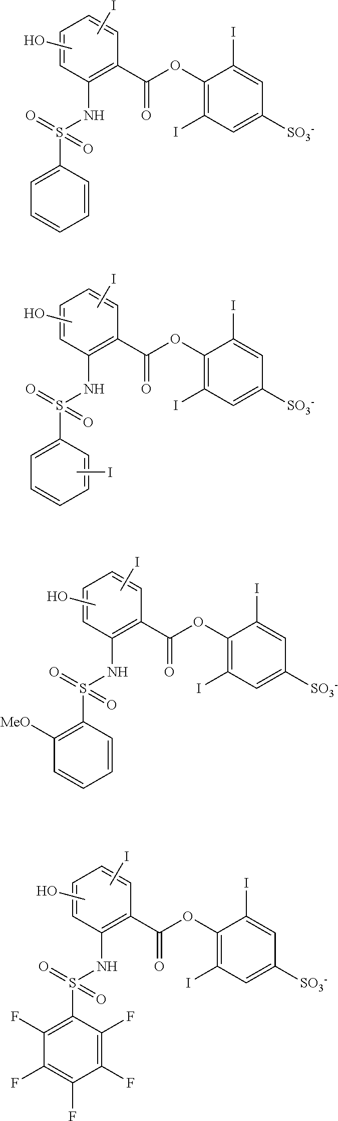

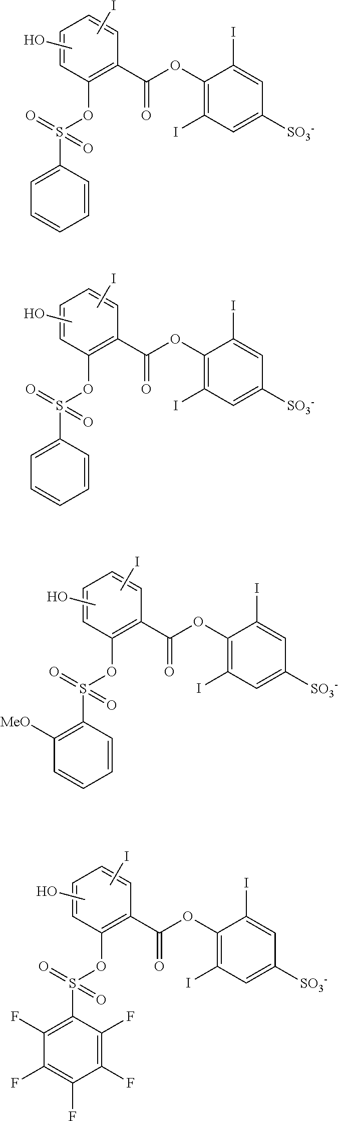

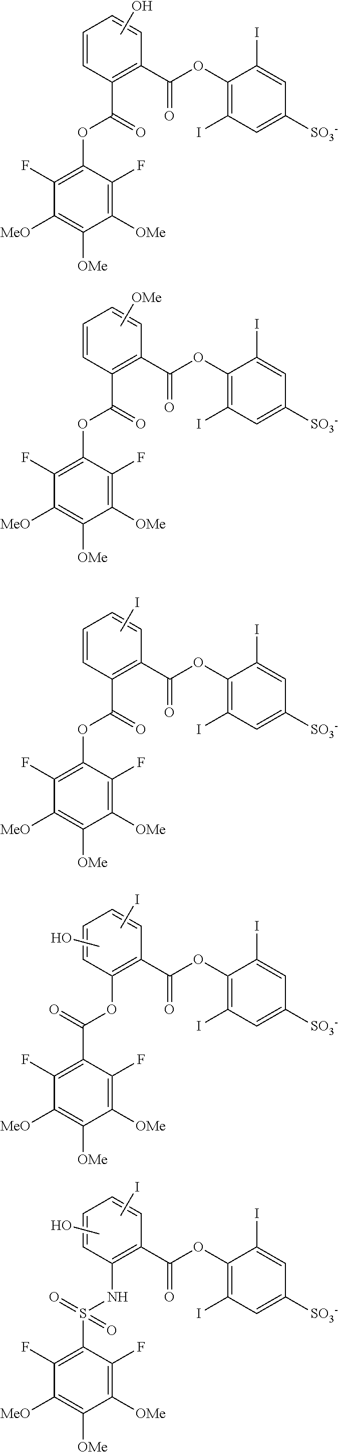

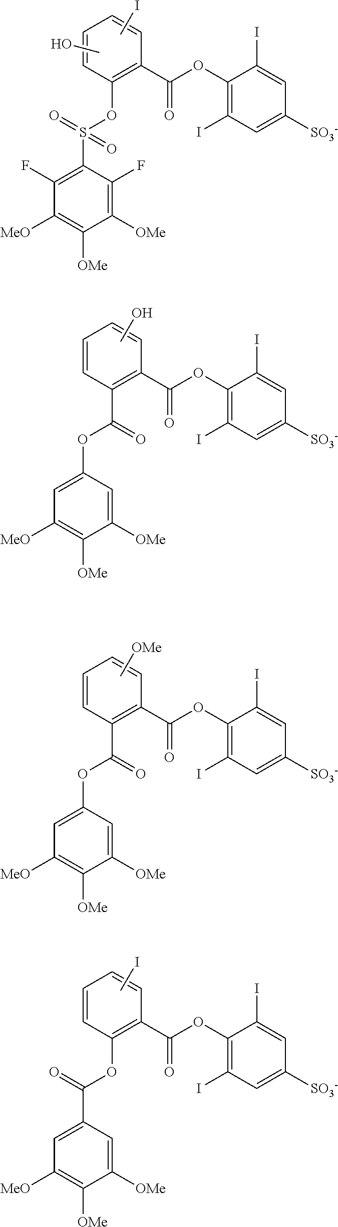

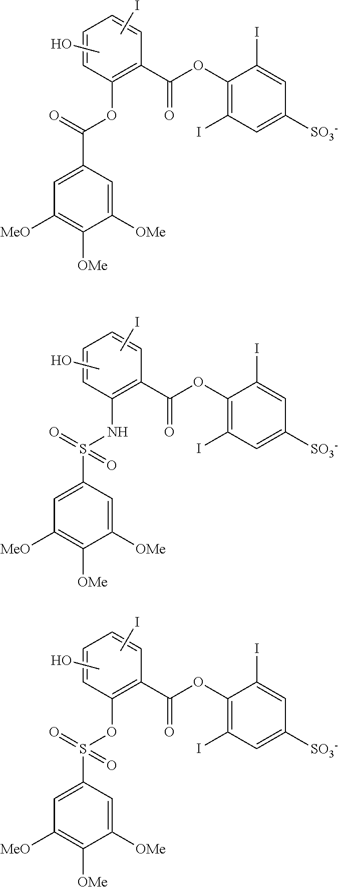

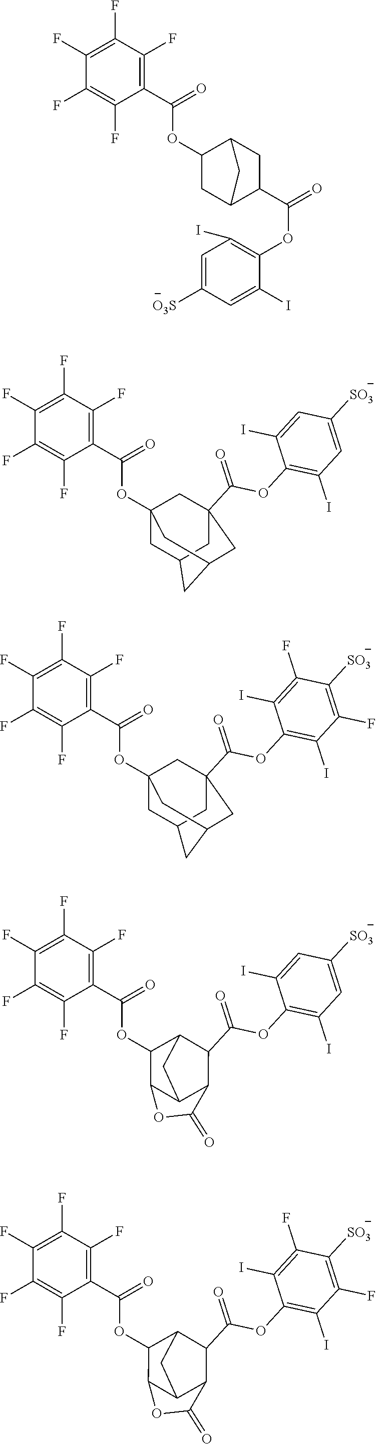

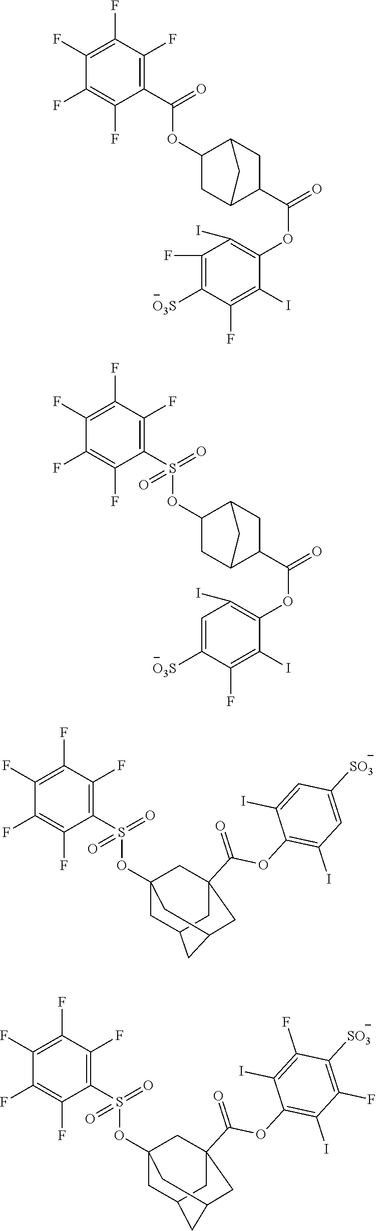

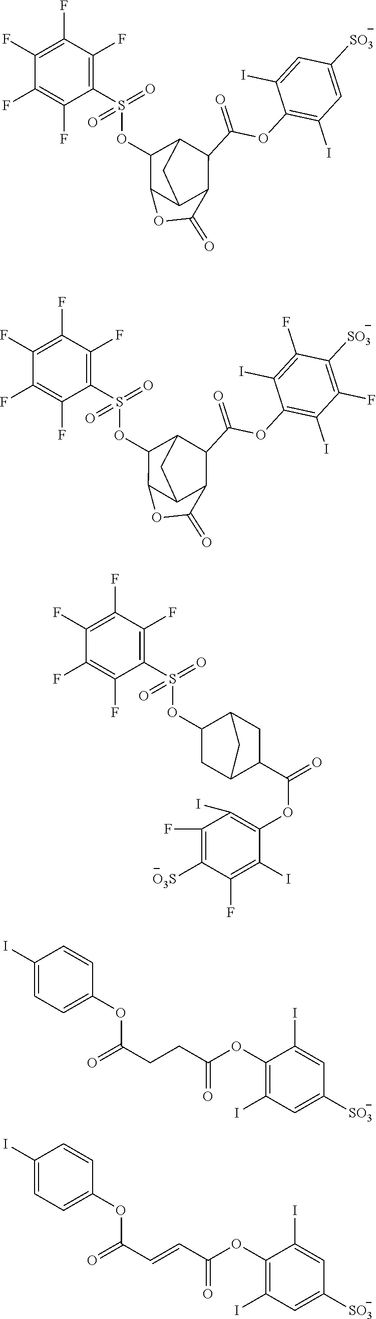

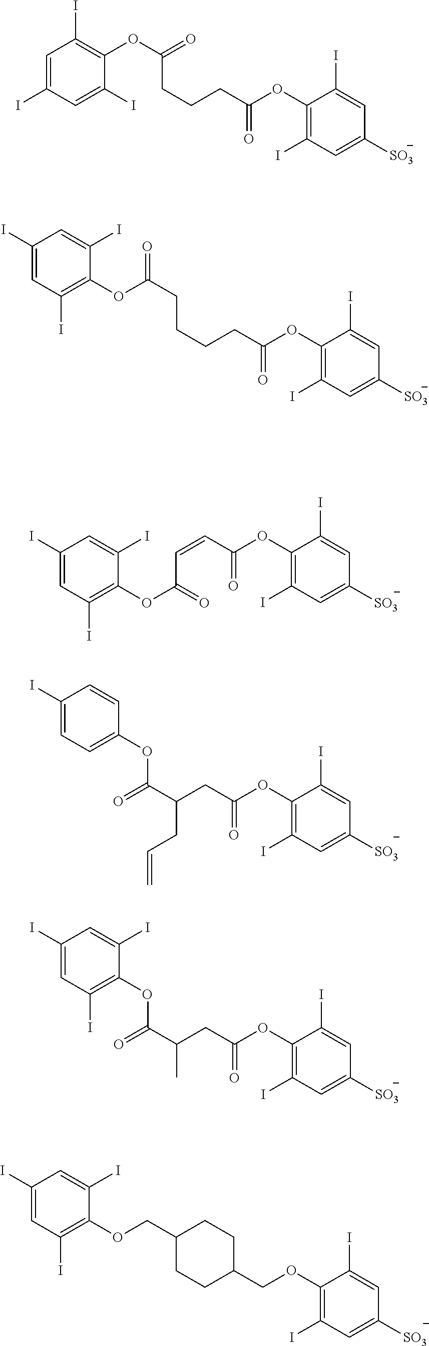

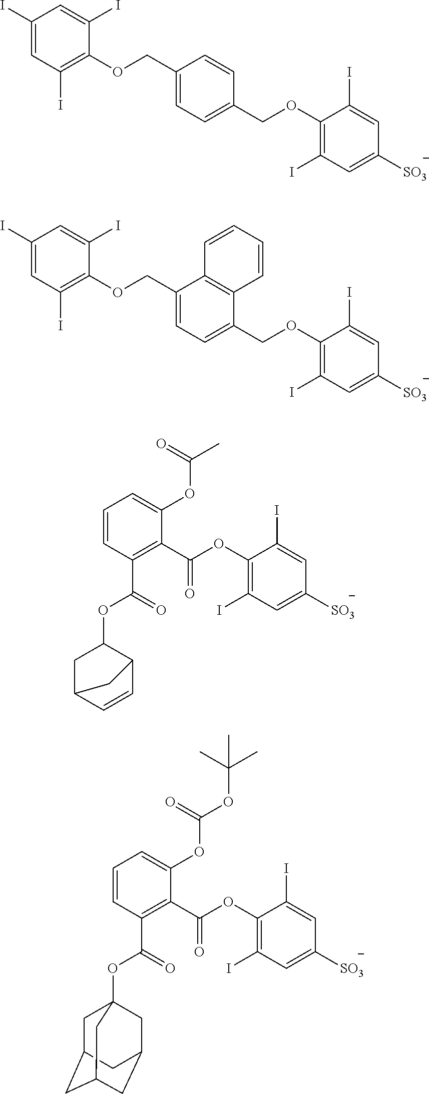

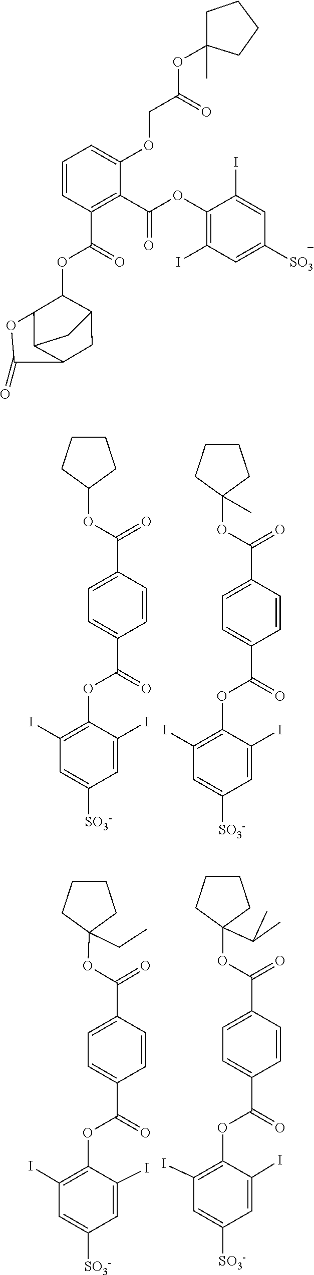

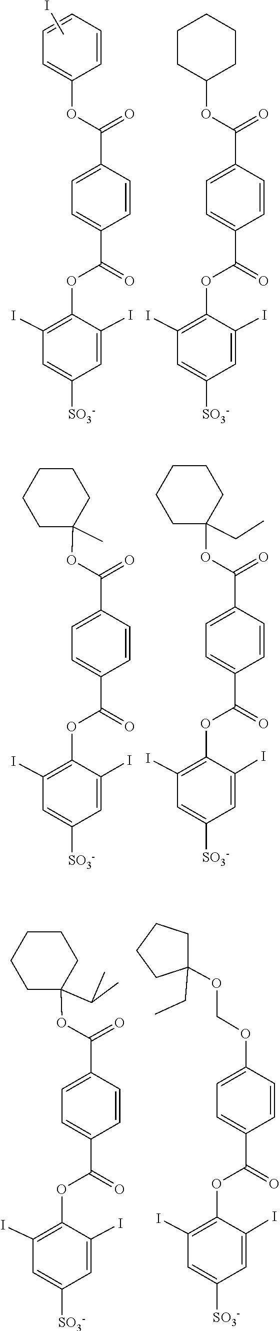

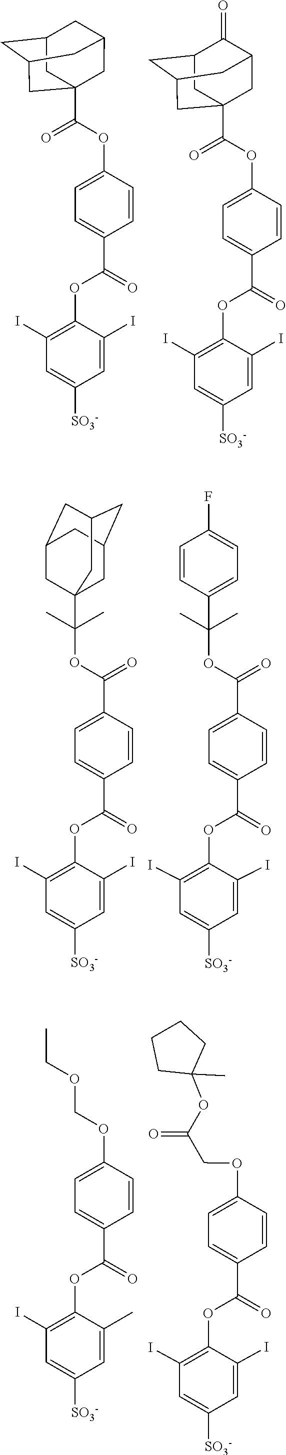

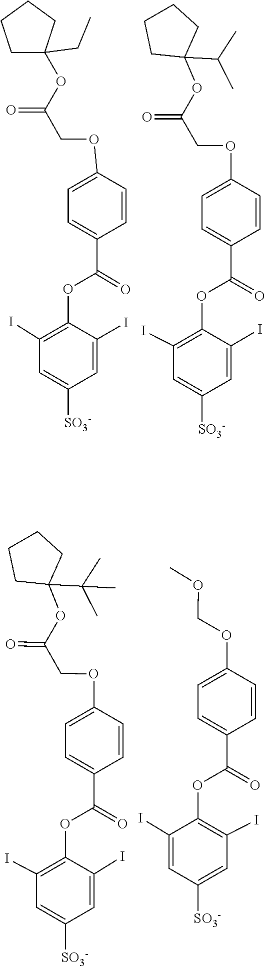

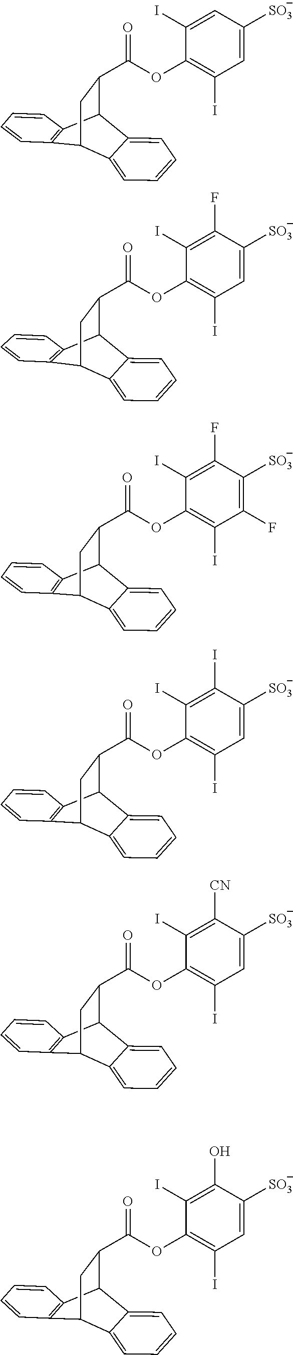

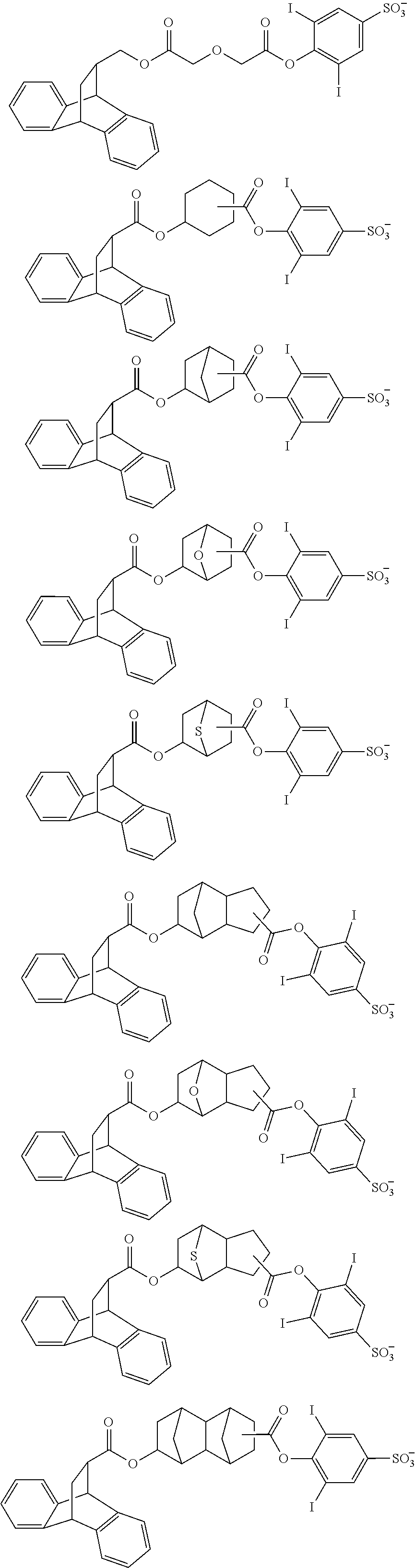

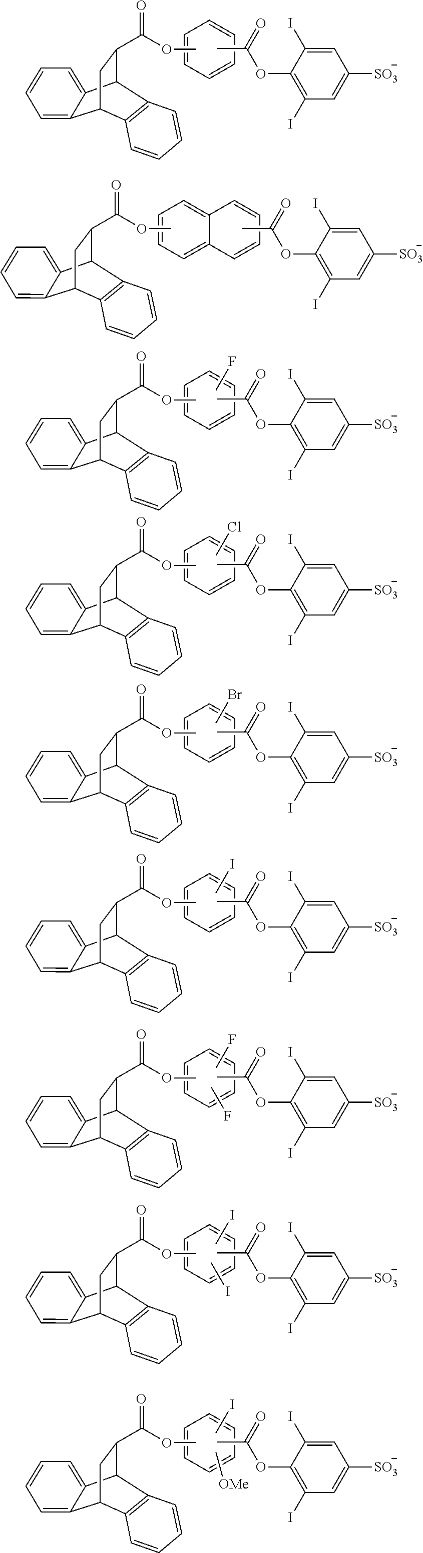

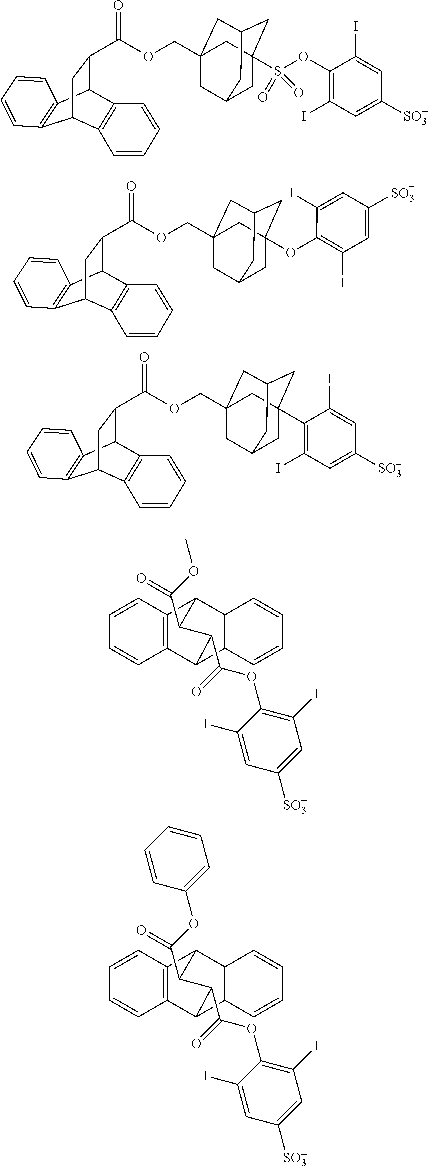

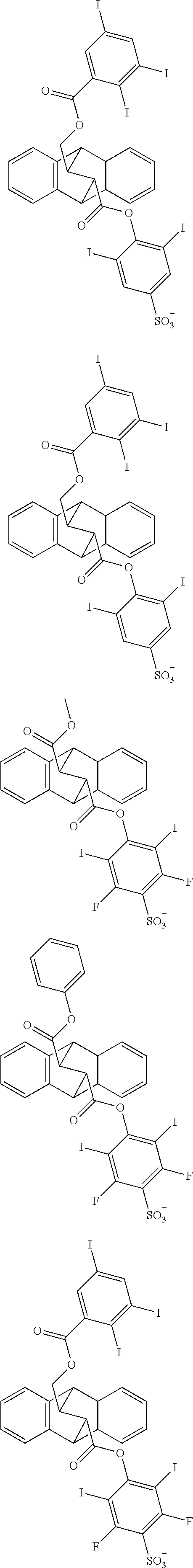

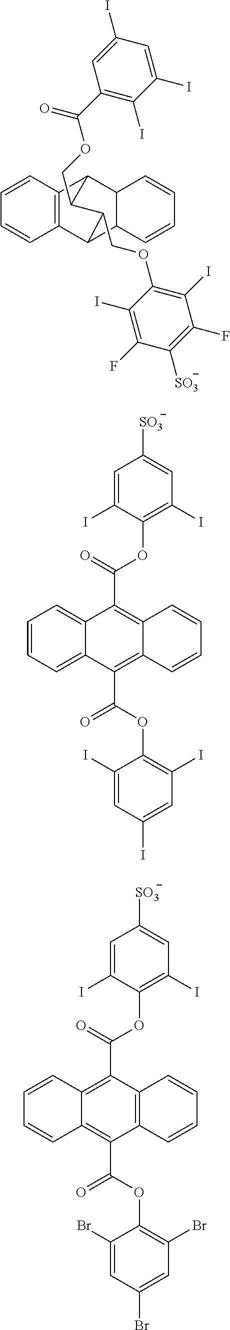

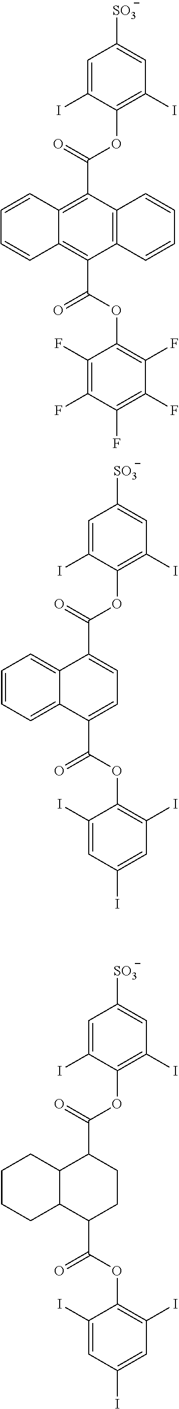

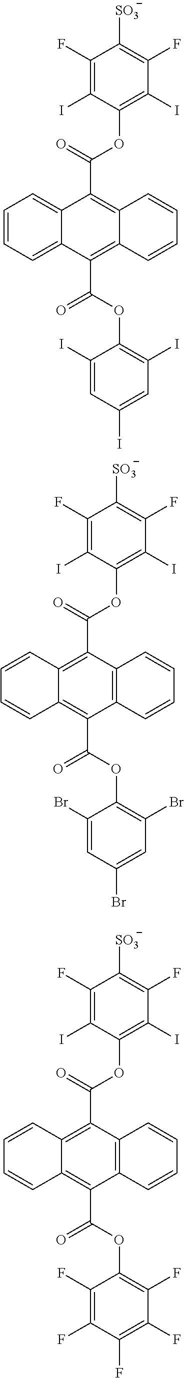

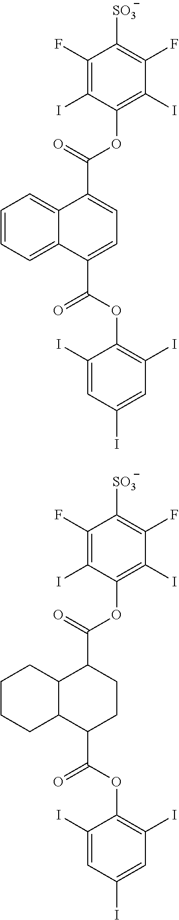

In the formula (A), Z⁺ represents an onium cation. The onium cation is a sulfonium cation represented by the following formula (Z-1) or an iodonium cation represented by the following formula (Z-2).

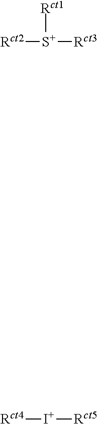

In the formulae (Z-1) and (Z-2), R^{ct1} to R^{ct5} each independently represent a halogen atom or a hydrocarbyl group having 1 to 30 carbon atoms and optionally containing a heteroatom.

Specific examples of the halogen atom represented by R^{ct1} to R^{ct5} include a fluorine atom, a chlorine atom, a bromine atom, and an iodine atom.

The hydrocarbyl group represented by R^{ct1} to R^{ct5} may be saturated or unsaturated, and may be linear, branched, or cyclic. Specific examples thereof include: alkyl groups having 1 to 30 carbon atoms, such as a methyl group, an ethyl group, an n-propyl group, an isopropyl group, an n-butyl group, an isobutyl group, a sec-butyl group, and a tert-butyl group; cyclic saturated hydrocarbyl groups having 3 to 30 carbon atoms, such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; alkenyl groups having 2 to 30 carbon atoms, such as a vinyl group, an allyl group, a propenyl group, a butenyl group, and a hexenyl group; cyclic unsaturated hydrocarbyl groups having 3 to 30 carbon atoms, such as a cyclohexenyl group; aryl groups having 6 to 30 carbon atoms, such as a phenyl group, a naphthyl group, and a thienyl group; aralkyl groups having 7 to 30 carbon atoms, such as a benzyl group, a 1-phenylethyl group, and a 2-phenylethyl group; groups which are combinations of these groups; etc. Aryl groups are preferable. Furthermore, part or all of the hydrogen atoms of the hydrocarbyl group may be substituted with a group containing a heteroatom, such as an oxygen atom, a sulfur atom, a nitrogen atom, and a halogen atom, and part of the —CH₂— of the hydrocarbyl group may be substituted with a group containing a heteroatom such as an oxygen atom, a sulfur atom, or a nitrogen atom. The resulting hydrocarbyl group may contain a hydroxy group, a fluorine atom, a chlorine atom, a bromine atom, an iodine atom, a cyano group, a nitro group, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, etc.

Furthermore, R^{ct1} and R^{ct2} may be bonded to each other to form a ring together with the sulfur atom bonded thereto. In this case, specific examples of the structure of the ring include ones represented by the following formulae.

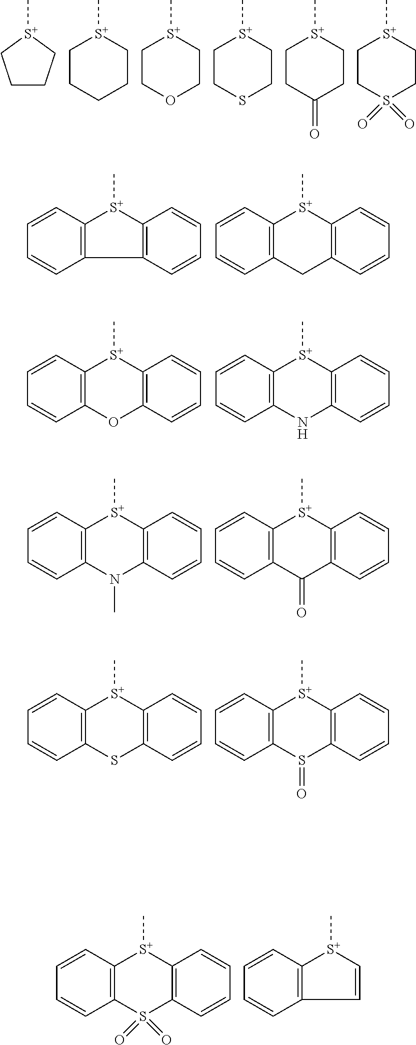

In the formulae, a broken line represents an attachment point to R^{ct3}.

Specific examples of the sulfonium cation represented by the formula (Z-1) include ones shown below, but are not limited thereto.

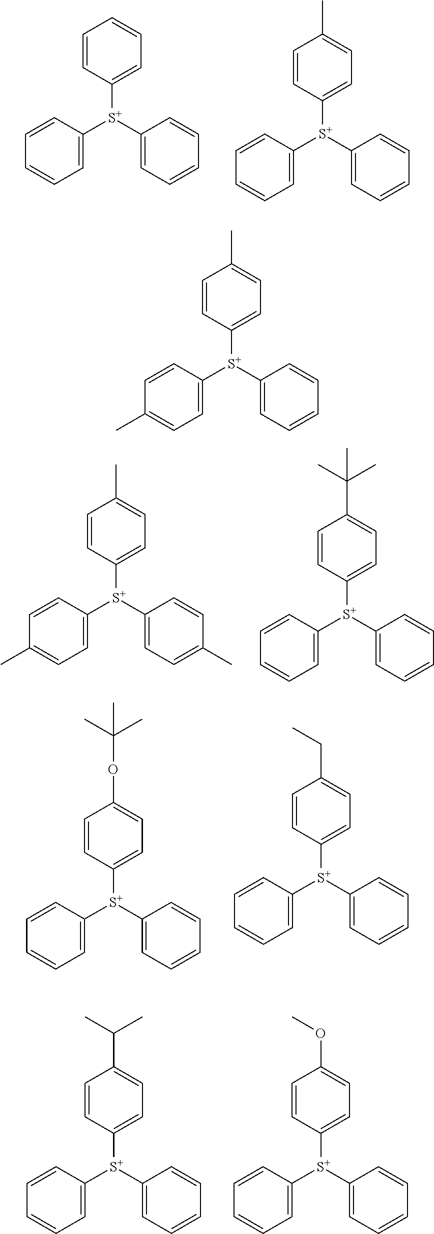

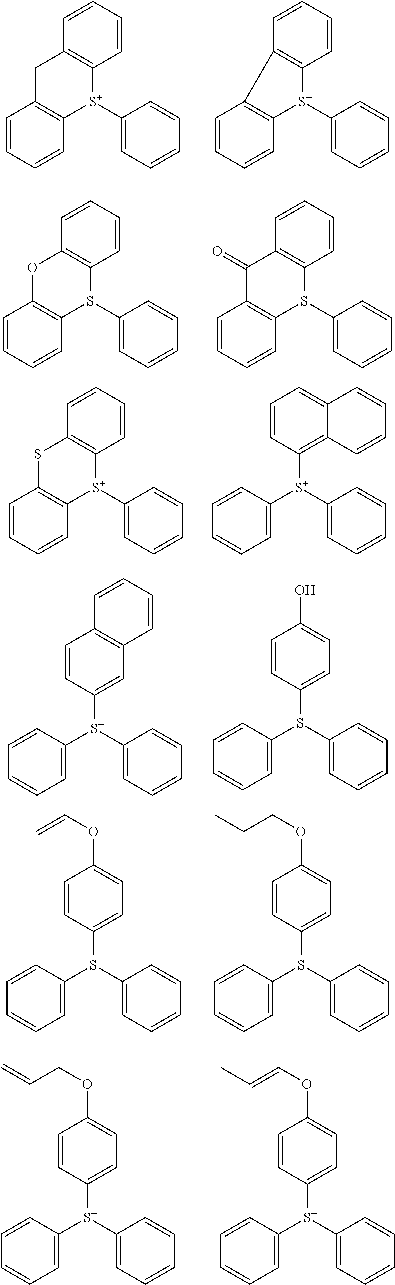

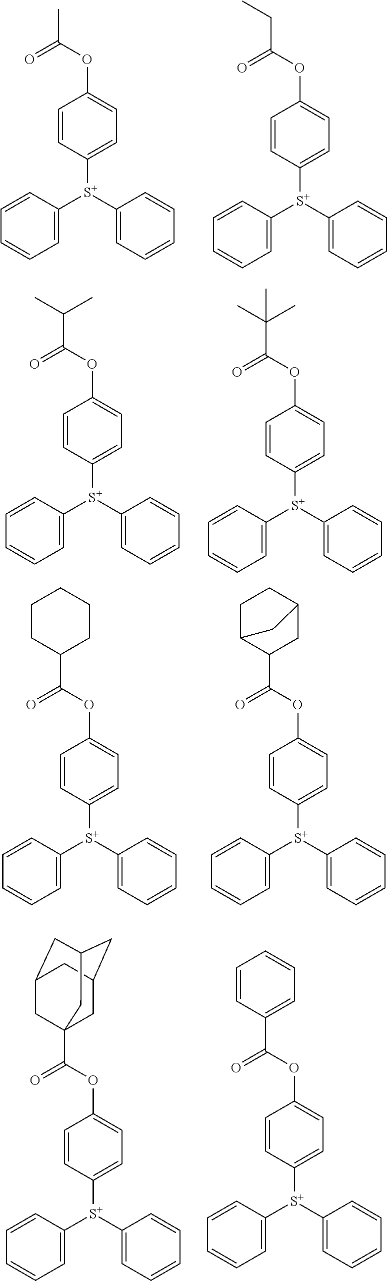

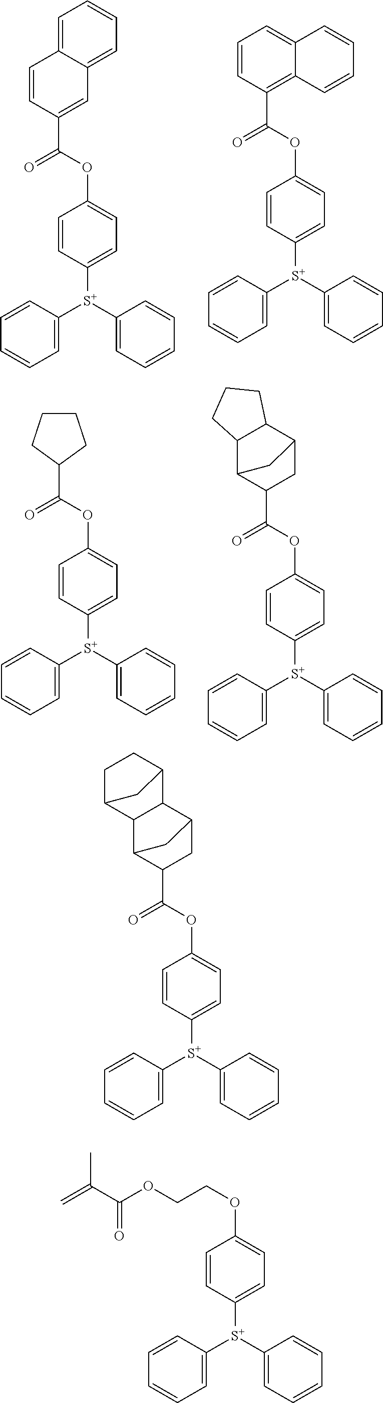 [SMILES: O=C(Oc1ccc([S+](c2ccccc2)c2ccccc2)cc1)c1ccc2ccccc2c1 ; O=C(Oc1ccc([S+](c2ccccc2)c2ccccc2)cc1)c1cccc2ccccc12 ; O=C(Oc1ccc([S+](c2ccccc2)c2ccccc2)cc1)C1CCCC1 ; O=C(Oc1ccc([S+](c2ccccc2)c2ccccc2)cc1)C1CC2CC1C1CCCC21 ; O=C(Oc1ccc([S+](c2ccccc2)c2ccccc2)cc1)C1CC2CC1C1C3CCC(C3)C21 ; C=C(C)C(=O)OCCOc1ccc([S+](c2ccccc2)c2ccccc2)cc1]

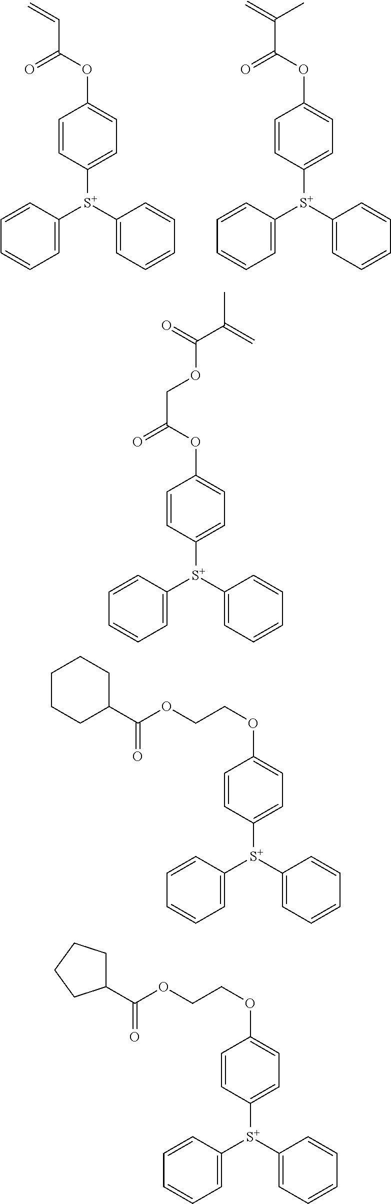 [SMILES: C=CC(=O)Oc1ccc([S+](c2ccccc2)c2ccccc2)cc1 ; C=C(C)C(=O)Oc1ccc([S+](c2ccccc2)c2ccccc2)cc1 ; C=C(C)C(=O)OCC(=O)Oc1ccc([S+](c2ccccc2)c2ccccc2)cc1 ; O=C(OCCOc1ccc([S+](c2ccccc2)c2ccccc2)cc1)C1CCCCC1 ; O=C(OCCOc1ccc([S+](c2ccccc2)c2ccccc2)cc1)C1CCCC1]

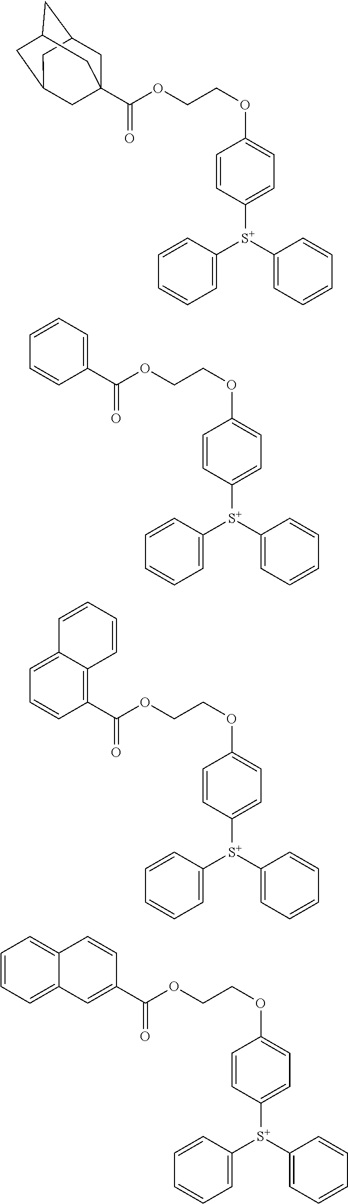 [SMILES: O=C(OCCOc1ccc([S+](c2ccccc2)c2ccccc2)cc1)C12CC3CC(CC(C3)C1)C2 ; O=C(OCCOc1ccc([S+](c2ccccc2)c2ccccc2)cc1)c1ccccc1 ; O=C(OCCOc1ccc([S+](c2ccccc2)c2ccccc2)cc1)c1cccc2ccccc12 ; O=C(OCCOc1ccc([S+](c2ccccc2)c2ccccc2)cc1)c1ccc2ccccc2c1]

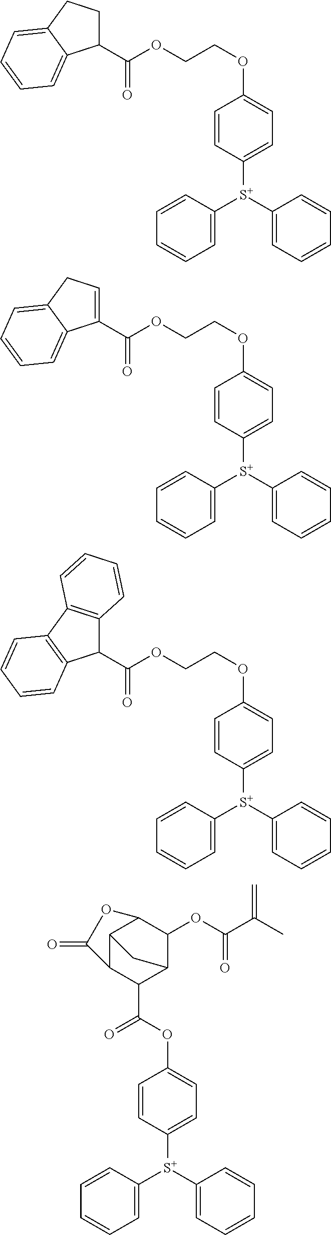 [SMILES: O=C(OCCOc1ccc([S+](c2ccccc2)c2ccccc2)cc1)C1CCc2ccccc21 ; O=C(OCCOc1ccc([S+](c2ccccc2)c2ccccc2)cc1)C1=CCc2ccccc21 ; O=C(OCCOc1ccc([S+](c2ccccc2)c2ccccc2)cc1)C1c2ccccc2-c2ccccc21 ; C=C(C)C(=O)OC1C2CC3C1OC(=O)C3C2C(=O)Oc1ccc([S+](c2ccccc2)c2ccccc2)cc1]

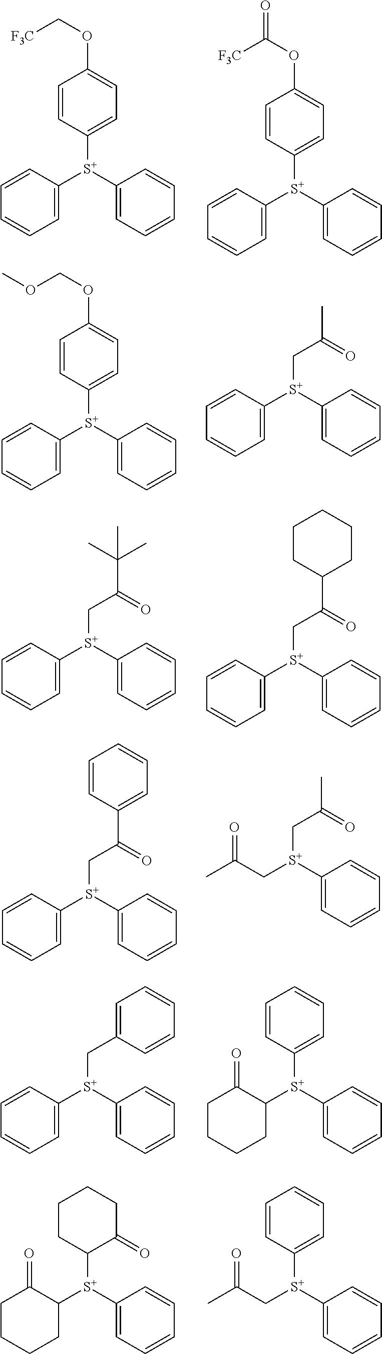

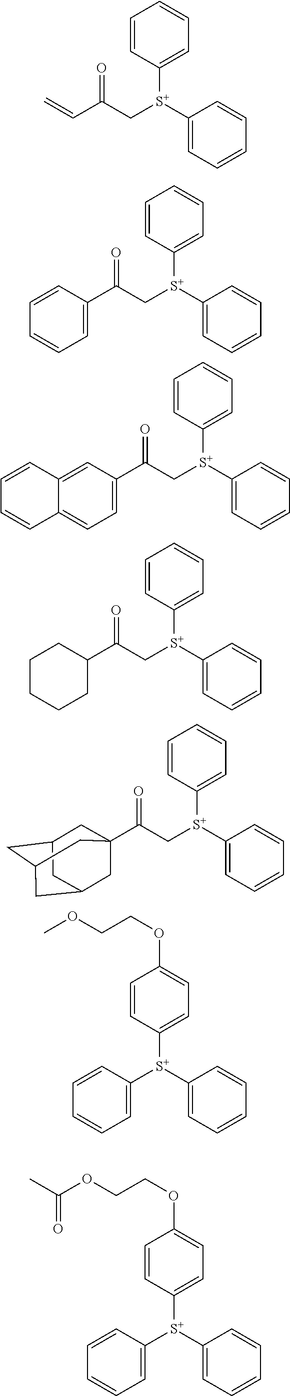

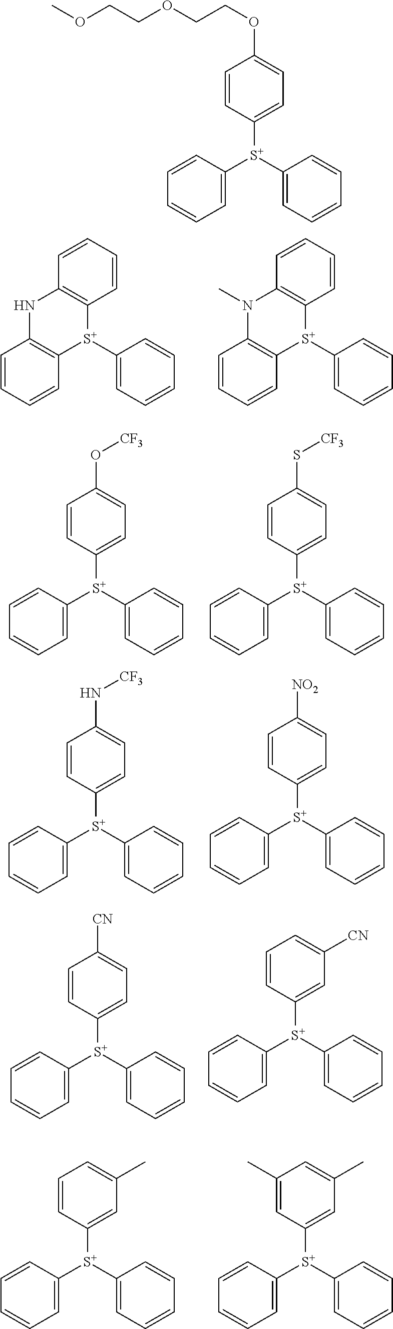

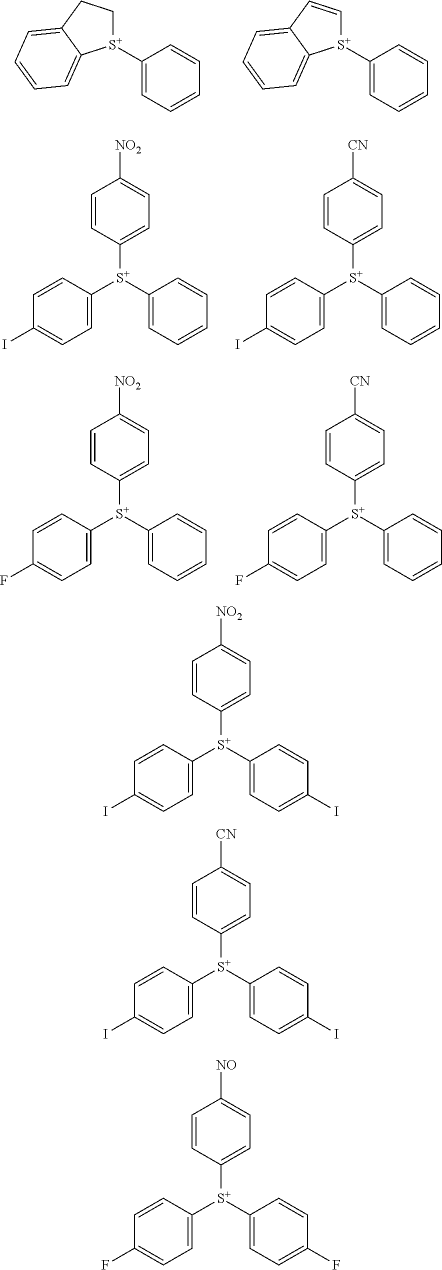

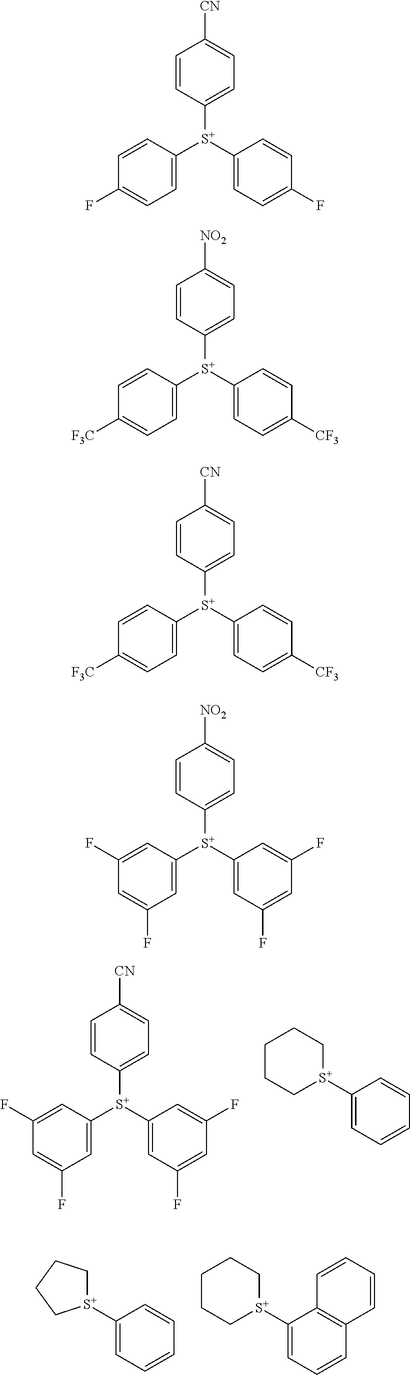

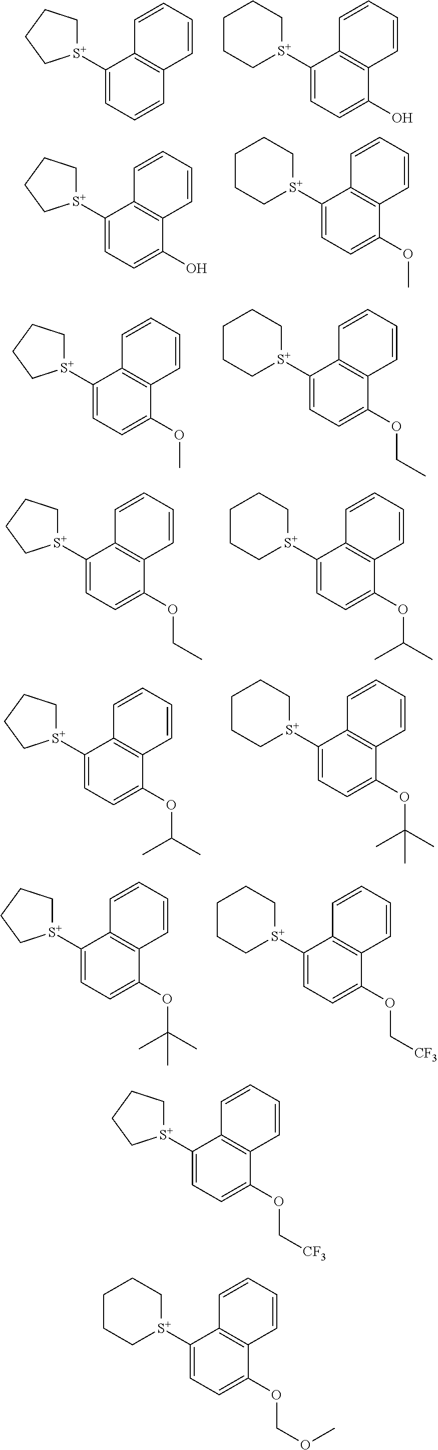

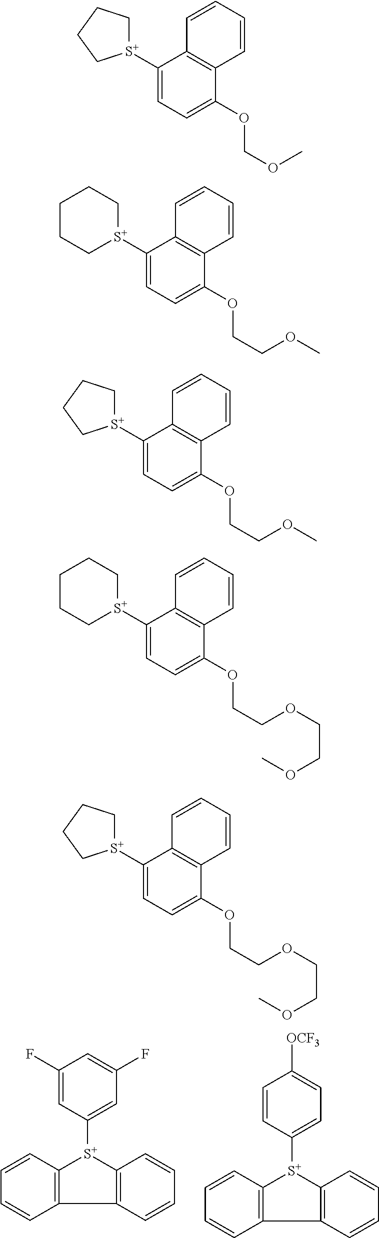

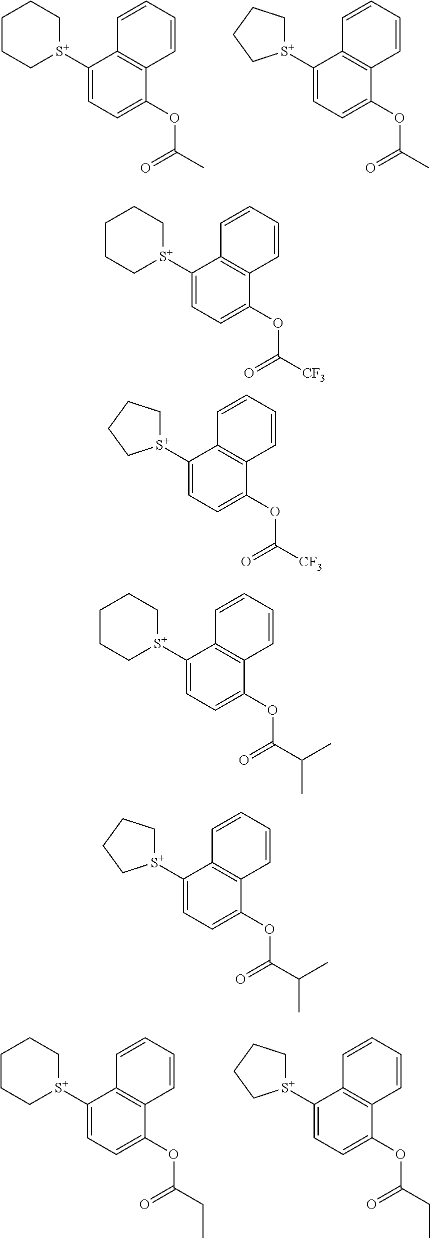

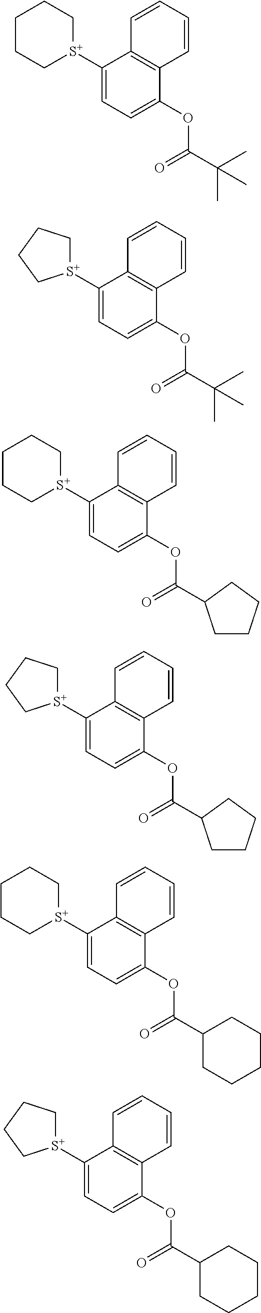 [SMILES: CC(C)(C)C(=O)Oc1ccc([S+]2CCCCC2)c2ccccc12 ; CC(C)(C)C(=O)Oc1ccc([S+]2CCCC2)c2ccccc12 ; O=C(Oc1ccc([S+]2CCCCC2)c2ccccc12)C1CCCC1 ; O=C(Oc1ccc([S+]2CCCC2)c2ccccc12)C1CCCC1 ; O=C(Oc1ccc([S+]2CCCCC2)c2ccccc12)C1CCCCC1 ; O=C(Oc1ccc([S+]2CCCC2)c2ccccc12)C1CCCCC1]

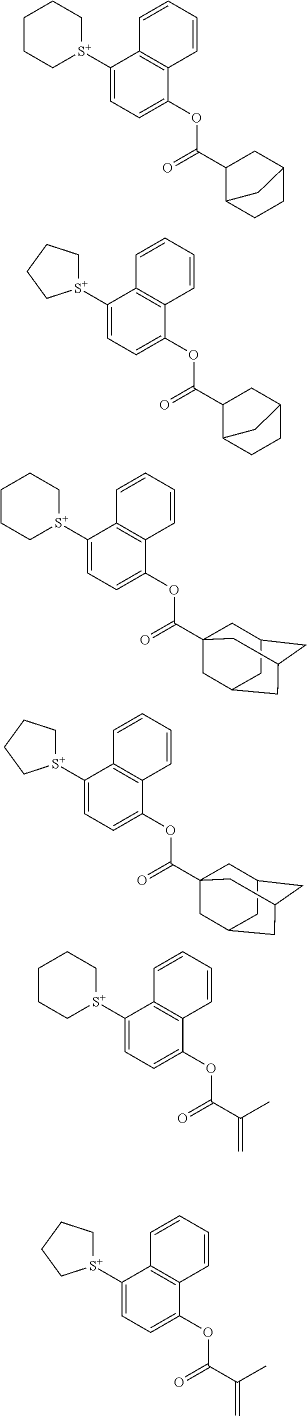 [SMILES: O=C(Oc1ccc([S+]2CCCCC2)c2ccccc12)C1CC2CCC1C2 ; O=C(Oc1ccc([S+]2CCCC2)c2ccccc12)C1CC2CCC1C2 ; O=C(Oc1ccc([S+]2CCCCC2)c2ccccc12)C12CC3CC(CC(C3)C1)C2 ; O=C(Oc1ccc([S+]2CCCC2)c2ccccc12)C12CC3CC(CC(C3)C1)C2 ; C=C(C)C(=O)Oc1ccc([S+]2CCCCC2)c2ccccc12 ; C=C(C)C(=O)Oc1ccc([S+]2CCCC2)c2ccccc12]

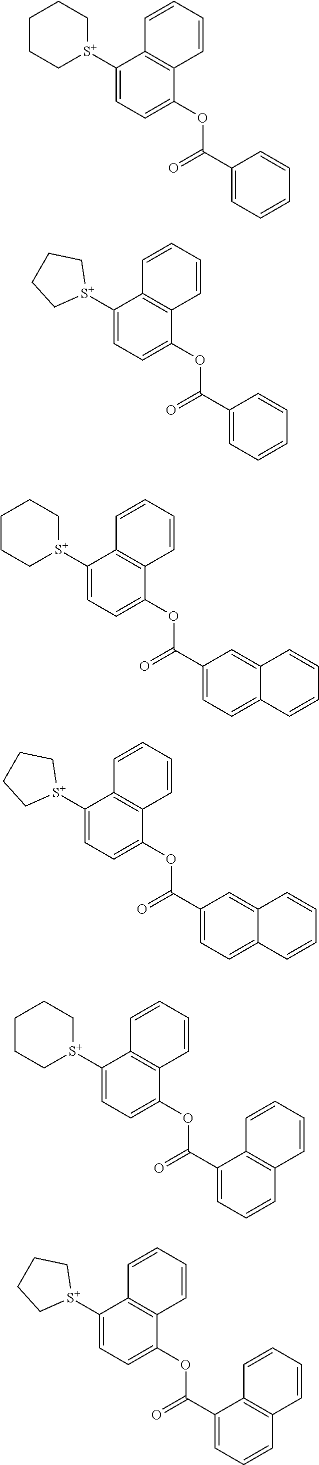 [SMILES: O=C(Oc1ccc([S+]2CCCCC2)c2ccccc12)c1ccccc1 ; O=C(Oc1ccc([S+]2CCCC2)c2ccccc12)c1ccccc1 ; O=C(Oc1ccc([S+]2CCCCC2)c2ccccc12)c1ccc2ccccc2c1 ; O=C(Oc1ccc([S+]2CCCC2)c2ccccc12)c1ccc2ccccc2c1 ; O=C(Oc1ccc([S+]2CCCCC2)c2ccccc12)c1cccc2ccccc12 ; O=C(Oc1ccc([S+]2CCCC2)c2ccccc12)c1cccc2ccccc12]

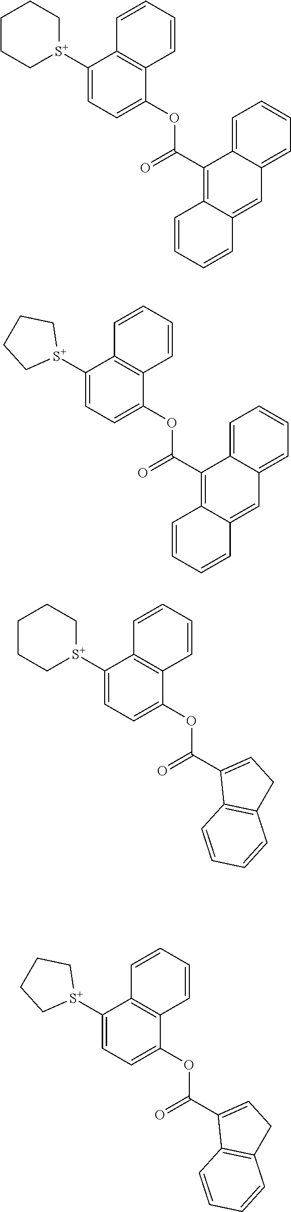 [SMILES: O=C(Oc1ccc([S+]2CCCCC2)c2ccccc12)c1c2ccccc2cc2ccccc12 ; O=C(Oc1ccc([S+]2CCCC2)c2ccccc12)c1c2ccccc2cc2ccccc12 ; O=C(Oc1ccc([S+]2CCCCC2)c2ccccc12)C1=CCc2ccccc21 ; O=C(Oc1ccc([S+]2CCCC2)c2ccccc12)C1=CCc2ccccc21]

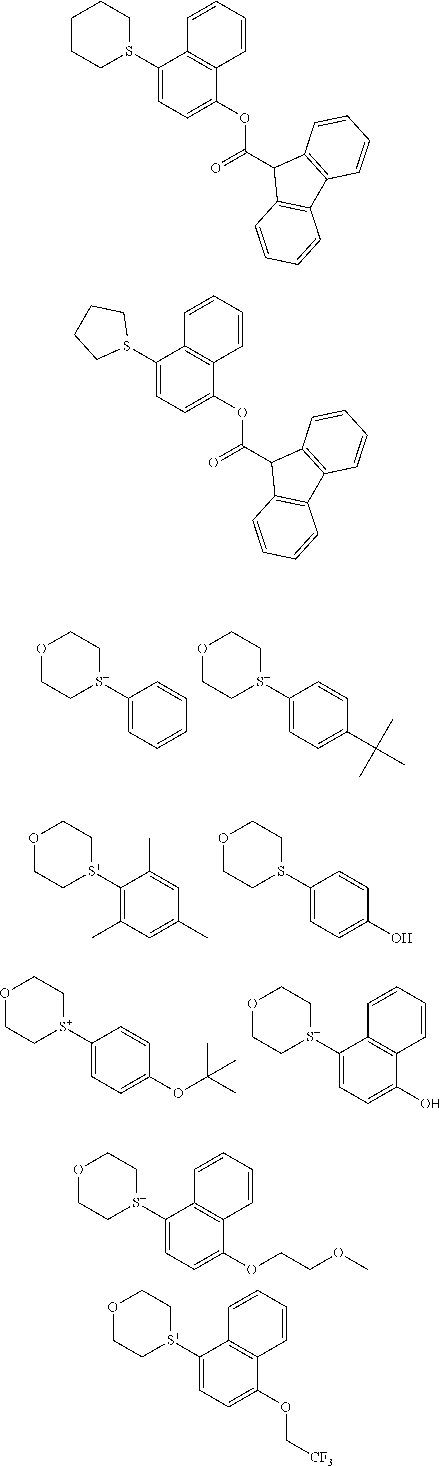

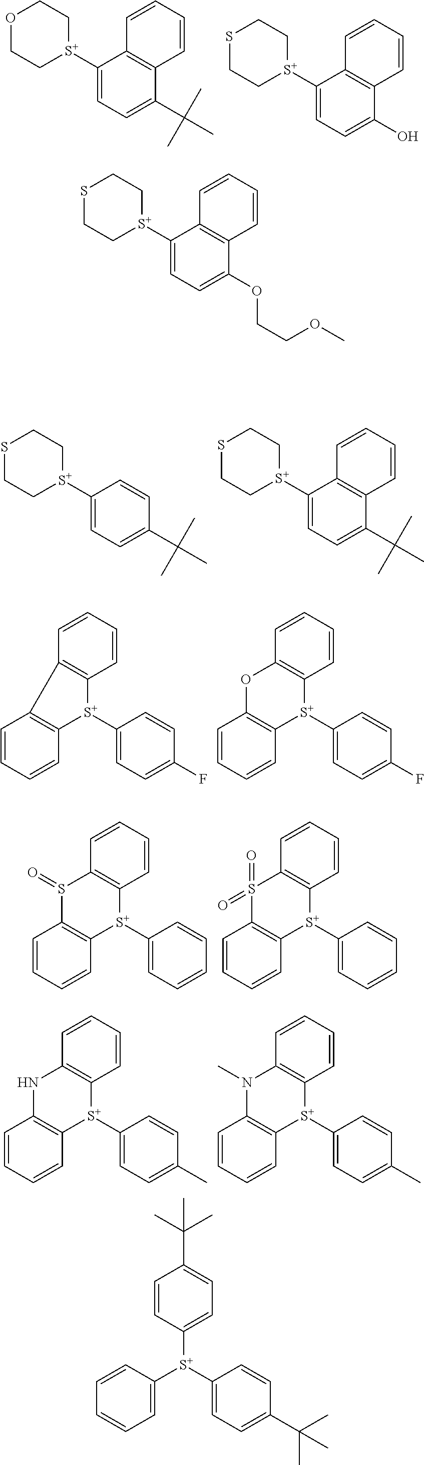

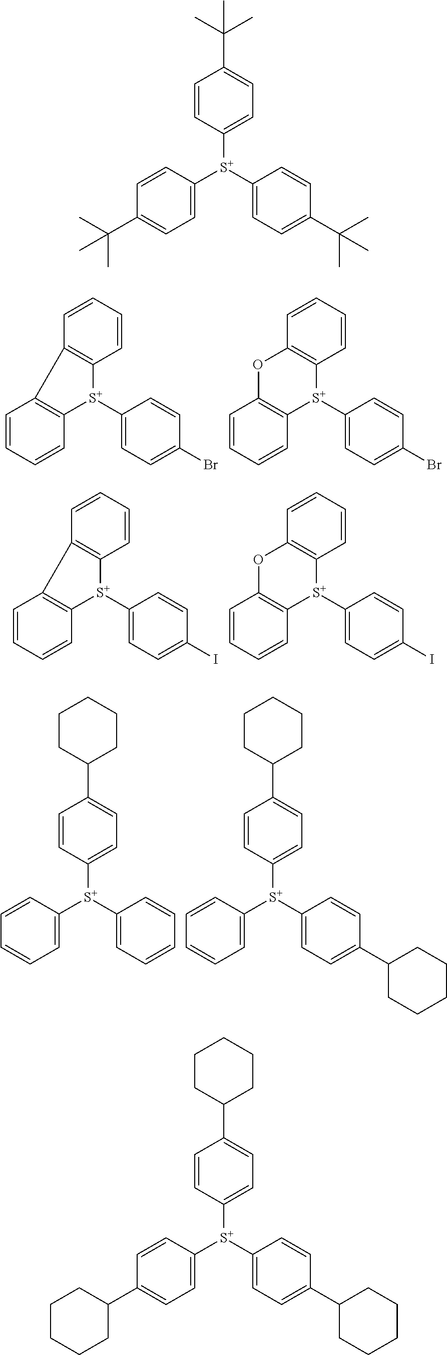

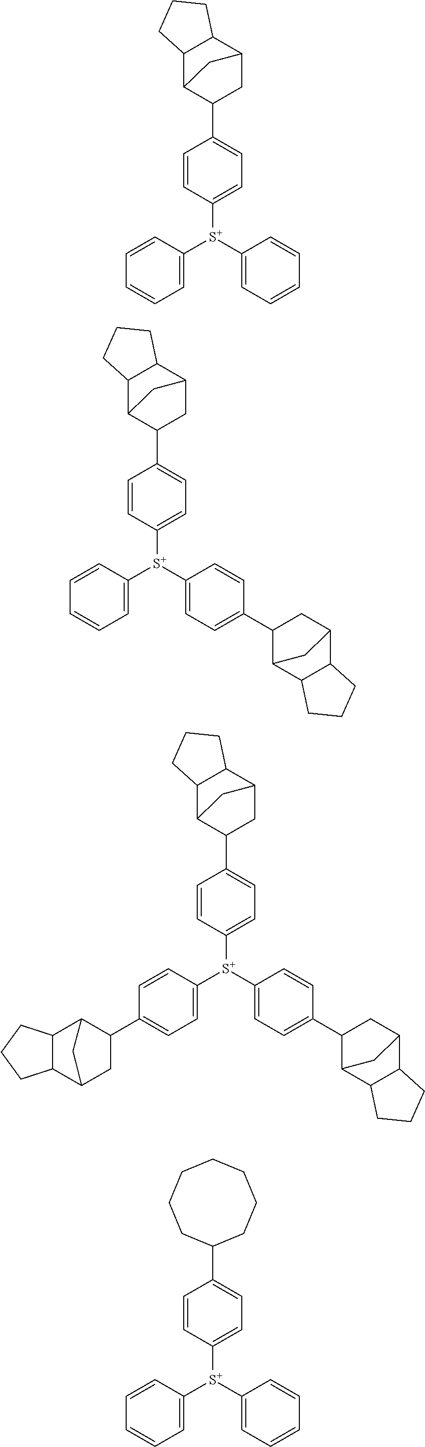 [SMILES: c1ccc([S+](c2ccccc2)c2ccc(C3CC4CC3C3CCCC43)cc2)cc1 ; c1ccc([S+](c2ccc(C3CC4CC3C3CCCC43)cc2)c2ccc(C3CC4CC3C3CCCC43)cc2)cc1 ; c1cc([S+](c2ccc(C3CC4CC3C3CCCC43)cc2)c2ccc(C3CC4CC3C3CCCC43)cc2)ccc1C1CC2CC1C1CCCC21 ; c1ccc([S+](c2ccccc2)c2ccc(C3CCCCCCC3)cc2)cc1]

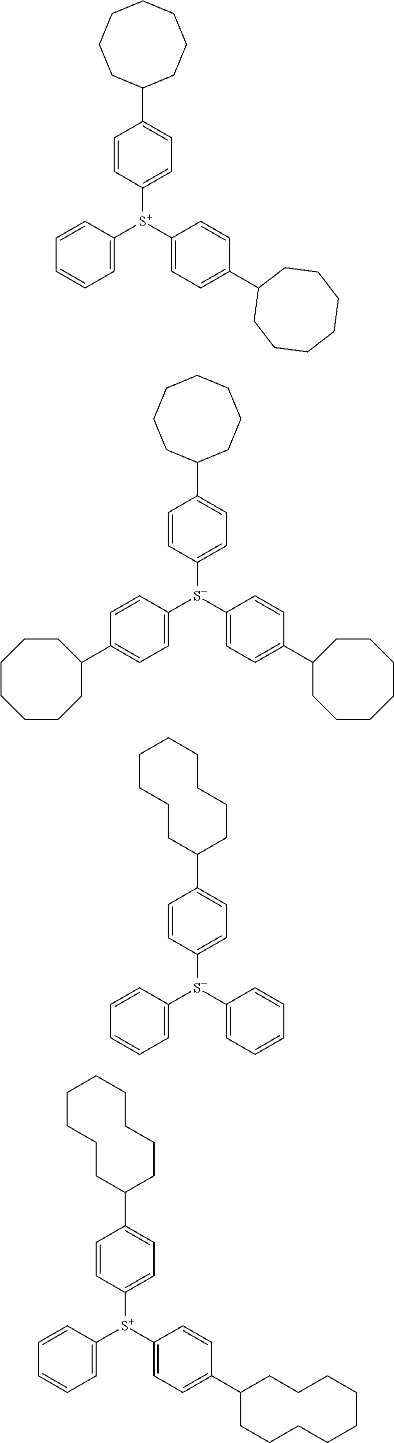 [SMILES: c1ccc([S+](c2ccc(C3CCCCCCC3)cc2)c2ccc(C3CCCCCCC3)cc2)cc1 ; c1cc([S+](c2ccc(C3CCCCCCC3)cc2)c2ccc(C3CCCCCCC3)cc2)ccc1C1CCCCCCC1 ; c1ccc([S+](c2ccccc2)c2ccc(C3CCCCCCCCC3)cc2)cc1 ; c1ccc([S+](c2ccc(C3CCCCCCCCC3)cc2)c2ccc(C3CCCCCCCCC3)cc2)cc1]

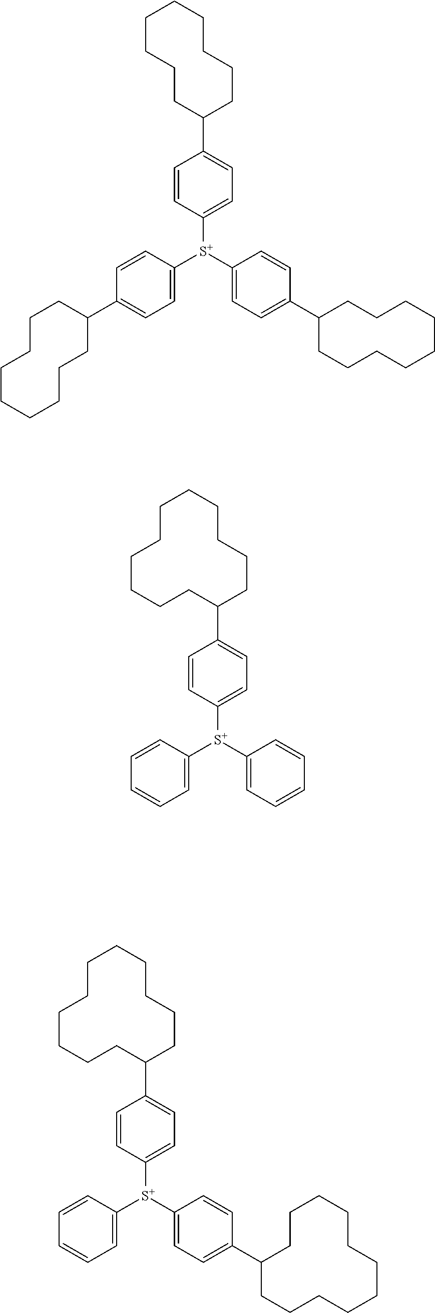 [SMILES: c1cc([S+](c2ccc(C3CCCCCCCCC3)cc2)c2ccc(C3CCCCCCCCC3)cc2)ccc1C1CCCCCCCCC1 ; c1ccc([S+](c2ccccc2)c2ccc(C3CCCCCCCCCCC3)cc2)cc1 ; c1ccc([S+](c2ccc(C3CCCCCCCCCCC3)cc2)c2ccc(C3CCCCCCCCCCC3)cc2)cc1]

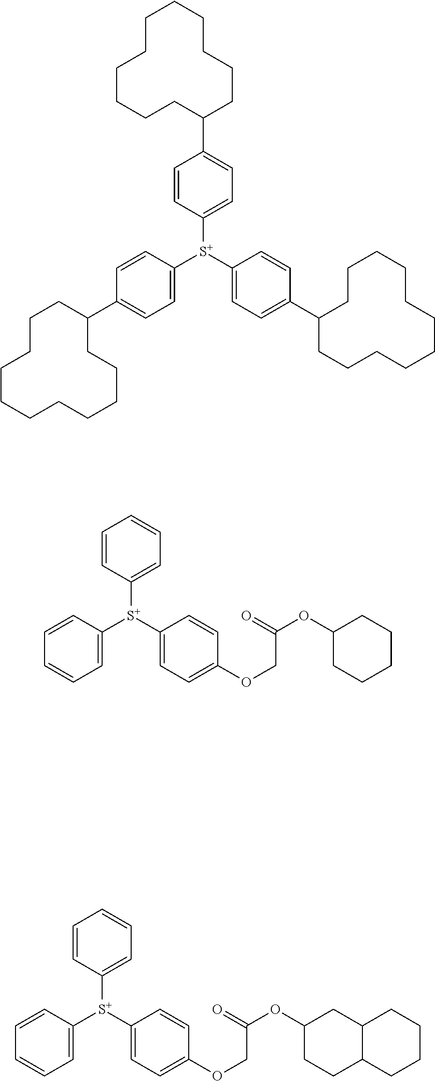 [SMILES: c1cc([S+](c2ccc(C3CCCCCCCCCCC3)cc2)c2ccc(C3CCCCCCCCCCC3)cc2)ccc1C1CCCCCCCCCCC1 ; O=C(COc1ccc([S+](c2ccccc2)c2ccccc2)cc1)OC1CCCCC1 ; O=C(COc1ccc([S+](c2ccccc2)c2ccccc2)cc1)OC1CCC2CCCCC2C1]

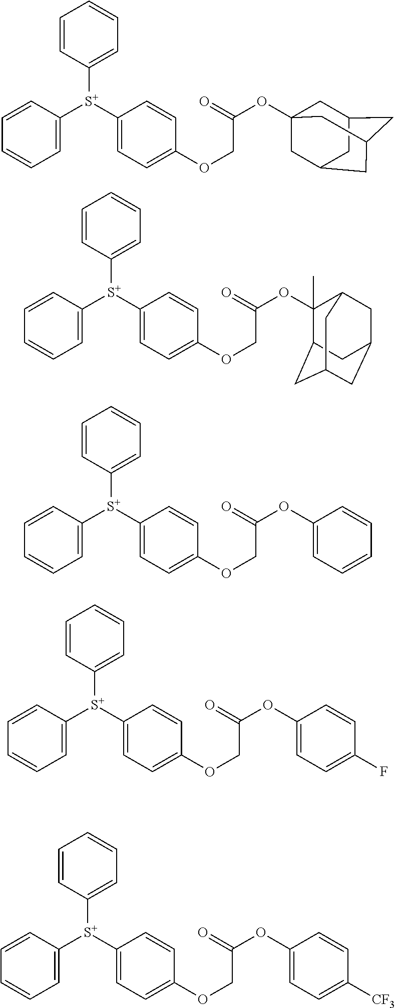 [SMILES: O=C(COc1ccc([S+](c2ccccc2)c2ccccc2)cc1)OC12CC3CC(CC(C3)C1)C2 ; CC1(OC(=O)COc2ccc([S+](c3ccccc3)c3ccccc3)cc2)C2CC3CC(C2)CC1C3 ; O=C(COc1ccc([S+](c2ccccc2)c2ccccc2)cc1)Oc1ccccc1 ; O=C(COc1ccc([S+](c2ccccc2)c2ccccc2)cc1)Oc1ccc(F)cc1 ; O=C(COc1ccc([S+](c2ccccc2)c2ccccc2)cc1)Oc1ccc(C(F)(F)F)cc1]

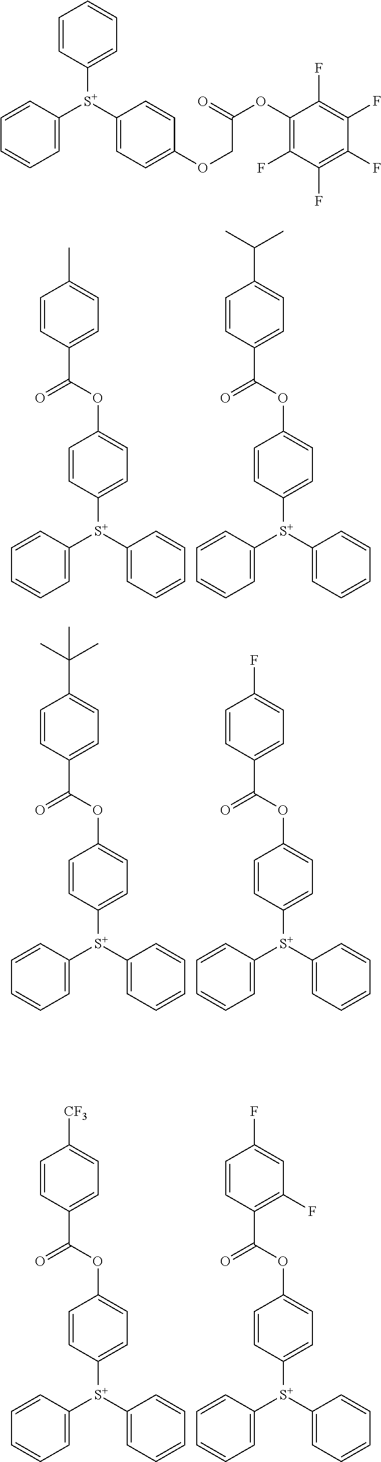

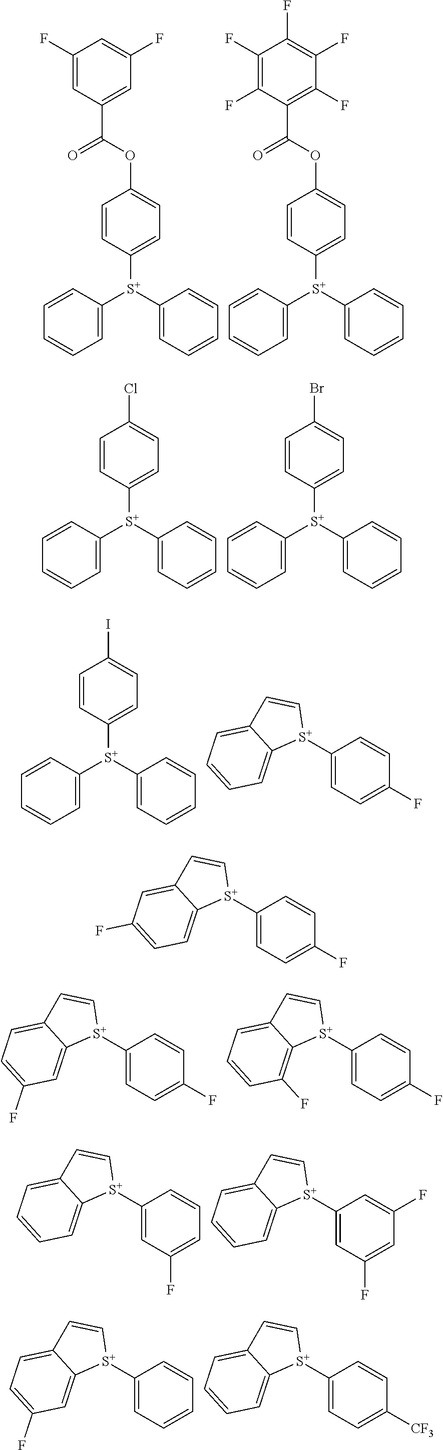

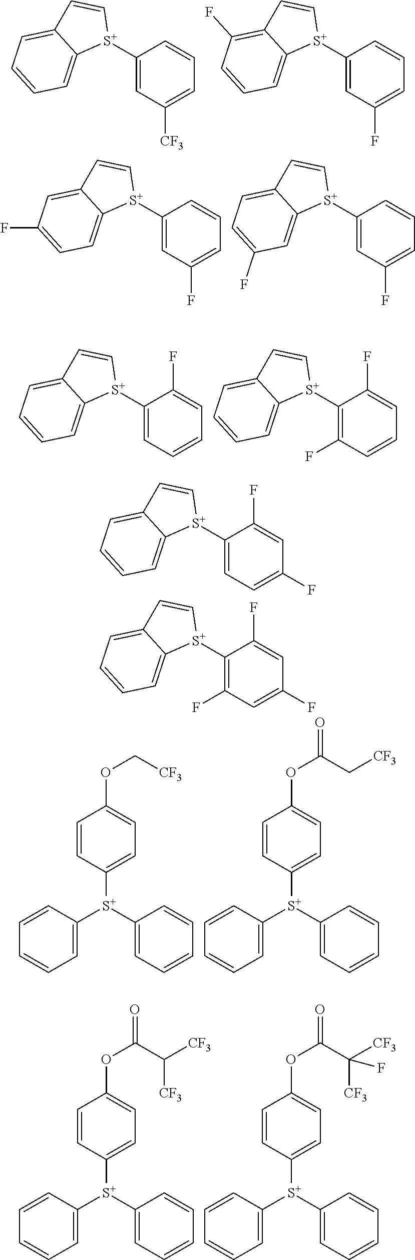

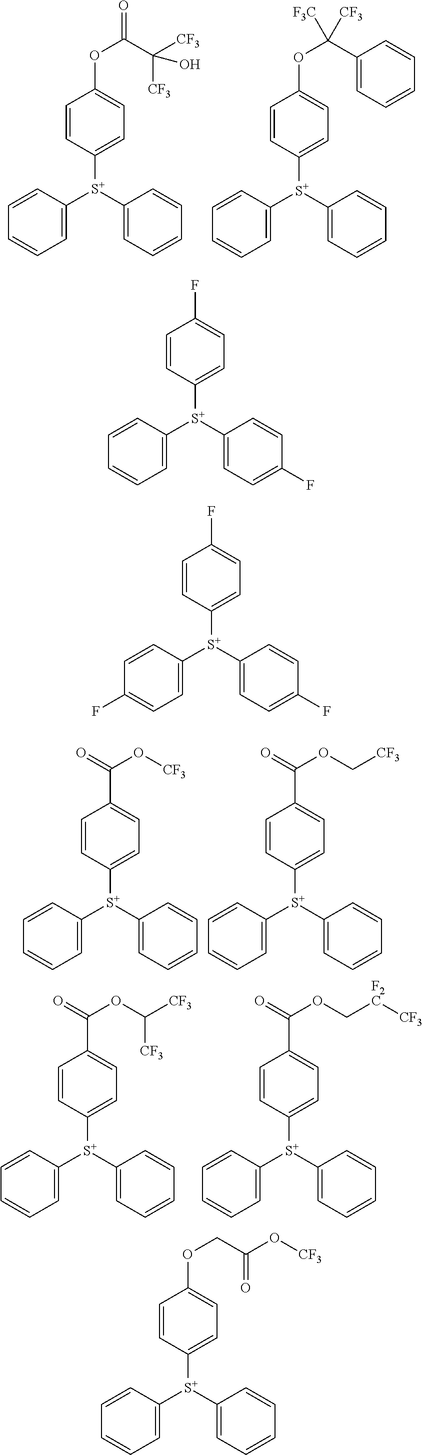

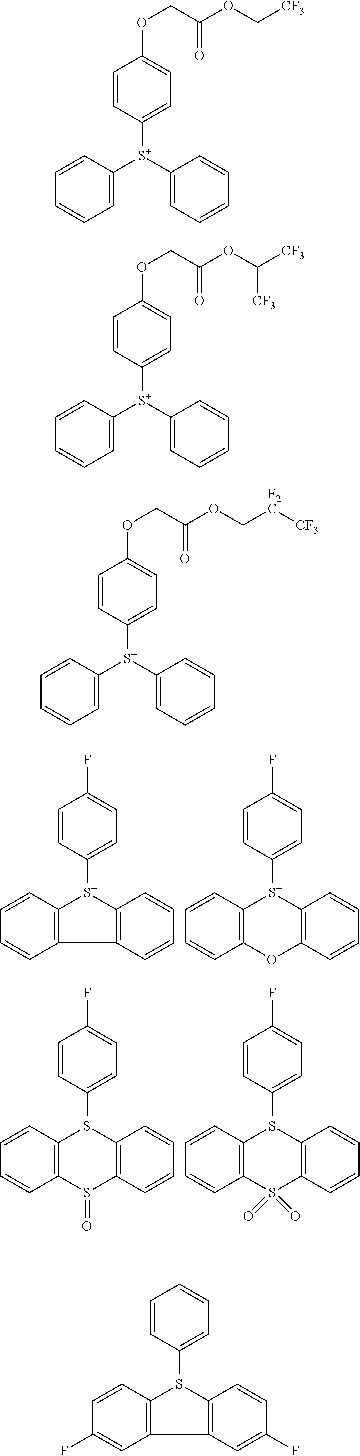

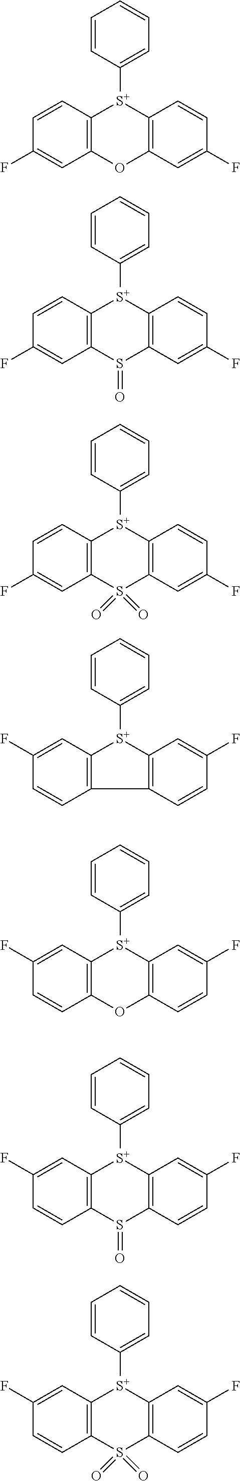

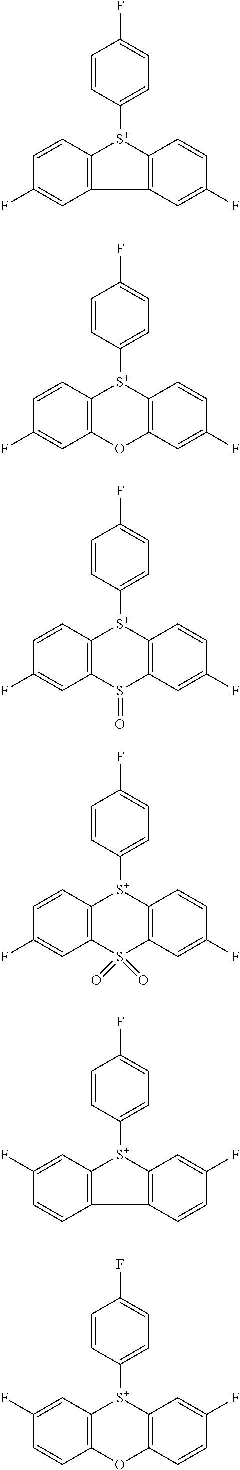 [SMILES: Fc1ccc(-[s+]2c3ccc(F)cc3c3cc(F)ccc32)cc1 ; Fc1ccc([S+]2c3ccc(F)cc3Oc3cc(F)ccc32)cc1 ; O=S1c2cc(F)ccc2[S+](c2ccc(F)cc2)c2ccc(F)cc21 ; O=S1(=O)c2cc(F)ccc2[S+](c2ccc(F)cc2)c2ccc(F)cc21 ; Fc1ccc(-[s+]2c3cc(F)ccc3c3ccc(F)cc32)cc1 ; Fc1ccc([S+]2c3cc(F)ccc3Oc3ccc(F)cc32)cc1]

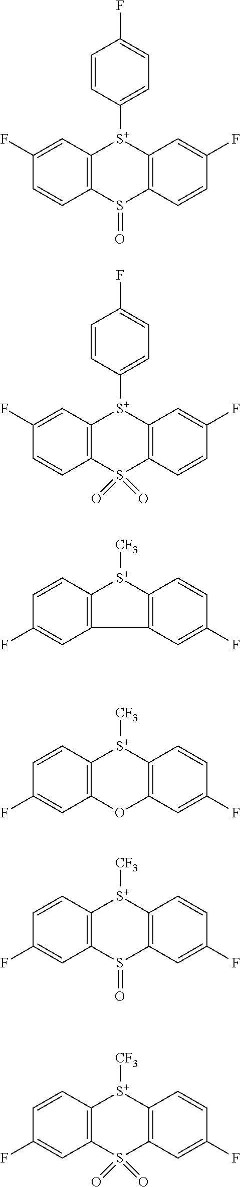 [SMILES: O=S1c2ccc(F)cc2[S+](c2ccc(F)cc2)c2cc(F)ccc21 ; O=S1(=O)c2ccc(F)cc2[S+](c2ccc(F)cc2)c2cc(F)ccc21 ; Fc1ccc2c(c1)c1cc(F)ccc1[s+]2C(F)(F)F ; Fc1ccc2c(c1)Oc1cc(F)ccc1[S+]2C(F)(F)F ; O=S1c2cc(F)ccc2[S+](C(F)(F)F)c2ccc(F)cc21 ; O=S1(=O)c2cc(F)ccc2[S+](C(F)(F)F)c2ccc(F)cc21]

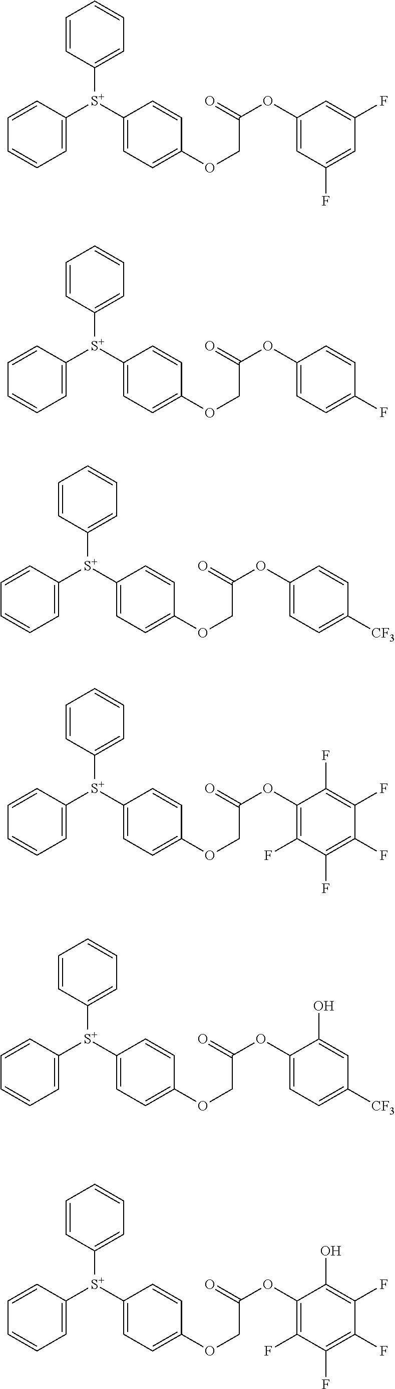 [SMILES: O=C(COc1ccc([S+](c2ccccc2)c2ccccc2)cc1)Oc1cc(F)cc(F)c1 ; O=C(COc1ccc([S+](c2ccccc2)c2ccccc2)cc1)Oc1ccc(F)cc1 ; O=C(COc1ccc([S+](c2ccccc2)c2ccccc2)cc1)Oc1ccc(C(F)(F)F)cc1 ; O=C(COc1ccc([S+](c2ccccc2)c2ccccc2)cc1)Oc1c(F)c(F)c(F)c(F)c1F ; O=C(COc1ccc([S+](c2ccccc2)c2ccccc2)cc1)Oc1ccc(C(F)(F)F)cc1O ; O=C(COc1ccc([S+](c2ccccc2)c2ccccc2)cc1)Oc1c(O)c(F)c(F)c(F)c1F]

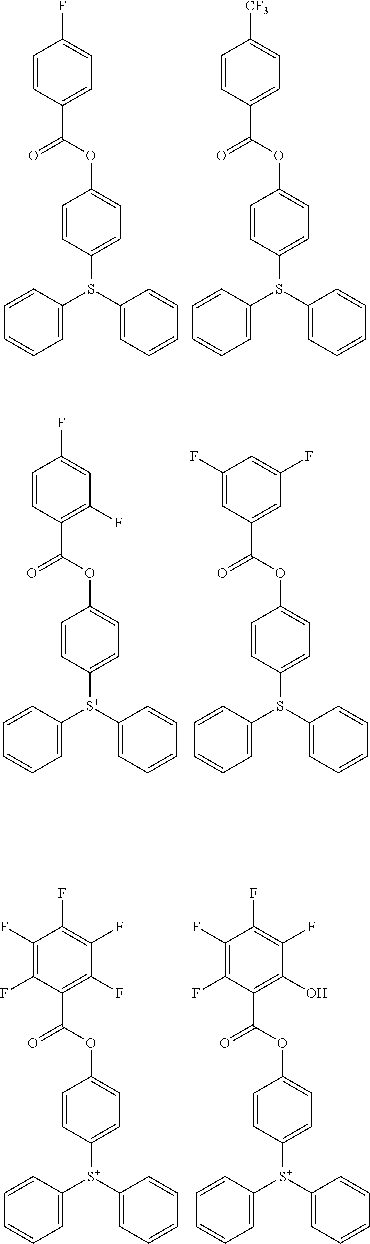 [SMILES: O=C(Oc1ccc([S+](c2ccccc2)c2ccccc2)cc1)c1ccc(F)cc1 ; O=C(Oc1ccc([S+](c2ccccc2)c2ccccc2)cc1)c1ccc(C(F)(F)F)cc1 ; O=C(Oc1ccc([S+](c2ccccc2)c2ccccc2)cc1)c1ccc(F)cc1F ; O=C(Oc1ccc([S+](c2ccccc2)c2ccccc2)cc1)c1cc(F)cc(F)c1 ; O=C(Oc1ccc([S+](c2ccccc2)c2ccccc2)cc1)c1c(F)c(F)c(F)c(F)c1F ; O=C(Oc1ccc([S+](c2ccccc2)c2ccccc2)cc1)c1c(O)c(F)c(F)c(F)c1F]

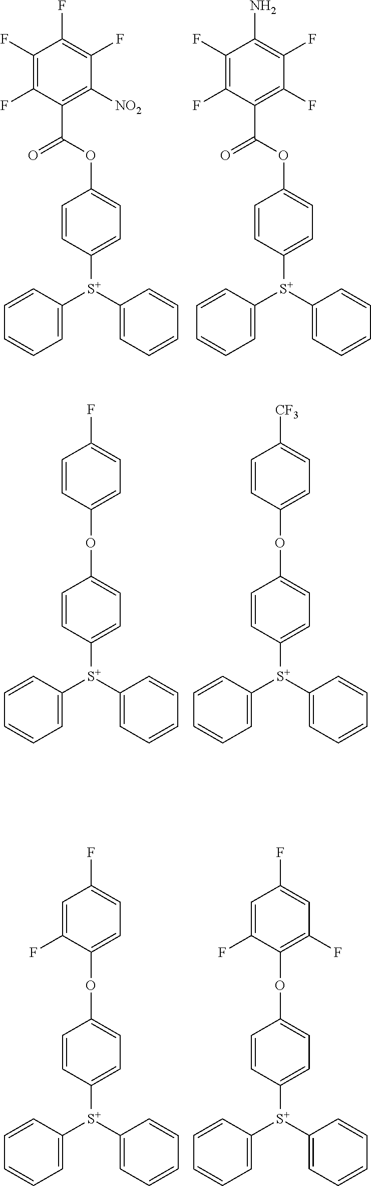 [SMILES: O=C(Oc1ccc([S+](c2ccccc2)c2ccccc2)cc1)c1c(F)c(F)c(F)c(F)c1[N+](=O)[O-] ; Nc1c(F)c(F)c(C(=O)Oc2ccc([S+](c3ccccc3)c3ccccc3)cc2)c(F)c1F ; Fc1ccc(Oc2ccc([S+](c3ccccc3)c3ccccc3)cc2)cc1 ; FC(F)(F)c1ccc(Oc2ccc([S+](c3ccccc3)c3ccccc3)cc2)cc1 ; Fc1ccc(Oc2ccc([S+](c3ccccc3)c3ccccc3)cc2)c(F)c1 ; Fc1cc(F)c(Oc2ccc([S+](c3ccccc3)c3ccccc3)cc2)c(F)c1]

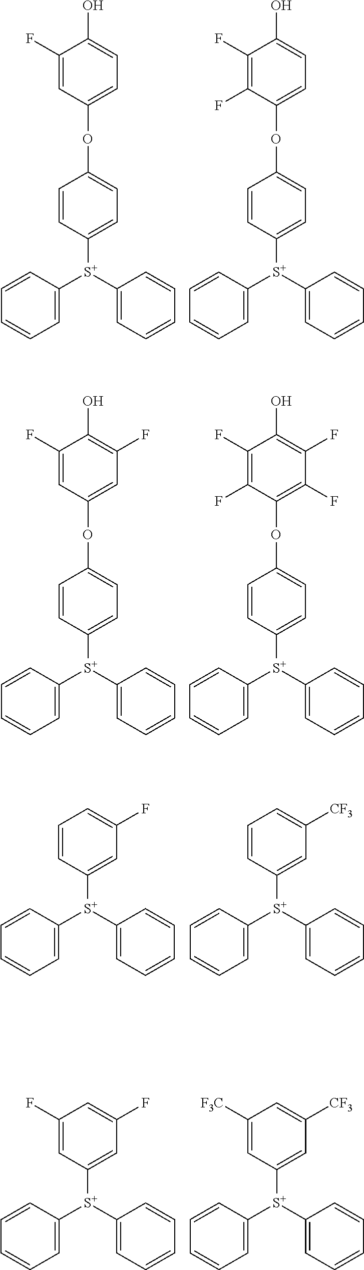

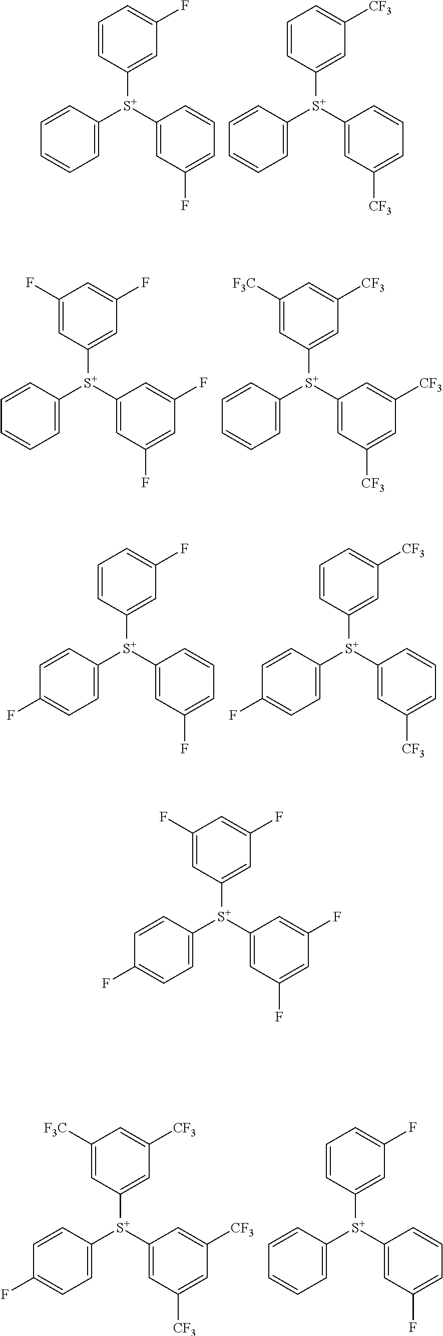

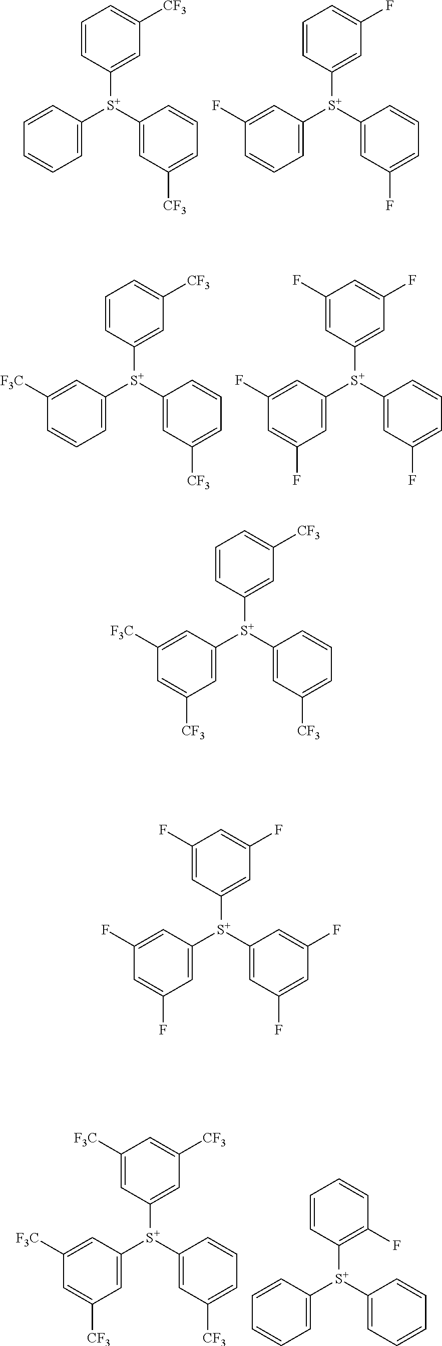

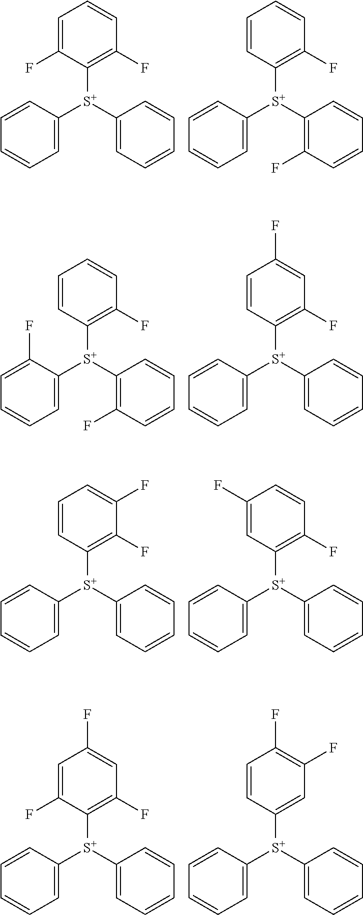

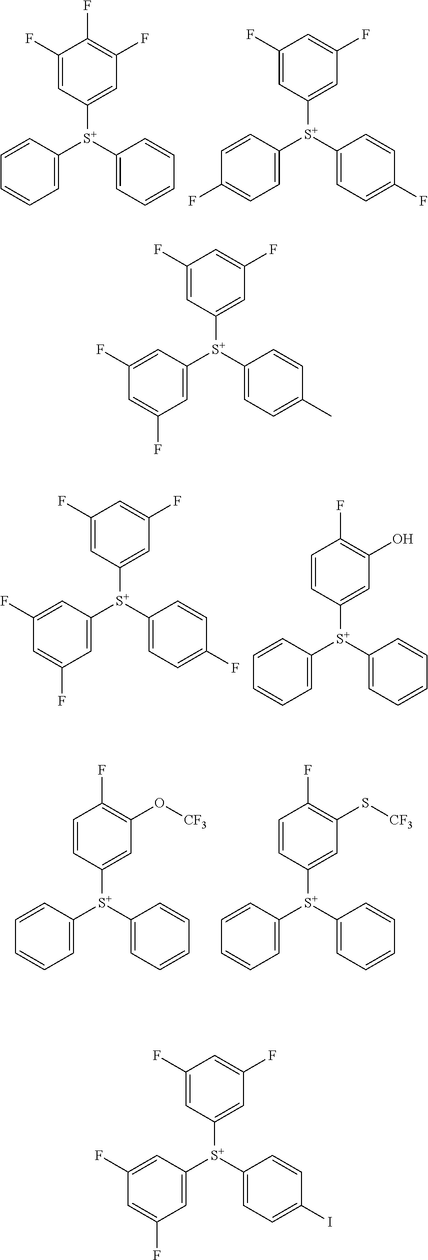

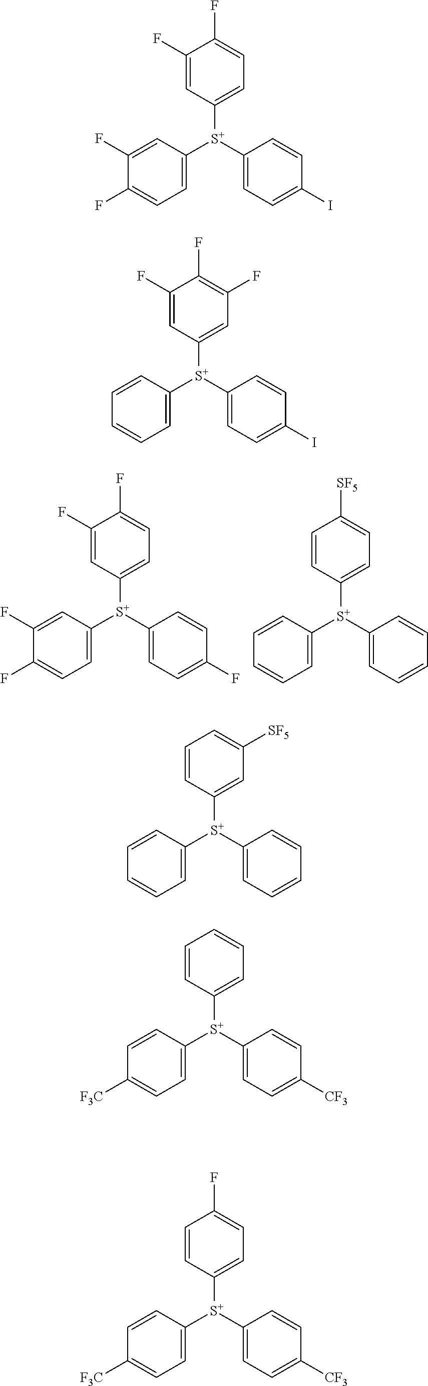

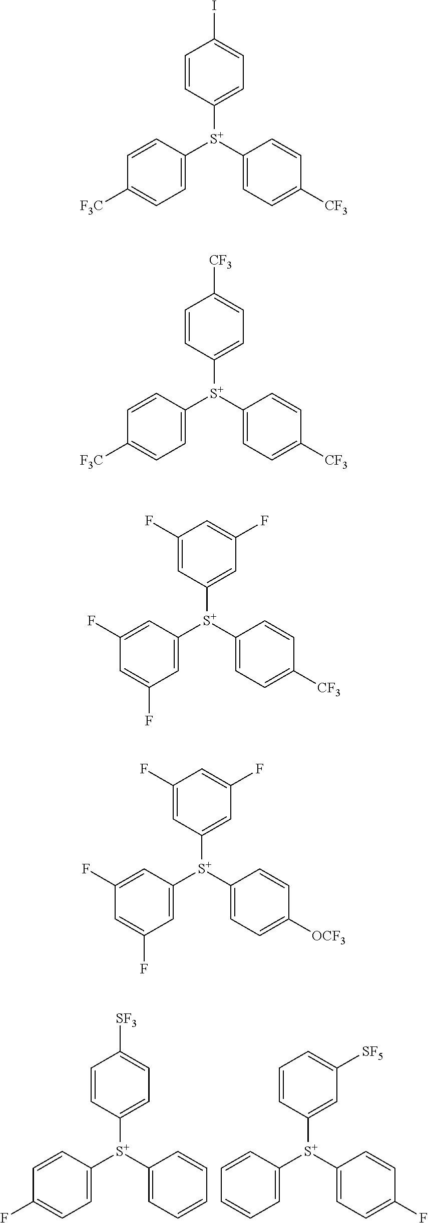

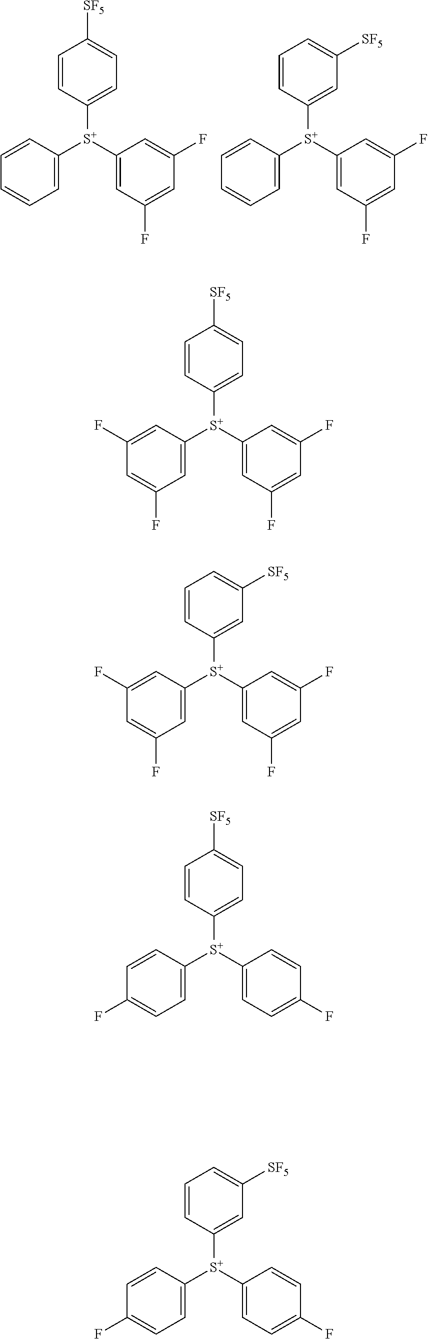

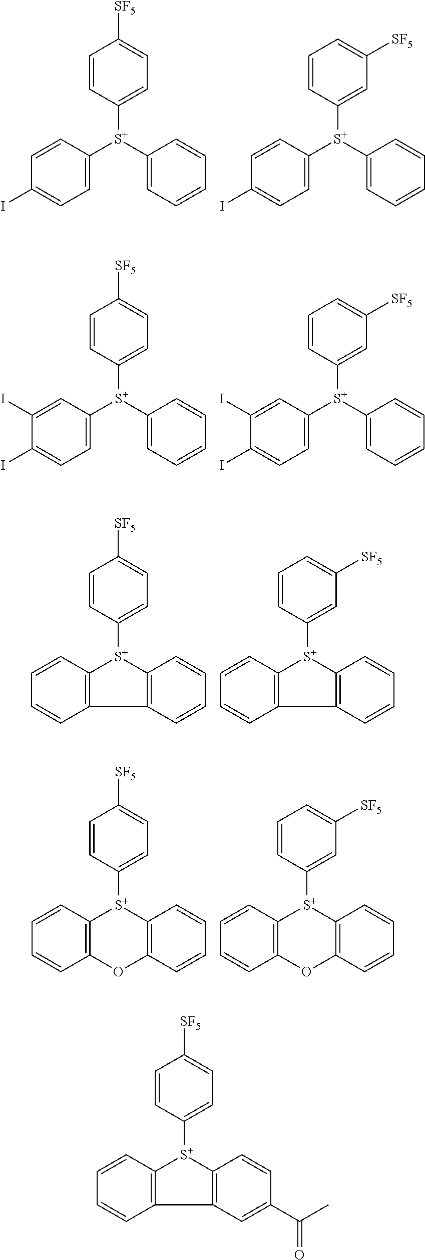

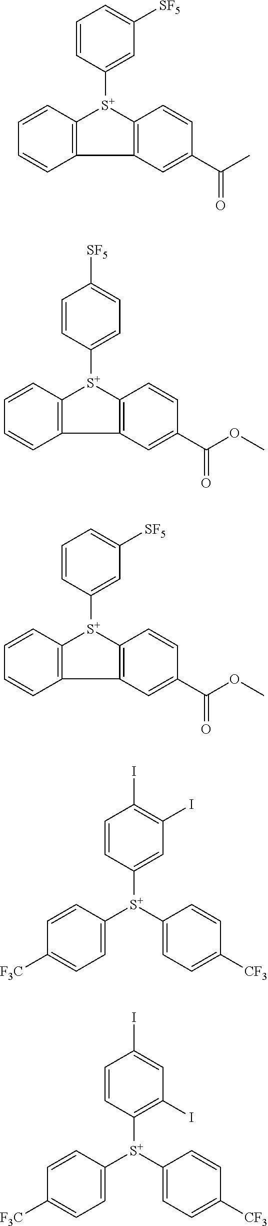

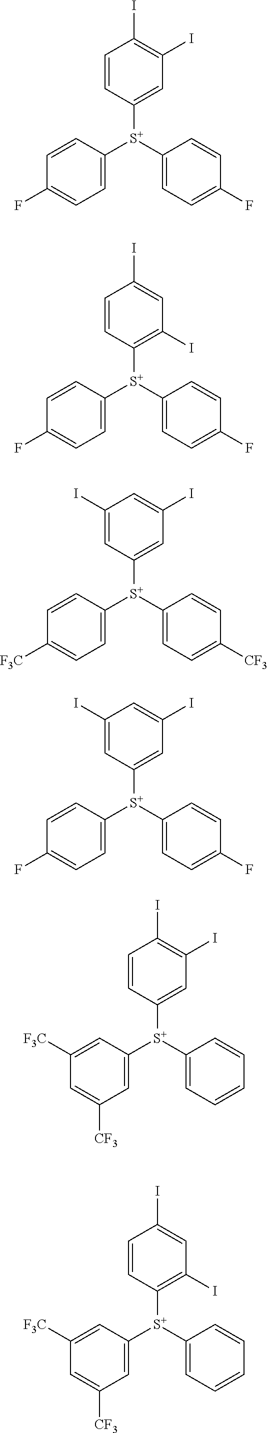 [SMILES: Fc1ccc([S+](c2ccc(F)cc2)c2ccc(I)c(I)c2)cc1 ; Fc1ccc([S+](c2ccc(F)cc2)c2ccc(I)cc2I)cc1 ; FC(F)(F)c1ccc([S+](c2ccc(C(F)(F)F)cc2)c2cc(I)cc(I)c2)cc1 ; Fc1ccc([S+](c2ccc(F)cc2)c2cc(I)cc(I)c2)cc1 ; FC(F)(F)c1cc([S+](c2ccccc2)c2ccc(I)c(I)c2)cc(C(F)(F)F)c1 ; FC(F)(F)c1cc([S+](c2ccccc2)c2ccc(I)cc2I)cc(C(F)(F)F)c1]

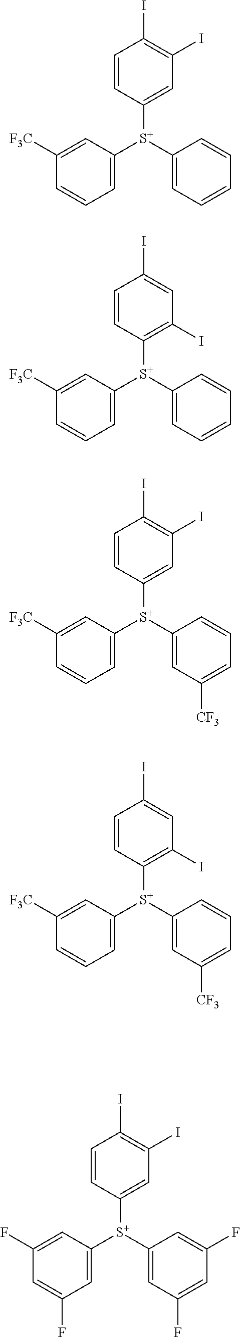 [SMILES: FC(F)(F)c1cccc([S+](c2ccccc2)c2ccc(I)c(I)c2)c1 ; FC(F)(F)c1cccc([S+](c2ccccc2)c2ccc(I)cc2I)c1 ; FC(F)(F)c1cccc([S+](c2cccc(C(F)(F)F)c2)c2ccc(I)c(I)c2)c1 ; FC(F)(F)c1cccc([S+](c2cccc(C(F)(F)F)c2)c2ccc(I)cc2I)c1 ; Fc1cc(F)cc([S+](c2cc(F)cc(F)c2)c2ccc(I)c(I)c2)c1]

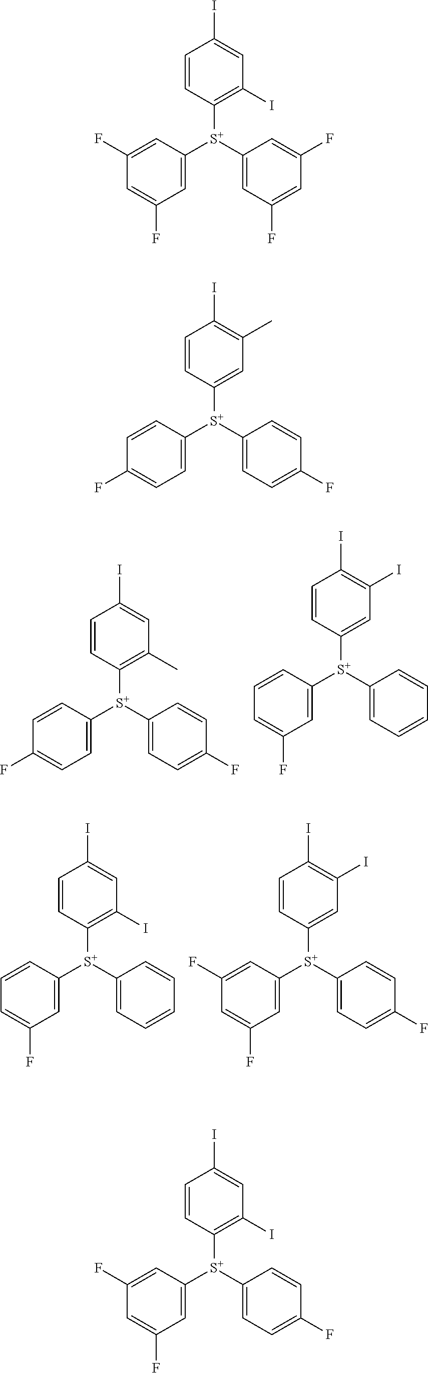

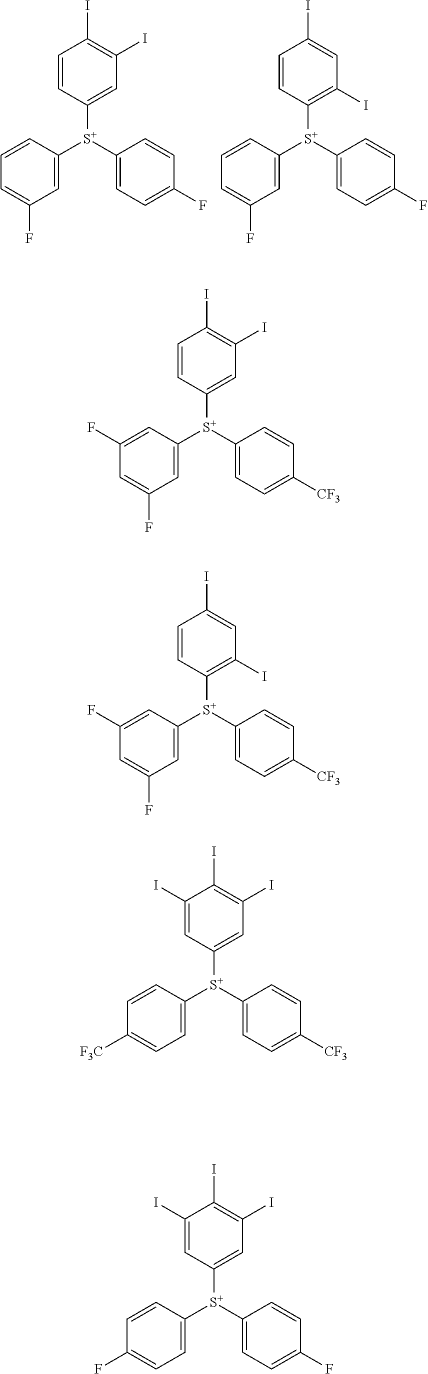 [SMILES: Fc1ccc([S+](c2cccc(F)c2)c2ccc(I)c(I)c2)cc1 ; Fc1ccc([S+](c2cccc(F)c2)c2ccc(I)cc2I)cc1 ; Fc1cc(F)cc([S+](c2ccc(C(F)(F)F)cc2)c2ccc(I)c(I)c2)c1 ; Fc1cc(F)cc([S+](c2ccc(C(F)(F)F)cc2)c2ccc(I)cc2I)c1 ; FC(F)(F)c1ccc([S+](c2ccc(C(F)(F)F)cc2)c2cc(I)c(I)c(I)c2)cc1 ; Fc1ccc([S+](c2ccc(F)cc2)c2cc(I)c(I)c(I)c2)cc1]

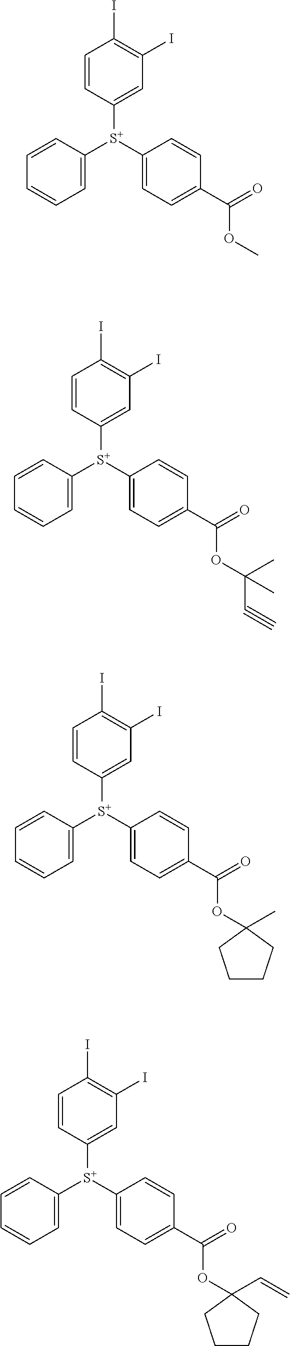 [SMILES: COC(=O)c1ccc([S+](c2ccccc2)c2ccc(I)c(I)c2)cc1 ; C#CC(C)(C)OC(=O)c1ccc([S+](c2ccccc2)c2ccc(I)c(I)c2)cc1 ; CC1(OC(=O)c2ccc([S+](c3ccccc3)c3ccc(I)c(I)c3)cc2)CCCC1 ; C=CC1(OC(=O)c2ccc([S+](c3ccccc3)c3ccc(I)c(I)c3)cc2)CCCC1]

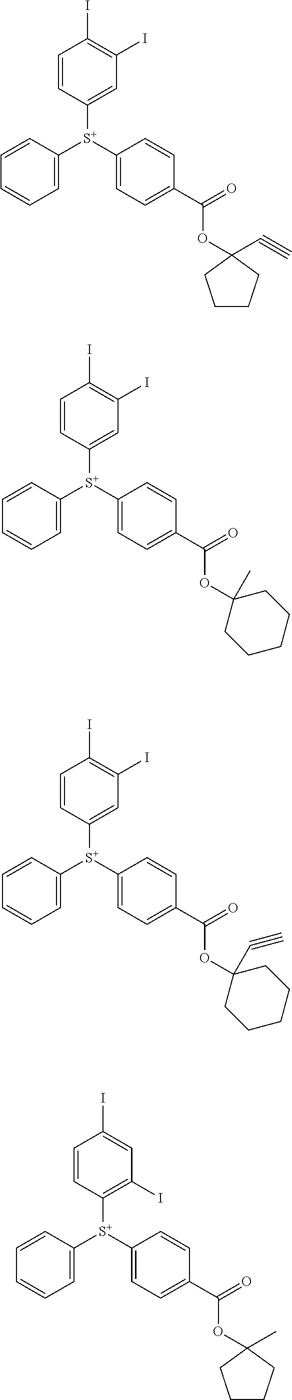 [SMILES: C#CC1(OC(=O)c2ccc([S+](c3ccccc3)c3ccc(I)c(I)c3)cc2)CCCC1 ; CC1(OC(=O)c2ccc([S+](c3ccccc3)c3ccc(I)c(I)c3)cc2)CCCCC1 ; C#CC1(OC(=O)c2ccc([S+](c3ccccc3)c3ccc(I)c(I)c3)cc2)CCCCC1 ; CC1(OC(=O)c2ccc([S+](c3ccccc3)c3ccc(I)cc3I)cc2)CCCC1]

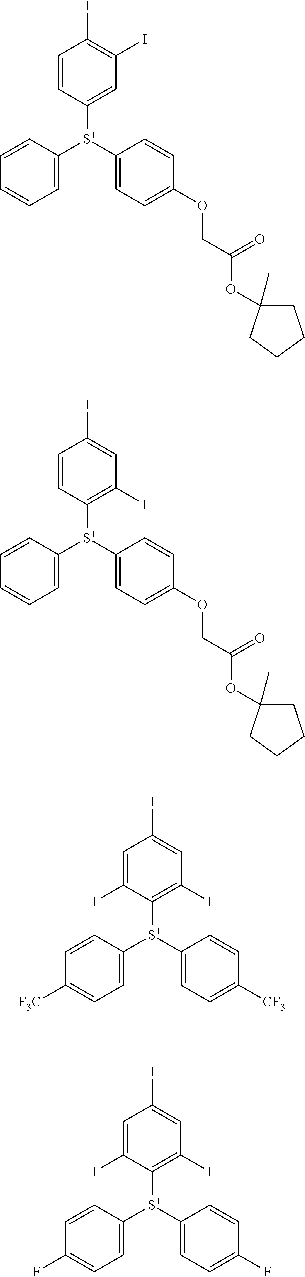 [SMILES: CC1(OC(=O)COc2ccc([S+](c3ccccc3)c3ccc(I)c(I)c3)cc2)CCCC1 ; CC1(OC(=O)COc2ccc([S+](c3ccccc3)c3ccc(I)cc3I)cc2)CCCC1 ; FC(F)(F)c1ccc([S+](c2ccc(C(F)(F)F)cc2)c2c(I)cc(I)cc2I)cc1 ; Fc1ccc([S+](c2ccc(F)cc2)c2c(I)cc(I)cc2I)cc1]

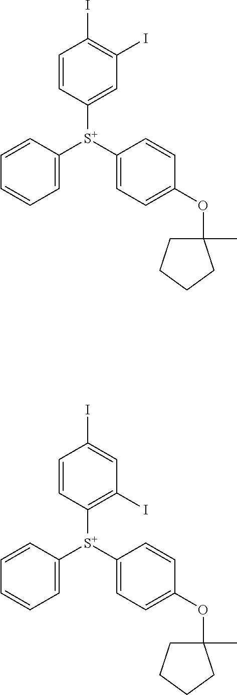 [SMILES: CC1(Oc2ccc([S+](c3ccccc3)c3ccc(I)c(I)c3)cc2)CCCC1 ; CC1(Oc2ccc([S+](c3ccccc3)c3ccc(I)cc3I)cc2)CCCC1]

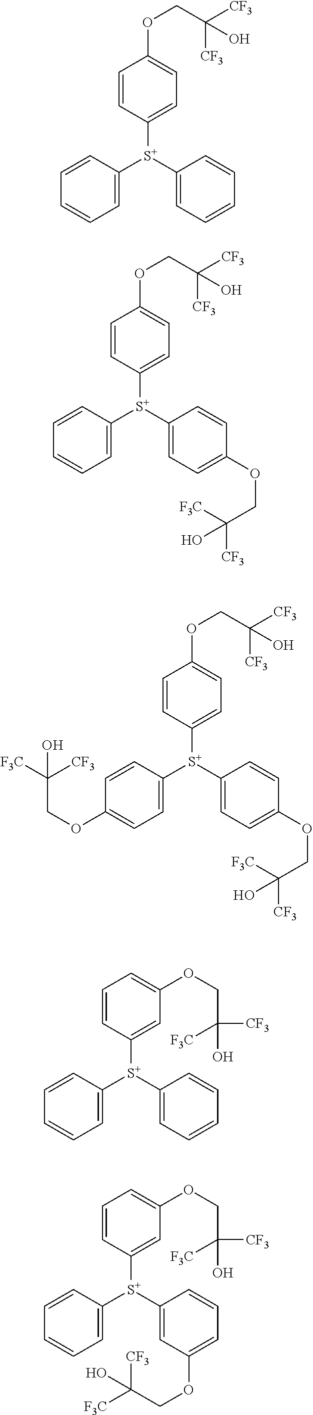 [SMILES: OC(COc1ccc([S+](c2ccccc2)c2ccccc2)cc1)(C(F)(F)F)C(F)(F)F ; OC(COc1ccc([S+](c2ccccc2)c2ccc(OCC(O)(C(F)(F)F)C(F)(F)F)cc2)cc1)(C(F)(F)F)C(F)(F)F ; OC(COc1ccc([S+](c2ccc(OCC(O)(C(F)(F)F)C(F)(F)F)cc2)c2ccc(OCC(O)(C(F)(F)F)C(F)(F)F)cc2)cc1)(C(F)(F)F)C(F)(F)F ; OC(COc1cccc([S+](c2ccccc2)c2ccccc2)c1)(C(F)(F)F)C(F)(F)F ; OC(COc1cccc([S+](c2ccccc2)c2cccc(OCC(O)(C(F)(F)F)C(F)(F)F)c2)c1)(C(F)(F)F)C(F)(F)F]

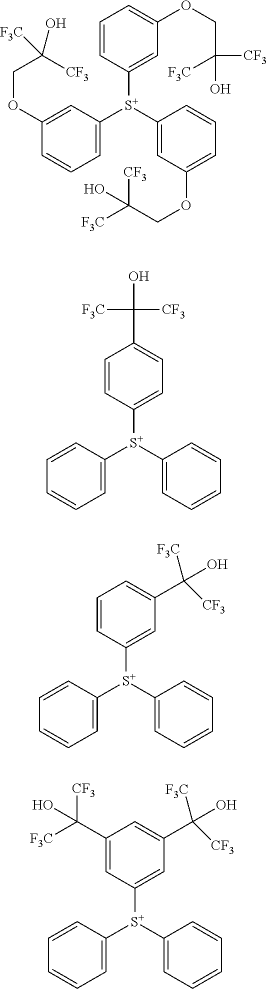 [SMILES: OC(COc1cccc([S+](c2cccc(OCC(O)(C(F)(F)F)C(F)(F)F)c2)c2cccc(OCC(O)(C(F)(F)F)C(F)(F)F)c2)c1)(C(F)(F)F)C(F)(F)F ; OC(c1ccc([S+](c2ccccc2)c2ccccc2)cc1)(C(F)(F)F)C(F)(F)F ; OC(c1cccc([S+](c2ccccc2)c2ccccc2)c1)(C(F)(F)F)C(F)(F)F ; OC(c1cc([S+](c2ccccc2)c2ccccc2)cc(C(O)(C(F)(F)F)C(F)(F)F)c1)(C(F)(F)F)C(F)(F)F]

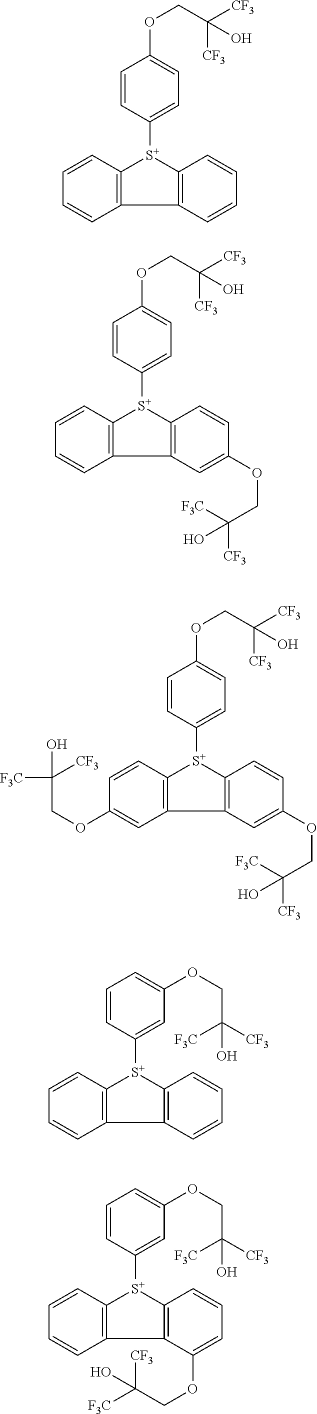 [SMILES: OC(COc1ccc(-[s+]2c3ccccc3c3ccccc32)cc1)(C(F)(F)F)C(F)(F)F ; OC(COc1ccc(-[s+]2c3ccccc3c3cc(OCC(O)(C(F)(F)F)C(F)(F)F)ccc32)cc1)(C(F)(F)F)C(F)(F)F ; OC(COc1ccc(-[s+]2c3ccc(OCC(O)(C(F)(F)F)C(F)(F)F)cc3c3cc(OCC(O)(C(F)(F)F)C(F)(F)F)ccc32)cc1)(C(F)(F)F)C(F)(F)F ; OC(COc1cccc(-[s+]2c3ccccc3c3ccccc32)c1)(C(F)(F)F)C(F)(F)F ; OC(COc1cccc(-[s+]2c3ccccc3c3c(OCC(O)(C(F)(F)F)C(F)(F)F)cccc32)c1)(C(F)(F)F)C(F)(F)F]

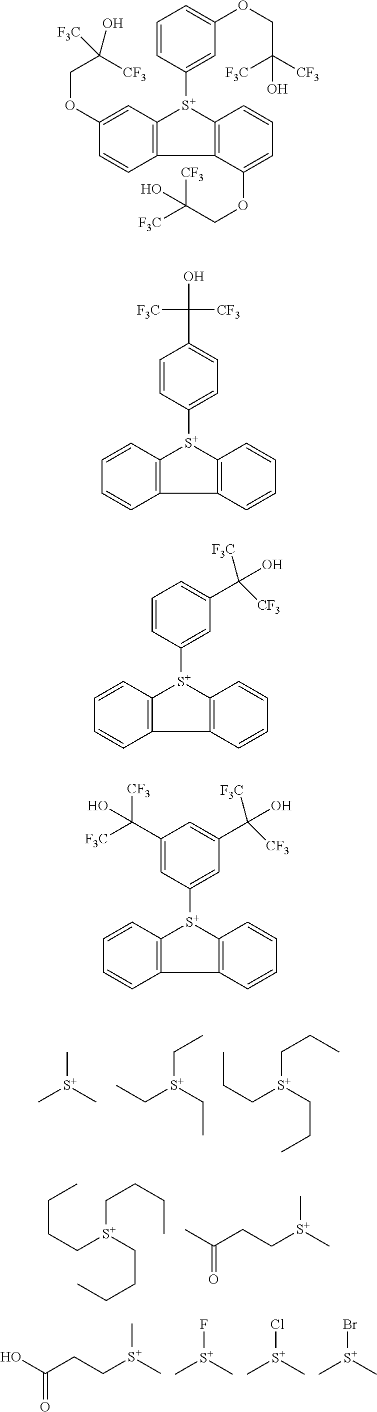

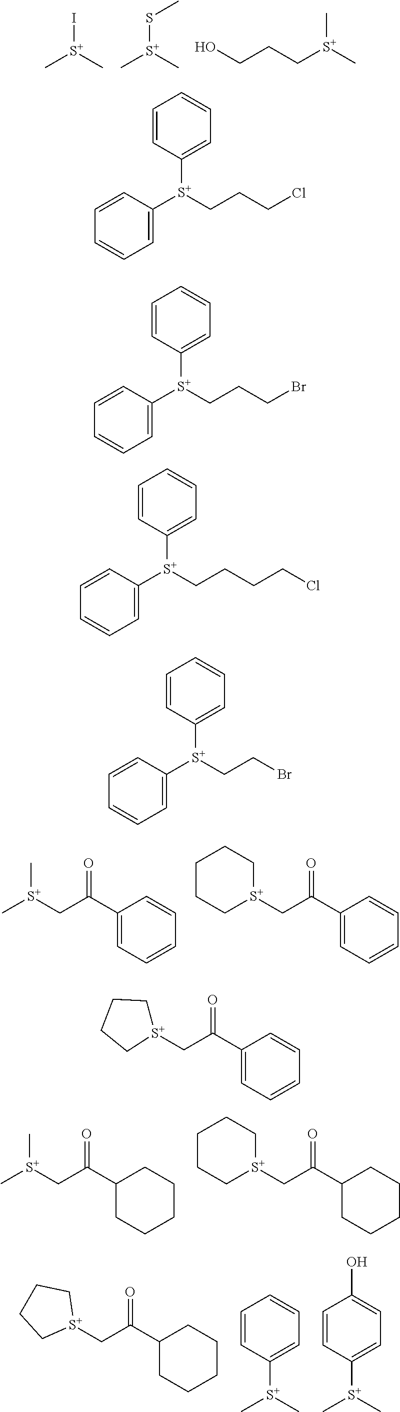

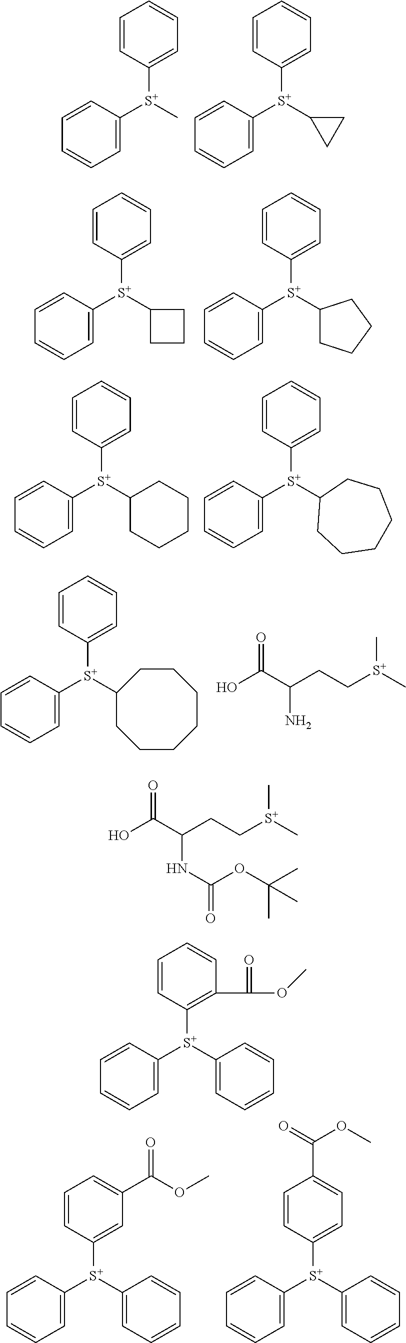

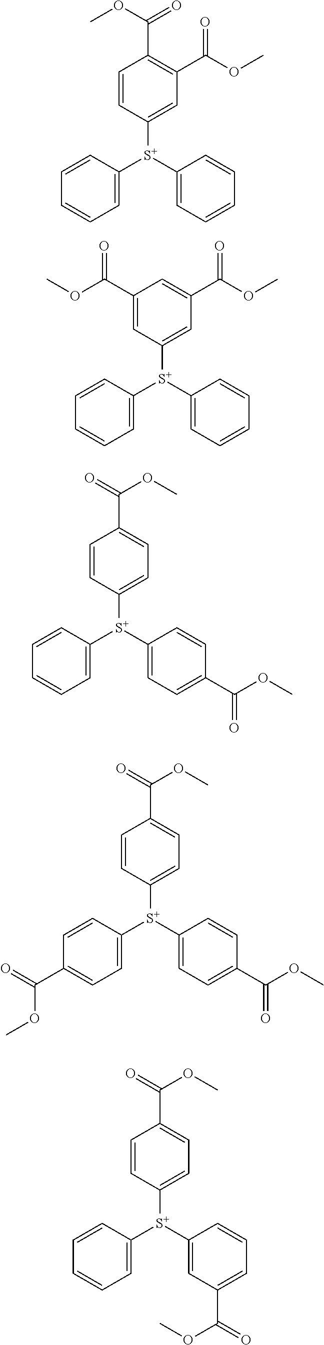

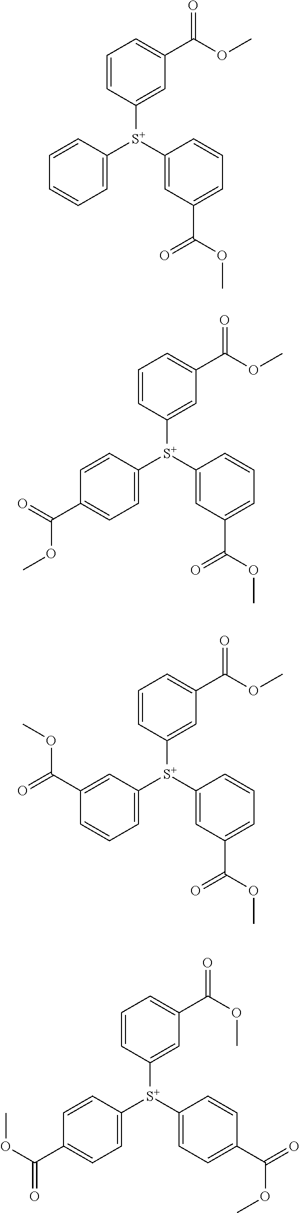 [SMILES: COC(=O)c1cccc([S+](c2ccccc2)c2cccc(C(=O)OC)c2)c1 ; COC(=O)c1ccc([S+](c2cccc(C(=O)OC)c2)c2cccc(C(=O)OC)c2)cc1 ; COC(=O)c1cccc([S+](c2cccc(C(=O)OC)c2)c2cccc(C(=O)OC)c2)c1 ; COC(=O)c1ccc([S+](c2ccc(C(=O)OC)cc2)c2cccc(C(=O)OC)c2)cc1]

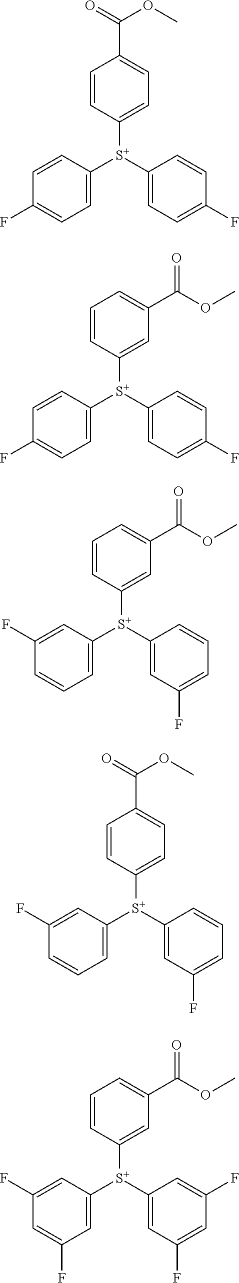 [SMILES: COC(=O)c1ccc([S+](c2ccc(F)cc2)c2ccc(F)cc2)cc1 ; COC(=O)c1cccc([S+](c2ccc(F)cc2)c2ccc(F)cc2)c1 ; COC(=O)c1cccc([S+](c2cccc(F)c2)c2cccc(F)c2)c1 ; COC(=O)c1ccc([S+](c2cccc(F)c2)c2cccc(F)c2)cc1 ; COC(=O)c1cccc([S+](c2cc(F)cc(F)c2)c2cc(F)cc(F)c2)c1]

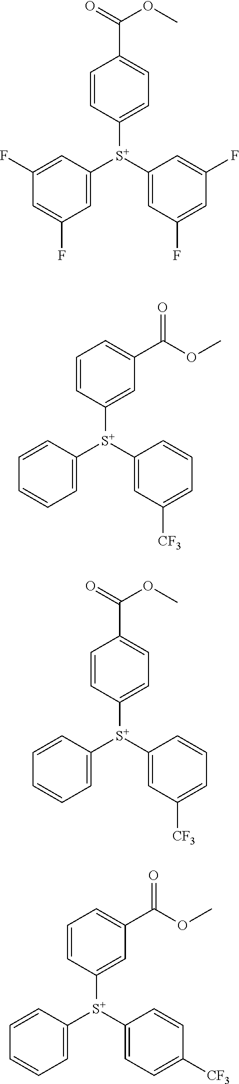 [SMILES: COC(=O)c1ccc([S+](c2cc(F)cc(F)c2)c2cc(F)cc(F)c2)cc1 ; COC(=O)c1cccc([S+](c2ccccc2)c2cccc(C(F)(F)F)c2)c1 ; COC(=O)c1ccc([S+](c2ccccc2)c2cccc(C(F)(F)F)c2)cc1 ; COC(=O)c1cccc([S+](c2ccccc2)c2ccc(C(F)(F)F)cc2)c1]

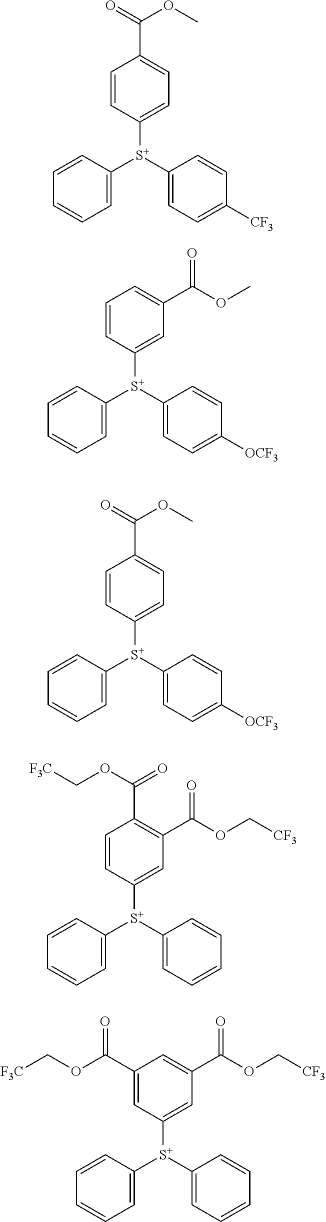 [SMILES: COC(=O)c1ccc([S+](c2ccccc2)c2ccc(C(F)(F)F)cc2)cc1 ; COC(=O)c1cccc([S+](c2ccccc2)c2ccc(OC(F)(F)F)cc2)c1 ; COC(=O)c1ccc([S+](c2ccccc2)c2ccc(OC(F)(F)F)cc2)cc1 ; O=C(OCC(F)(F)F)c1ccc([S+](c2ccccc2)c2ccccc2)cc1C(=O)OCC(F)(F)F ; O=C(OCC(F)(F)F)c1cc(C(=O)OCC(F)(F)F)cc([S+](c2ccccc2)c2ccccc2)c1]

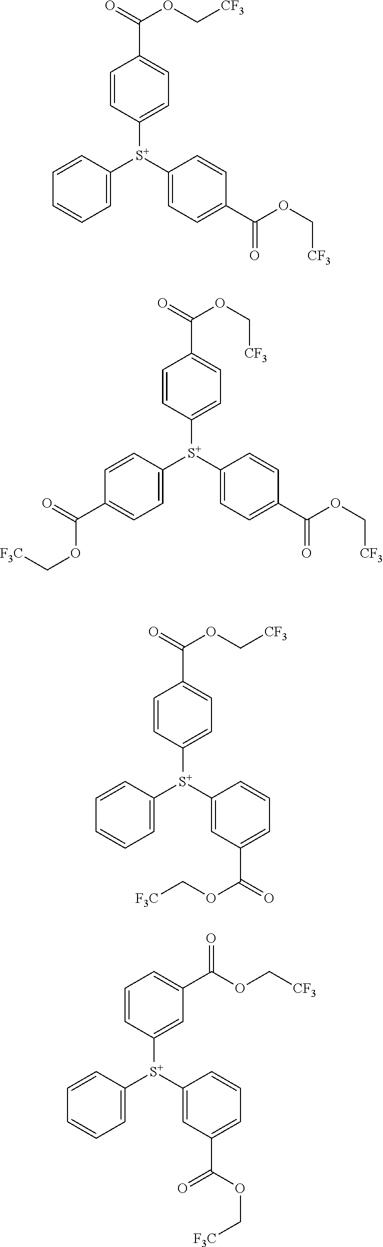 [SMILES: O=C(OCC(F)(F)F)c1ccc([S+](c2ccccc2)c2ccc(C(=O)OCC(F)(F)F)cc2)cc1 ; O=C(OCC(F)(F)F)c1ccc([S+](c2ccc(C(=O)OCC(F)(F)F)cc2)c2ccc(C(=O)OCC(F)(F)F)cc2)cc1 ; O=C(OCC(F)(F)F)c1ccc([S+](c2ccccc2)c2cccc(C(=O)OCC(F)(F)F)c2)cc1 ; O=C(OCC(F)(F)F)c1cccc([S+](c2ccccc2)c2cccc(C(=O)OCC(F)(F)F)c2)c1]

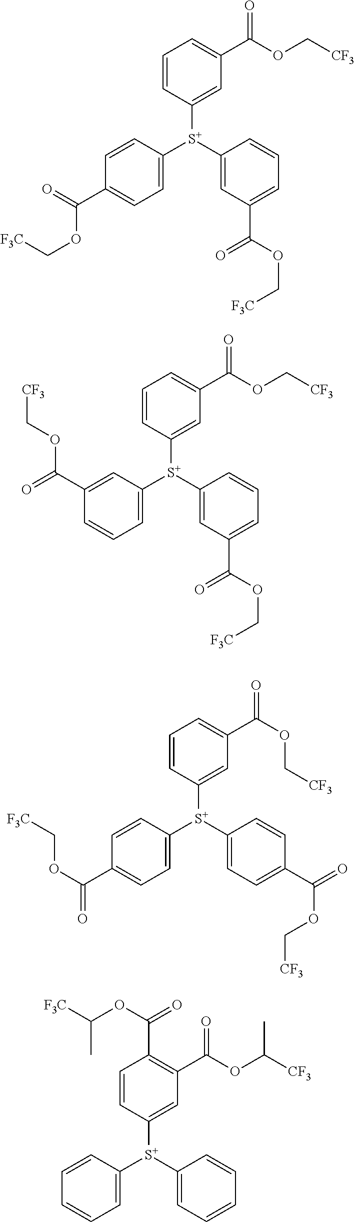 [SMILES: O=C(OCC(F)(F)F)c1ccc([S+](c2cccc(C(=O)OCC(F)(F)F)c2)c2cccc(C(=O)OCC(F)(F)F)c2)cc1 ; O=C(OCC(F)(F)F)c1cccc([S+](c2cccc(C(=O)OCC(F)(F)F)c2)c2cccc(C(=O)OCC(F)(F)F)c2)c1 ; O=C(OCC(F)(F)F)c1ccc([S+](c2ccc(C(=O)OCC(F)(F)F)cc2)c2cccc(C(=O)OCC(F)(F)F)c2)cc1 ; CC(OC(=O)c1ccc([S+](c2ccccc2)c2ccccc2)cc1C(=O)OC(C)C(F)(F)F)C(F)(F)F]

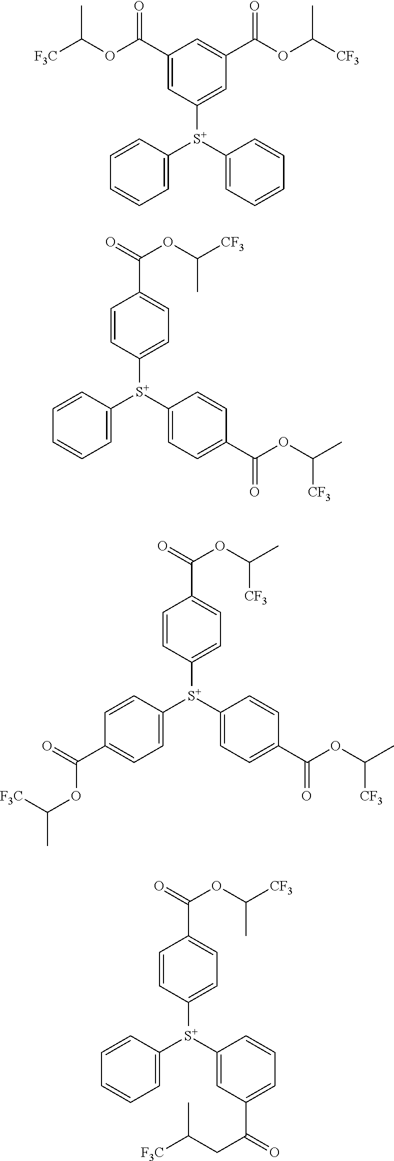 [SMILES: CC(OC(=O)c1cc(C(=O)OC(C)C(F)(F)F)cc([S+](c2ccccc2)c2ccccc2)c1)C(F)(F)F ; CC(OC(=O)c1ccc([S+](c2ccccc2)c2ccc(C(=O)OC(C)C(F)(F)F)cc2)cc1)C(F)(F)F ; CC(OC(=O)c1ccc([S+](c2ccc(C(=O)OC(C)C(F)(F)F)cc2)c2ccc(C(=O)OC(C)C(F)(F)F)cc2)cc1)C(F)(F)F ; CC(CC(=O)c1cccc([S+](c2ccccc2)c2ccc(C(=O)OC(C)C(F)(F)F)cc2)c1)C(F)(F)F]

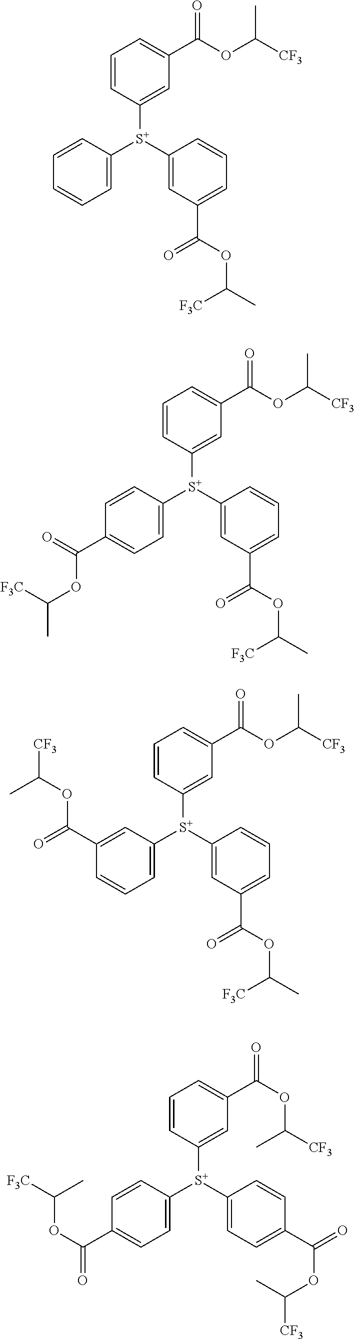 [SMILES: CC(OC(=O)c1cccc([S+](c2ccccc2)c2cccc(C(=O)OC(C)C(F)(F)F)c2)c1)C(F)(F)F ; CC(OC(=O)c1ccc([S+](c2cccc(C(=O)OC(C)C(F)(F)F)c2)c2cccc(C(=O)OC(C)C(F)(F)F)c2)cc1)C(F)(F)F ; CC(OC(=O)c1cccc([S+](c2cccc(C(=O)OC(C)C(F)(F)F)c2)c2cccc(C(=O)OC(C)C(F)(F)F)c2)c1)C(F)(F)F ; CC(OC(=O)c1ccc([S+](c2ccc(C(=O)OC(C)C(F)(F)F)cc2)c2cccc(C(=O)OC(C)C(F)(F)F)c2)cc1)C(F)(F)F]

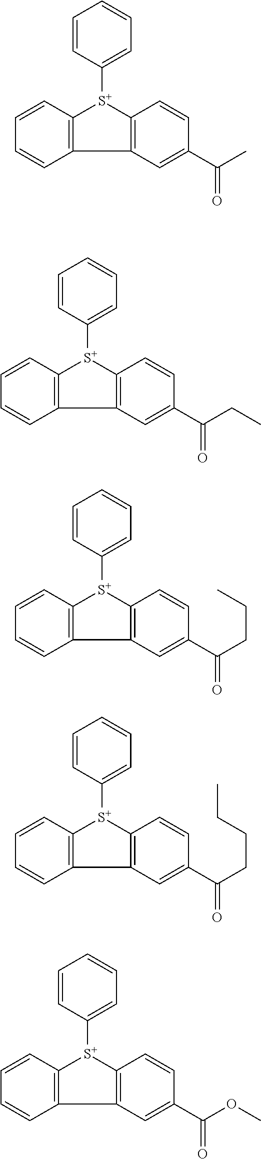 [SMILES: CC(=O)c1ccc2c(c1)c1ccccc1[s+]2-c1ccccc1 ; CCC(=O)c1ccc2c(c1)c1ccccc1[s+]2-c1ccccc1 ; CCCC(=O)c1ccc2c(c1)c1ccccc1[s+]2-c1ccccc1 ; CCCCC(=O)c1ccc2c(c1)c1ccccc1[s+]2-c1ccccc1 ; COC(=O)c1ccc2c(c1)c1ccccc1[s+]2-c1ccccc1]

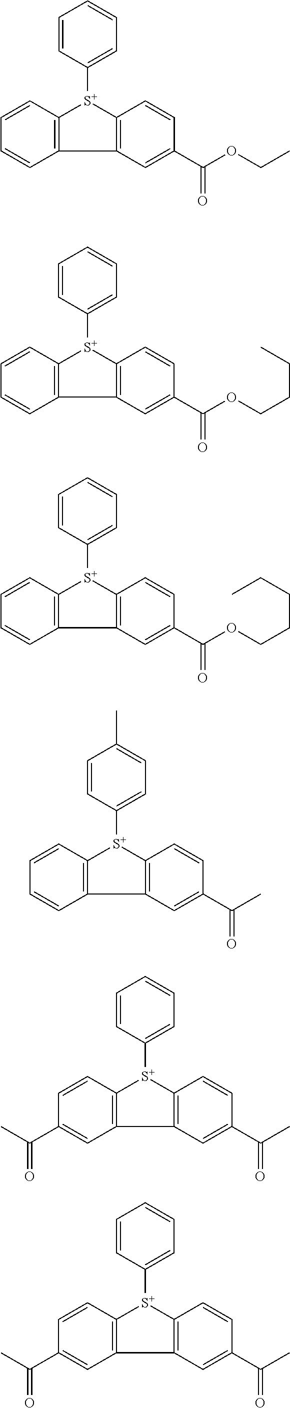

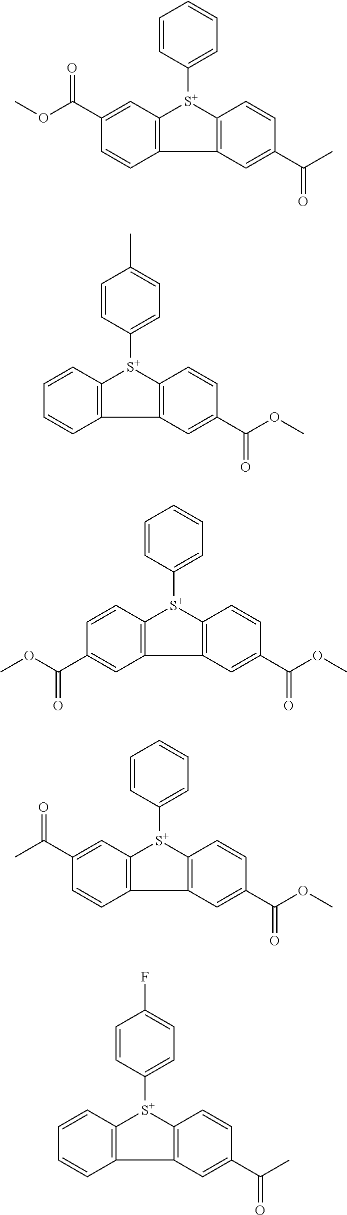

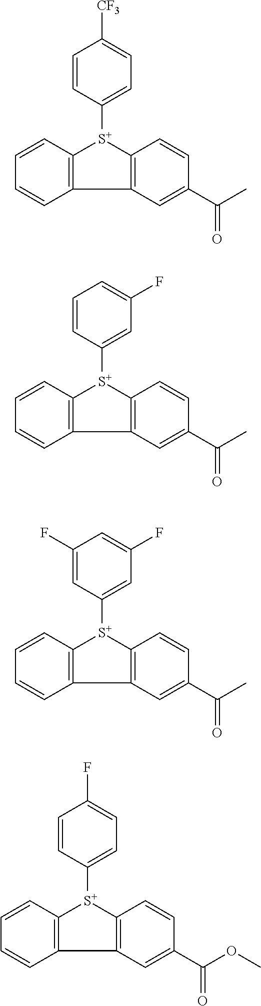 [SMILES: CC(=O)c1ccc2c(c1)c1ccccc1[s+]2-c1ccc(C(F)(F)F)cc1 ; CC(=O)c1ccc2c(c1)c1ccccc1[s+]2-c1cccc(F)c1 ; CC(=O)c1ccc2c(c1)c1ccccc1[s+]2-c1cc(F)cc(F)c1 ; COC(=O)c1ccc2c(c1)c1ccccc1[s+]2-c1ccc(F)cc1]

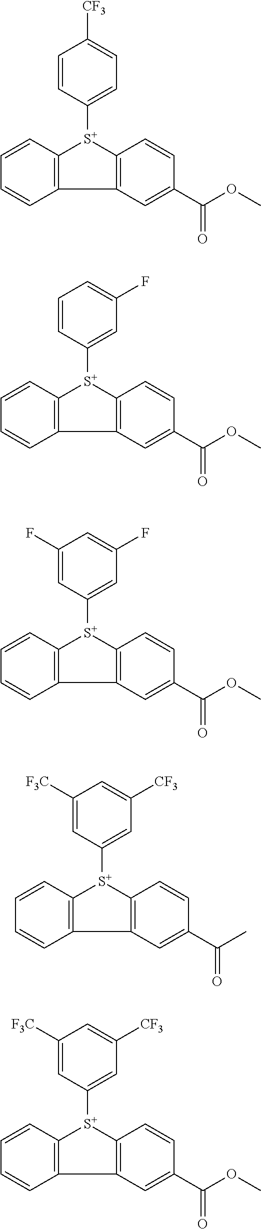 [SMILES: COC(=O)c1ccc2c(c1)c1ccccc1[s+]2-c1ccc(C(F)(F)F)cc1 ; COC(=O)c1ccc2c(c1)c1ccccc1[s+]2-c1cccc(F)c1 ; COC(=O)c1ccc2c(c1)c1ccccc1[s+]2-c1cc(F)cc(F)c1 ; CC(=O)c1ccc2c(c1)c1ccccc1[s+]2-c1cc(C(F)(F)F)cc(C(F)(F)F)c1 ; COC(=O)c1ccc2c(c1)c1ccccc1[s+]2-c1cc(C(F)(F)F)cc(C(F)(F)F)c1]

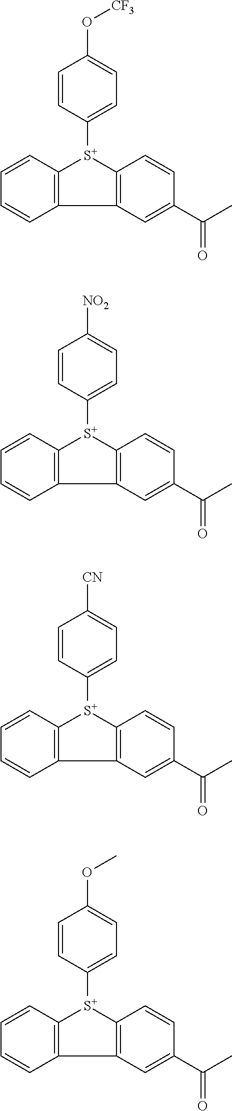 [SMILES: CC(=O)c1ccc2c(c1)c1ccccc1[s+]2-c1ccc(OC(F)(F)F)cc1 ; CC(=O)c1ccc2c(c1)c1ccccc1[s+]2-c1ccc([N+](=O)[O-])cc1 ; CC(=O)c1ccc2c(c1)c1ccccc1[s+]2-c1ccc(C#N)cc1 ; COc1ccc(-[s+]2c3ccccc3c3cc(C(C)=O)ccc32)cc1]

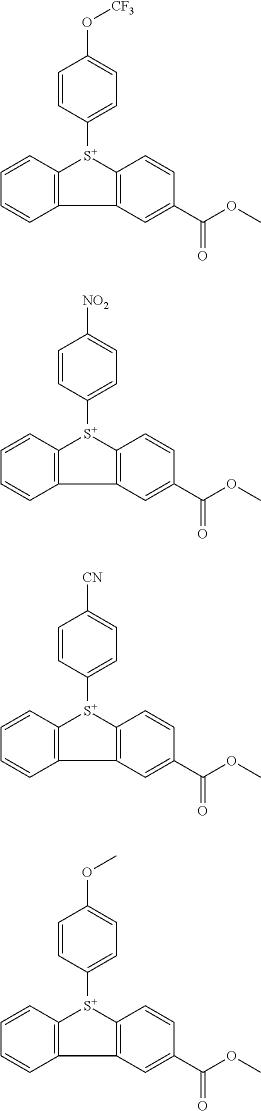 [SMILES: COC(=O)c1ccc2c(c1)c1ccccc1[s+]2-c1ccc(OC(F)(F)F)cc1 ; COC(=O)c1ccc2c(c1)c1ccccc1[s+]2-c1ccc([N+](=O)[O-])cc1 ; COC(=O)c1ccc2c(c1)c1ccccc1[s+]2-c1ccc(C#N)cc1 ; COC(=O)c1ccc2c(c1)c1ccccc1[s+]2-c1ccc(OC)cc1]

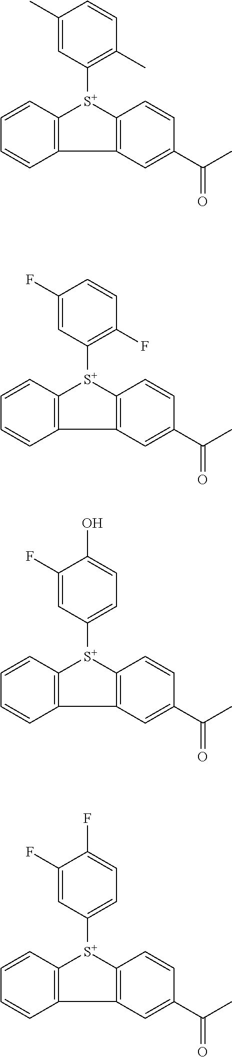 [SMILES: CC(=O)c1ccc2c(c1)c1ccccc1[s+]2-c1cc(C)ccc1C ; CC(=O)c1ccc2c(c1)c1ccccc1[s+]2-c1cc(F)ccc1F ; CC(=O)c1ccc2c(c1)c1ccccc1[s+]2-c1ccc(O)c(F)c1 ; CC(=O)c1ccc2c(c1)c1ccccc1[s+]2-c1ccc(F)c(F)c1]

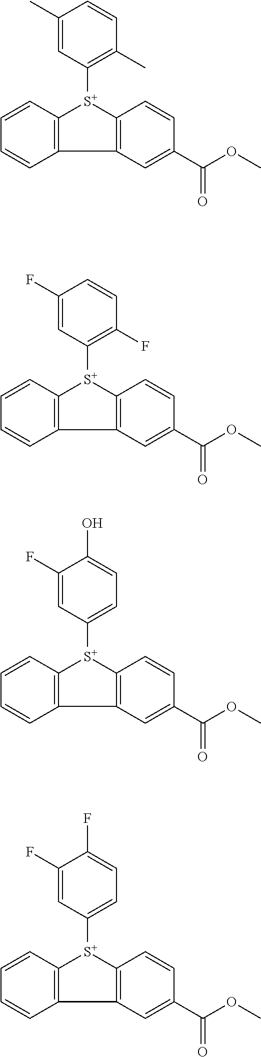 [SMILES: COC(=O)c1ccc2c(c1)c1ccccc1[s+]2-c1cc(C)ccc1C ; COC(=O)c1ccc2c(c1)c1ccccc1[s+]2-c1cc(F)ccc1F ; COC(=O)c1ccc2c(c1)c1ccccc1[s+]2-c1ccc(O)c(F)c1 ; COC(=O)c1ccc2c(c1)c1ccccc1[s+]2-c1ccc(F)c(F)c1]

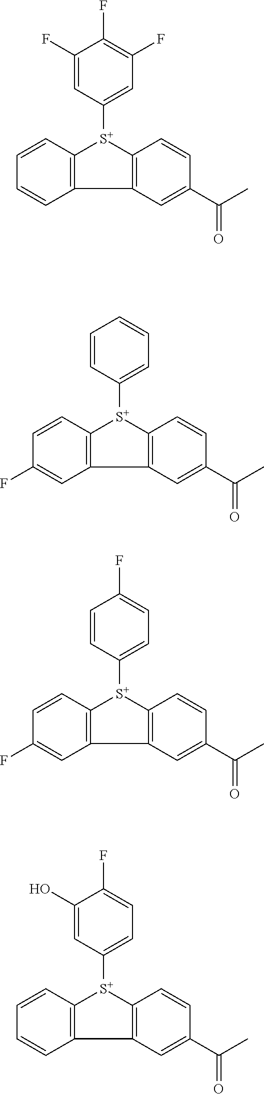 [SMILES: CC(=O)c1ccc2c(c1)c1ccccc1[s+]2-c1cc(F)c(F)c(F)c1 ; CC(=O)c1ccc2c(c1)c1cc(F)ccc1[s+]2-c1ccccc1 ; CC(=O)c1ccc2c(c1)c1cc(F)ccc1[s+]2-c1ccc(F)cc1 ; CC(=O)c1ccc2c(c1)c1ccccc1[s+]2-c1ccc(F)c(O)c1]

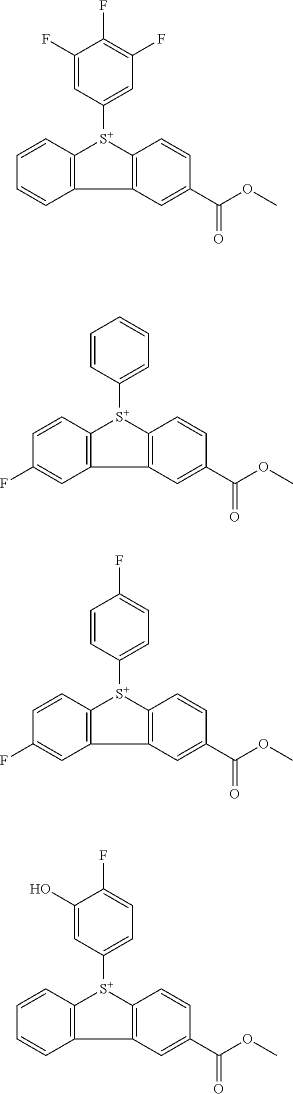 [SMILES: COC(=O)c1ccc2c(c1)c1ccccc1[s+]2-c1cc(F)c(F)c(F)c1 ; COC(=O)c1ccc2c(c1)c1cc(F)ccc1[s+]2-c1ccccc1 ; COC(=O)c1ccc2c(c1)c1cc(F)ccc1[s+]2-c1ccc(F)cc1 ; COC(=O)c1ccc2c(c1)c1ccccc1[s+]2-c1ccc(F)c(O)c1]

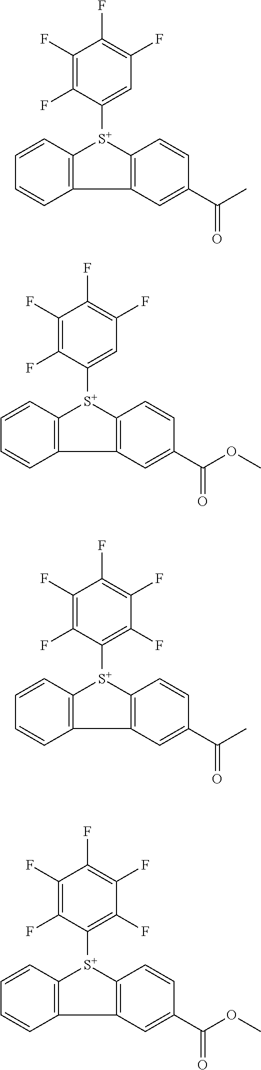 [SMILES: CC(=O)c1ccc2c(c1)c1ccccc1[s+]2-c1cc(F)c(F)c(F)c1F ; COC(=O)c1ccc2c(c1)c1ccccc1[s+]2-c1cc(F)c(F)c(F)c1F ; CC(=O)c1ccc2c(c1)c1ccccc1[s+]2-c1c(F)c(F)c(F)c(F)c1F ; COC(=O)c1ccc2c(c1)c1ccccc1[s+]2-c1c(F)c(F)c(F)c(F)c1F]

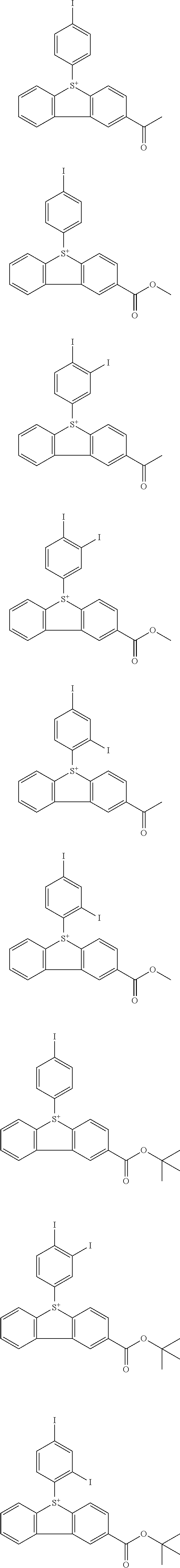

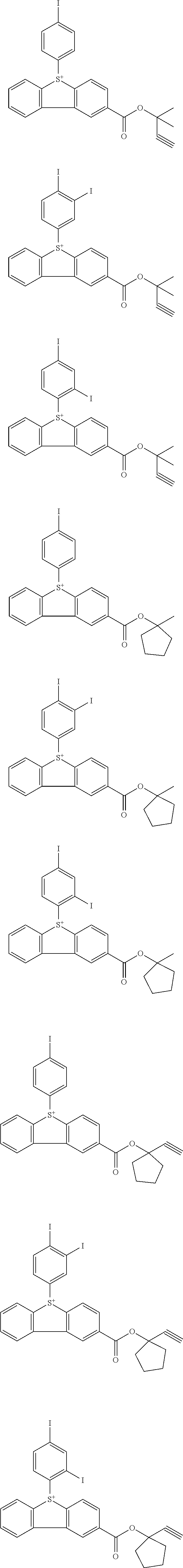

Specific examples of the iodonium cation represented by the formula (Z-2) include ones shown below, but are not limited thereto.

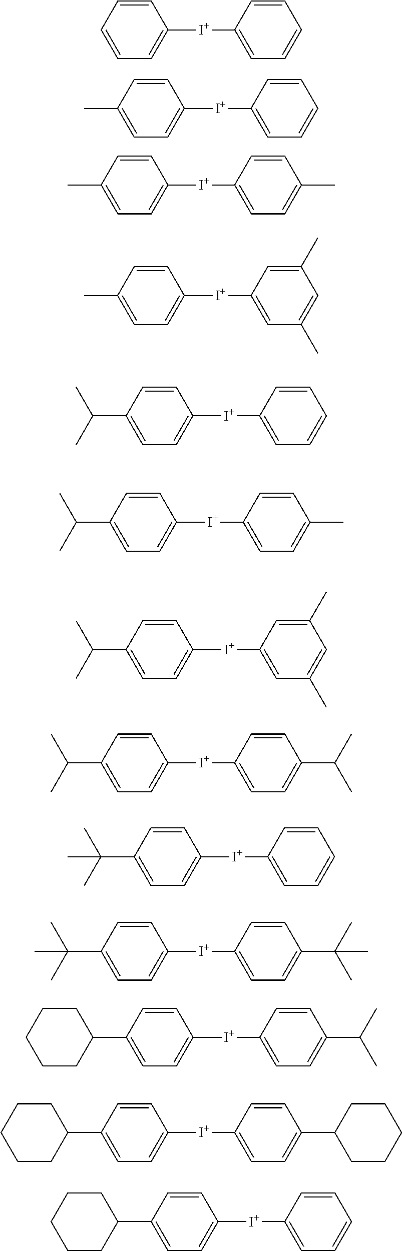

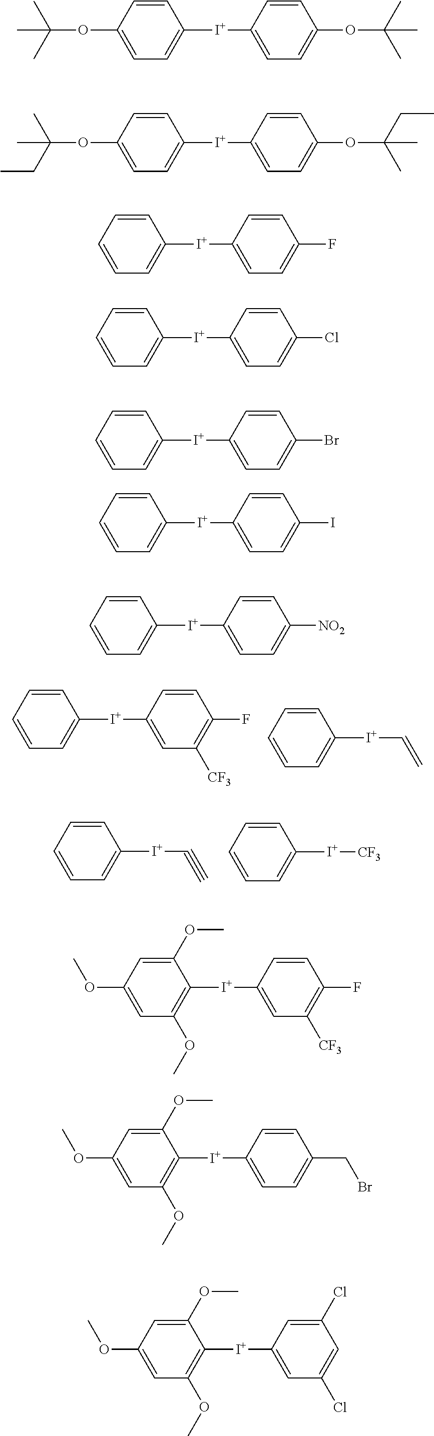

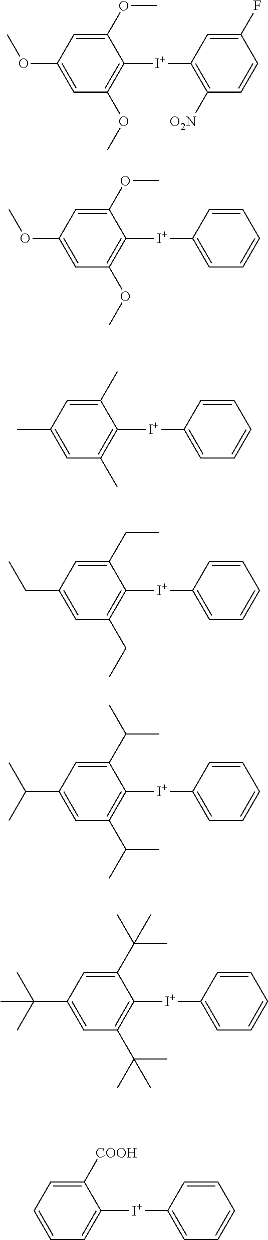

Specific examples of the inventive onium salt include any combination of the above-described anions and the cations.

As a method for synthesizing the onium salt (sulfonium salt and iodonium salt) represented by the formula (A), for example, there is a method of: synthesizing an arylsulfonic acid having a substituent which is an iodine atom by a reaction between an aryl compound having a substituent which is an iodine atom and fuming sulfuric acid; forming an ammonium salt or an alkali metal salt by a neutralization reaction with an amine compound; and performing salt exchange between the ammonium salt and a sulfonium salt or iodonium salt including a halide anion.

### [Photo-Acid Generator]

The present invention also provides a photo-acid generator which includes the above-described onium salt. The inventive onium salt can be used suitably as a photo-acid generator.

### [Chemically-Amplified Resist Composition]

The present invention also provides a chemically-amplified resist composition containing the above-described photo-acid generator. In the inventive chemically-amplified resist composition, the contained amount of the onium salt (sulfonium salt or iodonium salt) represented by the formula (A) is preferably 0.01 to 1000 parts by mass, more preferably 0.05 to 500 parts by mass, based on 100 parts by mass of the base polymer described below, from the viewpoints of sensitivity and the effect of suppressing acid diffusion.

The inventive chemically-amplified resist composition may contain a base polymer and other components, in addition to the above-described photo-acid generator. In the following, each component will be described in detail.

### [Base Polymer]

In the case of a positive resist material, the base polymer contained in the inventive chemically-amplified resist composition includes a repeating unit containing an acid-labile group. As the repeating unit containing an acid-labile group, a repeating unit represented by the following formula (a1) (hereinafter, also referred to as repeating unit-a1) or a repeating unit represented by the following formula (a2) (hereinafter, also referred to as repeating unit-a2) is preferable.

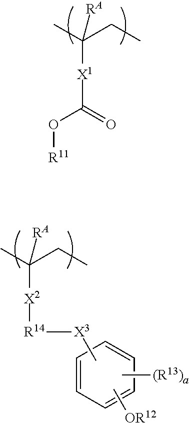

In the formulae (a1) and (a2), each R^{A} independently represents a hydrogen atom or a methyl group;
X¹ represents a single bond, a phenylene group, a naphthylene group, or a linking group having 1 to 12 carbon atoms and containing at least one kind selected from an ester bond, an ether bond, and a lactone ring, the phenylene group, the naphthylene group, and the linking group optionally containing at least one selected from a hydroxy group, a halogen atom, a saturated hydrocarbyloxy group having 1 to 8 carbon atoms, and a saturated hydrocarbylcarbonyloxy group having 2 to 8 carbon atoms;
X² represents a single bond or an ester bond;
X³ represents a single bond, an ether bond, or an ester bond;
R¹¹ and R¹² each independently represent an acid-labile group;
R¹³ represents a saturated hydrocarbyl group having 1 to 4 carbon atoms, a halogen atom, a saturated hydrocarbylcarbonyl group having 2 to 5 carbon atoms, a cyano group, or a saturated hydrocarbyloxycarbonyl group having 2 to 5 carbon atoms;
R¹⁴ represents a single bond or an alkanediyl group having 1 to 6 carbon atoms, the alkanediyl group optionally containing at least one selected from a hydroxy group, a saturated hydrocarbyloxy group having 1 to 8 carbon atoms, a saturated hydrocarbylcarbonyloxy group having 2 to 8 carbon atoms, an ether bond, and an ester bond; and
“a” represents an integer of 0 to 4.

Specific examples of a monomer to give the repeating unit-a1 include ones shown below, but are not limited thereto. Note that, in the following formulae, R^{A} and R¹¹ are as defined above.

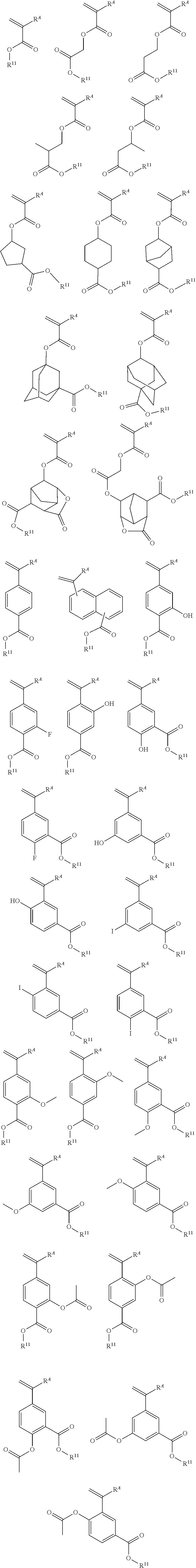

Specific examples of a monomer to give the repeating unit-a2 include ones shown below, but are not limited thereto. Note that, in the following formulae, R^{A} and R¹² are as defined above.

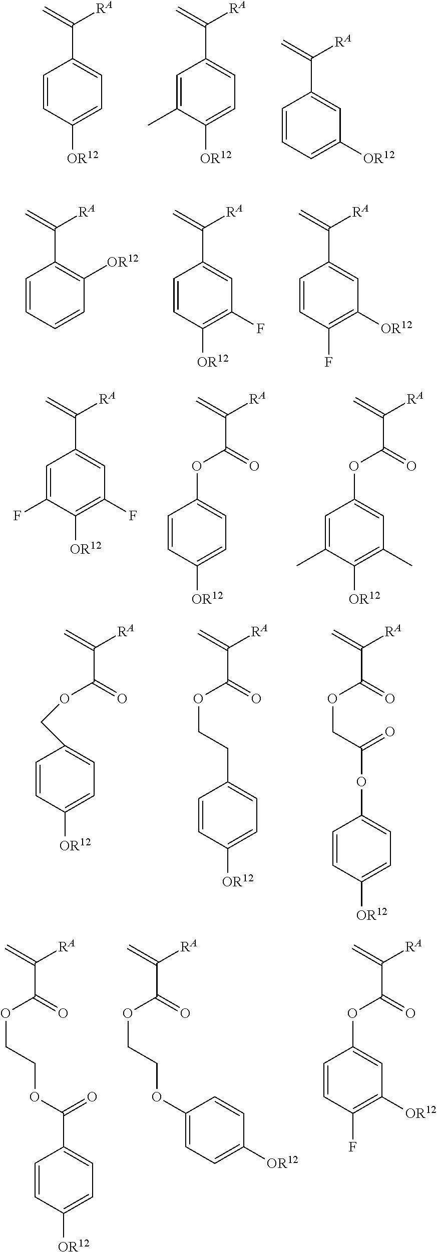

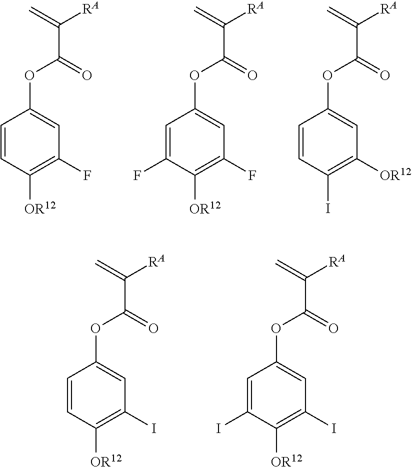

Various acid-labile groups can be selected as the acid-labile groups represented by R¹¹ or R¹². Examples thereof include ones represented by the following formulae (AL-1) to (AL-3).

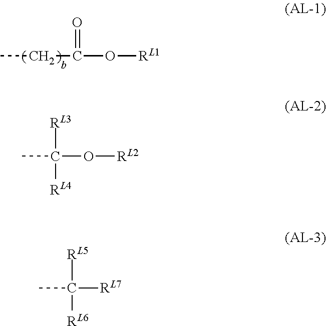

In the formulae, a broken line represents an attachment point.

In the formula (AL-1), “b” represents an integer of 0 to 6. R^{L1} represents: a tertiary hydrocarbyl group having 4 to 20, preferably 4 to 15 carbon atoms; a trihydrocarbylsilyl group in which hydrocarbyl groups are each a saturated hydrocarbyl group having 1 to 6 carbon atoms; a saturated hydrocarbyl group having 4 to 20 carbon atoms and containing a carbonyl group, an ether bond, or an ester bond; or a group represented by the formula (AL-3). Note that a tertiary hydrocarbyl group means a group obtained by a hydrogen atom being removed from a tertiary carbon atom of a hydrocarbon.

The tertiary hydrocarbyl group represented by R^{L1} may be saturated or unsaturated, and may be branched or cyclic. Specific examples thereof include a tert-butyl group, a tert-pentyl group, a 1,1-diethylpropyl group, a 1-ethylcyclopentyl group, a 1-butylcyclopentyl group, a 1-ethylcyclohexyl group, a 1-butylcyclohexyl group, a 1-ethyl-2-cyclopentenyl group, a 1-ethyl-2-cyclohexenyl group, a 2-methyl-2-adamantyl group, etc. Examples of the trihydrocarbylsilyl group include a trimethylsilyl group, a triethylsilyl group, a dimethyl-tert-butylsilyl group, etc. The saturated hydrocarbyl group containing a carbonyl group, an ether bond, or an ester bond may be linear, branched, or cyclic, and is preferably cyclic. Specific examples thereof include a 3-oxocyclohexyl group, a 4-methyl-2-oxooxan-4-yl group, a 5-methyl-2-oxooxolan-5-yl group, a 2-tetrahydropyranyl group, a 2-tetrahydrofuranyl group, etc.

Examples of the acid-labile group represented by the formula (AL-1) include a tert-butoxycarbonyl group, a tert-butoxycarbonylmethyl group, a tert-pentyloxycarbonyl group, a tert-pentyloxycarbonylmethyl group, a 1,1-diethylpropyloxycarbonyl group, a 1,1-diethylpropyloxycarbonylmethyl group, a 1-ethylcyclopentyloxycarbonyl group, a 1-ethylcyclopentyloxycarbonylmethyl group, a 1-ethyl-2-cyclopentenyloxycarbonyl group, a 1-ethyl-2-cyclopentenyloxycarbonylmethyl group, a 1-ethoxyethoxycarbonylmethyl group, a 2-tetrahydropyranyloxycarbonylmethyl group, a 2-tetrahydrofuranyloxycarbonylmethyl group, etc.

Examples of the acid-labile group represented by the formula (AL-1) further include groups represented by the following formulae (AL-1)-1 to (AL-1)-10.

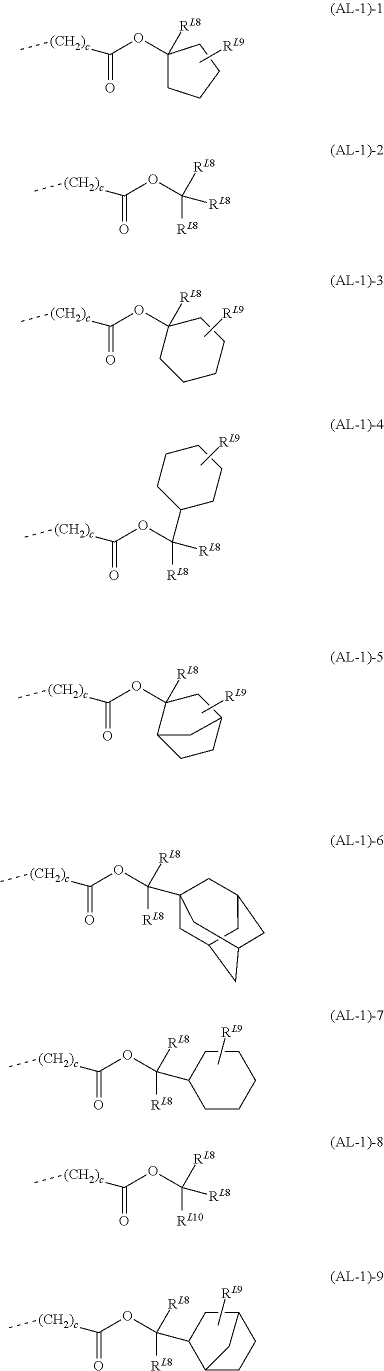

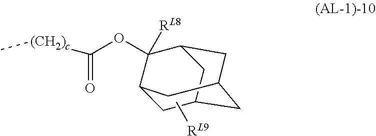

In the formulae, a broken line represents an attachment point.

In the formulae (AL-1)-1 to (AL-1)-10, “b” is as defined above. Each R^{L8} independently represents a saturated hydrocarbyl group having 1 to 10 carbon atoms or an aryl group having 6 to 20 carbon atoms. R^{L9} represents a hydrogen atom or a saturated hydrocarbyl group having 1 to 10 carbon atoms. R^{L10} represents a saturated hydrocarbyl group having 2 to 10 carbon atoms or an aryl group having 6 to 20 carbon atoms. The saturated hydrocarbyl groups may be linear, branched, or cyclic.

In the formula (AL-2), R^{L2} and R^{L3} each independently represent a hydrogen atom or a saturated hydrocarbyl group having 1 to 18, preferably 1 to 10 carbon atoms. The saturated hydrocarbyl group may be linear, branched, or cyclic. Specific examples thereof include a methyl group, an ethyl group, an n-propyl group, an isopropyl group, an n-butyl group, an isobutyl group, a sec-butyl group, a tert-butyl group, a cyclopentyl group, a cyclohexyl group, a 2-ethylhexyl group, an n-octyl group, etc.

In the formula (AL-2), R^{L4} represents a hydrocarbyl group having 1 to 18, preferably 1 to 10 carbon atoms, and optionally contains a heteroatom. The hydrocarbyl group may be saturated or unsaturated, and may be linear, branched, or cyclic. Examples of the hydrocarbyl group include saturated hydrocarbyl groups having 1 to 18 carbon atoms, etc., and part of the hydrogen atoms thereof may be substituted with a hydroxy group, an alkoxy group, an oxo group, an amino group, an alkylamino group, etc. Examples of such substituted saturated hydrocarbyl groups include ones shown below, etc.

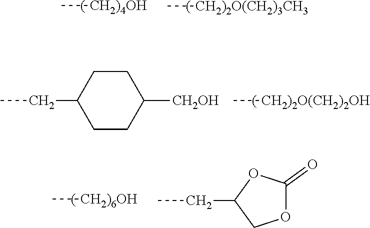

In the formulae, a broken line represents an attachment point.

R^{L2} and R^{L3}, R^{L2} and R^{L4}, or R^{L3} and R^{L4} optionally bond with each other to form a ring together with the carbon atom bonded therewith, or together with the carbon atom and the oxygen atom. In this case, R^{L2} and R^{L3}, R^{L2} and R^{L4}, or R^{L3} and R^{L4}, involved in the ring formation, each independently represent an alkanediyl group having 1 to 18, preferably 1 to 10 carbon atoms. The number of carbon atoms in the ring obtained by bonding these is preferably 3 to 10, more preferably 4 to 10.

Examples of the linear or branched ones among the acid-labile groups shown by the formula (AL-2) include ones represented by the following formulae (AL-2)-1 to (AL-2)-69, but are not limited thereto. Note that, in the following formulae, a broken line represents an attachment point.

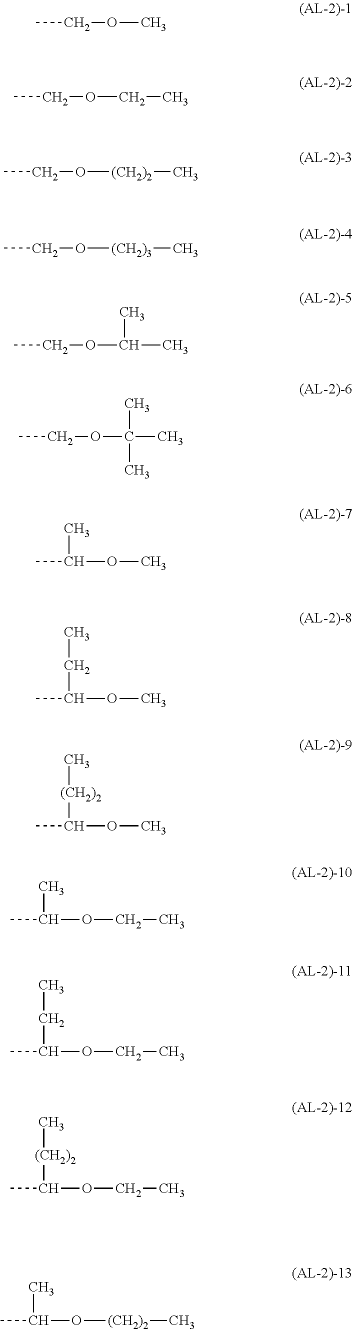

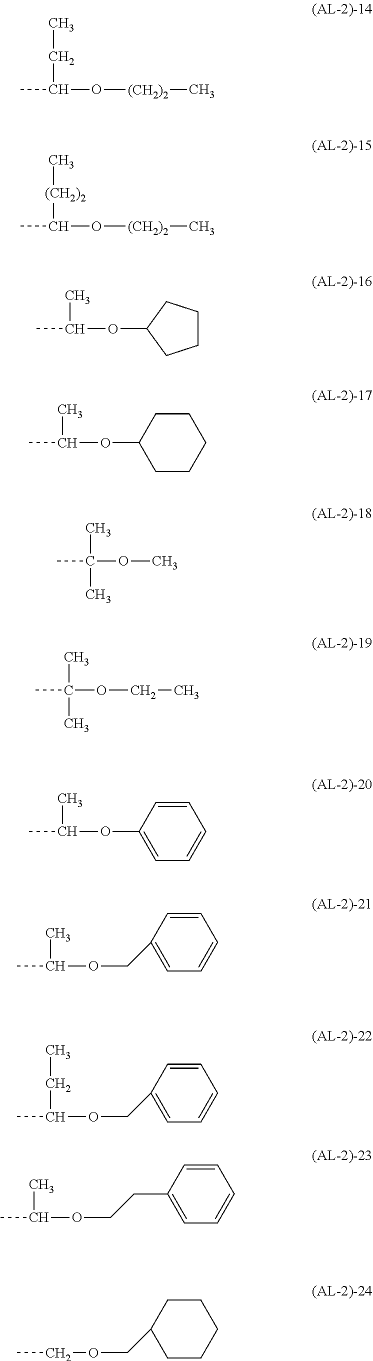

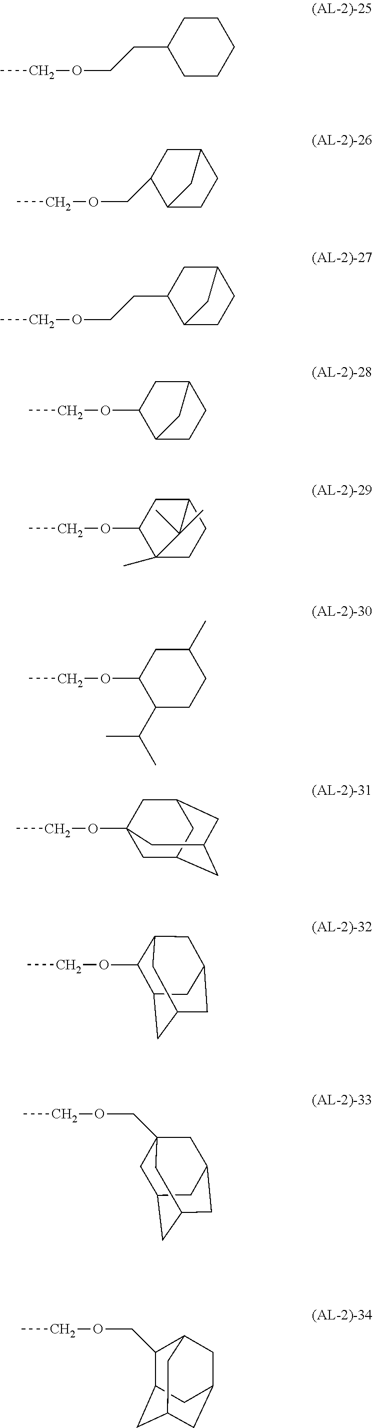

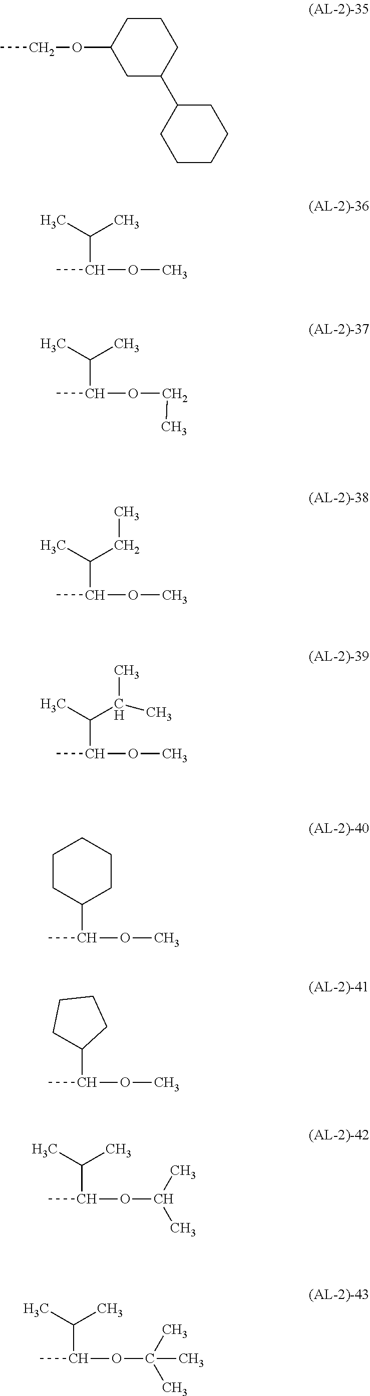

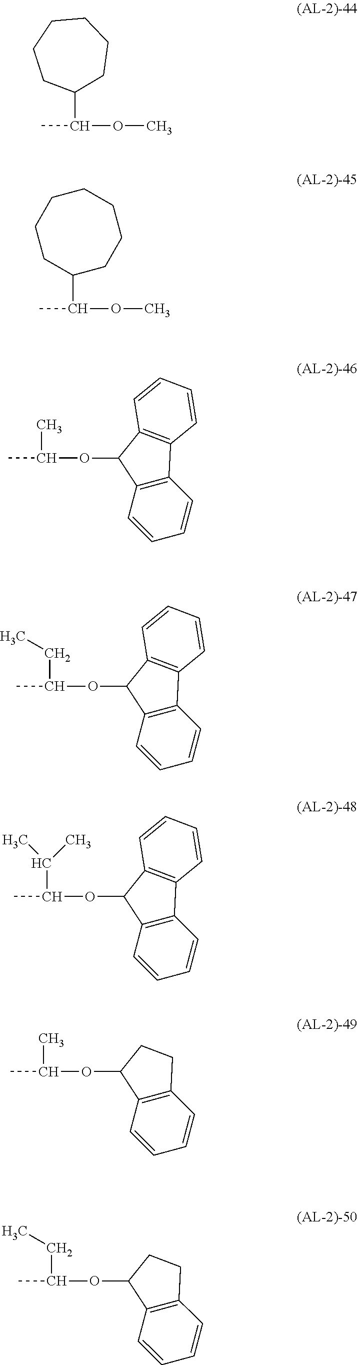

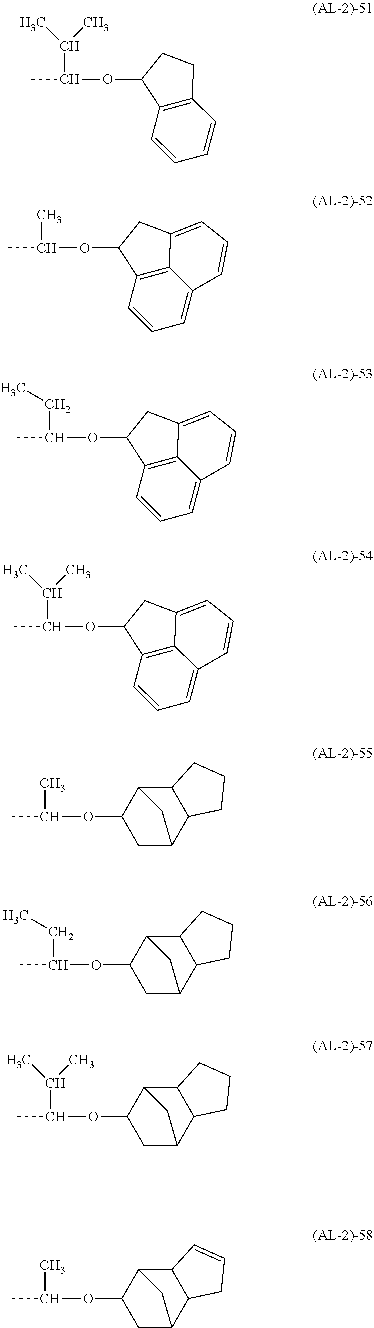

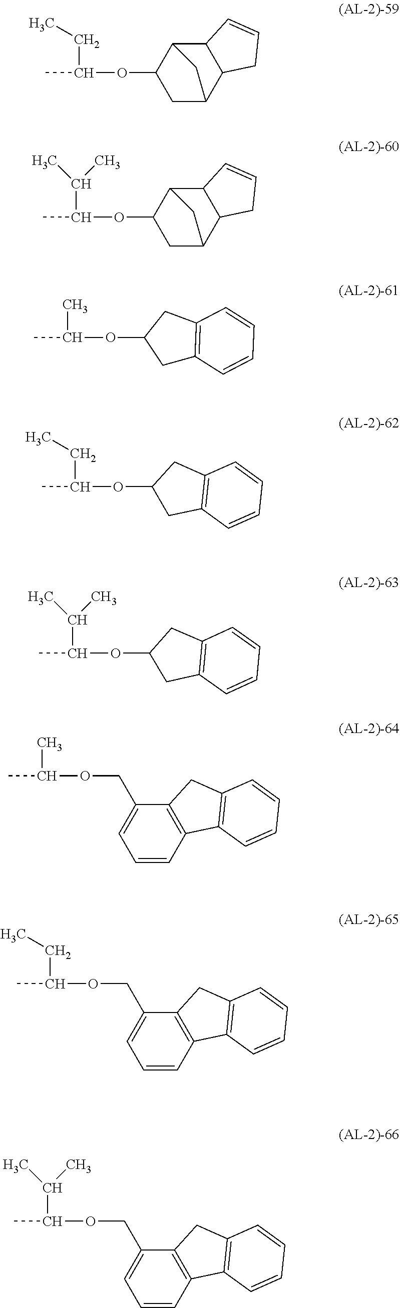

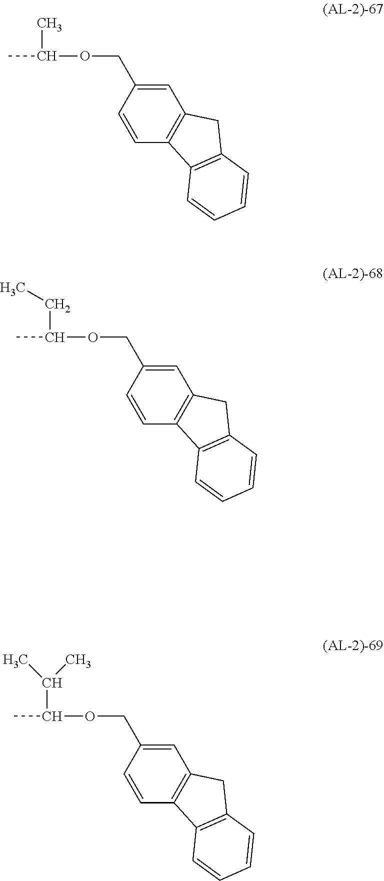

Examples of the cyclic ones among the acid-labile groups represented by the formula (AL-2) include a tetrahydrofuran-2-yl group, a 2-methyltetrahydrofuran-2-yl group, a tetrahydropyran-2-yl group, a 2-methyltetrahydropyran-2-yl group, etc.

In addition, examples of the acid-labile groups include groups represented by the following formula (AL-2a) or (AL-2b). The acid-labile group may crosslink the base polymer intermolecularly or intramolecularly.

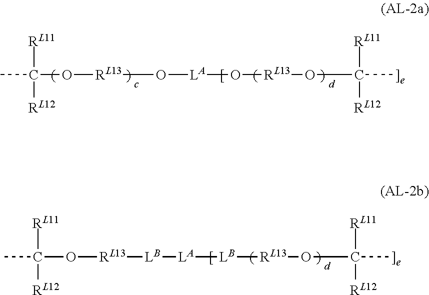

In the formulae, a broken line represents an attachment point.

In the formula (AL-2a) or (AL-2b), R^{L11} and R^{L12} each independently represent a hydrogen atom or a saturated hydrocarbyl group having 1 to 8 carbon atoms. The saturated hydrocarbyl group may be linear, branched, or cyclic. Alternatively, R^{L11} and R^{L12} may bond with each other to form a ring together with the carbon atom bonded therewith. In this case, R^{L11} and R^{L12} each independently represent an alkanediyl group having 1 to 8 carbon atoms. Each R^{L13} independently represents a saturated hydrocarbylene group having 1 to 10 carbon atoms. The saturated hydrocarbylene group may be linear, branched, or cyclic. “c” and “d” each independently represent an integer of 0 to 10, preferably an integer of 0 to 5, and “e” represents an integer of 1 to 7, preferably an integer of 1 to 3.

In the formula (AL-2a) or (AL-2b), L^{A} represents an aliphatic saturated hydrocarbon group having a valency of e+1 and having 1 to 50 carbon atoms, an alicyclic saturated hydrocarbon group having a valency of e+1 and having 3 to 50 carbon atoms, an aromatic hydrocarbon group having a valency of e+1 and having 6 to 50 carbon atoms, or a heterocyclic group having a valency of e+1 and having 3 to 50 carbon atoms. Furthermore, part of the —CH₂— of these groups may be substituted with a group containing a heteroatom, and part of the hydrogen atoms of these groups may be substituted with a hydroxy group, a carboxy group, an acyl group, or a fluorine atom. L^{A} is preferably a saturated hydrocarbon group, such as a saturated hydrocarbylene group, a trivalent saturated hydrocarbon group, and a tetravalent saturated hydrocarbon group, each of which has 1 to 20 carbon atoms, an arylene group having 6 to 30 carbon atoms, etc. The saturated hydrocarbon groups may be linear, branched, or cyclic. L^{B} represents —C(═O)—O—, —NH—C(═O)—O—, or —NH—C(═O)—NH—.

Examples of the crosslinking acetal group represented by the formula (AL-2a) or (AL-2b) include groups represented by the following formulae (AL-2)-70 to (AL-2)-77, etc.

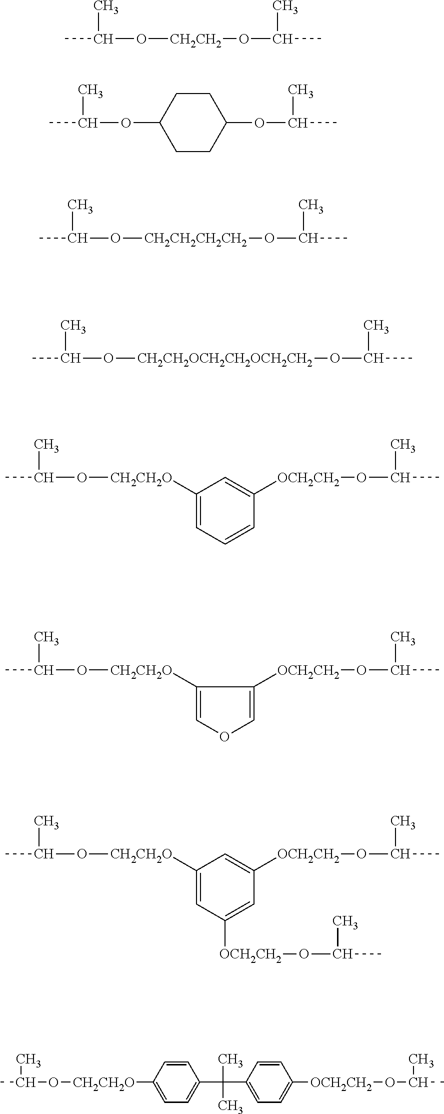

In the formulae, a broken line represents an attachment point.

In the formula (AL-3), R^{L5} represents a hydrogen atom or a hydrocarbyl group having 1 to 20 carbon atoms, and the hydrocarbyl group optionally contains a heteroatom, such as an oxygen atom, a sulfur atom, a nitrogen atom, and a fluorine atom. R^{L6} and R^{L7} each independently represent a hydrocarbyl group having 1 to 20 carbon atoms, and optionally contain a heteroatom, such as an oxygen atom, a sulfur atom, a nitrogen atom, and a fluorine atom. The hydrocarbyl group may be saturated or unsaturated, and may be linear, branched, or cyclic. Specific examples thereof include alkyl groups having 1 to 20 carbon atoms, cyclic saturated hydrocarbyl groups having 3 to 20 carbon atoms, alkenyl groups having 2 to 20 carbon atoms, cyclic unsaturated hydrocarbyl groups having 3 to 20 carbon atoms, aryl groups having 6 to 10 carbon atoms, etc. Alternatively, R^{L5} and R^{L6}, R^{L5} and R^{L7}, or R^{L6} and R^{L7} may bond with each other to form an alicyclic group having 3 to 20 carbon atoms, together with the carbon atom bonded therewith.

Examples of the group represented by the formula (AL-3) include a tert-butyl group, a 1,1-diethylpropyl group, a 1-ethylnorbornyl group, a 1-methylcyclopentyl group, a 1-ethylcyclopentyl group, a 1-isopropylcyclopentyl group, a 1-methylcyclohexyl group, a 2-(2-methyl) adamantyl group, a 2-(2-ethyl) adamantyl group, a tert-pentyl group, etc.

Examples of the group represented by the formula (AL-3) also include groups represented by the following formulae (AL-3)-1 to (AL-3)-22.

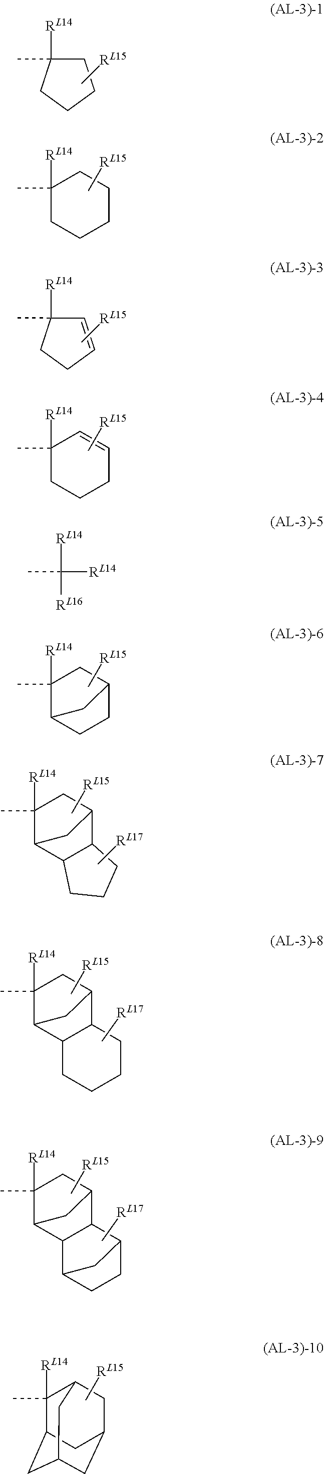

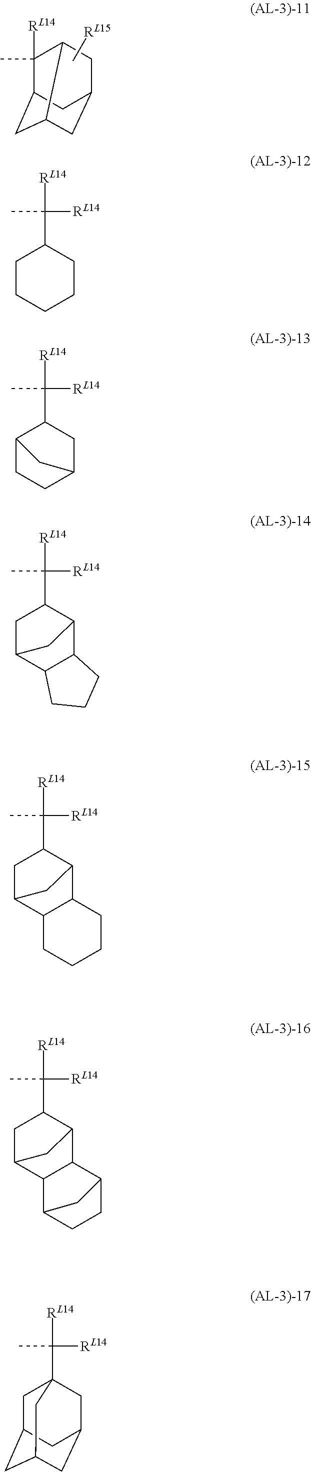

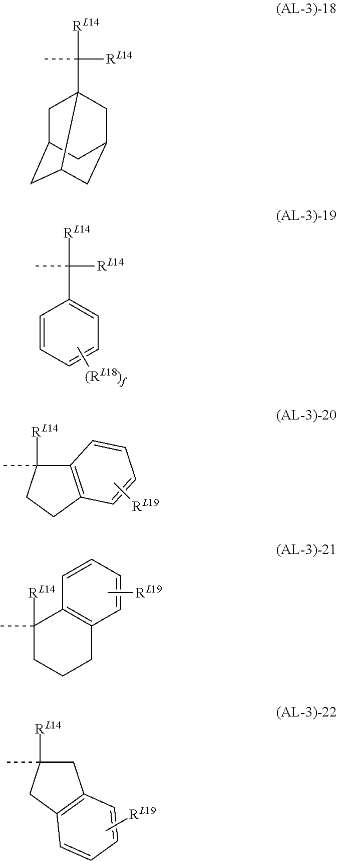

In the formulae, a broken line represents an attachment point.

In the formulae (AL-3)-1 to (AL-3)-22, each R^{L14} independently represents a hydrogen atom, an aliphatic hydrocarbyl group having 1 to 8 carbon atoms, or an aryl group having 6 to 20 carbon atoms. R^{L15} and R^{L17} each independently represent a hydrogen atom or a saturated hydrocarbyl group having 1 to 20 carbon atoms. R^{L16} represents an aryl group having 6 to 20 carbon atoms. The saturated hydrocarbyl groups may be linear, branched, or cyclic. Furthermore, as the aryl group, a phenyl group etc. are preferable. R^{L18} represents a fluorine atom, an iodine atom, a nitro group, or a trifluoromethyl group. Each R^{L19} independently represents a hydrogen atom, a fluorine atom, an iodine atom, a nitro group, a saturated hydrocarbyl group having 1 to 8 carbon atoms, or a hydrocarbyloxy group having 1 to 8 carbon atoms. “f” represents an integer of 1 to 5.

Examples of the acid-labile group further include groups represented by the following formula (AL-3)-23 or (AL-3)-24. The acid-labile group may crosslink the polymer intramolecularly or intermolecularly.

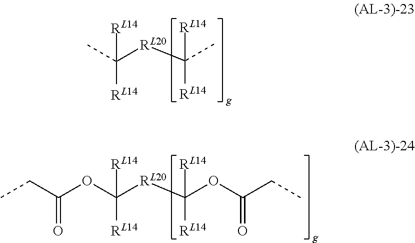

In the formulae, a broken line represents an attachment point.

In the formulae (AL-3)-23 and (AL-3)-24, R^{L14} is as defined above. R^{L20} represents a saturated or unsaturated hydrocarbylene group having a valency of g+1 and having 1 to 20 carbon atoms or represents an arylene group having a valency of g+1 and having 6 to 20 carbon atoms, and optionally contains a heteroatom such as an oxygen atom, a sulfur atom, and a nitrogen atom. The saturated or unsaturated hydrocarbylene group may be linear, branched, or cyclic. “g” represents an integer of 1 to 3.

Besides these acid-labile groups, it is also possible to use aromatic-group-containing acid-labile groups disclosed in JP5565293B2, JP5434983B2, JP5407941B2, JP5655756B2, and JP5655755B2.

The above-described base polymer may include a repeating unit-b containing a phenolic hydroxy group as an adhesive group. Specific examples of a monomer to give the repeating unit-b include ones shown below, but are not limited thereto. Note that, in the following formulae, R^{A} is as defined above.

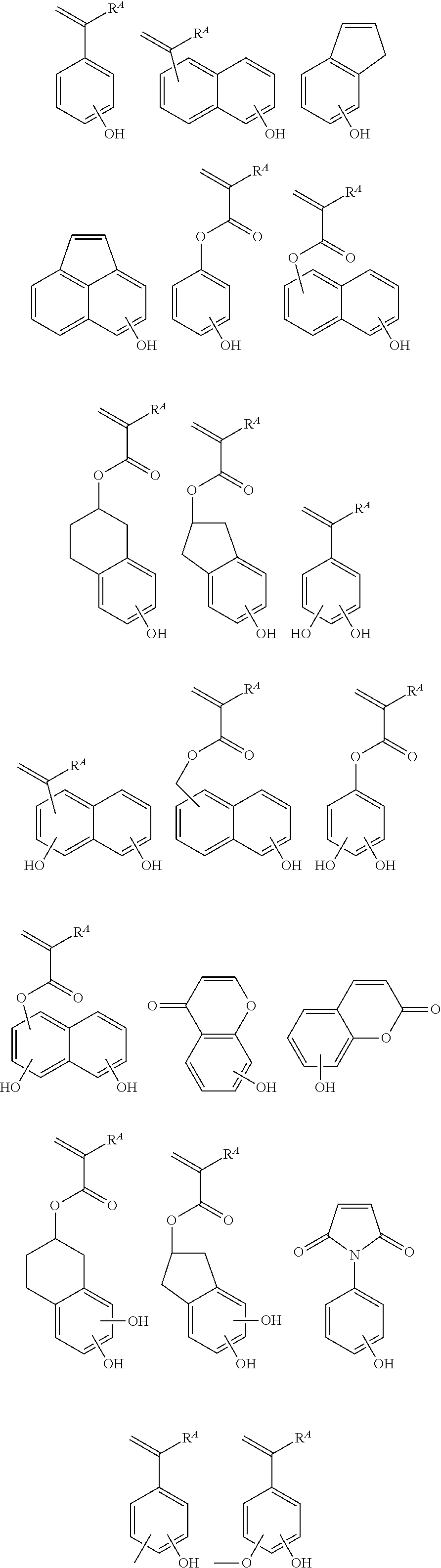

The base polymer may include a repeating unit-c containing, as a different adhesive group, a hydroxy group other than a phenolic hydroxy group, a lactone ring, a sultone ring, an ether bond, an ester bond, a sulfonic acid ester bond, a carbonyl group, a sulfonyl group, a cyano group, or a carboxy group. Specific examples of a monomer to give the repeating unit-c include ones shown below, but are not limited thereto. Note that, in the following formulae, R^{A} is as defined above.

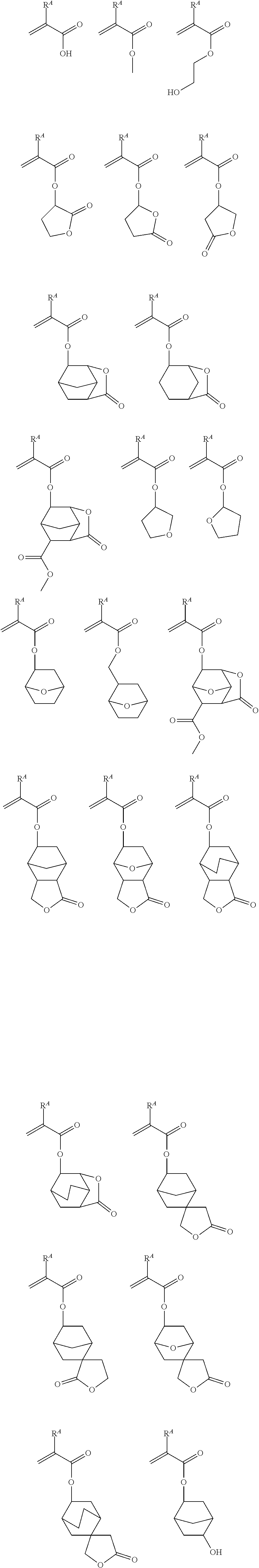

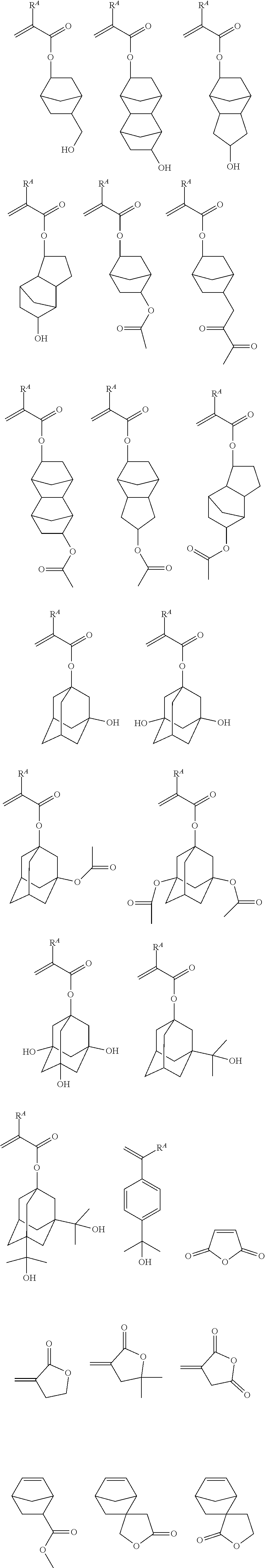

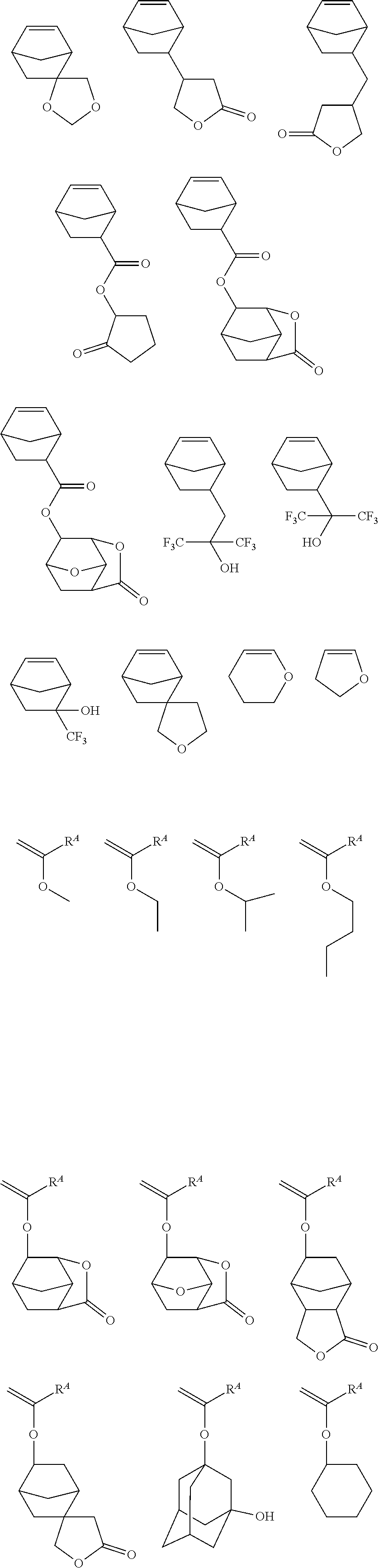

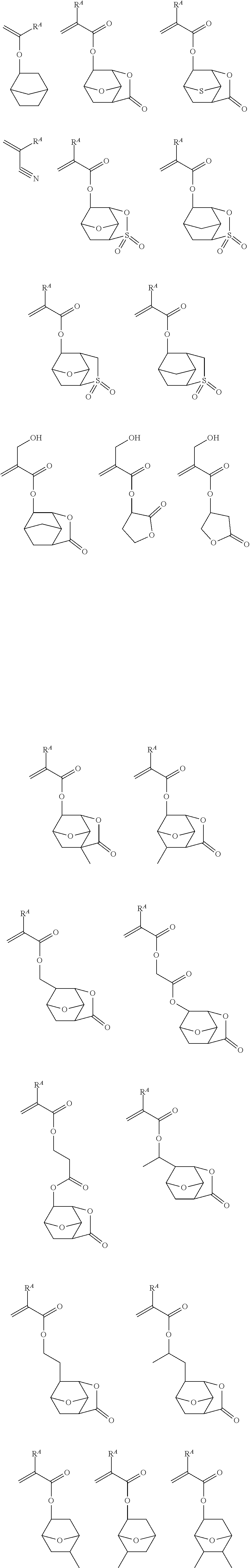

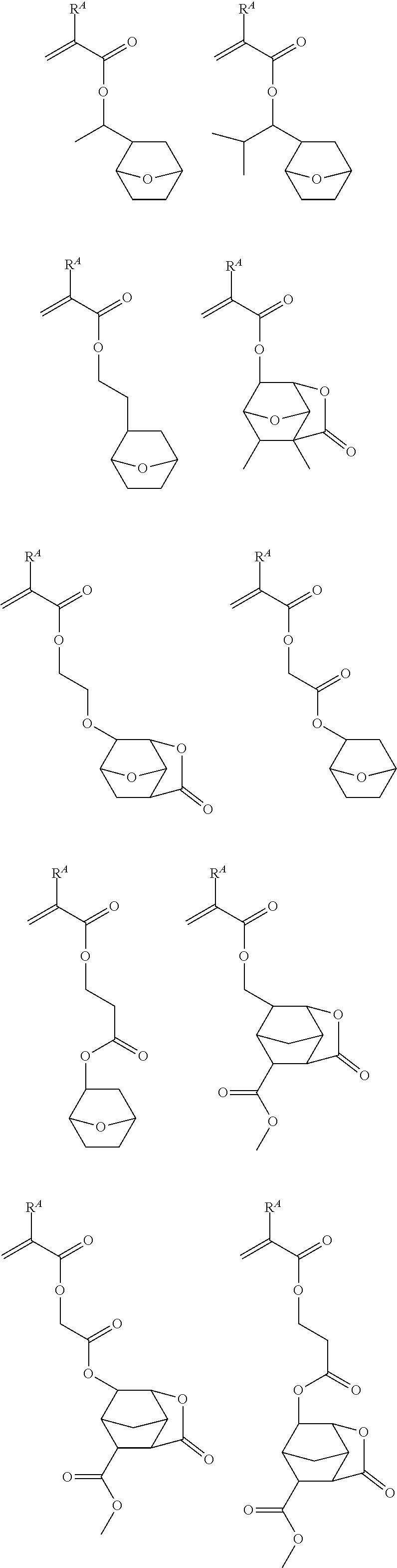

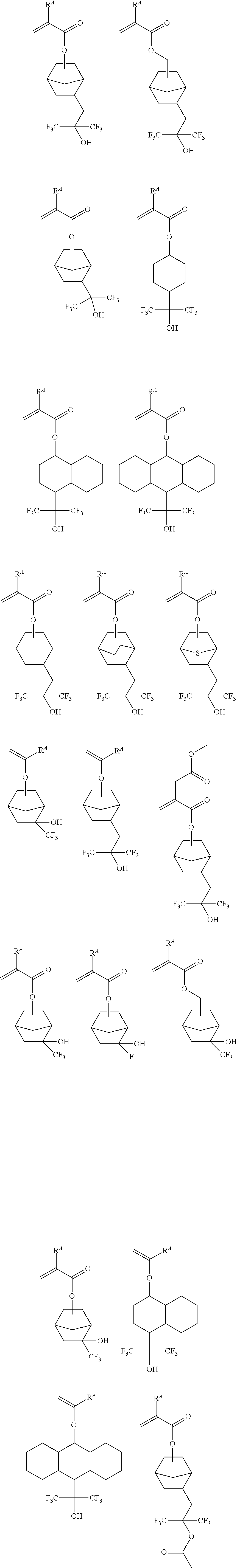

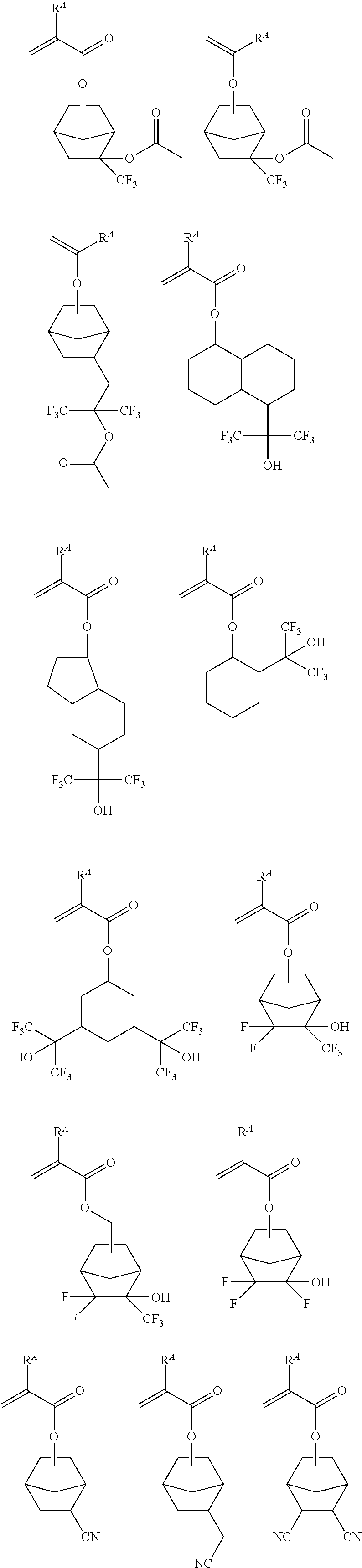

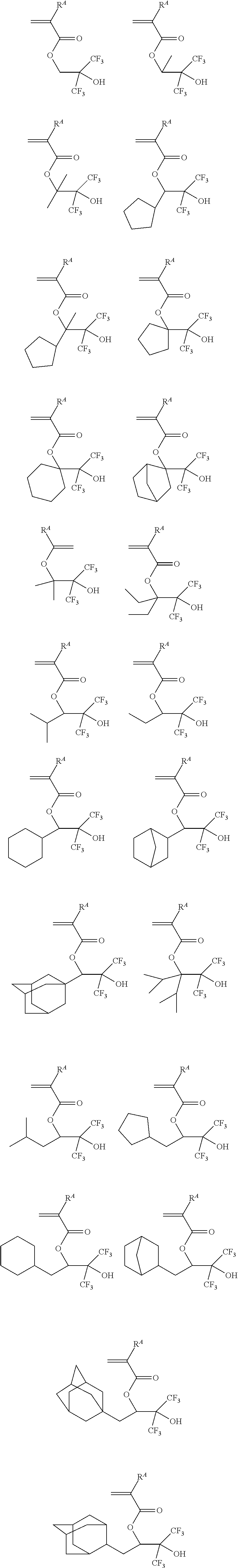

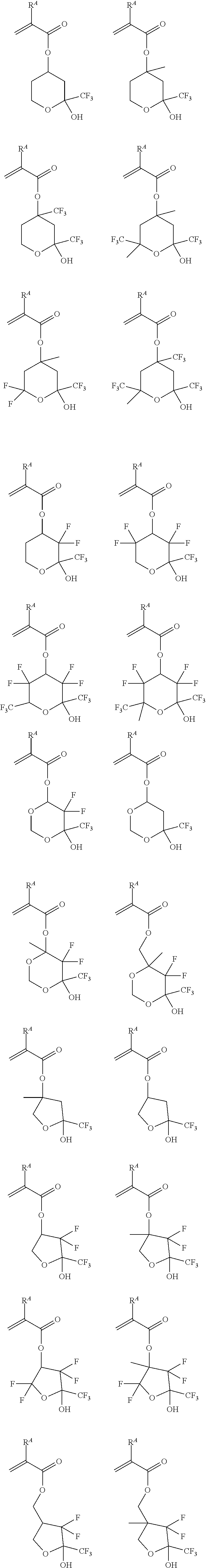

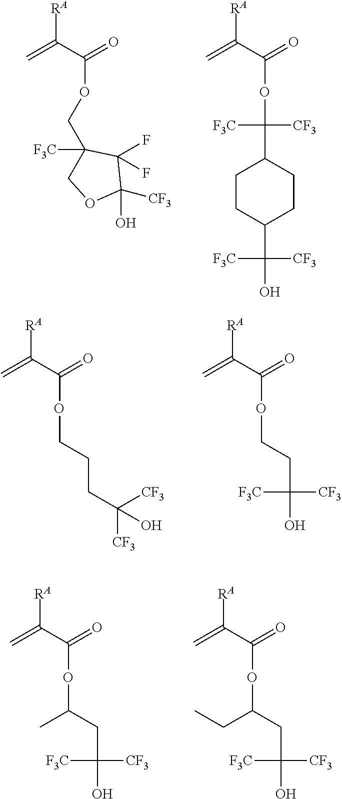

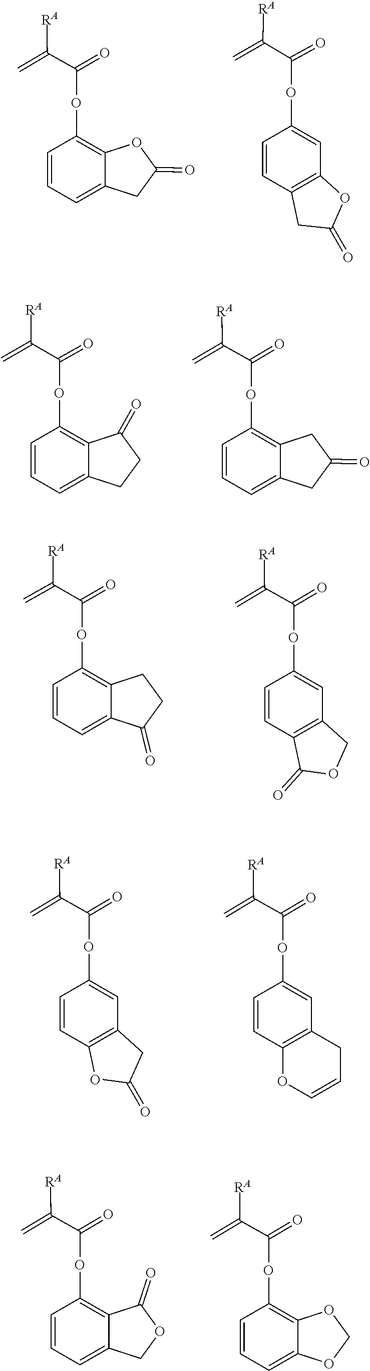

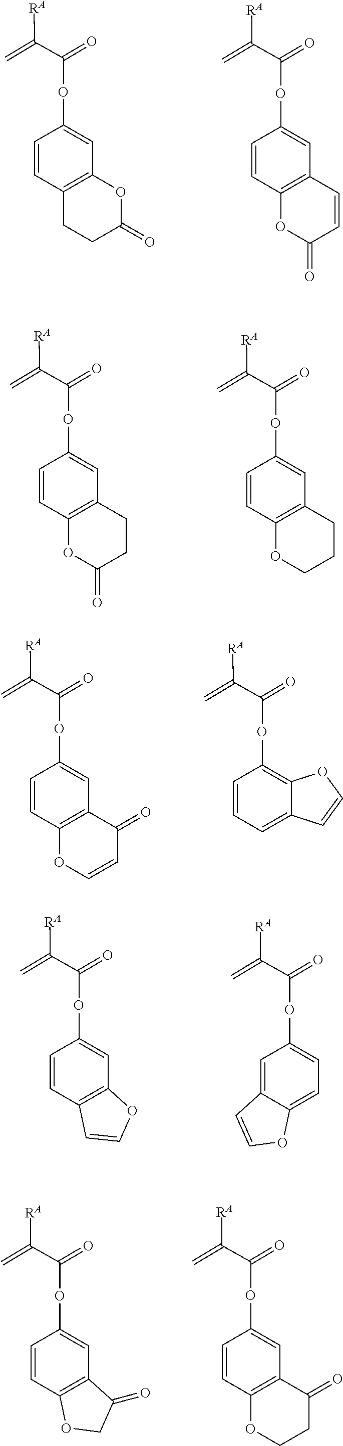

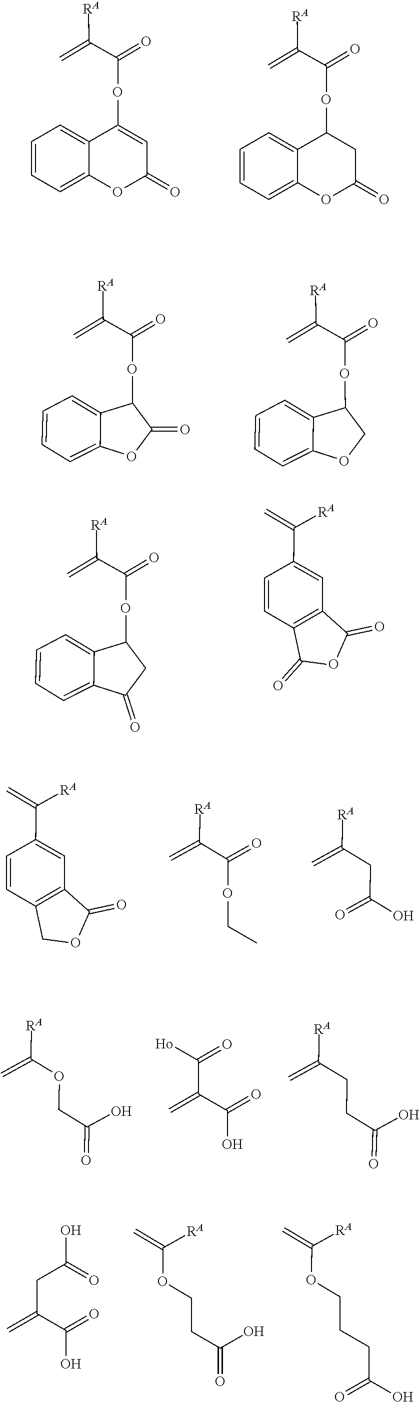

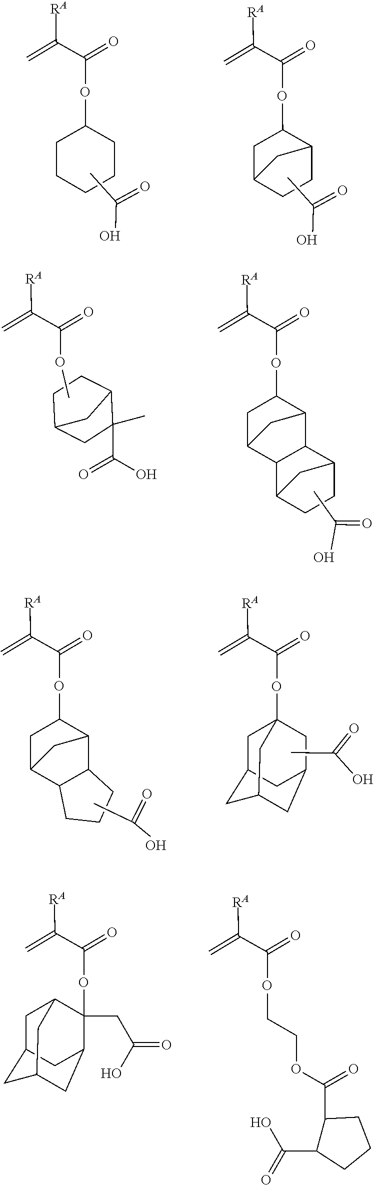

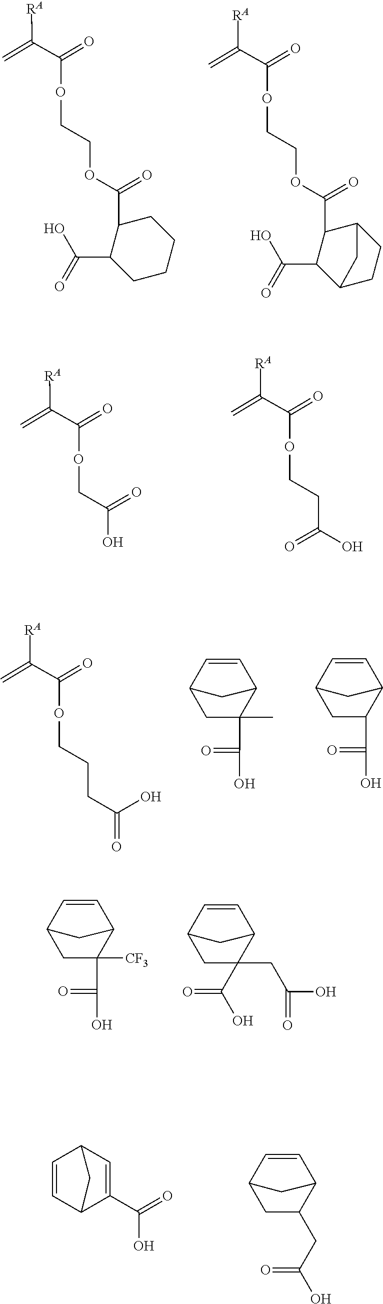

The base polymer may include a repeating unit-d derived from indene, benzofuran, benzothiophene, acenaphthylene, chromone, coumarin, norbornadiene, or a derivative thereof. Specific examples of a monomer to give the repeating unit-d include ones shown below, but are not limited thereto.

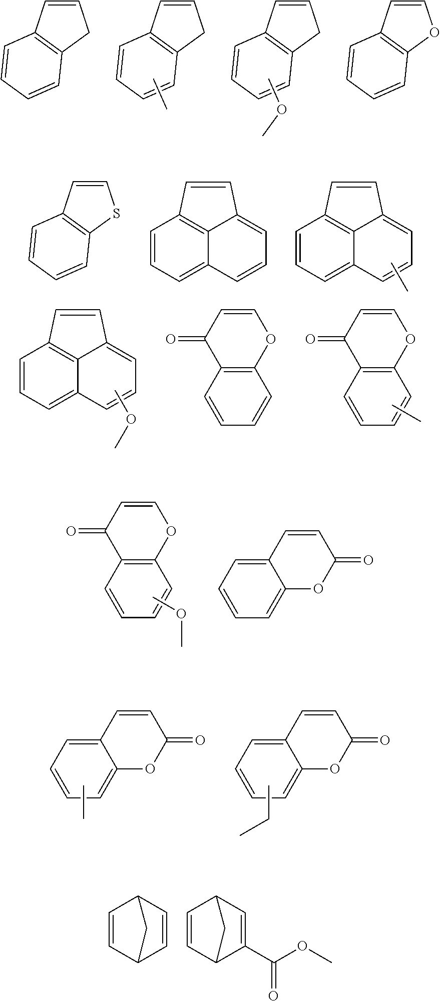

The base polymer may include a repeating unit-e derived from styrene, vinylnaphthalene, vinylanthracene, vinylpyrene, methyleneindane, vinylpyridine, or vinylcarbazole.

The base polymer may include a repeating unit-f derived from an onium salt including a polymerizable unsaturated bond. Specific examples of preferable repeating units-f include a repeating unit represented by the following formula (f1) (hereinafter, also referred to as repeating unit-f1), a repeating unit represented by the following formula (f2) (hereinafter, also referred to as repeating unit-f2), and a repeating unit represented by the following formula (f3) (hereinafter, also referred to as repeating unit-f3). Incidentally, one kind of the repeating units-f1 to -f3 may be used, or two or more kinds thereof may be used in combination.

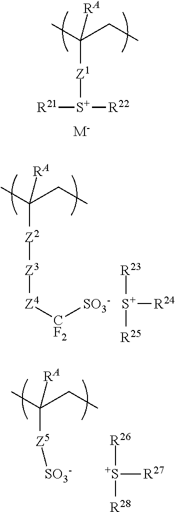

In the formulae (f1) to (f3), each R^{A} independently represents a hydrogen atom or a methyl group. Z¹ represents a single bond, an aliphatic hydrocarbylene group having 1 to 6 carbon atoms, a phenylene group, a naphthylene group, a group having 7 to 18 carbon atoms derived from a combination of these groups, —O—Z¹¹—, —C(═O)—O—Z¹¹—, or —C(═O)—NH—Z¹¹—. Z¹¹ represents an aliphatic hydrocarbylene group having 1 to 6 carbon atoms, a phenylene group, a naphthylene group, or a group having 7 to 18 carbon atoms derived from a combination of these groups, Z¹¹ optionally containing a carbonyl group, an ester bond, an ether bond, or a hydroxy group. Z² represents a single bond or an ester bond. Z³ represents a single bond, —Z³¹—C(═O)—O—, —Z³¹—O—, or —Z³¹—O—C(═O)—. Z³¹ represents an aliphatic hydrocarbylene group having 1 to 12 carbon atoms, a phenylene group, or a group having 7 to 18 carbon atoms derived from a combination of these groups, Z³¹ optionally containing a carbonyl group, an ester bond, an ether bond, an iodine atom, or a bromine atom. Z⁴ represents a methylene group, a 2,2,2-trifluoro-1,1-ethanediyl group, or a carbonyl group. Z⁵ represents a single bond, a methylene group, an ethylene group, a phenylene group, a fluorinated phenylene group, a phenylene group having a substituent which is a trifluoromethyl group, —O—Z⁵¹—, —C(═O)—O—Z⁵¹—, or —C(═O)—NH—Z⁵¹—. Z⁵¹ represents an aliphatic hydrocarbylene group having 1 to 6 carbon atoms, a phenylene group, a fluorinated phenylene group, or a phenylene group having a substituent which is a trifluoromethyl group, Z⁵¹ optionally containing a carbonyl group, an ester bond, an ether bond, a hydroxy group, or a halogen atom.

In the formulae (f1) to (f3), R²¹ to R²⁸ each independently represent a halogen atom or a hydrocarbyl group having 1 to 20 carbon atoms and optionally containing a heteroatom. Specific examples of the halogen atom include a fluorine atom, a chlorine atom, a bromine atom, and an iodine atom. The hydrocarbyl group may be saturated or unsaturated, and may be linear, branched, or cyclic. Specific examples thereof include those given as examples of the hydrocarbyl groups represented by R^{ct1} to R^{ct5} in the description of the formulae (Z-1) and (Z-2). Furthermore, part or all of the hydrogen atoms of the hydrocarbyl group may be substituted with a group containing a heteroatom, such as an oxygen atom, a sulfur atom, a nitrogen atom, and a halogen atom, and part of the —CH₂— of the hydrocarbyl group may be substituted with a group containing a heteroatom such as an oxygen atom, a sulfur atom, or a nitrogen atom. The resulting hydrocarbyl group may contain a hydroxy group, a fluorine atom, a chlorine atom, a bromine atom, an iodine atom, a cyano group, a nitro group, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, etc. Furthermore, R²³ and R²⁴ or R²⁶ and R²⁷ may be bonded to each other to form a ring together with the sulfur atom bonded thereto. In this case, specific examples of the ring include those given in the description of the formula (Z-1) as examples of the ring that may be formed by R^{ct1} and R^{ct2} being bonded to each other together with the sulfur atom bonded thereto.

In the formula (f1), M-represents a non-nucleophilic counter ion. Specific examples of the non-nucleophilic counter ion include: halide ions, such as chloride and bromide ions; fluoroalkylsulfonate ions, such as triflate, 1,1,1-trifluoroethanesulfonate, and nonafluorobutanesulfonate ions; arylsulfonate ions, such as tosylate, benzenesulfonate, 4-fluorobenzenesulfonate, and 1,2,3,4,5-pentafluorobenzenesulfonate ions; alkylsulfonate ions, such as mesylate and butanesulfonate ions; imide ions, such as bis(trifluoromethylsulfonyl)imide, bis(perfluoroethylsulfonyl)imide, and bis(perfluorobutylsulfonyl)imide ions; and methide ions such as tris(trifluoromethylsulfonyl) methide and tris(perfluoroethylsulfonyl) methide ions.

Specific examples of the non-nucleophilic counter ion further include: sulfonate ions represented by the following formula (f1-1), having a substituting fluorine atom at α position; sulfonate ions represented by the following formula (f1-2), having a substituting fluorine atom at α position and having a substituting trifluoromethyl group at β position; etc.

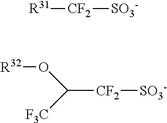

In the formula (f1-1), R³¹ represents a hydrogen atom or a hydrocarbyl group having 1 to 20 carbon atoms, the hydrocarbyl group optionally containing at least one selected from an ether bond, an ester bond, a carbonyl group, a lactone ring, and a fluorine atom.

In the formula (f1-2), R³² represents a hydrogen atom, a hydrocarbyl group having 1 to 30 carbon atoms, or a hydrocarbylcarbonyl group having 2 to 30 carbon atoms, the hydrocarbyl group and the hydrocarbylcarbonyl group optionally containing at least one selected from an ether bond, an ester bond, a carbonyl group, and a lactone ring.

The hydrocarbyl group and the hydrocarbyl moiety of the hydrocarbylcarbonyl group represented by R³¹ or R³² may be saturated or unsaturated, and may be linear, branched, or cyclic. Specific examples thereof include: alkyl groups, such as a methyl group, an ethyl group, a propyl group, an isopropyl group, a butyl group, an isobutyl group, a sec-butyl group, a tert-butyl group, a pentyl group, a neopentyl group, a hexyl group, a heptyl group, a 2-ethylhexyl group, a nonyl group, an undecyl group, a tridecyl group, a pentadecyl group, a heptadecyl group, and an icosyl group; cyclic saturated hydrocarbyl groups, such as a cyclopentyl group, a cyclohexyl group, a 1-adamantyl group, a 2-adamantyl group, a 1-adamantylmethyl group, a norbornyl group, a norbornylmethyl group, a tricyclodecanyl group, a tetracyclododecanyl group, a tetracyclododecanylmethyl group, and a dicyclohexylmethyl group; alkenyl groups, such as an allyl group; cyclic unsaturated hydrocarbyl groups, such as a 3-cyclohexenyl group; aryl groups, such as a phenyl group, a 1-naphthyl group, and a 2-naphthyl group; aralkyl groups, such as a benzyl group and a diphenylmethyl group; etc.

Furthermore, part or all of the hydrogen atoms of these groups may be substituted with a group containing a heteroatom, such as an oxygen atom, a sulfur atom, a nitrogen atom, and a halogen atom, and part of the carbon atoms of the groups may be substituted with a group containing a heteroatom, such as an oxygen atom, a sulfur atom, and a nitrogen atom. The resulting group may contain a hydroxy group, a cyano group, a carbonyl group, an ether bond, an ester bond, a sulfonic acid ester bond, a carbonate bond, a lactone ring, a sultone ring, a carboxylic anhydride, a haloalkyl group, etc. Specific examples of the hydrocarbyl group containing a heteroatom include a tetrahydrofuryl group, a methoxymethyl group, an ethoxymethyl group, a methylthiomethyl group, an acetamidomethyl group, a trifluoroethyl group, a (2-methoxyethoxy)methyl group, an acetoxymethyl group, a 2-carboxy-1-cyclohexyl group, a 2-oxopropyl group, a 4-oxo-1-adamantyl group, and a 3-oxocyclohexyl group.

Specific examples of a cation of a monomer to give the repeating unit-f1 include ones shown below, but are not limited thereto. Note that, in the following formulae, R^{A} is as defined above.

[chemical structure]

Specific examples of a cation of a monomer to give the repeating unit-f2 or -f3 include ones given as examples of the sulfonium cation represented by the formula (Z-1).

Specific examples of a monomer to give the repeating unit-f2 include ones shown below, but are not limited thereto. Note that, in the following formulae, R^{A} is as defined above.

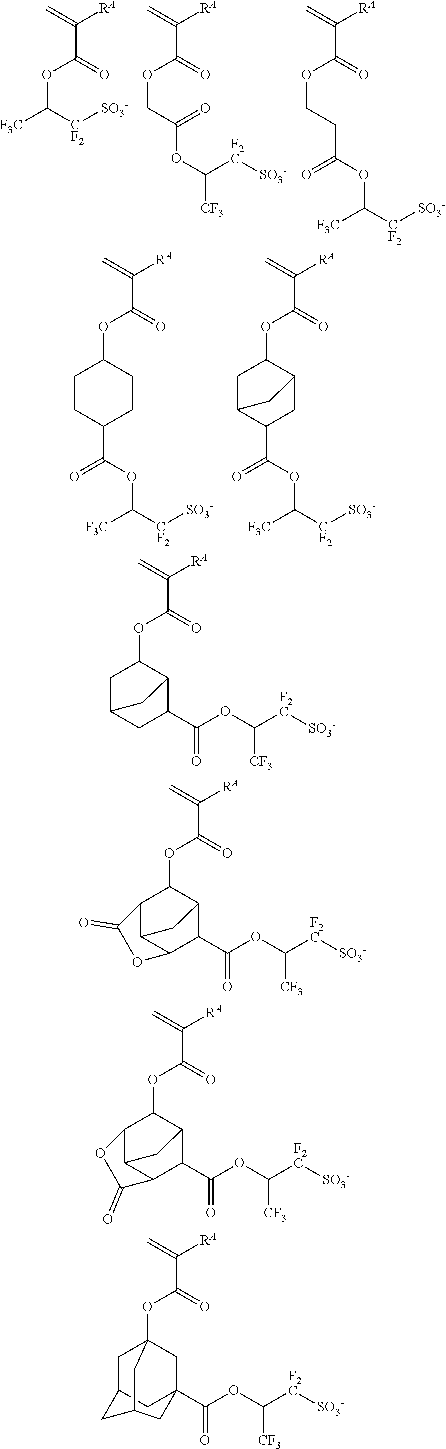

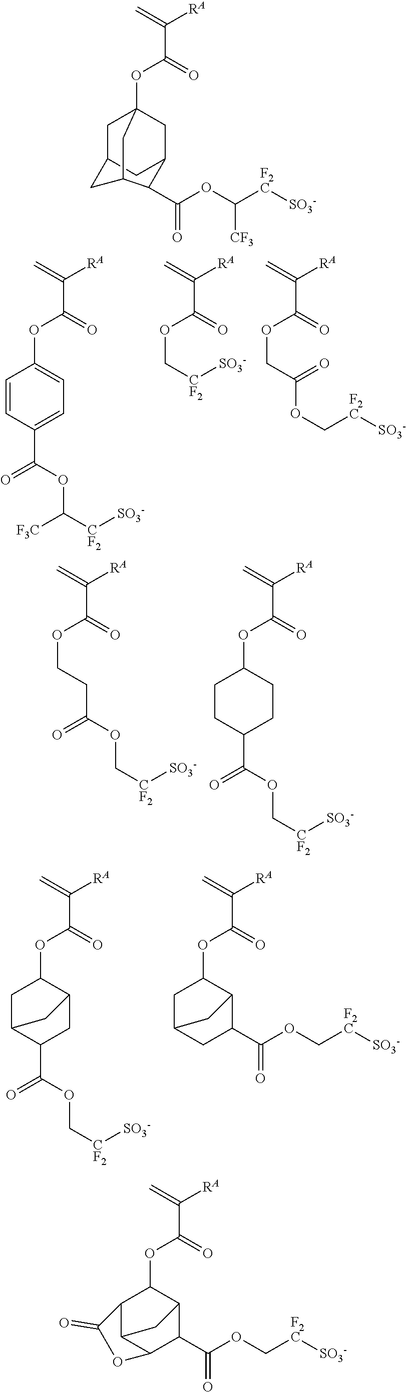

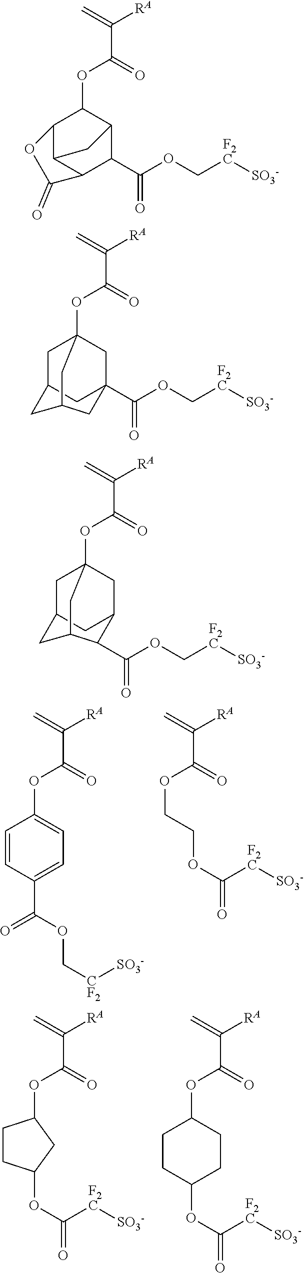

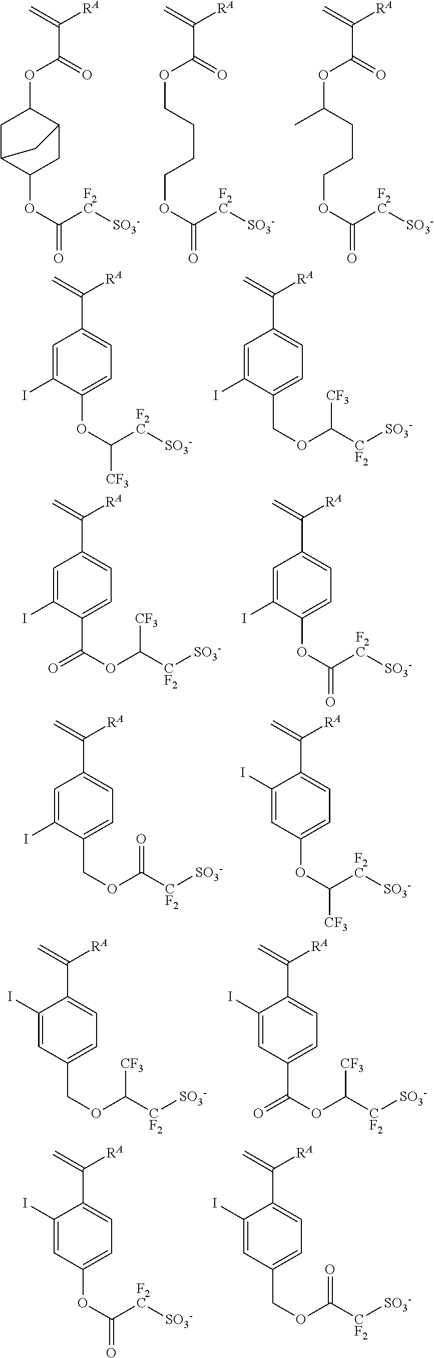

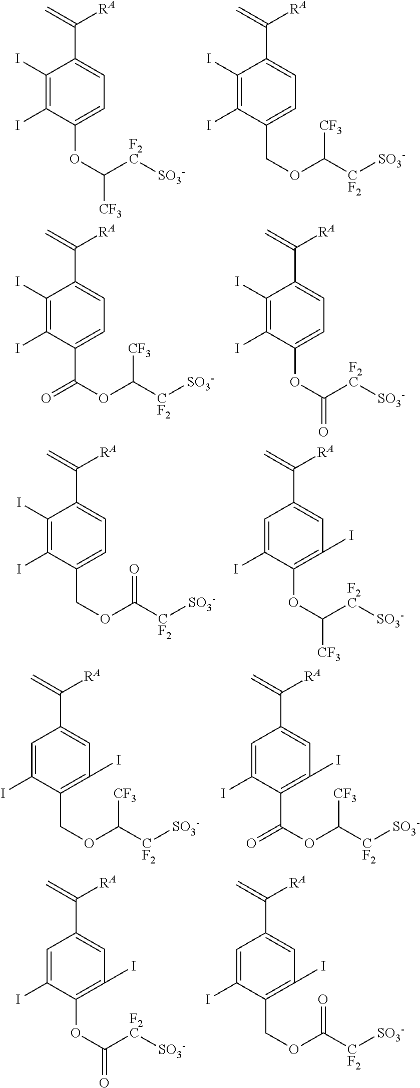

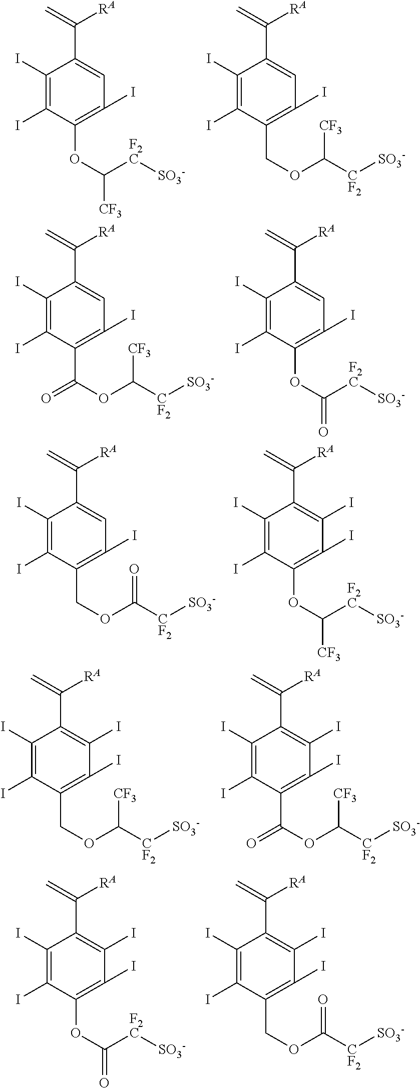

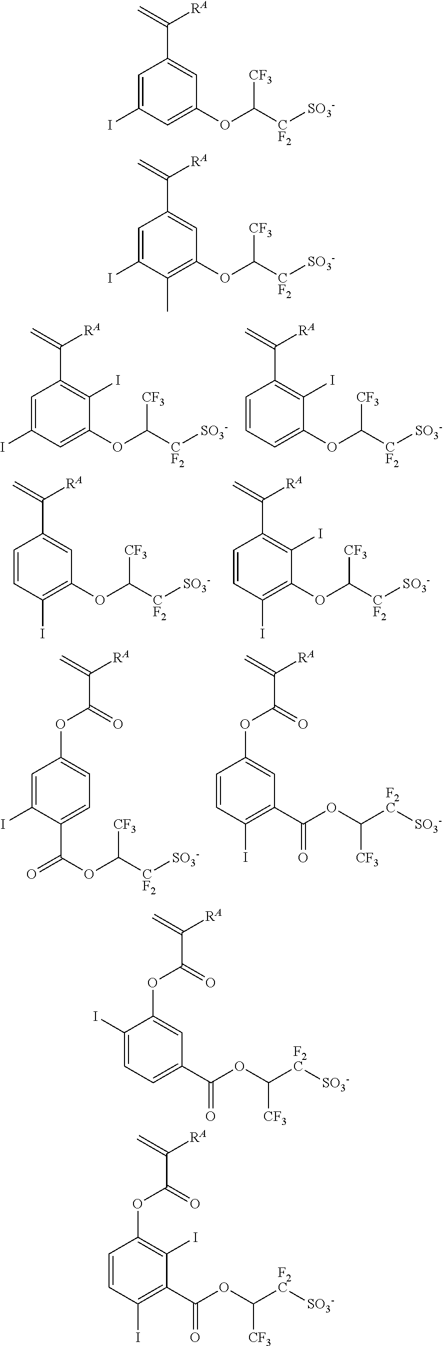

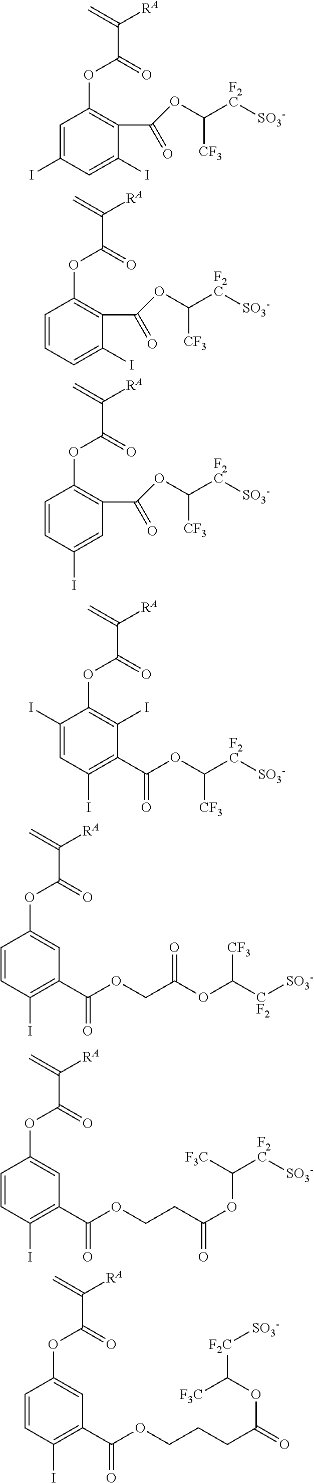

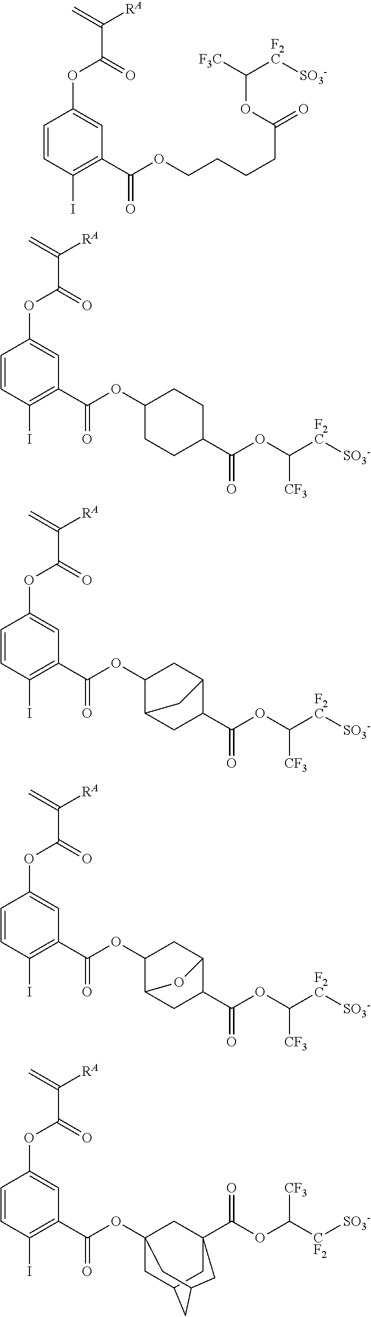

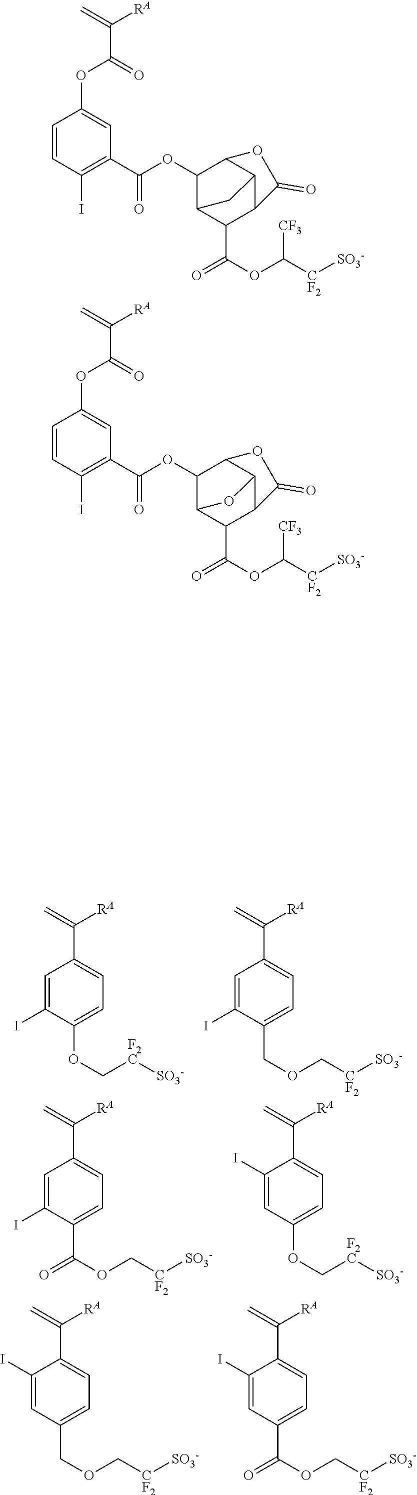

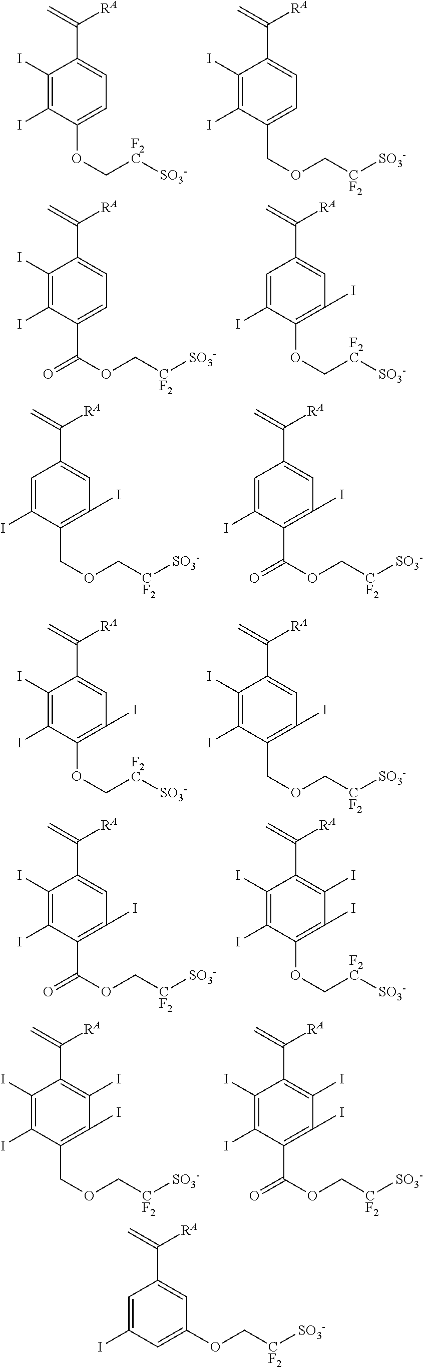

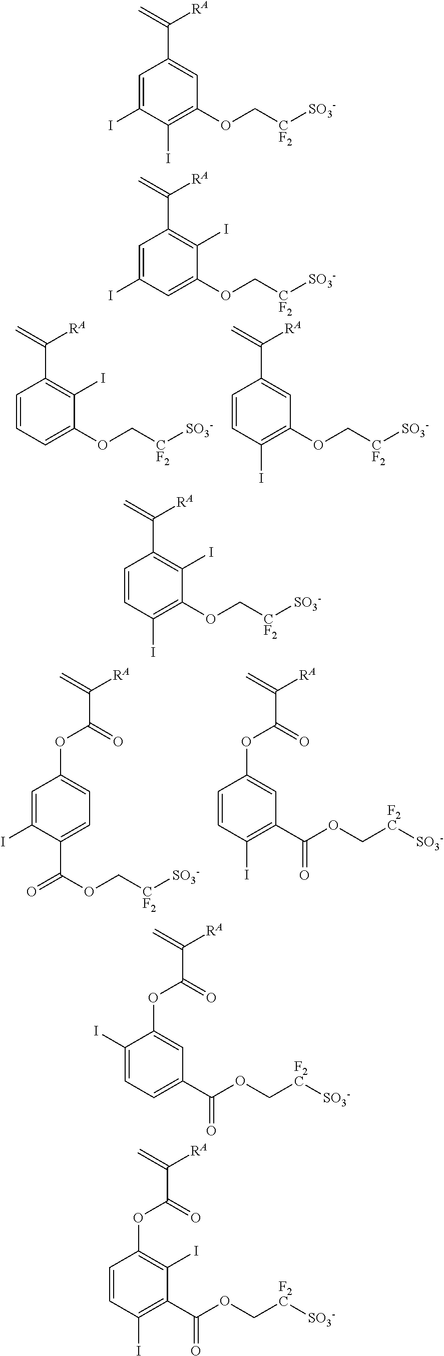

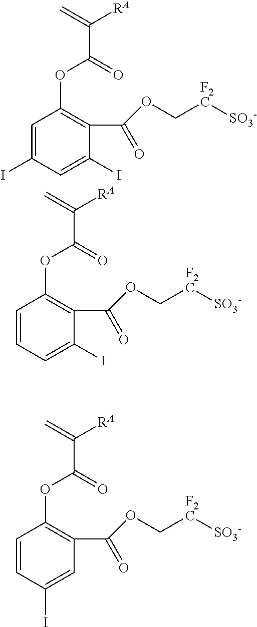

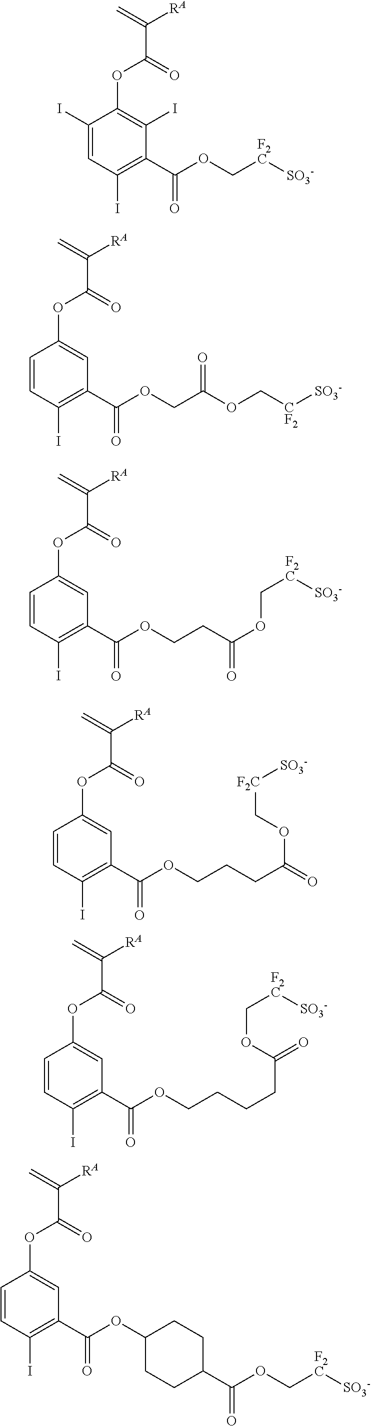

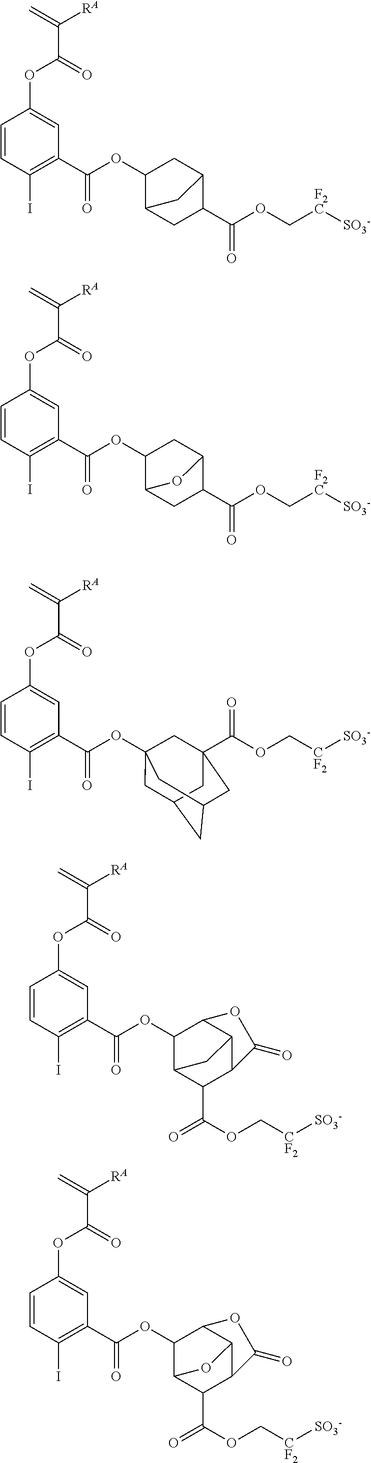

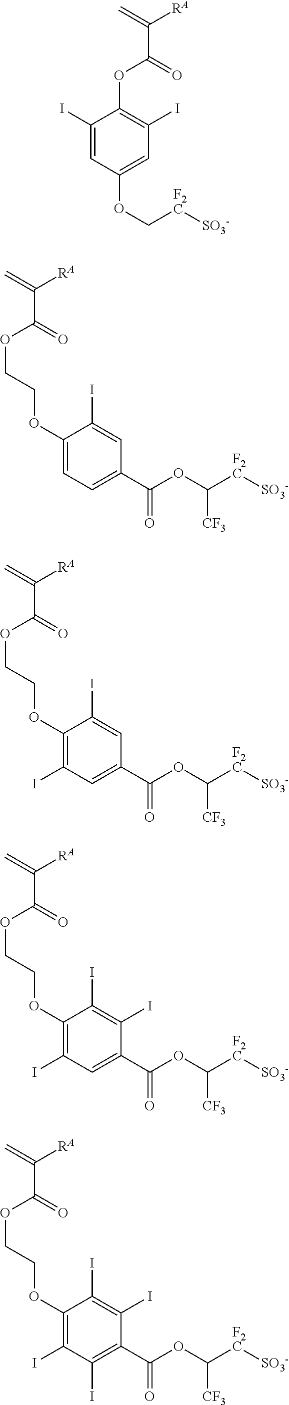

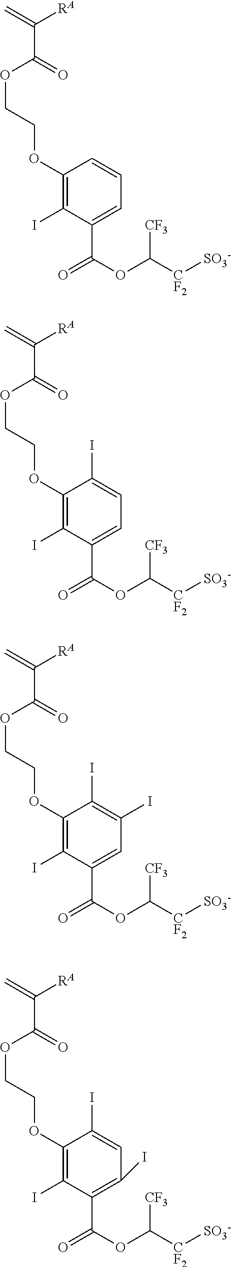

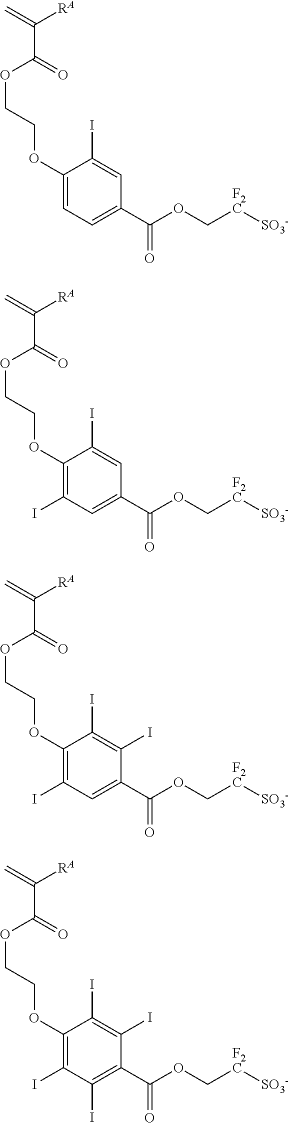

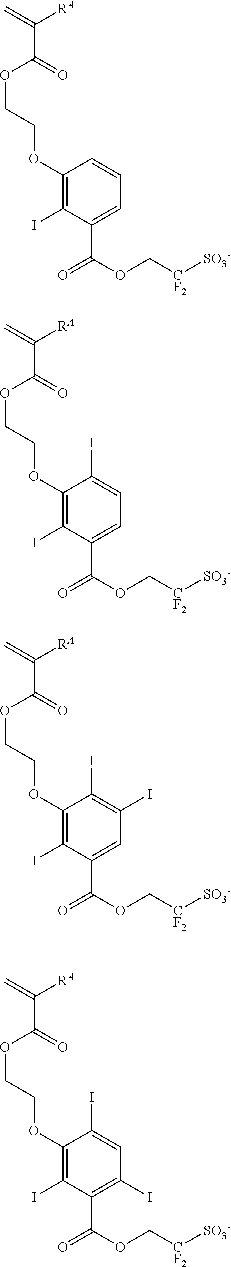

Specific examples of a monomer to give the repeating unit-f3 include ones shown below, but are not limited thereto. Note that, in the following formulae, R^{A} is as defined above.

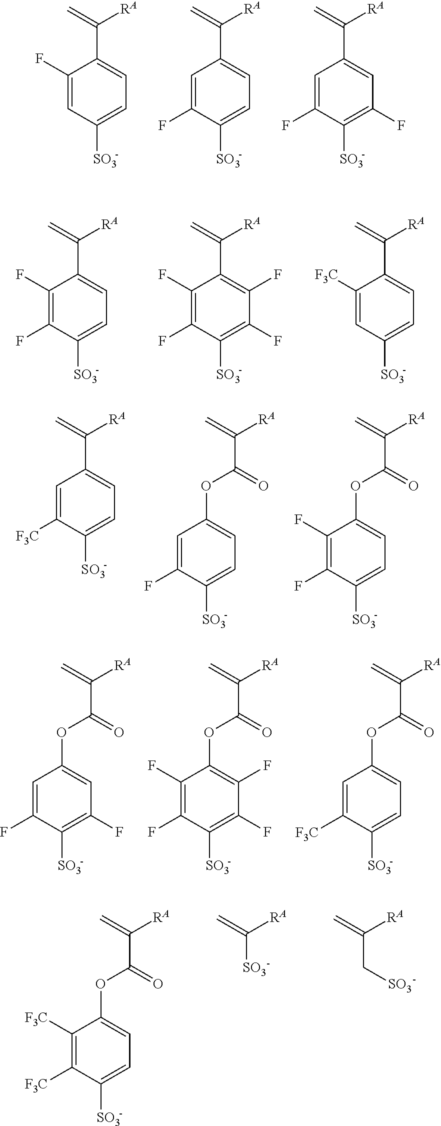

The repeating units-f1 to -f3 function as acid generators. Acid diffusion is reduced by the acid generator being bonded to the polymer main chain, and it is possible to prevent the degradation of resolution due to blurring caused by acid diffusion. In addition, LWR and CDU can be improved by the acid generator being dispersed uniformly.

The repeating unit-a1 or -a2, containing an acid-labile group, is essential to a base polymer for a positive resist material. In this case, the content ratios of the repeating units-a1, -a2, -b, -c, -d, -e, and -f are preferably 0≤a1<1.0, 0≤a2<1.0, 0<a1+a2<1.0, 0<b≤0.9, 0≤c≤0.9, 0≤d≤0.8, 0≤e≤0.8, and 0≤f≤0.5; more preferably 0≤a1≤0.9, 0≤a2≤0.9, 0.1≤a1+a2≤0.9, 0≤b≤0.8, 0≤c≤0.8, 0≤d≤0.7, 0≤e≤0.7, and 0≤f≤0.4; and further preferably 0≤a1≤0.8, 0≤a2≤0.8, 0.1≤a1+a2≤0.8, 0≤b≤0.75, 0≤c≤0.75, 0≤d≤0.6, 0≤e≤0.6, and 0≤f≤0.3. Incidentally, when the repeating unit-f is at least one kind selected from the repeating units-f1 to -f3, f=f1+f2+f3. In addition, a1+a2+b+c+d+e+f=1.0.

Meanwhile, an acid-labile group is not necessarily necessary to a base polymer for a negative resist material. Examples of such a base polymer include ones which include the repeating unit-b, and further include the repeating units-c, -d, -e, and/or -f as necessary. The content ratios of these repeating units are preferably 0<b≤1.0, 0≤c≤0.9, 0≤d≤0.8, 0≤e≤0.8, and 0≤f≤0.5; more preferably 0.2≤b≤1.0, 0≤c≤0.8, 0≤d≤0.7, 0≤e≤0.7, and 0≤f≤0.4; and further preferably 0.3≤b≤1.0, 0≤c≤0.75, 0≤d≤0.6, 0≤e≤0.6, and 0≤f≤0.3. Incidentally, when the repeating unit-f is at least one kind selected from the repeating units-f1 to -f3, f=f1+f2+f3. In addition, b+c+d+e+f=1.0.

To synthesize the base polymer, for example, the monomers to give the repeating units described above can be subjected to heat polymerization in an organic solvent to which a radical polymerization initiator has been added.

Specific examples of the organic solvent used in the polymerization include toluene, benzene, tetrahydrofuran (THF), diethyl ether, and dioxane. Specific examples of the polymerization initiator include 2,2′-azobisisobutyronitrile (AIBN), 2,2′-azobis(2,4-dimethylvaleronitrile), dimethyl 2,2-azobis(2-methylpropionate), benzoyl peroxide, and lauroyl peroxide. The temperature during the polymerization is preferably 50 to 80° C. The reaction time is preferably 2 to 100 hours, more preferably 5 to 20 hours.

In a case where a monomer containing a hydroxy group is copolymerized, it is possible to substitute the hydroxy group with an acetal group susceptible to deprotection with acid, such as an ethoxyethoxy group, prior to the polymerization and perform the deprotection with weak acid and water after the polymerization. Alternatively, it is possible to substitute the hydroxy group with an acetyl group, a formyl group, a pivaloyl group, or the like prior to the polymerization and perform alkaline hydrolysis after the polymerization.

In a case where hydroxystyrene or hydroxyvinylnaphthalene is copolymerized, acetoxystyrene or acetoxyvinylnaphthalene may be used in place of hydroxystyrene or hydroxyvinylnaphthalene; after the polymerization, the acetoxy group may be deprotected by the alkaline hydrolysis as described above to convert the acetoxystyrene or acetoxyvinylnaphthalene to hydroxystyrene or hydroxyvinylnaphthalene.

As a base in the alkaline hydrolysis, it is possible to use ammonia water, triethylamine, etc. In addition, the reaction temperature is preferably-20 to 100° C., more preferably 0 to 60° C. The reaction time is preferably 0.2 to 100 hours, more preferably 0.5 to 20 hours.

The above-described base polymer preferably has a weight-average molecular weight (Mw) of 1000 to 500000, more preferably 2000 to 30000 in terms of polystyrene as measured by gel permeation chromatography (GPC) using THF as an eluent. When the Mw is in the above range, the heat resistance of the resist film and the solubility of the resist film in an alkaline developer are favorable. When GPC is measured, GPC is normally performed at a room temperature of around 23° C., but may be performed at a temperature higher or lower than this.

Furthermore, if the base polymer has a wide molecular weight distribution (Mw/Mn), there are risks that foreign matters may be found on the pattern after the exposure and that pattern profile may be degraded due to low-molecular-weight and high-molecular-weight polymers being present. The finer the pattern rule, the stronger the influences of Mw and Mw/Mn. Hence, in order to obtain a resist material suitably used for finer pattern dimensions, the base polymer preferably has a narrow dispersity Mw/Mn of 1.0 to 2.0, particularly preferably 1.0 to 1.5.

The base polymer may include two or more polymers having different composition ratios, Mw, and Mw/Mn.

### [Organic Solvent]

The inventive chemically-amplified resist composition may contain an organic solvent. This organic solvent is not particularly limited, as long as it is capable of dissolving each of the above-described components and each of the components described below. Specific examples of the organic solvent include ones disclosed in paragraphs [0144] and [0145] of JP2008-111103A: ketones, such as cyclohexanone, cyclopentanone, methyl-2-n-pentyl ketone, and 2-heptanone; alcohols, such as 3-methoxybutanol, 3-methyl-3-methoxybutanol, 1-methoxy-2-propanol, 1-ethoxy-2-propanol, and diacetone alcohol; ethers, such as propylene glycol monomethyl ether, ethylene glycol monomethyl ether, propylene glycol monoethyl ether, ethylene glycol monoethyl ether, propylene glycol dimethyl ether, and diethylene glycol dimethyl ether; esters, such as propylene glycol monomethyl ether acetate, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; and lactones, such as γ-butyrolactone.

In the inventive chemically-amplified resist composition, the contained amount of the organic solvent is preferably 100 to 10000 parts by mass, more preferably 200 to 8000 parts by mass, based on 100 parts by mass of the base polymer. One kind of the organic solvent may be used, or two or more kinds thereof may be used in mixture.

### [Quencher]

The inventive chemically-amplified resist composition may contain a quencher. Note that a quencher means a compound that is capable of trapping an acid that is generated from an acid generator in a resist material to prevent the acid from diffusing to unexposed portions.

Examples of the quencher include conventional basic compounds. Specific examples of the conventional basic compounds include primary, secondary, and tertiary aliphatic amines, mixed amines, aromatic amines, heterocyclic amines, nitrogen-containing compounds having a carboxy group, nitrogen-containing compounds having a sulfonyl group, nitrogen-containing compounds having a hydroxy group, nitrogen-containing compounds having a hydroxyphenyl group, alcoholic nitrogen-containing compounds, amides, imides, carbamates, etc. Particularly preferable are primary, secondary, and tertiary amine compounds disclosed in paragraphs [0146] to [0164] of JP2008-111103A; especially amine compounds having a hydroxy group, an ether bond, an ester bond, a lactone ring, a cyano group, or a sulfonic acid ester bond; compounds having a carbamate bond disclosed in JP3790649B2; etc. Adding such a basic compound can, for example, further suppress the acid diffusion rate in the resist film and correct the shape.

Other examples of the quencher include onium salts, such as sulfonium salts, iodonium salts, and ammonium salts of sulfonic acids, carboxylic acids, and fluorinated alkoxides which are not fluorinated at position as disclosed in JP2008-158339A. While x-fluorinated sulfonic acid, imide acid, or methide acid is necessary to deprotect the acid-labile group of carboxylic acid ester, a sulfonic acid, a carboxylic acid, or a fluorinated alcohol not fluorinated at α position is released by salt exchange with the onium salt. Such a sulfonic acid, carboxylic acid, and fluorinated alcohol not fluorinated at α position hardly induce a deprotection reaction, and thus function as quenchers.

Specific examples of such quenchers include compounds represented by the following formula (4) (onium salts of sulfonic acid not fluorinated at α position), compounds represented by the following formula (5) (onium salts of carboxylic acids), and compounds represented by the following formula (6) (onium salts of alkoxides).

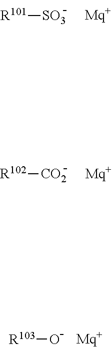

In the formula (4), R¹⁰¹ represents a hydrogen atom or a hydrocarbyl group having 1 to 40 carbon atoms and optionally containing a heteroatom, but excludes groups in which a hydrogen atom bonded to the carbon atom at the α position of the sulfo group is substituted with a fluorine atom or a fluoroalkyl group.

The hydrocarbyl group having 1 to 40 carbon atoms represented by R¹⁰¹ may be saturated or unsaturated, and may be linear, branched, or cyclic. Specific examples thereof include: alkyl groups having 1 to 40 carbon atoms, such as a methyl group, an ethyl group, an n-propyl group, an isopropyl group, an n-butyl group, an isobutyl group, a sec-butyl group, a tert-butyl group, an n-pentyl group, a tert-pentyl group, an n-hexyl group, an n-octyl group, a 2-ethylhexyl group, an n-nonyl group, and an n-decyl group; cyclic saturated hydrocarbyl groups having 3 to 40 carbon atoms, such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a tricyclo[5.2.1.0^{2,6}]decyl group, an adamantyl group, and an adamantylmethyl group; alkenyl groups having 2 to 40 carbon atoms, such as a vinyl group, an allyl group, a propenyl group, a butenyl group, and a hexenyl group; cyclic unsaturated aliphatic hydrocarbyl groups having 3 to 40 carbon atoms, such as a cyclohexenyl group; aryl groups having 6 to 40 carbon atoms, such as a phenyl group, a naphthyl group, alkylphenyl groups (a 2-methylphenyl group, a 3-methylphenyl group, a 4-methylphenyl group, a 4-ethylphenyl group, a 4-tert-butylphenyl group, a 4-n-butylphenyl group, etc.), dialkylphenyl or trialkylphenyl groups (a 2,4-dimethylphenyl group, a 2,4,6-triisopropylphenyl group, etc.), alkylnaphthyl groups (a methylnaphthyl group, an ethylnaphthyl group, etc.), and dialkylnaphthyl groups (a dimethylnaphthyl group, a diethylnaphthyl group, etc.); aralkyl groups having 7 to 40 carbon atoms, such as a benzyl group, a 1-phenylethyl group, and a 2-phenylethyl group; etc.

Moreover, part or all of the hydrogen atoms of the hydrocarbyl groups may be substituted with a group containing a heteroatom, such as an oxygen atom, a sulfur atom, a nitrogen atom, or a halogen atom, and part of the —CH₂— of the hydrocarbyl groups may be substituted with a group containing a heteroatom, such as an oxygen atom, a sulfur atom, or a nitrogen atom. The resulting hydrocarbyl groups may contain a hydroxy group, a cyano group, a carbonyl group, an ether bond, an ester bond, a sulfonic acid ester bond, a carbonate bond, a lactone ring, a sultone ring, carboxylic anhydride (—C(═O)—O—C(═O)—), a haloalkyl group, etc. Specific examples of the hydrocarbyl group containing a heteroatom include: heteroaryl groups, such as a thienyl group; alkoxyphenyl groups, such as a 4-hydroxyphenyl group, a 4-methoxyphenyl group, a 3-methoxyphenyl group, a 2-methoxyphenyl group, a 4-ethoxyphenyl group, a 4-tert-butoxyphenyl group, and a 3-tert-butoxyphenyl group; alkoxynaphthyl groups, such as a methoxynaphthyl group, an ethoxynaphthyl group, an n-propoxynaphthyl group, and an n-butoxynaphthyl group; dialkoxynaphthyl groups, such as a dimethoxynaphthyl group and a diethoxynaphthyl group; aryloxoalkyl groups, such as 2-aryl-2-oxoethyl groups including a 2-phenyl-2-oxoethyl group, a 2-(1-naphthyl)-2-oxoethyl group, and a 2-(2-naphthyl)-2-oxoethyl group; etc.

In the formula (5), R¹⁰² represents a hydrocarbyl group having 1 to 40 carbon atoms and optionally containing a heteroatom. Specific examples of the hydrocarbyl group represented by R¹⁰² include those given as examples of the hydrocarbyl group represented by R¹⁰¹. Furthermore, other specific examples thereof include fluorinated alkyl groups, such as a trifluoromethyl group, a trifluoroethyl group, a 2, 2,2-trifluoro-1-methyl-1-hydroxyethyl group, and a 2, 2,2-trifluoro-1-(trifluoromethyl)-1-hydroxyethyl group; fluorinated aryl groups, such as a pentafluorophenyl group and a 4-trifluoromethylphenyl group; etc.

In the formula (6), R¹⁰³ represents a saturated hydrocarbyl group having 1 to 8 carbon atoms and having at least three fluorine atoms or represents an aryl group having 6 to 10 carbon atoms and having at least three fluorine atoms, and may contain a nitro group.

In the formulae (4), (5), and (6), Mq⁺ represents an onium cation. As the onium cation, a sulfonium cation, an iodonium cation, or an ammonium cation is preferable, and a sulfonium cation is more preferable. Specific examples of the sulfonium cation include those given as examples of the sulfonium cation represented by the formula (Z-1).

As the quencher, it is also possible to use suitably a sulfonium salt of a carboxylic acid containing an iodinated benzene ring, represented by the following formula (7).

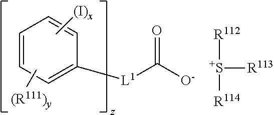

In the formula (7), “x” represents an integer of 1 to 5. “y” represents an integer of 0 to 3. “z” represents an integer of 1 to 3.

In the formula (7), R¹¹¹ represents a hydroxy group, a fluorine atom, a chlorine atom, a bromine atom, an amino group, a nitro group, or a cyano group; a saturated hydrocarbyl group having 1 to 6 carbon atoms, a saturated hydrocarbyloxy group having 1 to 6 carbon atoms, a saturated hydrocarbylcarbonyloxy group having 2 to 6 carbon atoms, or a saturated hydrocarbylsulfonyloxy group having 1 to 4 carbon atoms, part or all of the hydrogen atoms of each group optionally being substituted with a halogen atom; or —N(R^{111A})—C(═O)—R^{111B} or —N(R^{111A})—C(═O)—O—R^{111B}. R^{111A} represents a hydrogen atom or a saturated hydrocarbyl group having 1 to 6 carbon atoms. R^{111B} represents a saturated hydrocarbyl group having 1 to 6 carbon atoms or an unsaturated aliphatic hydrocarbyl group having 2 to 8 carbon atoms. When “y” and/or “z” is 2 or more, the Rills may be identical to or different from each other.

In the formula (7), L¹ represents a single bond or a linking group having a valency of z+1 and having 1 to 20 carbon atoms, and optionally contains at least one kind selected from an ether bond, a carbonyl group, an ester bond, an amide bond, a sultone ring, a lactam ring, a carbonate bond, a halogen atom, a hydroxy group, and a carboxy group. The saturated hydrocarbyl group, the saturated hydrocarbyloxy group, the saturated hydrocarbylcarbonyloxy group, and the saturated hydrocarbylsulfonyloxy group may each be linear, branched, or cyclic.

In the formula (7), R¹¹², R¹¹³, and R¹¹⁴ each independently represent a halogen atom or a hydrocarbyl group having 1 to 20 carbon atoms and optionally containing a heteroatom. The hydrocarbyl group may be saturated or unsaturated, and may be linear, branched, or cyclic. Specific examples thereof include those given as examples of the hydrocarbyl groups represented by R^{ct1} to R^{ct5} in the description of the formulae (Z-1) and (Z-2).

Specific examples of the compound represented by the formula (7) include ones disclosed in JP2017-219836A and JP2021-91666A.

Other examples of the quencher include polymer-type quenchers disclosed in JP2008-239918A. The quenchers enhance the rectangularity of a resist pattern by being oriented on the resist film surface. The polymer-type quencher also has effects of preventing rounding of a pattern top and film thickness loss of a pattern when a top coat for immersion exposure is applied.

Furthermore, it is also possible to use, as a quencher: betaine type sulfonium salts disclosed in JP6848776B2 and JP2020-37544A; methide acids containing no fluorine atoms disclosed in JP2020-55797A; sulfonium salts of sulfonamides disclosed in JP5807552B2; sulfonium salts of sulfonamides containing an iodine atom disclosed in JP2019-211751A; phenol; halogen; and acid generators that generate carbonic acid.

When the inventive chemically-amplified resist composition contains the quencher, the contained amount is preferably 0 to 5 parts by mass, more preferably 0 to 4 parts by mass, based on 100 parts by mass of the base polymer. One kind of the quencher may be used, or two or more kinds thereof may be used in combination.

### [Other Components]

The above-described composition may contain acid generators other than the onium salt represented by the formula (A) (hereinafter, referred to as other acid generators), a surfactant, a dissolution inhibitor, a crosslinking agent, a water-repellency enhancer, an acetylene alcohol, etc. in addition to the above-described components.

Examples of the other acid generators include compounds that generate acids in response to actinic light or radiation (photo-acid generators). The photo-acid generator component is not particularly limited, as long as the compound generates an acid upon irradiation with a high-energy beam. An acid generator that generates a sulfonic acid, imide acid, or methide acid is preferable. Specific examples of suitable photo-acid generators include sulfonium salt, iodonium salt, sulfonyldiazomethane, N-sulfonyloxyimide, oxime-O-sulfonate type acid generators, etc. Specific examples of the acid generator include ones disclosed in paragraphs [01222] to [0142] of JP2008-111103A, JP2018-5224A, and JP2018-25789A. When the inventive chemically-amplified resist composition contains the other acid generators, the contained amount is preferably 0 to 200 parts by mass, preferably 0.1 to 100 parts by mass, based on 100 parts by mass of the base polymer.

Specific examples of the surfactant include ones disclosed in paragraphs [0165] and [0166] of JP2008-111103A. Adding a surfactant can further enhance or control the coatability of the resist material. When the inventive chemically-amplified resist composition contains the surfactant, the contained amount is preferably 0.0001 to 10 parts by mass based on 100 parts by mass of the base polymer. One kind of the surfactant may be used, or two or more kinds thereof may be used in combination.

When the inventive chemically-amplified resist composition is a positive type, blending a dissolution inhibitor can further increase the difference in dissolution rate between exposed and unexposed portions, and further enhance the resolution. Specific examples of the dissolution inhibitor include a compound which contains two or more phenolic hydroxy groups in a molecule, and in which 0 to 100 mol % of all the hydrogen atoms of the phenolic hydroxy groups are substituted with acid-labile groups; or a compound which contains a carboxy group in a molecule, and in which 50 to 100 mol % of all the hydrogen atoms of such carboxy groups are substituted with acid-labile groups on average. In addition, the compounds each have a molecular weight of preferably 100 to 1000, more preferably 150 to 800. Specific examples include compounds obtained by substituting acid-labile groups for hydrogen atoms of hydroxy groups or carboxy groups of bisphenol A, trisphenol, phenolphthalein, cresol novolak, naphthalenecarboxylic acid, adamantanecarboxylic acid, or cholic acid; etc. Examples are disclosed in paragraphs [0155] to [0178] of JP 2008-122932 A.

When the inventive chemically-amplified resist composition is a positive type and contains the dissolution inhibitor, the contained amount is preferably 0 to 50 parts by mass, more preferably 5 to 40 parts by mass, based on 100 parts by mass of the base polymer. One kind of the dissolution inhibitor can be used, or two or more kinds thereof can be used in combination.

Meanwhile, when the inventive chemically-amplified resist composition is a negative type, a negative pattern can be obtained by reducing the dissolution rate in exposed portions by adding a crosslinking agent. Specific examples of the crosslinking agent include: epoxy compounds, melamine compounds, guanamine compounds, and glycoluril compounds, each having a substituent which is at least one group selected from a methylol group, an alkoxymethyl group, and an acyloxymethyl group; or compounds including a double bond, such as urea compounds, isocyanate compounds, azide compounds, and alkenyloxy groups. These may be contained as additives, but may also be introduced as a pendant group in a polymer side chain. A compound containing a hydroxy group can also be contained as a crosslinking agent.

Specific examples of the epoxy compounds include tris(2,3-epoxypropyl) isocyanurate, trimethylolmethane triglycidyl ether, trimethylolpropane triglycidyl ether, and triethylolethane triglycidyl ether.

Specific examples of the melamine compounds include hexamethylolmelamine, hexamethoxymethylmelamine, and compounds in which 1 to 6 methylol groups of hexamethylolmelamine have been methoxymethylated or mixtures thereof; hexamethoxyethylmelamine, hexaacyloxymethylmelamine, and compounds in which 1 to 6 methylol groups of hexamethylolmelamine have been acyloxymethylated or mixtures thereof.

Specific examples of the guanamine compounds include: tetramethylolguanamine, tetramethoxymethylguanamine, and compounds in which 1 to 4 methylol groups of tetramethylolguanamine have been methoxymethylated or mixtures thereof; and tetramethoxyethylguanamine, tetraacyloxyguanamine, and compounds in which 1 to 4 methylol groups of tetramethylolguanamine have been acyloxymethylated or mixtures thereof.

Specific examples of the glycoluril compounds include: tetramethylolglycoluril, tetramethoxyglycoluril, tetramethoxymethylglycoluril, and compounds in which 1 to 4 methylol groups of tetramethylolglycoluril have been methoxymethylated or mixtures thereof; and compounds in which 1 to 4 methylol groups of tetramethylolglycoluril have been acyloxymethylated or mixtures thereof. Specific examples of the urea compounds include: tetramethylolurea, tetramethoxymethylurea, and compounds in which 1 to 4 methylol groups of tetramethylolurea have been methoxymethylated or mixtures thereof; and tetramethoxyethylurea.

Specific examples of the isocyanate compounds include tolylenediisocyanate, diphenylmethanediisocyanate, hexamethylenediisocyanate, and cyclohexanediisocyanate.

Specific examples of the azide compounds include 1,1′-biphenyl-4,4′-bisazide, 4,4′-methylidenebisazide, and 4,4′-oxybisazide.

Specific examples of the compounds containing an alkenyloxy group include ethylene glycol divinyl ether, triethylene glycol divinyl ether, 1,2-propanediol divinyl ether, 1,4-butanediol divinyl ether, tetramethylene glycol divinyl ether, neopentyl glycol divinyl ether, trimethylolpropane trivinyl ether, hexanediol divinyl ether, 1,4-cyclohexanediol divinyl ether, pentaerythritol trivinyl ether, pentaerythritol tetravinyl ether, sorbitol tetravinyl ether, sorbitol pentavinyl ether, and trimethylolpropane trivinyl ether.

When the inventive chemically-amplified resist composition is a negative type and contains the crosslinking agent, the contained amount is preferably 0.1 to 50 parts by mass, more preferably 1 to 40 parts by mass, based on 100 parts by mass of the base polymer. One kind of the crosslinking agent may be used, or two or more kinds thereof may be used in combination.

The water-repellency enhancer improves the water-repellency of the resist film surface, and can be employed in immersion lithography with no top coat. The water-repellency enhancer is preferably a polymer containing a fluorinated alkyl group, a polymer containing a 1,1,1,3,3,3-hexafluoro-2-propanol residue with a particular structure, etc., preferably ones exemplified in JP2007-297590A, JP2008-111103A, etc. The water-repellency enhancer needs to be dissolved in an alkaline developer or an organic solvent developer. The water-repellency enhancer having a particular 1,1,1,3,3,3-hexafluoro-2-propanol residue mentioned above has favorable solubility to developers. A polymer including a repeating unit with an amino group or amine salt as a water-repellency enhancer exhibits high effects of preventing acid evaporation during PEB to prevent opening failure of a hole pattern after development. When the inventive chemically-amplified resist composition contains the water-repellency enhancer, the contained amount is preferably 0 to 20 parts by mass, more preferably 0.5 to 10 parts by mass, based on 100 parts by mass of the base polymer. One kind of the water-repellency enhancer may be used, or two or more kinds thereof may be used in combination.

Specific examples of the acetylene alcohol include ones disclosed in paragraphs [0179] to [0182] of JP2008-122932A. When the inventive chemically-amplified resist composition contains the acetylene alcohol, the contained amount is preferably 0 to 5 parts by mass based on 100 parts by mass of the base polymer. One kind of the acetylene alcohol may be used, or two or more kinds thereof may be used in combination.

### [Patterning Process]

When the inventive chemically-amplified resist composition is used for manufacturing various integrated circuits, known lithography techniques can be applied. Examples of the patterning process include a method including the steps of: forming a resist film on a substrate by using the above-described chemically-amplified resist composition; exposing the resist film by using a high-energy beam; and developing the exposed resist film by using a developer.

Firstly, the inventive chemically-amplified resist composition is applied onto a substrate (Si, SiO₂, SiN, SiON, TiN, WSi, BPSG, SOG, organic antireflective film, etc.) for manufacturing an integrated circuit or a substrate (Cr, Cro, CrON, MoSi₂, SiO₂, etc.) for manufacturing a mask circuit by an appropriate coating process, such as spin coating, roll coating, flow coating, dip coating, spray coating, and doctor coating, so that the coating film has a thickness of 0.01 to 2 μm. The coating film is prebaked on a hot plate preferably at 60 to 150° C. for 10 seconds to 30 minutes, more preferably at 80 to 120° C. for 30 seconds to 20 minutes. Thus, a resist film is formed.

Then, the resist film is exposed using a high-energy beam. Specific examples of the high-energy beam include ultraviolet ray, deep ultraviolet ray, EB (electron beam), EUV (extreme ultraviolet ray) having a wavelength of 3 to 15 nm, X-ray, soft X-ray, excimer laser beam, γ-ray, synchrotron radiation, etc. When ultraviolet ray, deep ultraviolet ray, EUV, X-ray, soft X-ray, excimer laser beam, γ-ray, synchrotron radiation, or the like is employed as the high-energy beam, the irradiation is performed directly or while using a mask for forming a target pattern at an exposure dose of preferably about 1 to 200 mJ/cm², more preferably about 10 to 100 mJ/cm². When EB is employed as the high-energy beam, the exposure dose is preferably about 0.1 to 300 μC/cm², more preferably about 0.5 to 200 μC/cm², and the writing is performed directly or while using a mask for forming a target pattern. Incidentally, the inventive chemically-amplified resist composition is particularly suitable for fine patterning with a KrF excimer laser beam, having a wavelength of 248 nm, an ArF excimer laser beam, having a wavelength of 193 nm, an EB, EUV, X-ray, soft X-ray, γ-ray, or synchrotron radiation among the high-energy beams, and is particularly suitable for fine patterning with an EB or EUV.

After the exposure, PEB may be performed on a hot plate or in an oven, preferably at 30 to 150° C. for 10 seconds to 30 minutes, more preferably at 50 to 120° C. for 30 seconds to 20 minutes. Alternatively, PEB may not be performed.

After the exposure or PEB, the exposed resist film can be developed using a developer of 0.1 to 10 mass %, preferably 2 to 5 mass %, aqueous alkaline solution of a material, such as tetramethylammonium hydroxide (TMAH), tetraethylammonium hydroxide, tetrapropylammonium hydroxide, and tetrabutylammonium hydroxide, for 3 seconds to 3 minutes, preferably 5 seconds to 2 minutes, by a conventional technique, such as a dip method, a puddle method, or a spray method. Thereby, the target pattern is formed. In the case of a positive resist material, the portion irradiated with the light is dissolved by the developer, while the unexposed portion remains undissolved. Thus, the target positive pattern is formed on the substrate. A case with a negative resist material is the reverse of the case with the positive resist material, and the portion irradiated with the light becomes insoluble in a developer, while the unexposed portion dissolves.

It is also possible to obtain a negative pattern by organic solvent development using a positive resist material containing a base polymer containing an acid-labile group. Specific examples of the developer used in this event include 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, phenylmethyl acetate, benzyl formate, phenylethyl formate, methyl 3-phenylpropionate, benzyl propionate, phenylethyl acetate, 2-phenylethyl acetate, etc. One of these organic solvents can be used, or two or more thereof can be used in mixture.

When the development is completed, rinsing is performed. The rinsing liquid is preferably a solvent that is miscible with the developer but does not dissolve the resist film. As such a solvent, it is preferable to use an alcohol having 3 to 10 carbon atoms, an ether compound having 8 to 12 carbon atoms, an alkane, alkene, alkyne, or aromatic solvent, each having 6 to 12 carbon atoms, etc.

Specific examples of the alcohol having 3 to 10 carbon atoms include n-propyl alcohol, isopropyl alcohol, 1-butyl alcohol, 2-butyl alcohol, isobutyl alcohol, tert-butyl alcohol, 1-pentanol, 2-pentanol, 3-pentanol, tert-pentyl alcohol, neopentyl alcohol, 2-methyl-1-butanol, 3-methyl-1-butanol, 3-methyl-3-pentanol, cyclopentanol, 1-hexanol, 2-hexanol, 3-hexanol, 2,3-dimethyl-2-butanol, 3,3-dimethyl-1-butanol, 3,3-dimethyl-2-butanol, 2-ethyl-1-butanol, 2-methyl-1-pentanol, 2-methyl-2-pentanol, 2-methyl-3-pentanol, 3-methyl-1-pentanol, 3-methyl-2-pentanol, 3-methyl-3-pentanol, 4-methyl-1-pentanol, 4-methyl-2-pentanol, 4-methyl-3-pentanol, cyclohexanol, 1-octanol, etc.

Specific examples of the ether compound having 8 to 12 carbon atoms include di-n-butyl ether, diisobutyl ether, di-sec-butyl ether, di-n-pentyl ether, diisopentyl ether, di-sec-pentyl ether, di-tert-pentyl ether, di-n-hexyl ether, etc.

Specific examples of the alkane having 6 to 12 carbon atoms include hexane, heptane, octane, nonane, decane, undecane, dodecane, methylcyclopentane, dimethylcyclopentane, cyclohexane, methylcyclohexane, dimethylcyclohexane, cycloheptane, cyclooctane, cyclononane, etc. Specific examples of the alkene having 6 to 12 carbon atoms include hexene, heptene, octene, cyclohexene, methylcyclohexene, dimethylcyclohexene, cycloheptene, cyclooctene, etc. Specific examples of the alkyne having 6 to 12 carbon atoms include hexyne, heptyne, octyne, etc.

Specific examples of the aromatic solvent include toluene, xylene, ethylbenzene, isopropylbenzene, tert-butylbenzene, mesitylene, etc.

The rinsing can reduce resist pattern collapse and defect formation. Meanwhile, the rinsing is not necessarily essential, and the amount of the solvent used can be reduced by not performing the rinsing.

After the development, a hole pattern or trench pattern can be shrunk by thermal flow, RELACS process, or DSA process. A shrink agent is applied onto the hole pattern, and the shrink agent undergoes crosslinking on the resist film surface by diffusion of the acid catalyst from the resist film during baking, so that the shrink agent is attached to sidewalls of the hole pattern. The baking temperature is preferably 70 to 180° C., more preferably 80 to 170° C. The baking time is preferably 10 to 300 seconds. The extra shrink agent is removed, and thus, the hole pattern is shrunk.

### EXAMPLES

Hereinafter, the present invention will be specifically described with reference to Synthesis Examples, Examples, and Comparative Examples, but the present invention is not limited to the following Examples. Incidentally, the apparatus used was as follows.
MALDI TOF-MS: S3000 manufactured by JEOL Ltd.

### [Synthesis Example] Synthesis of Onium Salts (Photo-Acid Generators PAG-1 to PAG-21)

### Synthesis of Onium Salt a-1 (Photo-Acid Generator PAG-1)

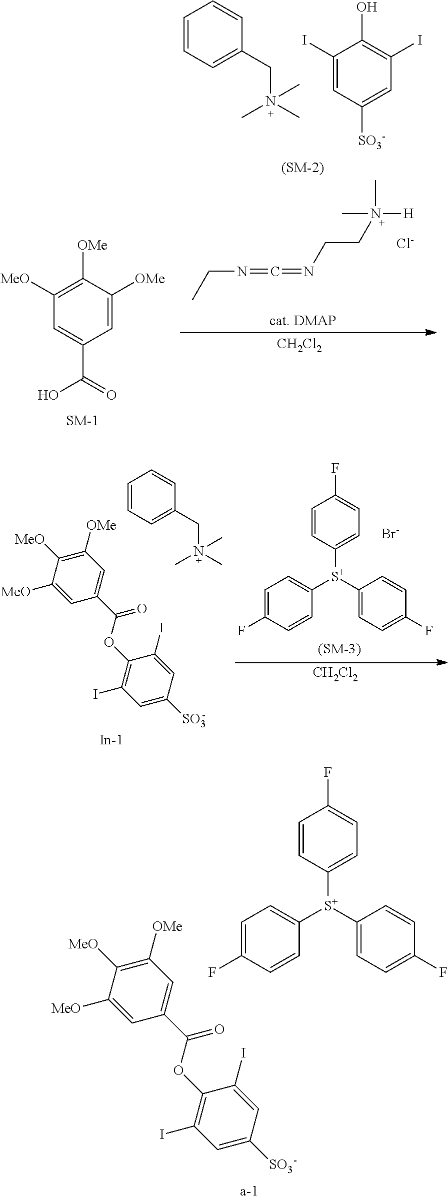

### (1) Synthesis of Intermediate In-1

Under a nitrogen atmosphere, a raw material SM-1 (6.8 g), a raw material SM-2 (20 g), DMAP (0.40 g), and methylene chloride (80 g) were added to a reaction container and cooled in an ice bath. While maintaining the temperature inside the reaction container at 20° C. or lower, 1-ethyl-3-(3-dimethylaminopropyl) carbodiimide hydrochloride (9.3 g) was added to the mixture while still in the state of a powder. After the addition, the temperature was raised to room temperature, and the mixture was aged for 12 hours. After the aging, water was added to the reaction product to terminate the reaction, a common aqueous work-up was performed, the solvent was distilled off, and then diisopropyl ether was added to wash the residue. Thus, 26.3 g of intermediate In-1 was obtained as a white solid.

### (2) Synthesis of Onium Salt a-1

Under a nitrogen atmosphere, the intermediate In-1 (26.3 g), a raw material SM-3 (14 g), and methylene chloride (300 g) were added to a reaction container and stirred for 30 minutes. Then, the organic layer was collected, washed with water, and then concentrated under reduced pressure. The concentrated liquid was recrystallized with methyl-tert-butyl ether to obtain 28.7 g of onium salt a-1, being the target product, as a white crystal (96% yield).

### MALDI TOF-MS:

POSITIVE M⁺ 317 (C₁₈H₁₂F₃S⁺ equivalent)
NEGATIVE M⁻ 619 (C₁₆H₁₃I₂O₈S⁻ equivalent)

The structures of onium salt acid generators PAG-1 to PAG-21, used in the resist materials, are shown below. PAG-2 to PAG-21 were synthesized using corresponding raw materials and known organic synthesis reactions.

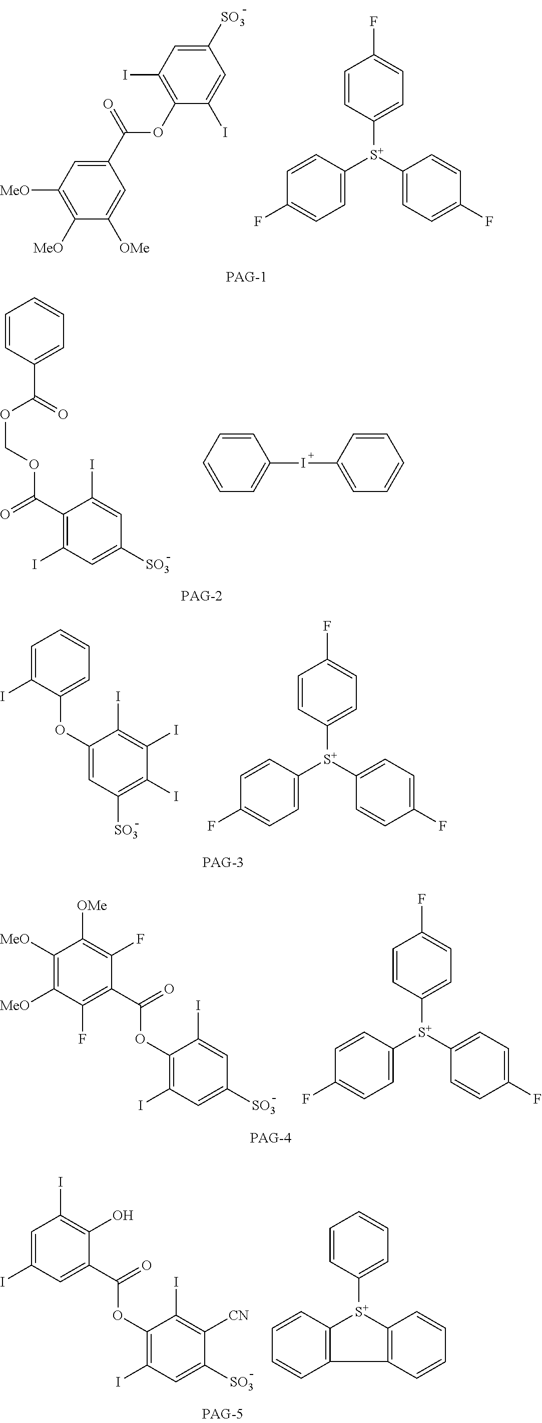

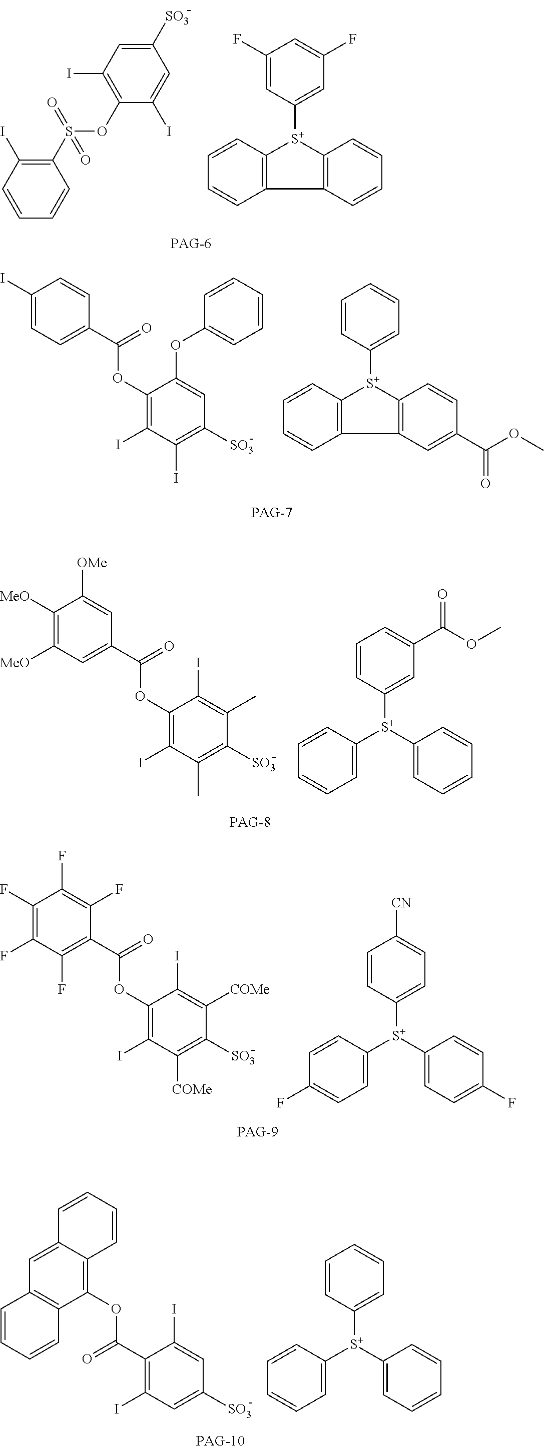

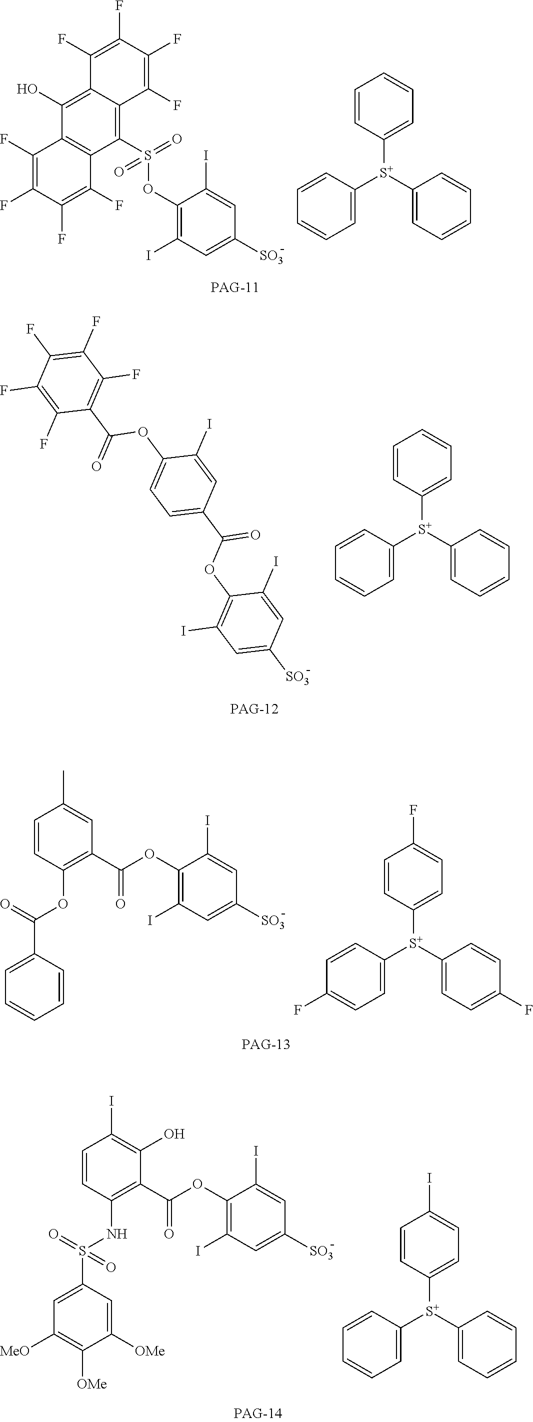

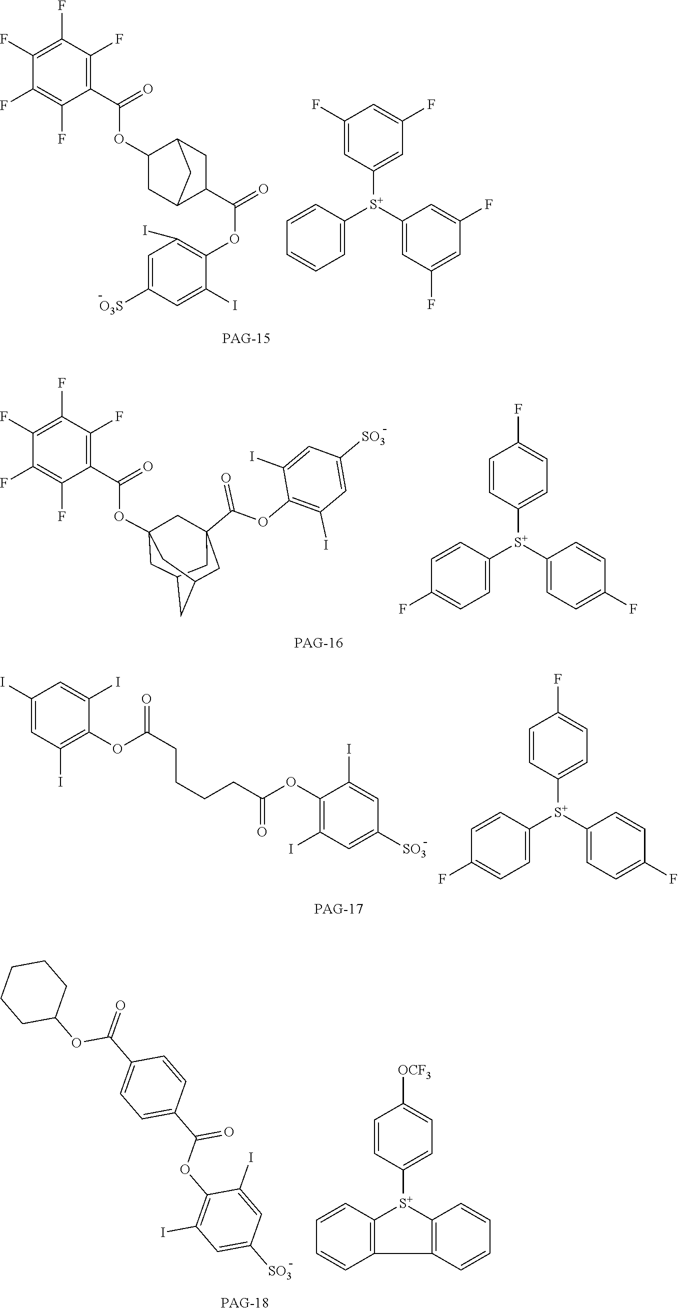

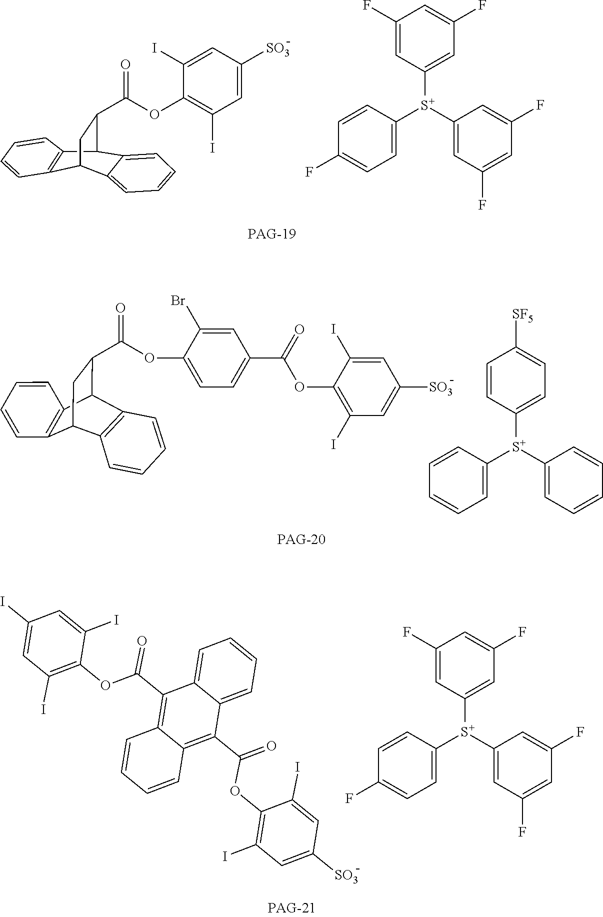

### [Synthesis Example] Synthesis of Base Polymers (Polymers P-1 to P-4)

A copolymerization reaction was performed in a solvent, THF, with a combination of the monomers, the product was added to methanol, the precipitated solid was washed with hexane, and then isolation and drying were performed to obtain base polymers (polymers P-1 to P-4) having the composition shown below. The composition of the obtained base polymers was confirmed by ¹H-NMR and Mw and Mw/Mn were confirmed by GPC (eluent: THF, standard: polystyrene).

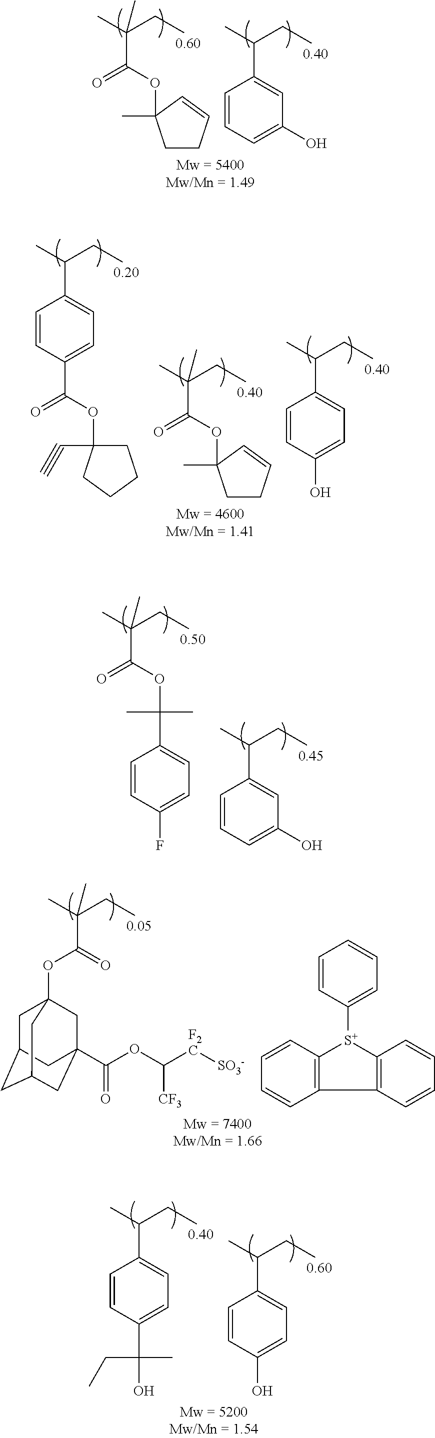

### Examples 1 to 24 and Comparative Examples 1 to 8: Preparation of Resist Materials and Evaluation thereof

### (1) Preparation of Resist Materials

According to the composition shown in Table 1, components were dissolved, and the resulting solution was filtered through a filter having a pore size of 0.2 μm. In this manner, resist materials were prepared.

The components in Table 1 are as follows.

### Organic Solvents:

PGMEA (propylene glycol monomethyl ether acetate)
EL (ethyl lactate)
DAA (diacetone alcohol)
PGME (propylene glycol monomethyl ether)
   Comparative Acid Generators: cPAG-1 to cPAG-8

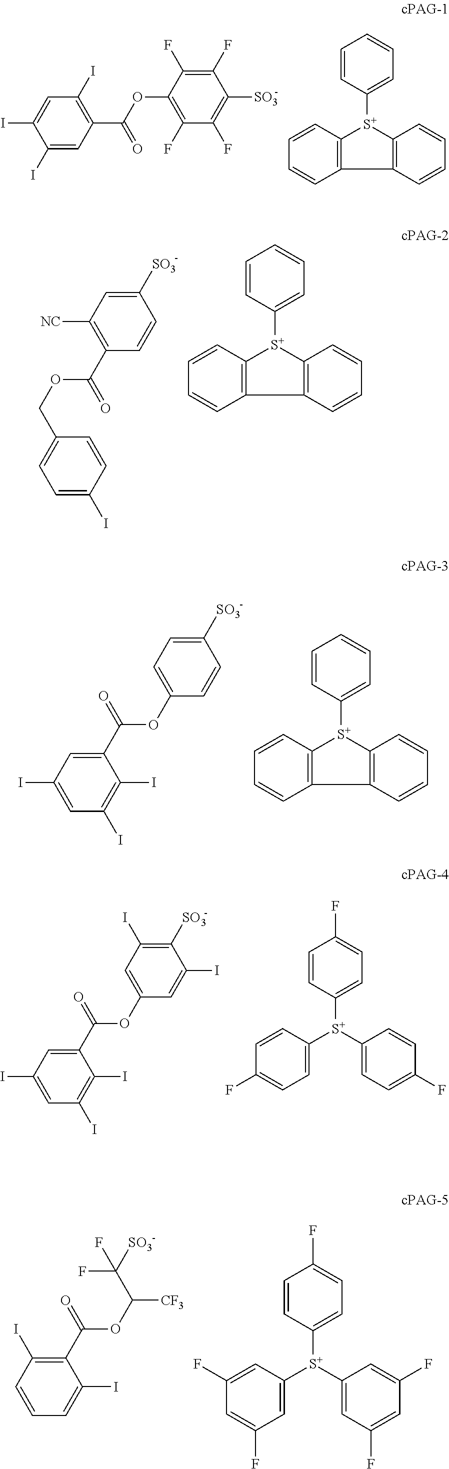

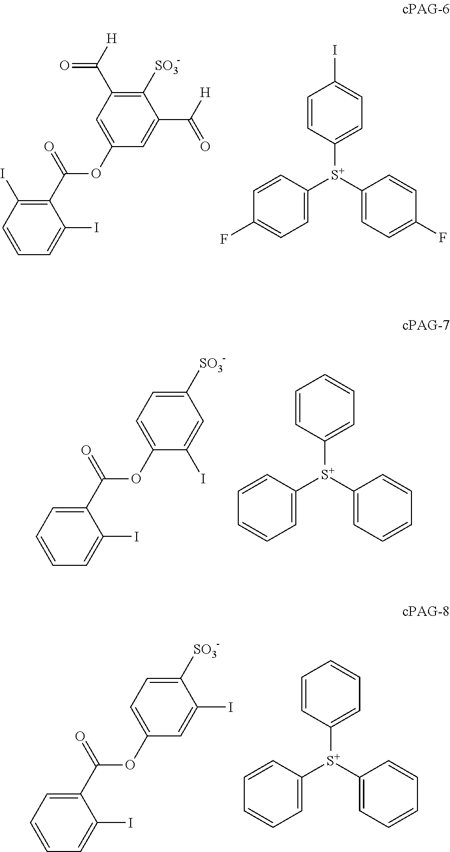

### Quenchers: Q-1 and Q-2

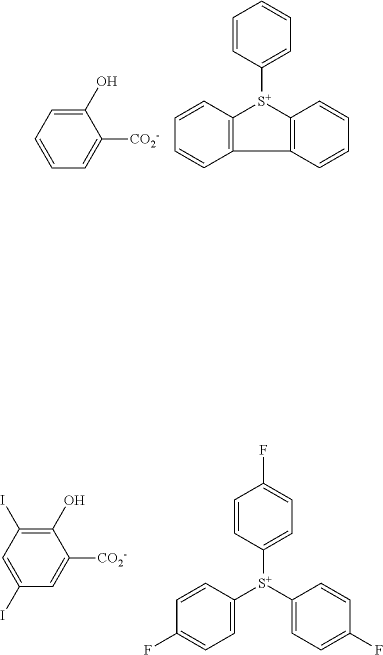

### (2) EUV Lithography Evaluation 1

Each of the resist materials shown in Tables 1 and 2 was respectively applied by spin-coating on a Si substrate on which a silicon-containing spin-on hard mask SHB-A940, manufactured by Shin-Etsu Chemical Co., Ltd. (silicon content of 43 mass %), was formed with 20 nm in film thickness. Then, the substrate was prebaked at 105° C. for 60 seconds using a hot plate to produce each resist film having a film thickness of 50 nm. The resist film was exposed using an EUV scanner NXE3400 (NA 0.33, σ 0.9/0.6, quadrupole illumination, 40 nm in pitch on wafer size, hole pattern mask with +20% bias), manufactured by ASML Holding N.V. Then, PEB was performed at a temperature shown in Tables 1 and 2 for 60 seconds using a hot plate. Thereafter, development was performed with a 2.38 mass % aqueous TMAH solution for 30 seconds to form a hole pattern with 20 nm in size in Examples 1 to 24 and Comparative Examples 1 to 8.

Using a length-measurement SEM (CG6300), manufactured by Hitachi High-Technologies Corporation, an exposure dose when the holes were formed with a size of 20 nm was measured to specify this value as sensitivity. Sizes of fifty holes were measured in this time, and a tripled value (3σ) of a standard variation (σ) calculated from the results was determined as CDU. The results are shown in Tables 1 and 2.

TABLE 1

| | | Acid | | Organic | | | |
|---|---|---|---|---|---|---|---|
| | Polymer | generator | Quencher | solvent | | | |
| | (parts by | (parts by | (parts by | (parts by | PEB | Sensitivity | CDU |
| | mass) | mass) | mass) | mass) | (°C) | (mJ/cm²) | (nm) |
| Example | P-1 | PAG-1 | Q-1 | PGMEA (2000) | 100 | 31 | 3.1 |
| 1 | (80) | (30.4) | (4.0) | DAA (500) | | | |
| Example | P-1 | PAG-2 | Q-1 | PGMEA (500) | 100 | 32 | 3.3 |
| 2 | (80) | (21.7) | (4.0) | EL (2000) | | | |
| Example | P-1 | PAG-3 | Q-1 | PGMEA (500) | 100 | 36 | 3.2 |
| 3 | (80) | (26.8) | (4.0) | EL (2000) | | | |
| Example | P-1 | PAG-4 | Q-1 | PGMEA (500) | 100 | 33 | 3.2 |
| 4 | (80) | (24.3) | (4.0) | EL (2000) | | | |
| Example | P-1 | PAG-5 | Q-1 | PGMEA (500) | 100 | 33 | 3.3 |
| 5 | (80) | (27.1) | (4.0) | EL (2000) | | | |
| Example | P-1 | PAG-6 | Q-1 | PGMEA (500) | 100 | 33 | 3.2 |
| 6 | (80) | (24.7) | (4.0) | EL (2000) | | | |
| Example | P-1 | PAG-7 | Q-1 | PGMEA (500) | 100 | 36 | 3.2 |
| 7 | (80) | (26.7) | (4.0) | EL (2000) | | | |
| Example | P-1 | PAG-8 | Q-1 | PGMEA (500) | 100 | 32 | 3.2 |
| 8 | (80) | (24.2) | (4.0) | EL (2000) | | | |
| Example | P-1 | PAG-9 | Q-1 | PGMEA (500) | 100 | 33 | 3.2 |
| 9 | (80) | (25.7) | (4.0) | EL (2000) | | | |
| Example | P-1 | PAG-10 | Q-1 | PGMEA (500) | 100 | 33 | 3.3 |
| 10 | (80) | (22.3) | (4.0) | EL (2000) | | | |
| Example | P-1 | PAG-11 | Q-1 | PGMEA (500) | 100 | 34 | 3.3 |
| 11 | (80) | (27.2) | (4.0) | EL (2000) | | | |
| Example | P-1 | PAG-12 | Q-1 | PGMEA (500) | 100 | 35 | 3.4 |
| 12 | (80) | (28.2) | (4.0) | EL (2000) | | | |
| Example | P-1 | PAG-13 | Q-1 | PGME (500) | 100 | 34 | 3.2 |
| 13 | (80) | (32.6) | (4.0) | EL (2000) | | | |
| Example | P-1 | PAG-14 | Q-1 | PGME (500) | 100 | 32 | 3.2 |
| 14 | (80) | (27.3) | (4.0) | EL (2000) | | | |
| Example | P-1 | PAG-15 | Q-1 | PGME (500) | 100 | 33 | 3.2 |
| 15 | (80) | (27.3) | (4.0) | EL (2000) | | | |
| Example | P-1 | PAG-16 | Q-1 | PGME (500) | 100 | 31 | 3.2 |
| 16 | (80) | (27.9) | (4.0) | EL (2000) | | | |
| Example | P-1 | PAG-17 | Q-1 | PGME (500) | 100 | 33 | 3.3 |
| 17 | (80) | (33.1) | (4.0) | EL (2000) | | | |
| Example | P-1 | PAG-18 | Q-1 | PGME (500) | 100 | 34 | 3.4 |
| 18 | (80) | (25.0) | (4.0) | EL (2000) | | | |
| Example | P-1 | PAG-19 | Q-1 | PGMEA (500) | 100 | 33 | 3.1 |
| 19 | (80) | (25.3) | (4.0) | EL (2000) | | | |
| Example | P-1 | PAG-20 | Q-1 | PGMEA (500) | 100 | 34 | 3.4 |
| 20 | (80) | (31.2) | (4.0) | EL (2000) | | | |
| Example | P-1 | PAG-21 | Q-1 | PGMEA (500) | 100 | 35 | 3.3 |
| 21 | (80) | (37.0) | (4.0) | EL (2000) | | | |
| Example | P-2 | PAG-1 | Q-2 | PGMEA (500) | 100 | 34 | 3.4 |
| 22 | (80) | (30.3) | (7.4) | EL (2000) | | | |
| Example | P-3 | PAG-1 | Q-2 | PGMEA (500) | 100 | 30 | 3.5 |
| 23 | (80) | (30.3) | (7.4) | EL (2000) | | | |
| Example | P-4 | PAG-1 | Q-1 | PGMEA (500) | 100 | 34 | 3.5 |
| 24 | (80) | (30.3) | (4.0) | EL (2000) | | | |

TABLE 2

| | | Acid | | Organic | | | |
|---|---|---|---|---|---|---|---|
| | Polymer | generator | Quencher | solvent | | | |
| | (parts by | (parts by | (parts by | (parts by | PEB | Sensitivity | CDU |
| | mass) | mass) | mass) | mass) | (°C) | (mJ/cm²) | (nm) |
| Comparative | P-1 | cPAG-1 | Q-1 | PGMEA (2000) | 100 | 37 | 4.1 |
| Example 1 | (80) | (24.7) | (4.0) | DAA (500) | | | |
| Comparative | P-1 | cPAG-2 | Q-1 | PGMEA (500) | 100 | 40 | 4.8 |
| Example 2 | (80) | (17.6) | (4.0) | EL (2000) | | | |
| Comparative | P-1 | cPAG-3 | Q-1 | PGMEA (500) | 100 | 54 | 4.4 |
| Example 3 | (80) | (22.9) | (4.0) | EL (2000) | | | |
| Comparative | P-4 | cPAG-4 | Q-1 | PGMEA (2000) | 100 | 38 | 3.9 |
| Example 4 | (80) | (30.6) | (4.0) | DAA (500) | | | |
| Comparative | P-4 | cPAG-5 | Q-1 | PGMEA (2000) | 100 | 38 | 5.0 |
| Example 5 | (80) | (23.5) | (4.0) | DAA (500) | | | |
| Comparative | P-4 | cPAG-6 | Q-1 | PGMEA (2000) | 100 | 45 | 5.0 |
| Example 6 | (80) | (25.3) | (4.0) | DAA (500) | | | |
| Comparative | P-4 | cPAG-7 | Q-1 | PGMEA (2000) | 100 | 37 | 4.1 |
| Example 7 | (80) | (19.8) | (4.0) | DAA (500) | | | |
| Comparative | P-4 | cPAG-8 | Q-1 | PGMEA (2000) | 100 | 38 | 4.3 |
| Example 8 | (80) | (19.8) | (4.0) | DAA (500) | | | |

From the results shown in Tables 1 and 2, it was found that the inventive onium salt had high sensitivity and favorable CDU. On the other hand, it was not possible to achieve favorable lithography properties in Comparative Examples 1 to 8, where used were: cPAG-1 to -3, -5, and -6, in which the anion moiety was not an iodobenzenesulfonic acid; cPAG-4, in which the anion moiety was a benzenesulfonic acid having multiple substituents which were iodine atoms but both of the carbon atoms in the ortho positions of the sulfonate anion were bonded to iodine atoms; and cPAG-7 and -8, in which the benzenesulfonic acid of the anion moiety had only one iodine atom.

### (3) EUV Lithography Evaluation (2)

Each of the chemically-amplified resist compositions shown in Tables 3 and 4 was respectively applied by spin-coating on a Si substrate on which a silicon-containing spin-on hard mask SHB-A940, manufactured by Shin-Etsu Chemical Co., Ltd. (silicon content of 43 mass %), was formed with 20 nm in film thickness. Then, the substrate was prebaked at 100° C. for 60 seconds using a hot plate to produce each resist film having a film thickness of 50 nm. Each resist film was exposed using an EUV scanner NXE3300 (NA 0.33, σ 0.9/0.6, dipole illumination), manufactured by ASML Holding N.V. The exposure was performed with an LS pattern with 18 nm on wafer size and 36 nm in pitch, and with changing an exposure dose and focus (exposure dose pitch: 1 mJ/cm², focus pitch: 0.020 μm). After the exposure, PEB was performed at a temperature shown in Tables 3 and 4 for 60 seconds. Thereafter, puddle development with a 2.38 mass % aqueous TMAH solution for 30 seconds, rinse with a surfactant-containing rinse material, and spin-drying were performed to obtain a positive pattern. The obtained LS pattern was observed with a length-measurement SEM (CG6300), manufactured by Hitachi High-Technologies Corporation, to evaluate sensitivity, LWR, and collapse limit in accordance with the following methods. The results are shown in Tables 3 and 4.

### [Sensitivity Evaluation]

An optimum exposure dose Eop (mJ/cm²) to yield the LS pattern with 18 nm in line width and 36 nm in pitch was determined to specify this value as sensitivity. The smaller this value, the higher the sensitivity.

### [LWR Evaluation]

In the LS pattern obtained by irradiation at Eop, sizes in ten positions in the longitudinal direction of the line were measured. From the results, a tripled value (3σ) of a standard variation (σ) was determined as LWR. A smaller LWR value can yield a pattern with smaller roughness and uniform line width.

### [Collapse Limit Evaluation of Line Pattern]

The line size of the LS pattern for each exposure dose at the optimum focus was measured in ten positions in the longitudinal direction. The thinnest line size obtained without collapse was obtained as the collapse limit. The smaller the collapse limit value, the better the collapse limit.

TABLE 3

| | | Acid | | Organic | | | | |
|---|---|---|---|---|---|---|---|---|
| | Polymer | generator | Quencher | solvent | | | | Collapse |
| | (parts by | (parts by | (parts by | (parts by | PEB | Sensitivity | LWR | limit |
| | mass) | mass) | mass) | mass) | (°C) | (mJ/cm²) | (nm) | (nm) |
| Example | P-1 | PAG-1 | Q-1 | PGMEA (2000) | 100 | 34 | 3.1 | 10.8 |
| 1 | (80) | (30.4) | (4.0) | DAA (500) | | | | |
| Example | P-1 | PAG-2 | Q-1 | PGMEA (500) | 100 | 35 | 3.3 | 10.6 |
| 2 | (80) | (21.7) | (4.0) | EL (2000) | | | | |
| Example | P-1 | PAG-3 | Q-1 | PGMEA (500) | 100 | 39 | 3.2 | 11.5 |
| 3 | (80) | (26.8) | (4.0) | EL (2000) | | | | |
| Example | P-1 | PAG-4 | Q-1 | PGMEA (500) | 100 | 36 | 3.2 | 10.5 |
| 4 | (80) | (24.3) | (4.0) | EL (2000) | | | | |
| Example | P-1 | PAG-5 | Q-1 | PGMEA (500) | 100 | 35 | 3.3 | 10.4 |
| 5 | (80) | (27.1) | (4.0) | EL (2000) | | | | |
| Example | P-1 | PAG-6 | Q-1 | PGMEA (500) | 100 | 36 | 3.2 | 10.3 |
| 6 | (80) | (24.7) | (4.0) | EL (2000) | | | | |
| Example | P-1 | PAG-7 | Q-1 | PGMEA (500) | 100 | 39 | 3.2 | 11.2 |
| 7 | (80) | (26.7) | (4.0) | EL (2000) | | | | |
| Example | P-1 | PAG-8 | Q-1 | PGMEA (500) | 100 | 35 | 3.2 | 10.2 |
| 8 | (80) | (24.2) | (4.0) | EL (2000) | | | | |
| Example | P-1 | PAG-9 | Q-1 | PGMEA (500) | 100 | 34 | 3.2 | 10.5 |
| 9 | (80) | (25.7) | (4.0) | EL (2000) | | | | |
| Example | P-1 | PAG-10 | Q-1 | PGMEA (500) | 100 | 35 | 3.3 | 10.6 |
| 10 | (80) | (22.3) | (4.0) | EL (2000) | | | | |
| Example | P-1 | PAG-11 | Q-1 | PGMEA (500) | 100 | 36 | 3.3 | 11.0 |
| 11 | (80) | (27.2) | (4.0) | EL (2000) | | | | |
| Example | P-1 | PAG-12 | Q-1 | PGMEA (500) | 100 | 38 | 3.4 | 10.8 |
| 12 | (80) | (28.2) | (4.0) | EL (2000) | | | | |
| Example | P-1 | PAG-13 | Q-1 | PGME (500) | 100 | 35 | 3.2 | 10.7 |
| 13 | (80) | (32.6) | (4.0) | EL (2000) | | | | |
| Example | P-1 | PAG-14 | Q-1 | PGME (500) | 100 | 34 | 3.2 | 10.6 |
| 14 | (80) | (27.3) | (4.0) | EL (2000) | | | | |
| Example | P-1 | PAG-15 | Q-1 | PGME (500) | 100 | 34 | 3.2 | 10.5 |
| 15 | (80) | (27.3) | (4.0) | EL (2000) | | | | |
| Example | P-1 | PAG-16 | Q-1 | PGME (500) | 100 | 36 | 3.2 | 10.4 |
| 16 | (80) | (27.9) | (4.0) | EL (2000) | | | | |
| Example | P-1 | PAG-17 | Q-1 | PGME (500) | 100 | 35 | 3.3 | 10.8 |
| 17 | (80) | (33.1) | (4.0) | EL (2000) | | | | |
| Example | P-1 | PAG-18 | Q-1 | PGME (500) | 100 | 36 | 3.4 | 10.4 |
| 18 | (80) | (25.0) | (4.0) | EL (2000) | | | | |
| Example | P-1 | PAG-19 | Q-1 | PGMEA (500) | 100 | 34 | 3.1 | 10.5 |
| 19 | (80) | (25.3) | (4.0) | EL (2000) | | | | |
| Example | P-1 | PAG-20 | Q-1 | PGMEA (500) | 100 | 35 | 3.4 | 10.6 |
| 20 | (80) | (31.2) | (4.0) | EL (2000) | | | | |
| Example | P-1 | PAG-21 | Q-1 | PGMEA (500) | 100 | 36 | 3.3 | 10.6 |
| 21 | (80) | (37.0) | (4.0) | EL (2000) | | | | |
| Example | P-2 | PAG-1 | Q-2 | PGMEA (500) | 100 | 35 | 3.4 | 10.5 |
| 22 | (80) | (30.3) | (7.4) | EL (2000) | | | | |
| Example | P-3 | PAG-1 | Q-2 | PGMEA (500) | 100 | 36 | 3.5 | 10.4 |
| 23 | (80) | (30.3) | (7.4) | EL (2000) | | | | |
| Example | P-4 | PAG-1 | Q-1 | PGMEA (500) | 100 | 35 | 3.5 | 10.8 |
| 24 | (80) | (30.3) | (4.0) | EL (2000) | | | | |

TABLE 4

| | | Acid | | Organic | | | | |
|---|---|---|---|---|---|---|---|---|
| | Polymer | generator | Quencher | solvent | | | | Collapse |
| | (parts | (parts by | (parts by | (parts by | PEB | Sensitivity | LWR | limit |
| | by mass) | mass) | mass) | mass) | (°C) | (mJ/cm²) | (nm) | (nm) |
| Comparative | P-1 | cPAG-1 | Q-1 | PGMEA (2000) | 100 | 45 | 3.7 | 11.7 |
| Example 1 | (80) | (24.7) | (4.0) | DAA (500) | | | | |
| Comparative | P-1 | cPAG-2 | Q-1 | PGMEA (500) | 100 | 40 | 4.5 | 11.7 |
| Example 2 | (80) | (17.6) | (4.0) | EL (2000) | | | | |
| Comparative | P-1 | cPAG-3 | Q-1 | PGMEA (500) | 100 | 51 | 4.3 | 11.9 |
| Example 3 | (80) | (22.9) | (4.0) | EL (2000) | | | | |
| Comparative | P-4 | cPAG-4 | Q-1 | PGMEA (2000) | 100 | 40 | 3.9 | 13.4 |
| Example 4 | (80) | (30.6) | (4.0) | DAA (500) | | | | |
| Comparative | P-4 | cPAG-5 | Q-1 | PGMEA (2000) | 100 | 41 | 4.4 | 13.5 |
| Example 5 | (80) | (23.5) | (4.0) | DAA (500) | | | | |
| Comparative | P-4 | cPAG-6 | Q-1 | PGMEA (2000) | 100 | 45 | 4.5 | 13.7 |
| Example 6 | (80) | (25.3) | (4.0) | DAA (500) | | | | |
| Comparative | P-4 | cPAG-7 | Q-1 | PGMEA (2000) | 100 | 42 | 4.0 | 11.6 |
| Example 7 | (80) | (19.8) | (4.0) | DAA (500) | | | | |
| Comparative | P-4 | cPAG-8 | Q-1 | PGMEA (2000) | 100 | 44 | 4.4 | 12.5 |
| Example 8 | (80) | (19.8) | (4.0) | DAA (500) | | | | |

From the results shown in Tables 3 and 4, it was found that the chemically-amplified resist compositions containing a photo-acid generator derived from the inventive onium salt had favorable sensitivity and excellent LWR. Furthermore, the collapse limit was small, and it was observed that the compositions were resistant to pattern collapse even in fine pattern formation. Thus, it was shown that the inventive chemically-amplified resist composition is suitable as a material for EUV lithography. On the other hand, it was not possible to achieve favorable lithography properties in Comparative Examples 1 to 8, where used were: cPAG-1 to -3, -5, and -6, in which the anion moiety was not an iodobenzenesulfonic acid; cPAG-4, in which the anion moiety was a benzenesulfonic acid having multiple substituents which were iodine atoms but both of the carbon atoms in the ortho positions of the sulfonate anion were bonded to iodine atoms; and cPAG-7 and -8, in which the benzenesulfonic acid of the anion moiety had only one iodine atom.

The present description includes the following inventions.
[1]: An onium salt represented by the following formula (A),

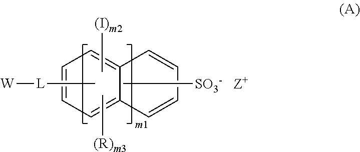

wherein “m1” represents an integer of 0 or 1, “m2” represents an integer of 2 or 3 when “m1” is 0 and represents an integer of 2, 3, 4, or 5 when “m1” is 1, and “m3” represents an integer of 0, 1, or 2 when “m1” is 0 and represents an integer of 0, 1, 2, 3, or 4 when “m1” is 1, provided that when “m1” is 0, 2≤m2+m3≤4 and when “m1” is 1, 2≤m2+m3≤6;
R represents a halogen atom other than an iodine atom, a nitro group, a hydroxy group, a cyano group, a hydrocarbyl group having 1 to 20 carbon atoms and optionally containing a heteroatom, a hydrocarbylcarbonyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, when “m3” is 2 or more, the Rs being identical to or different from each other and the Rs optionally being bonded to each other to form a ring together with the carbon atoms bonded thereto, provided that not both of the two carbon atoms adjacent to the carbon atom having a substituent which is the sulfonic acid “SO₃⁻” are bonded to iodine atoms;
L represents a single bond, an ester group, an ether group, an amide group, a sulfonic acid ester group, or a linear, branched, or cyclic alkylene group having 1 to 12 carbon atoms, the alkylene group optionally containing one or more kinds selected from an ester group, an ether group, an amide group, and a sulfonic acid ester group;
W represents a hydrocarbyl group having 6 to 60 carbon atoms and containing at least one aromatic ring and optionally contains a heteroatom, the aromatic ring optionally having a substituent which is a halogen atom, a nitro group, a hydroxy group, a cyano group, a pentafluorosulfanyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom; and
Z⁺ represents a sulfonium cation represented by the following formula (Z-1) or an iodonium cation represented by the following formula (Z-2),

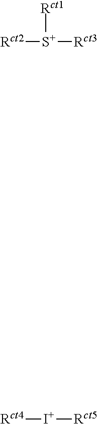

wherein R^{ct1} to R^{ct5} each independently represent a halogen atom or a hydrocarbyl group having 1 to 30 carbon atoms and optionally containing a heteroatom, the R^{ct1} and the R^{ct2} optionally being bonded to each other to form a ring together with the sulfur atom bonded thereto.
[2]: The onium salt according to [1], represented by the following formula (A1),

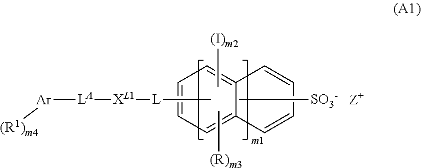

wherein “m4” represents 0 to 9; X^{L1} represents a single bond or a hydrocarbylene group having 1 to 40 carbon atoms and optionally containing a heteroatom; L^{A} represents a single bond or a linear, branched, or cyclic alkyl group having 1 to 12 carbon atoms and optionally containing one or more kinds selected from an ester group, an ether group, an amide group, and a sulfonic acid ester group; Ar represents an aromatic hydrocarbon group; R¹ represents a halogen atom, a nitro group, a hydroxy group, a cyano group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, when “m4” is 2 or more, the R¹s being identical to or different from each other and the R¹s optionally being bonded to each other to form a ring together with the carbon atoms bonded thereto; and “m1” to “m3”, L, R, and Z⁺ are as defined above.
[3]: The onium salt according to [2], represented by the following formula (A2),

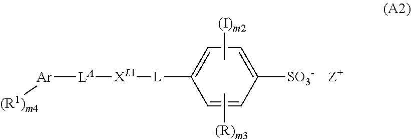

wherein “m2” to “m4”, L, L^{A}, X^{L1}, R, R¹, Ar, and Z⁺are as defined above.
[4]: The onium salt according to [2] or [3], represented by the following formula (A3),

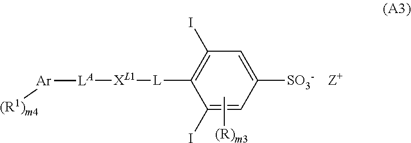

wherein “m3”, “m4”, L, L^{A}, X^{L1}, R, R¹, Ar, and Z⁺ are as defined above.
[5]: A photo-acid generator comprising the onium salt according to any one of [1] to [4].
[6]: A chemically-amplified resist composition comprising the photo-acid generator according to [5].
[7]: The chemically-amplified resist composition according to [6], further comprising a base polymer.
[8]: The chemically-amplified resist composition according to [7], wherein the base polymer includes a repeating unit represented by the following formula (a1) or (a2),

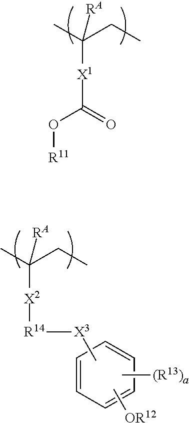

wherein each R^{A} independently represents a hydrogen atom or a methyl group;
X¹ represents a single bond, a phenylene group, a naphthylene group, or a linking group having 1 to 12 carbon atoms and containing at least one kind selected from an ester bond, an ether bond, and a lactone ring, the phenylene group, the naphthylene group, and the linking group optionally containing at least one selected from a hydroxy group, a halogen atom, a saturated hydrocarbyloxy group having 1 to 8 carbon atoms, and a saturated hydrocarbylcarbonyloxy group having 2 to 8 carbon atoms;
X² represents a single bond or an ester bond;
X³ represents a single bond, an ether bond, or an ester bond;
R¹¹ and R¹² each independently represent an acid-labile group;
R¹³ represents a saturated hydrocarbyl group having 1 to 4 carbon atoms, a halogen atom, a saturated hydrocarbylcarbonyl group having 2 to 5 carbon atoms, a cyano group, or a saturated hydrocarbyloxycarbonyl group having 2 to 5 carbon atoms;
R¹⁴ represents a single bond or an alkanediyl group having 1 to 6 carbon atoms, the alkanediyl group optionally containing at least one selected from a hydroxy group, a saturated hydrocarbyloxy group having 1 to 8 carbon atoms, a saturated hydrocarbylcarbonyloxy group having 2 to 8 carbon atoms, an ether bond, and an ester bond; and
“a” represents an integer of 0 to 4.
[9]: The chemically-amplified resist composition according to any one of [6] to [8], further comprising at least one kind selected from an organic solvent, a quencher, a surfactant, and a dissolution inhibitor.
[10]: A patterning process comprising the steps of:
   forming a resist film on a substrate by using the chemically-amplified resist composition according to any one of [6] to [9];
   exposing the resist film by using a high-energy beam; and
   developing the exposed resist film by using a developer.
[11]: The patterning process according to [10], wherein the high-energy beam is a KrF excimer laser beam, an ArF excimer laser beam, an electron beam, or an extreme ultraviolet ray having a wavelength of 3 to 15 nm.

It should be noted that the present invention is not limited to the above-described embodiments. The embodiments are just examples, and any examples that have substantially the same feature and demonstrate the same functions and effects as those in the technical concept disclosed in claims of the present invention are included in the technical scope of the present invention.

1. An onium salt represented by the following formula (A), 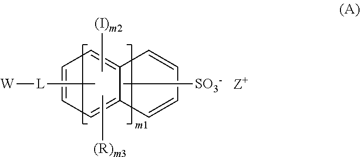
wherein “m1” represents an integer of 0 or 1, “m2” represents an integer of 2 or 3 when “m1” is 0 and represents an integer of 2, 3, 4, or 5 when “m1” is 1, and “m3” represents an integer of 0, 1, or 2 when “m1” is 0 and represents an integer of 0, 1, 2, 3, or 4 when “m1” is 1, provided that when “m1” is 0, 2≤m2+m3≤4 and when “m1” is 1, 2≤m2+m3≤6;
R represents a halogen atom other than an iodine atom, a nitro group, a hydroxy group, a cyano group, a hydrocarbyl group having 1 to 20 carbon atoms and optionally containing a heteroatom, a hydrocarbylcarbonyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, when “m3” is 2 or more, the Rs being identical to or different from each other and the Rs optionally being bonded to each other to form a ring together with the carbon atoms bonded thereto, provided that not both of the two carbon atoms adjacent to the carbon atom having a substituent which is the sulfonic acid “SO₃⁻” are bonded to iodine atoms;
L represents a single bond, an ester group, an ether group, an amide group, a sulfonic acid ester group, or a linear, branched, or cyclic alkylene group having 1 to 12 carbon atoms, the alkylene group optionally containing one or more kinds selected from an ester group, an ether group, an amide group, and a sulfonic acid ester group;
W represents a hydrocarbyl group having 6 to 60 carbon atoms and containing at least one aromatic ring and optionally contains a heteroatom, the aromatic ring optionally having a substituent which is a halogen atom, a nitro group, a hydroxy group, a cyano group, a pentafluorosulfanyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom; and
Z⁺ represents a sulfonium cation represented by the following formula (Z-1) or an iodonium cation represented by the following formula (Z-2), 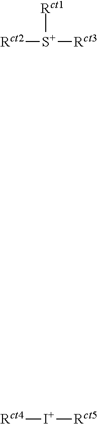
wherein R^{ct1} to R^{ct5} each independently represent a halogen atom or a hydrocarbyl group having 1 to 30 carbon atoms and optionally containing a heteroatom, the R^{ct1} and the R^{ct2} optionally being bonded to each other to form a ring together with the sulfur atom bonded thereto.

2. The onium salt according to claim 1, represented by the following formula (A1), 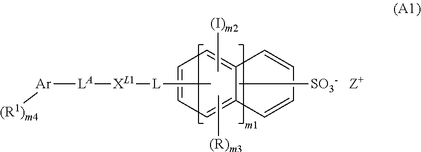
wherein “m4” represents 0 to 9; X^{L1} represents a single bond or a hydrocarbylene group having 1 to 40 carbon atoms and optionally containing a heteroatom; L^{A} represents a single bond or a linear, branched, or cyclic alkyl group having 1 to 12 carbon atoms and optionally containing one or more kinds selected from an ester group, an ether group, an amide group, and a sulfonic acid ester group; Ar represents an aromatic hydrocarbon group; R¹ represents a halogen atom, a nitro group, a hydroxy group, a cyano group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, when “m4” is 2 or more, the R's being identical to or different from each other and the R¹s optionally being bonded to each other to form a ring together with the carbon atoms bonded thereto; and “m1” to “m3”, L, R, and Z⁺ are as defined above.

3. The onium salt according to claim 2, represented by the following formula (A2), 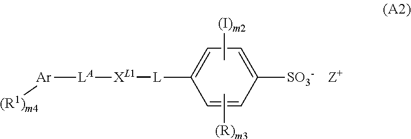
wherein “m2” to “m4”, L, L^{A}, X^{L1}, R, R¹, Ar, and Z⁺ are as defined above.

4. The onium salt according to claim 3, represented by the following formula (A3), 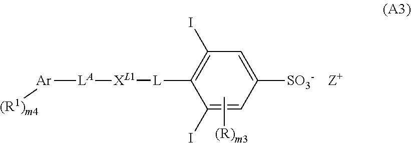
wherein “m3”, “m4”, L, L^{A}, X^{L1}, R, R¹, Ar, and Z⁺ are as defined above.

5. A photo-acid generator comprising the onium salt according to claim 1.

6. A photo-acid generator comprising the onium salt according to claim 2.

7. A photo-acid generator comprising the onium salt according to claim 3.

8. A photo-acid generator comprising the onium salt according to claim 4.

9. A chemically-amplified resist composition comprising the photo-acid generator according to claim 5.

10. A chemically-amplified resist composition comprising the photo-acid generator according to claim 6.

11. A chemically-amplified resist composition comprising the photo-acid generator according to claim 7.

12. A chemically-amplified resist composition comprising the photo-acid generator according to claim 8.

13. The chemically-amplified resist composition according to claim 9, further comprising a base polymer.

14. The chemically-amplified resist composition according to claim 10, further comprising a base polymer.

15. The chemically-amplified resist composition according to claim 11, further comprising a base polymer.

16. The chemically-amplified resist composition according to claim 12, further comprising a base polymer.

17. The chemically-amplified resist composition according to claim 13, wherein the base polymer includes a repeating unit represented by the following formula (a1) or (a2), 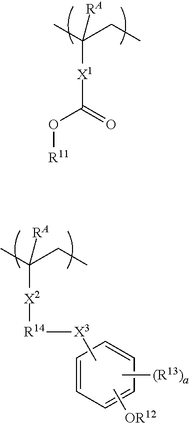
wherein each R^{A} independently represents a hydrogen atom or a methyl group;
X¹ represents a single bond, a phenylene group, a naphthylene group, or a linking group having 1 to 12 carbon atoms and containing at least one kind selected from an ester bond, an ether bond, and a lactone ring, the phenylene group, the naphthylene group, and the linking group optionally containing at least one selected from a hydroxy group, a halogen atom, a saturated hydrocarbyloxy group having 1 to 8 carbon atoms, and a saturated hydrocarbylcarbonyloxy group having 2 to 8 carbon atoms;
X² represents a single bond or an ester bond;
X³ represents a single bond, an ether bond, or an ester bond;
R¹¹ and R¹² each independently represent an acid-labile group;
R¹³ represents a saturated hydrocarbyl group having 1 to 4 carbon atoms, a halogen atom, a saturated hydrocarbylcarbonyl group having 2 to 5 carbon atoms, a cyano group, or a saturated hydrocarbyloxycarbonyl group having 2 to 5 carbon atoms;
R¹⁴ represents a single bond or an alkanediyl group having 1 to 6 carbon atoms, the alkanediyl group optionally containing at least one selected from a hydroxy group, a saturated hydrocarbyloxy group having 1 to 8 carbon atoms, a saturated hydrocarbylcarbonyloxy group having 2 to 8 carbon atoms, an ether bond, and an ester bond; and
“a” represents an integer of 0 to 4.

18. The chemically-amplified resist composition according to claim 9, further comprising at least one kind selected from an organic solvent, a quencher, a surfactant, and a dissolution inhibitor.

19. A patterning process comprising the steps of:
forming a resist film on a substrate by using the chemically-amplified resist composition according to claim 9;
exposing the resist film by using a high-energy beam; and
developing the exposed resist film by using a developer.

20. The patterning process according to claim 19, wherein the high-energy beam is a KrF excimer laser beam, an ArF excimer laser beam, an electron beam, or an extreme ultraviolet ray having a wavelength of 3 to 15 nm.
